(19) United States
(12) Patent Application Publication
(10) Pub. No.: US 2023/0406887 A1
(43) Pub. Date: Dec. 21, 2023

(54) ANTIGEN BINDING DOMAIN WITH REDUCED CLIPPING RATE

(71) Applicants: AMEGEN INC., Thousand Oaks, CA (US); AMGEN RESEARCH (MUNICH GMBH) GMBH, Munich (DE)

(72) Inventors: Johannes Brozy, Munich (DE); Markus Muenz, Munich (DE); Andrew Dykstra, Thousand Oaks, CA (US)

(21) Appl. No.: 18/251,133

(22) PCT Filed: Nov. 8, 2021
(86) PCT No.: PCT/EP2021/080880

§ 371 Date: Apr. 28, 2023

Related U.S. Application Data
(60) Provisional application No. 63/110,840, filed on Nov. 6, 2020.

(51) Int. Cl. C07K 14/00; A61P 35/00

(52) U.S. Cl. CPC C07K 14/001; C07K 2319/74; C07K 2319/01; A61P 35/00

(57) ABSTRACT

The invention relates to a polypeptide or polypeptide construct comprising a first target antigen binding domain, wherein said first target antigen binding domain comprises a VH and a VL variable region linked by a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20. The invention also relates to a method for improving stability of a polypeptide or polypeptide construct. Moreover, the invention relates to a polynucleotide encoding the polypeptide or polypeptide construct of the invention, a vector comprising said polynucleotide and a host cell transformed or transfected with said polynucleotide or with said vector. Moreover, the invention also provides for a process for the production of said polypeptide or polypeptide construct and a pharmaceutical composition comprising said polypeptide or polypeptide construct of the invention. Furthermore, the invention relates to medical uses of said polypeptide or polypeptide construct and kits comprising said polypeptide or polypeptide construct.

The invention relates to a polypeptide or polypeptide construct comprising a first target antigen binding domain, wherein said first target antigen binding domain comprises a VH and a VL variable region linked by a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20. The invention also relates to a method for improving stability of a polypeptide or polypeptide construct. Moreover, the invention relates to a polynucleotide encoding the polypeptide or polypeptide construct of the invention, a vector comprising said polynucleotide and a host cell transformed or transfected with said polynucleotide or with said vector. Moreover, the invention also provides for a process for the production of said polypeptide or polypeptide construct and a pharmaceutical composition comprising said polypeptide or polypeptide construct of the invention. Furthermore, the invention relates to medical uses of said polypeptide or polypeptide construct and kits comprising said polypeptide or polypeptide construct.

Clipping, also known as fragmentation, of formulated, purified biomolecules e.g. antibodies, T cell engager antibodies, etc. during liquid storage is a widely observed liability. Depending on the rate in which clipping occurs, a liquid formulation may not be feasible without compromises regarding the stability and therefore composition of the biomolecule. Such compromises are not always an option, in particular in highly regulated environments such as, e.g., in the pharmaceutical industry where it is of utmost importance to provide functional, uniform and stable medicines. Since biomolecules come in a variety of shapes, structures and functions, there is an ongoing need to improve their stability by way of decreasing the clipping rate, i.e. the rate at which clipping occurs. One way of trying to reduce clipping is, for example, the lyophilization of the biomolecule. However, a need for lyophilized formulation may significantly impact the commercial production flexibility and thus may result in higher cost of goods for manufacturing (COGM). Another option is to manipulate the biomolecule itself so that it becomes less prone to clipping and, inter alia, allow a liquid formulation of the biomolecule. A liquid formulation of biomolecules versus lyophilization eliminates, for example, the need for the error-prone reconstitution process of lyophilized material, thereby increasing safety and handling comfort. Clipping occurs in particular also in polypeptides or polypeptide constructs comprising antibody-derived binding domains, such as, e.g., scFvs. As such, there is an ongoing need to reduce the clipping rate of corresponding biomolecules.

The invention relates a polypeptide or polypeptide construct comprising a first target antigen binding domain, wherein said first target antigen binding domain comprises a VH and a VL variable region linked by a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20. Said peptide linker substitutes a S(G4S)n or (G4S)n linker linking said VH and VL variable region, wherein said substitution is preferably a conservative substitution, that is linker length remains the same in the substituted binding domain and the unmodified binding domain. Said substitution reduces the clipping rate of the antigen binding domain with the substitution as compared to said antigen binding domain without the substitution, i.e. with the S(G4S)n or (G4S)n linker. The clipping rate can be analysed by methods well known in the art, preferably reduced capillary electrophoresis (rCE-SDS) as described herein below and in the example section, to assess the amount of low molecular weight (LMW) species as a readout for the clipping rate.

The term “polypeptide construct” (alternatively referred to also as “compound” herein) refers to an antigen-binding (or epitope-binding) molecule comprising binding domains themselves comprising paratopes. In the context of the present invention, a polypeptide construct is understood as an organic polymer which comprises at least one continuous, unbranched amino acid chain that naturally is not existing, but was engineered. An example, and also preferred embodiment, of a polypeptide construct that is a single polypeptide is a BiTE® molecule that comprises a core structure comprising at least one functional target antigen binding domain together with at least one complete functional CD3 binding domain on a single polypeptide chain, wherein these domains are linked directly by flexible peptide (a “linker”) without any further inserted domain unlike, for example, Xmabs that comprise the target binder and the CD3 binder on different polypeptide chains. In the context of the present invention, such a polypeptide construct comprising more than one amino acid chain is likewise envisaged. It is preferred that the term “polypeptide” is used in connection with single chain forms of the compounds of the present invention, whereas “polypeptide construct” may preferably be more adequate to describe also polypeptides that comprise more than one polypeptide chain, for example two, three or four polypeptide chains. However, unless explicitly specified herein, both terms are used interchangeably herein. Preferably, the polypeptide or polypeptide construct of the invention is a single chain polypeptide or polypeptide construct. Additionally, the term “polypeptide construct” is also suitable to describe compounds of the invention that comprise one or more non-amino acid-based constituents. An amino acid chain of a polypeptide typically comprises at least 50 amino acids, preferably at least 100, 200, 300, 400 or 500 amino acids. It is also envisaged in the context of the present invention that an amino acid chain of a polymer is linked to an entity which is not composed of amino acids.

The polypeptides comprise structural and/or functional features based on the structure and/or function of an antibody, e.g., of a full-length immunoglobulin molecule. A polypeptide construct, hence, specifically and, preferably, selectively or immunospecifically binds to its target or antigen, more precisely to an epitope of said target or target antigen, and it comprises the heavy chain variable region (VH) and the light chain variable region (VL) naturally found in an antibody, or comprises domains derived therefrom. Accordingly, the constructs may alternatively be regarded as comprising paratope-structured and epitope-binding structures, such as those found in natural antibodies or fragments thereof. A polypeptide construct according to the invention comprises the minimum structural requirements of an antibody which allow for immunospecific target binding, i.e., a paratope that recognizes immunospecifically or immunoselectively an epitope on a target antigen unless specified differently. This minimum requirement may e.g. be defined by the presence of at least three light chain CDRs (i.e. CDR1, CDR2 and CDR3 of the VL region, also termed CDR-L1, CDRL2, and CDR-L3) and/or three heavy chain CDRs (i.e. CDR1, CDR2 and CDR3 of the VH region, also termed CDR-H1, CDR-H2 and CDR-H3), preferably of all six CDRs. A polypeptide construct may hence be characterized by the presence of three or, preferably, six CDRs in a binding domain, and the skilled person knows where (in which order) those CDRs are located within the paratopic binding structures. In accordance with the invention, and in relation to the first target antigen binding domain of the polypeptide or polypeptide construct, said paratopic binding structure is specified to be a target antigen binding domain characterized by the presence of a VH and VL region that, hence, comprise CDRs. Therefore, a polypeptide/polypeptide construct according to the invention comprises at least a paratopic binding structure being a binding domain binding selectively, immunospecifically and/or immunoselectively to a target antigen comprising VH and VL variable regions (with CDRs). Accordingly, a polypeptide/polypeptide construct according to the invention comprises a paratope selectively, immunospecifically and/or immunoselectively binding to a target antigen. The term “antigen-binding structure”, as used herein, refers to any polypeptide/polypeptide construct that comprises an antigen-binding structure or any molecule that has binding activity to a specified target antigen. Said antigen-binding structures or molecules are not limited to those derived from a living organism, and for example, they may be a polypeptide produced from an artificially designed sequence. They may also be any of a naturally occurring polypeptide, synthetic polypeptide, recombinant polypeptide, and such. As the antigen-binding structure in accordance with the present invention bind specifically to parts of an antigen, the antigen (epitope)-binding structure may also be broadly defined as “paratopic structure” herein. Accordingly, the polypeptides/polypeptide constructs according to the invention may also be defined as a domain comprising a paratope that are preferably immunospecifically or immunoselectively binding to a target antigen/target epitope; and in certain embodiments comprising at least a further paratope, preferably immunospecifically or immunoselectively, binding to a further, different or same, target antigen/target epitope. Therefore, whenever the present description refers to a domain of a construct or molecule of the present invention, the construct comprises at least one paratopic structure (or paratope) binding a target antigen, such as, preferably, CD3 and/or a tumor antigen, as specified herein, particularly according to any one of the appended claims. In certain embodiments, said construct comprises at least a further paratope/binding domain binding a further target antigen as defined herein.

The term “antibody” as used in accordance with the invention comprises full-length antibodies, also including camelid antibodies and other immunoglobulins generated by biotechnological or protein engineering methods or processes. These full-length antibodies may be for example monoclonal, recombinant, chimeric, deimmunized, humanized and human antibodies, as well as antibodies from other species such as mouse, hamster, rabbit, rat, goat, or non-human primates.

“Polypeptides/polypeptide constructs” of the present invention comprise a linker linking the VH and VL region of the binding domain, preferably resulting in a scFv, and/or, in other embodiments, comprise at least one further binding domain comprising a paratope, they do not occur naturally, and they are markedly different in their function from naturally occurring products. A polypeptide or polypeptide construct of the invention is hence an artificial “hybrid” molecule comprising an scFv and/or, in some embodiments, distinct paratopes/binding domains with different specificities and/or selectivities.

As indicated above, the polypeptides/polypeptide constructs of the invention may comprise more than one polypeptide chain, i.e. polypeptides comprising two or more polypeptide chains are also subject to the present invention, particularly polypeptides forming a three-dimensional protein-like structure that allows for the immunospecific binding to at least one target antigen. Therefore, the definition of the term “polypeptide construct” includes molecules consisting of only one polypeptide chain as well as molecules consisting of two, three, four or more polypeptide chains, which chains can be either identical (homodimers, homotrimers or homo oligomers) or different (heterodimer, heterotrimer or heterooligomer). Examples for the above identified antibodies and their fragments, variants, derivatives and constructs derived therefrom are described inter alia in Harlow and Lane, Antibodies: A laboratory manual, CSHL Press (1988); Kontermann and Dübel, Antibody Engineering, Springer, 2nd ed. 2010; and Little, Recombinant Antibodies for Immunotherapy, Cambridge University Press 2009.

“Polypeptides/polypeptide constructs” of the present invention may also comprise fragments of full-length antibodies, such as VH, VHH, VL, (s)dAb, Fv, light chain (VL-CL), Fd (VH-CH1), heavy chain, Fab, Fab′, F(ab′)2 or “rIgG” (“half antibody” consisting of a heavy chain and a light chain), whereas evidently not all of the foregoing fragments are applicable for the first target binding domain since it is defined to comprise a VH and a VL region linked by a peptide linker, but to the embodiments regarding the at least one further binding domain. Polypeptides/polypeptide constructs according to the invention may also comprise modified fragments of antibodies, also called antibody variants or antibody derivatives. Examples include, but are not limited to, scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” exemplified by a structure which is as follows: (VH-VL-CH3)2, (scFv-CH3)2, ((scFv)2-CH3+CH3), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL, that selectively and, preferably, specifically binds to an antigen or target independently of other variable regions or domains, whereas not all of the foregoing fragments are applicable for the first target antigen binding domain since it is defined to comprise a VH and a VL region, but to the embodiments regarding the at least one further binding domain. Further possible formats comprised in the polypeptides/polypeptide constructs according to the invention are cross bodies, maxi bodies, hetero Fc constructs, mono Fc constructs and scFc constructs. Examples for those formats will be described herein below.

Furthermore, the definition of the term “polypeptide construct” includes bivalent and polyvalent/multivalent polypeptides/polypeptide constructs as well as bispecific and polyspecific/multispecific polypeptides/polypeptide constructs, which selectively and, preferably, specifically bind to two, three or more antigenic structures (epitopes), through distinct binding domains. A polypeptide construct can have more binding valences than specificities, e.g. in a case where it has two binding domains for one target (CD3epsilon) and one binding domain for another target, for example those described herein below, or vice versa, in which case the polypeptide construct is trivalent and bispecific. In general, the term “bispecific” includes the meaning that a polypeptide construct binds to at least two different antigens, such as, preferably, CD3 and a further target antigen, preferably a tumor antigen, for example those specified herein below.

The terms “paratope”, “antigen-binding domain”, “epitope-binding domain”, “binding domain” or “domain which binds to . . . ” characterize, in connection with the present invention, a domain of the construct which selectively and, preferably, specifically or immunospecifically binds to/interacts with/recognizes an epitope on the target or target antigen. The terms “binding domain” or “domain which binds to . . . ” or “domain” as far as it relates to the herein described “constructs” characterizes in connection with the present invention a domain of the construct which immunospecifically binds to/interacts with/recognizes an epitope on the target or target antigen. The structure and function of the first binding domain (termed as first binding domain in the case of a polypeptide/polypeptide construct comprising a further, consequently second, third, and so on, binding domain), and preferably also the structure and/or function of any further binding domain (binding to for example to a target antigen such as a cell surface antigen, preferably a tumor antigen), is/are based on the structure and/or function of an antibody, e.g. of a full-length immunoglobulin polypeptide. The “binding domain” or “domain which binds to . . . ” may hence comprise the minimum structural requirements of an antibody which allow for immunospecific target binding. While the structural requirements of the first binding domain is specified to comprise a VH and VL region with corresponding three CDRs per region, said minimum structural requirement in any further binding domain may e.g. be defined by the presence of at least three light chain CDRs (i.e. CDR1, CDR2 and CDR3 of the VL region) and/or of three heavy chain CDRs (i.e. CDR1, CDR2 and CDR3 of the VH region), preferably of all six CDRs. A “domain which binds to” (or a “binding domain”) may typically comprise an antibody light chain variable region (VL) and an antibody heavy chain variable region (VH); however, it does not have to comprise both, but may comprise only one of VH or VL, if not defined otherwise. Fd fragments, for example, often retain some antigen-binding function of the intact antigen-binding domain. The terms “paratope”, “antigen-binding structure” and “epitope-binding structure, as used herein, refer also to a portion of an antibody (or a molecule according to the invention), which comprises a region that specifically binds and is complementary to the whole or a portion of an antigen or a part thereof, i.e. an antibody can only bind to a particular portion of the antigen. The particular portion is called “epitope”. An antigen-binding domain can be provided from one or more antibody variable domains. Preferably, the antigen-binding domains contain antibody variable region that comprising both the antibody light chain variable region (VL) and antibody heavy chain variable region (VH). Such preferable antigen-binding domains include, for example, “single-chain Fv (scFv)”, “single-chain antibody”, “Fv”, “single-chain Fv2 (scFv2)”, “Fab”, and “F (ab”)2”. In accordance with the invention, the first binding domain takes the form of a scFv.

Examples for the format of a “domain which binds to”, “domain comprising a paratope” (or “binding domain”, “antigen-binding structure”, “epitope-binding structure”) include, unless otherwise defined, but are not limited to, full-length antibodies, fragments of full-length antibodies (such as VH, VHH, VL), (s)dAb, Fv, light chain (VL-CL), Fd (VH-CH1), heavy chain, Fab, Fab′, F(ab′)2 or “r IgG” (“half antibody”)), antibody variants or derivatives such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” (selected from formats such as (VH-VL-CH3)2, (scFv-CH3)2, ((scFv)2-CH3+CH3)), ((scFv)2-CH3) or (scFv-CH3-scFv)2, multibodies such as triabodies or tetrabodies, and single domain antibodies such as nanobodies or single variable domain antibodies comprising merely one variable region, which might be VHH, VH or VL. It is understood herein, that the first binding domain defined as a scFv, so that some of the above formats can only relate to the at least further binding domain that may be comprised in the polypeptide or polypeptide construct of the invention. Further examples for the format of a “domain which binds to” (or a “binding domain”) include (1) an antibody fragment or variant comprising VL, VH, CL and CH1 (such as Fab); (2) an antibody fragment or variant comprising two linked Fab fragments (such as a F(ab′)2); (3) an antibody fragment or variant comprising VH and CH1 (such as Fd); (4) an antibody fragment or variant comprising VL and CL (such as the light chain); (5) an antibody fragment or variant comprising VL and VH (such as Fv); (5) a dAb fragment (Ward et al., (1989) Nature 341:544-546), which has a VH domain; (6) an antibody variant comprising at least three isolated CDRs of the heavy and/or the light chain; and (7) a single chain Fv (scFv). Examples for embodiments of constructs or binding domains according to the invention are e.g. described in WO 00/006605, WO 2005/040220, WO 2008/119567, WO 2010/037838, WO 2013/026837, WO 2013/026833, US 2014/0308285, US 2014/0302037, W O2014/144722, WO 2014/151910, and WO 2015/048272. In the context of the present invention, a paratope is understood as an antigen-binding site which is a part of a polypeptide as described herein and which recognizes and binds to an antigen. A paratope is typically a small region of about at least 5 amino acids. A paratope as understood herein typically comprises parts of antibody-derived heavy (VH) and light chain (VL) sequences. Each binding domain of a polypeptide according to the present invention is provided with a paratope comprising a set of 6 complementarity-determining regions (CDR loops) with three of each being comprised within the antibody-derived VH and VL sequence, respectively.

It is envisaged for the compounds, particularly for the constructs of the present invention that a) the construct is a single chain polypeptide or a single chain polypeptide construct, b) the first binding domain is in the format of an scFv, c) any further, such as a second binding and/or third domain is in the format of an scFv, d) the first and said further, such as said second and/or third domain are connected via a linker, preferably a peptide linker, such as the glycine/glutamine linker defined herein, and/or e) the construct comprises a domain providing an extended serum half-life, such as an Fc-based domain, or human serum albumin (HSA). Specific formats of corresponding compounds are described herein below. In the latter case namely regarding the HLE domain, the presence of which is a preferred embodiment, the term “polypeptide construct” makes clear that it may comprise more than a single peptide chain. A preferred Fc-based domain which extends the serum half-life (also termed “HLE” domain) comprises two polypeptide monomers, each comprising a hinge, a CH2 domain and a CH3 domain, wherein said two polypeptide monomers are fused to each other via a peptide linker; the format is in N-terminal to C-terminal order: hinge-CH2-CH3-linker-hinge-CH2-CH3.

The constructs of the present invention are preferably “in vitro generated constructs” and/or “recombinant constructs”. In the context of the present invention, the term “in vitro generated” refers to a construct according to the above definition where all or part of the binding domain or of a variable region (e.g., at least one CDR) is generated in a non-immune cell selection, e.g., in an in vitro phage display, on a protein chip or in any other method in which candidate amino acid sequences can be tested for their ability to bind to an antigen. This term thus preferably excludes sequences generated solely by genomic rearrangement in an immune cell in an animal. It is envisaged that the first and/or second domain of the construct is produced by or obtainable by phage display or library screening methods rather than by grafting CDR sequences from a pre-existing (monoclonal) antibody into a scaffold. A “recombinant construct” is a construct generated or produced using (inter alia) recombinant DNA technology or genetic engineering.

The constructs of the present invention are envisaged to be monoclonal. As used herein, polypeptides or constructs that are denominated “monoclonal” (mAb) are obtained from a population of substantially homogeneous antibodies/constructs, i.e., the individual antibodies/constructs comprised in the population are identical (in particular with respect to their amino acid sequence) except for possible naturally occurring mutations and/or post-translational modifications (e.g., isomerizations, amidations) that may be present in minor amounts. Monoclonal antibodies/constructs are highly specific, being directed against a single epitope within the antigen, in contrast to polyclonal antibody preparations which typically include different antibodies directed against different determinants (or epitopes). In addition to their specificity, monoclonal antibodies are advantageous in that they are synthesized by the hybridoma culture, hence uncontaminated by other immunoglobulins. The modifier “monoclonal” indicates the character of the antibody/construct as being obtained from a substantially homogeneous population of antibodies and is not to be construed as requiring production of the antibody by any specific method.

For the preparation of monoclonal antibodies, any technique providing antibodies produced by continuous cell line cultures can be used. For example, monoclonal antibodies to be used may be made by the hybridoma method first described by Koehler et al., Nature, 256: 495 (1975), or may be made by recombinant DNA methods (see, e.g., U.S. Pat. No. 4,816,567). Examples for further techniques to produce human monoclonal antibodies include the trioma technique, the human B-cell hybridoma technique (Kozbor, Immunology Today 4 (1983), 72) and the EBV-hybridoma technique (Cole et al., Monoclonal Antibodies and Cancer Therapy, Alan R. Liss, Inc. (1985), 77-96).

Hybridomas can then be screened using standard methods, such as enzyme-linked immunosorbent assay (ELISA) and surface plasmon resonance (BIACORE™) analysis, to identify one or more hybridomas that produce an antibody that selectively and, preferably, specifically or immunospecifically binds to a specified antigen. Any form of the relevant antigen may be used as the immunogen, e.g., recombinant antigen, naturally occurring forms, any variants or fragments thereof, as well as an antigenic peptide thereof. Surface plasmon resonance as employed in the BIAcore™ system can be used to increase the efficiency of phage antibodies/constructs which bind to an epitope of a target antigen (Schier, Human Antibodies Hybridomas 7 (1996), 97-105; Malmborg, J. Immunol. Methods 183 (1995), 7-13).

Another exemplary method of making constructs or binding domains includes screening protein expression libraries, e.g., phage display or ribosome display libraries. Phage display is described, for example, in Ladner et al., U.S. Pat. No. 5,223,409; Smith (1985) Science 228:1315-1317, Clackson et al., Nature, 352: 624-628 (1991) and Marks et al., J. Mol. Biol., 222: 581-597 (1991).

In addition to the use of display libraries, the relevant antigen can be used to immunize a non-human animal, e.g., a rodent (such as a mouse, hamster, rabbit or rat). In one embodiment, the non-human animal includes at least a part of a human immunoglobulin gene. For example, it is possible to engineer mouse strains deficient in mouse antibody production with large fragments of the human Ig (immunoglobulin) loci. Using the hybridoma technology, antigen-specific monoclonal antibodies derived from the genes with the desired specificity may be produced and selected. See, e.g., Xenomouse™, Green et al. (1994) Nature Genetics 7:13-21, US 2003-0070185, WO 96/34096, and WO 96/33735.

A monoclonal antibody can also be obtained from a non-human animal, and then modified, e.g., humanized, deimmunized, rendered chimeric etc., using recombinant DNA techniques known in the art. Examples of modified constructs or binding domains include humanized variants of non-human antibodies/constructs, “affinity matured” constructs or binding domains (see, e.g. Hawkins et al. J. Mol. Biol. 254, 889-896 (1992) and Lowman et al., Biochemistry 30, 10832-10837 (1991)) and antibody variants or mutants with altered effector function(s) (see, e.g., U.S. Pat. No. 5,648,260, Kontermann and Dubel (2010), loc. cit. and Little (2009), loc. cit.).

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody fragments, antibody variants, constructs or binding domains. Random mutations inside the CDRs are introduced using radiation, chemical mutagens or error-prone PCR. In addition, the genetic diversity can be increased by chain shuffling. Two or three rounds of mutation and selection using display methods like phage display usually results in antibodies, antibody fragments, antibody variants, constructs or binding domains with affinities in the low nanomolar range.

A preferred type of an amino acid substitutional variation of the constructs or binding domains of the invention involves substituting one or more residues within the hypervariable region of a parent antibody structure (e.g. a humanized or human antibody structure). Generally, the resulting variant(s) selected for further development will have improved biological properties relative to the parent antibody structure from which they are generated. A convenient way for generating such substitutional variants involves affinity maturation using phage display. Briefly, several sites of the hypervariable region (e. g. 6-7 sites) are mutated to generate all possible amino acid substitutions at each site. The variants thus generated are displayed in a monovalent fashion from filamentous phage particles as fusions to the gene Ill product of M13 packaged within each particle. The phage-displayed variants are then screened for their biological activity (e.g. binding affinity) as disclosed herein. To identify candidate hypervariable region sites contributing significantly to antigen binding (candidates for modification), alanine scanning mutagenesis can also be performed. Alternatively, or additionally, it may be beneficial to analyze a crystal structure of the complex between the antigen and the construct or the binding domain to identify contact points between the binding domain and its specific antigen. Such contact residues and neighbouring residues are candidates for substitution according to the techniques elaborated herein. Once such variants are generated, the panel of variants is subjected to screening as described herein and antibodies, their antigen-binding fragments, constructs or binding domains with superior properties in one or more relevant assays may be selected for further development.

The constructs and binding domains of the present invention specifically include “chimeric” versions in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments or variants of such antibodies, so long as they exhibit the desired biological activity (U.S. Pat. No. 4,816,567; Morrison et al., Proc. Natl. Acad. Sci. USA, 81: 6851-6855 (1984)). Chimeric constructs or binding domains of interest herein include “primitized” constructs comprising variable domain antigen-binding sequences derived from a non-human primate (e.g., Old World Monkey, Ape etc.) and human constant region sequences. A variety of approaches for making chimeric antibodies or constructs have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, polypeptide construct, antibody fragment, antibody variant or binding domain may also be modified by specific deletion of human T cell epitopes (a method called “deimmunization”) using methods disclosed for example in WO 98/52976 or WO 00/34317. Briefly, the heavy and light chain variable regions of an antibody, construct or binding domain can be analyzed for peptides that bind to MHC class II; these peptides represent potential T cell epitopes (as defined e.g. in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains or variable regions, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory (www2.mrc-lmb.cam.ac.uk/vbase/list2.php) provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, variants or fragments thereof, constructs and binding domains are based on immunoglobulins of mostly human sequences, which contain (a) minimal sequence(s) derived from non-human immunoglobulin. For the most part, humanized antibodies, variants or fragments thereof, constructs and binding domains are based on human immunoglobulins (recipient antibodies) in which residues from a hypervariable region or CDR are replaced by residues from a hypervariable region or CDR of a non-human species (donor antibody) such as a rodent (e.g. mouse, hamster, rat or rabbit) having the desired specificity, affinity, capacity and/or biological activity. In some instances, Fv framework region (FR) residues of the human immunoglobulin are replaced by corresponding non-human residues. Furthermore, “humanized” antibodies, variants or fragments thereof, constructs and binding domains as used herein may also comprise residues which are found neither in the recipient antibody nor the donor antibody. These modifications are made to further refine and optimize antibody performance. The humanized antibodies, variants or fragments thereof, constructs and binding domains may also comprise at least a portion of an immunoglobulin constant region (such as Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies, variants or fragments thereof, constructs and binding domains can be generated by replacing sequences of the (Fv) variable region that are not directly involved in antigen binding with equivalent sequences from human (Fv) variable regions. Exemplary methods for generating such molecules are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. These methods include isolating, manipulating, and expressing the nucleic acid sequences that encode all or part of immunoglobulin (Fv) variable regions from at least one of a heavy or light chain. Such nucleic acids may be obtained from a hybridoma producing an antibody against a predetermined target, as described above, as well as from other sources. The recombinant DNA encoding the humanized antibody, variant or fragment thereof, construct or binding domain can then be cloned into an appropriate expression vector.

Humanized antibodies, variants or fragments thereof, constructs and binding domains may also be produced using transgenic animals such as mice that express human heavy and light chain genes but are incapable of expressing the endogenous mouse immunoglobulin heavy and light chain genes. Winter describes an exemplary CDR grafting method that may be used to prepare the humanized molecules described herein (U.S. Pat. No. 5,225,539). All the CDRs of a given human sequence may be replaced with at least a portion of a non-human CDR, or only some of the CDRs may be replaced with non-human CDRs. It is only necessary to replace the number of CDRs required for binding of the humanized molecule to a predetermined antigen.

A humanized antibody, variant or fragment thereof, construct or binding domain can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

Human anti-mouse antibody (HAMA) responses have led the industry to prepare chimeric or otherwise humanized antibodies/constructs. It is however expected that certain human anti-chimeric antibody (HACA) responses will be observed, particularly in chronic or multi-dose utilizations of an antibody or construct. Thus, it would be desirable to provide constructs comprising a human binding domain against a target, to vitiate concerns and/or effects of HAMA or HACA response.

Therefore, according to one embodiment, the polypeptide construct having at least one further binding domain, said binding domain(s) are “human”. The term “human antibody”, “human construct” and “human binding domain” includes antibodies, constructs and binding domains, respectively, having antibody-derived regions such as variable and constant regions or domains which correspond substantially to human germline immunoglobulin sequences known in the art, including, for example, those described by Kabat et al. (1991) (loc. cit.). The human constructs or binding domains of the invention may include amino acid residues not encoded by human germline immunoglobulin sequences (e.g., mutations introduced by random or site-specific mutagenesis in vitro or by somatic mutation in vivo), for example in the CDRs, and particularly in CDR3. The human constructs or binding domains can have at least one, two, three, four, five, or more positions replaced with an amino acid residue that is not encoded by the human germline immunoglobulin sequence. The definition of human antibodies, constructs and binding domains as used herein also contemplates fully human antibodies, constructs and binding domains which include only non-artificially and/or genetically altered human sequences of antibodies as those can be derived by using technologies or systems such as the Xenomouse.

Polypeptides/polypeptide constructs comprising at least one human binding domain may avoid some of the problems associated with antibodies or constructs that possess non-human such as rodent (e.g. murine, rat, hamster or rabbit) variable and/or constant regions. The presence of such rodent derived proteins can lead to the rapid clearance of the antibodies or constructs or can lead to the generation of an immune response against the antibody or construct by a patient. To avoid the use of rodent-derived constructs, humanized or fully human constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or constructs derived therefrom are expected to minimize the immunogenic and allergic responses intrinsic to mouse or mouse-derivatized mAbs and thus to increase the efficacy and safety of the administered antibodies/constructs. The use of fully human antibodies or constructs can be expected to provide a substantial advantage in the treatment of chronic and recurring human diseases, such as inflammation, autoimmunity, and cancer, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization. The work of Green et al. was extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse model is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, Ser. No. 08/759,620; and U.S. Pat. Nos. 6,162,963; 6,150,584; 6,114,598; 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med. 188:483-495 (1998), EP 0 463 151 B1, WO94/02602, WO96/34096, WO98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

In some embodiments, the constructs of the invention are “isolated” or “substantially pure” constructs. “Isolated” or “substantially pure”, when used to describe the constructs disclosed herein, means a construct that has been identified, separated and/or recovered from a component of its production environment. Preferably, the construct is free or substantially free of association with all other components from its production environment. Contaminant components of its production environment, such as that resulting from recombinant transfected cells, are materials that could interfere with diagnostic or therapeutic uses for the construct, and may include enzymes, hormones, and other proteinaceous or non-proteinaceous compounds. It is understood that the isolated or substantially pure construct may constitute from 5% to 99.9% by weight of the total protein/polypeptide content in a given sample, depending on the circumstances. The desired construct may be produced at a significantly higher concentration using an inducible promoter or high expression promoter. The definition includes the production of a construct in a wide variety of organisms and/or host cells that are known in the art. In certain embodiments, the construct will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie Blue or, preferably, silver staining. Usually, however, an isolated construct will be prepared by at least one purification step.

According to one embodiment, the entire construct and/or the binding domains are in the form of one or more polypeptides or in the form of proteins. In addition to proteinaceous parts, such polypeptides or proteins may include non-proteinaceous parts (e.g. chemical linkers or chemical cross-linking agents such as glutaraldehyde).

Peptides are short chains of amino acid monomers linked by covalent peptide (amide) bonds. Hence, peptides fall under the broad chemical classes of biological oligomers and polymers. Amino acids that are part of a peptide or polypeptide chain are termed “residues” and can be consecutively numbered. All peptides except cyclic peptides have an N-terminal residue at one end and a C-terminal residue at the other end of the peptide. An oligopeptide consists of only a few amino acids (usually between two and twenty). A polypeptide is a longer, continuous, and unbranched peptide chain. Peptides are distinguished from proteins based on size, and as an arbitrary benchmark can be understood to contain approximately 50 or fewer amino acids. Proteins consist of one or more polypeptides, usually arranged in a biologically functional way. While aspects of the lab techniques applied to peptides versus polypeptides and proteins differ (e.g., the specifics of electrophoresis, chromatography, etc.), the size boundaries that distinguish peptides from polypeptides and proteins are not absolute. Therefore, in the context of the present invention, the terms “peptide”, “polypeptide” and “protein” may be used interchangeably, and the term “polypeptide” is often preferred.

Polypeptides may further form multimers such as dimers, trimers and higher oligomers, which consist of more than one polypeptide molecule, as mentioned above. Polypeptide molecules forming such dimers, trimers etc. may be identical or non-identical. The corresponding structures of higher order of such multimers are, consequently, termed homo- or heterodimers, homo- or heterotrimers etc. An example for a hereteromultimer is an antibody or immunoglobulin molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is accomplished e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

The terms “selectively” and, “preferably, selectively”, “(specifically or immunospecifically) binds to”, “(specifically or immunospecifically) recognizes”, or “(specifically or immunospecifically) reacts with” mean in accordance with this invention that a construct or a binding domain selectively interacts or (immuno-)specifically interacts with a given epitope on the target molecule (antigen), for example: CD3. This selective interaction or association occurs more frequently, more rapidly, with greater duration, with greater affinity, or with some combination of these parameters, to an epitope on the specific target (for example: CD3epsilon) than to alternative substances (non-target molecules, e.g., here CD3gamma, etc.). Because of the sequence similarity between homologous proteins in different species, a construct or a binding domain that selectively and/or immunospecifically binds to its target (such as a human target) may, however, cross-react with homologous target molecules from different species (such as, from non-human primates). The terms “selectively binds to”, “specific/immunospecific binding”, etc. can hence include the binding of a construct or binding domain to epitopes or structurally related epitopes in more than one species. In the context of the present invention, a polypeptide of the present invention binds to its respective target structure in a particular manner. Preferably, a polypeptide according to the present invention comprises one paratope per binding domain which specifically or immunospecifically binds to”, “(specifically or immunospecifically) recognizes”, or “(specifically or immunospecifically) reacts with” its respective target structure. This means in accordance with this invention that a polypeptide or a binding domain thereof interacts or (immuno-)specifically interacts with a given epitope on the target molecule (antigen), for example CD3epsilon, and in certain embodiments with a given epitope on at least one further, such as a second and/or a third target molecule. This interaction or association occurs more frequently, more rapidly, with greater duration, with greater affinity, or with some combination of these parameters, to an epitope on the specific target than to alternative substances (non-target molecules). Because of the sequence similarity between homologous proteins in different species, an antibody construct or a binding domain that immunospecifically binds to its target (such as a human target) may, however, cross-react with homologous target molecules from different species (such as, from non-human primates). The term “specific/immunospecific binding” can hence include the binding of an antibody construct or binding domain to epitopes and/or structurally related epitopes in more than one species. The term “(immuno-) selectively binds does exclude the binding to structurally related epitopes within one species.

In the context of the present invention, the term “epitope” refers to the part or region of the antigen that is selectively recognized/immunospecifically recognized by the binding structure, i.e. the paratope. An “epitope” is antigenic, and thus the term epitope is sometimes also referred to as “antigenic structure” or “antigenic determinant”. The part of the binding domain that binds to the epitope is called a paratope. Specific binding is believed to be accomplished by specific motifs in the amino acid sequence of the binding domain and the antigen. Thus, binding is achieved because of their primary, secondary and/or tertiary structure as well as the result of potential secondary modifications of said structures. The specific interaction of the paratope with its antigenic determinant may result in a simple binding of said site to the antigen. In some cases, the specific interaction may alternatively or additionally result in the initiation of a signal, e.g. due to the induction of a change of the conformation of the antigen, an oligomerization of the antigen, etc.

The epitopes of protein antigens are divided into two categories, conformational epitopes and linear epitopes, based on their structure and interaction with the paratope. A conformational epitope is composed of discontinuous sections of the antigen's amino acid sequence. These epitopes interact with the paratope based on the three-dimensional surface features and shape or tertiary structure (folding) of the antigen. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy. By contrast, linear epitopes interact with the paratope based on their primary structure. A linear epitope is formed by a continuous sequence of amino acids from the antigen and typically includes at least 3 or at least 4, and more usually, at least 5 or at least 6 or at least 7, for example, about 8 to about 10 amino acids in a unique sequence.

A method for epitope mapping for a given human target protein is described in the following: A pre-defined region (a contiguous amino acid stretch) within said given human target protein is exchanged/replaced with a corresponding region of a target protein paralogue (so long as the binding domain is not cross-reactive with the paralogue used). These human target/paralogue chimeras are expressed on the surface of host cells (such as CHO cells). Binding of the antibody or construct can be tested via FACS analysis. When the binding of the antibody or construct to the chimeric molecule is entirely abolished, or when a significant binding decrease is observed, it can be concluded that the region of human target which was removed from this chimeric molecule is relevant for the immunospecific epitope-paratope recognition. Said decrease in binding is preferably at least 10%, 20%, 30%, 40%, or 50%; more preferably at least 60%, 70%, or 80%, and most preferably 90%, 95% or even 100% in comparison to the binding to human (wild-type) target, whereby binding to the human target is set to be 100%. Alternatively, the above described epitope mapping analysis can be modified by introducing one or several point mutations into the sequence of the human target. These point mutations can e.g. reflect the differences between the human target and its paralogue.

A further method to determine the contribution of a specific residue of a target antigen to the recognition by a construct or binding domain is alanine scanning (see e.g. Morrison K L & Weiss G A. Curr Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired.

The interaction between the binding domain and the epitope of the target antigen implies that a binding domain exhibits appreciable or significant affinity for the epitope/the target antigen and, generally, does not exhibit significant affinity for proteins or antigens other than the target antigen—notwithstanding the above discussed cross-reactivity with homologous targets e.g. from other species. “Significant affinity” includes binding with an affinity (dissociation constant, KD) of ≤10-6 M. Preferably, binding is considered specific when the binding affinity is ≤10-7 M, ≤10-8 M, ≤10-9 M, ≤10-10 M, or even ≤10-11 M, or ≤10-12 M. Whether a binding domain (immuno-)specifically reacts with or binds to a target can be tested readily e.g. by comparing the affinity of said binding domain to its desired target protein or antigen with the affinity of said binding domain to non-target proteins or antigens. Preferably, a construct of the invention does not significantly bind to proteins or antigens other than the target antigen—unless any further binding domain(s) directed against a further target is/are deliberately introduced into the construct of the invention, in which case the binding of that binding domain to its specific target is also provided by the present invention.

It is envisaged that the affinity of the first domain is ≤100 nM, ≤90 nM, ≤80 nM, ≤70 nM, ≤60 nM, ≤50 nM, ≤40 nM, ≤30 nM, or ≤20 nM. These values are preferably measured in a cell-based assay, such as a Scatchard assay. Other methods of determining the affinity are also well-known. These values are preferably measured in a surface plasmon resonance assay, such as a Biacore assay.

The term “does not significantly bind” and “does not selectively bind” mean that a construct or binding domain of the present invention does not bind to a protein or antigen other than said target antigen, when said protein or antigen is expressed on the surface of a cell. The construct hence shows reactivity of ≤30%, preferably ≤20%, more preferably ≤10%, particularly preferably ≤9%, ≤8%, ≤7%, ≤6%, ≤5%, ≤4%, ≤3%, ≤2%, or ≤1% with proteins or antigens other than said target antigen (when said proteins or antigens are expressed on the surface of a cell), whereby binding to said target antigen, respectively, is set to be 100%. The “reactivity” can e.g. be expressed in an affinity value (see above).

It is envisaged that the construct of the invention (and more specifically the domain comprising a paratope/binding domain that binds to said first target antigen) does not bind or does not significantly bind to target antigen paralogues. It is also envisaged that the construct does not bind or does not significantly bind to (human or macaque/cyno) target antigen paralogues on the surface of a target cell.

The peptide linker is S(G4X)n and (G4X)n, wherein X is selected from the group consisting of Q, T, N, C, G, A, V, I, L, and M, and wherein n is an integer selected from integers 1 to 20. Preferably, X is selected from amino acids with polar uncharged side chains, namely Q, T, N, and amino acids with hydrophobic side chains, namely A, V, I, L, and M. In another preferred embodiment, X is selected from Q, T and N. Integer n is preferably selected from any integer in the range of 1 to 18, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 3, 1 to 2, such as integers 1, 2, 3, 4, 5, 6, 7, 8, 9, 10, 11, 12, 13, 14, 15, 16, 17, and 18, preferably integers 1, 3 and 6. Corresponding linkers sequences are defined in SEQ ID NOs: 2 to 77. Preferred linkers of SEQ ID NOs: 15, 34, 53, and 72, as outlined also herein below.

It was observed that (G4S)n or S(G4S)n linkers (wherein n has the same definition as recited herein above) used in the scFv context as a standard peptide linker in the art, namely linking the VH and VL regions, are prone to clipping and therefore compromise the stability of these molecules or molecules comprising corresponding scFvs. Substituting said G4S linker, i.e., said (G4S)n or S(G4S)n linker(s), with a linker as defined herein, preferably a S(G4Q)n or (G4Q)n linker, contributes to a decrease in the clipping rate (see example section, for example, example 2, FIG. 5, Table 1). In larger molecules comprising more than a VH and VL region linked by a linker, also the other linkers, such as for example those linking binding domains, e.g. in a diabody or (scFv)2, such as a BiTE® molecule, can be exchanged and lead to a further reduction of the clipping rate as shown in the example section. More specifically, molecule assessment data for BiTE® molecules has shown that following incubation for four weeks at 40° C. (simulating two years of liquid storage at 4° C.), the percentage of low molecular weight (LMW) species measured by reduced capillary electrophoresis (rCE-SDS) (as a preferred means for assessing the clipping rate) ranged from 16.6% to 24.1% for two exemplary BiTE® molecules. Depending on the resulting LMW fragments, the pharmacokinetics of BiTE molecules such as the observed half-life in vivo or the efficacy and safety of the BiTE molecules can be impacted potentially reducing the utility for patients. To better understand factors that contribute to the observed levels of clipping, exemplary BiTE molecules (targeted towards PSMA and CD33), respectively, were generated with the following modifications: G4Q linker (vs. G4S linker in standard BiTE HLE molecules), stabilized CD3 binding domain (vs. standard CD3 binding domain), introduction of an engineered Cys clamp (vs. no Cys clamp) within the CD3 binding domain, and/or removal of certain single chain Fc (scFc) D-P and hinge sites (vs. standard scFc). All variants were tested for their in vitro activity compared to reference controls to determine impacts of the sequence variants on the potency. Additionally, BiTE molecule variants were incubated for four weeks at 40° C. and the total percentage of LMW species were monitored with rCE-SDS. Peptide mapping was used to monitor site-specific clipping. It could be shown that the introduced modifications significantly decreased overall clipping (introduction of G4Q linkers decreased the percentage of LMW by 7.1%). Combinations of these modifications showed that overall clipping in BiTE molecules after thermal degradation can be significantly decreased. In addition, the stabilized CD3 binding domain, insertion of G4Q linkers, and CD3 Cys clamp modifications all contribute to the reduction of clipping in the linker domains. Accordingly, it could be demonstrated that the linkers as defined herein, as well as the further modifications reduce the appearance of LMWs due to clipping, i.e. the clipping rate is decreased. As such, a liquid formulation of the polypeptides or polypeptide constructs of the invention is a valid option due to a reduction in the clipping rate.

Herein below, it is further outlined which linkers should have the form of the linkers as defined herein to improve stability of a given polypeptide or polypeptide by decreasing the clipping rate, including not only scFv binding domains but also half-life extending domains that can be part of the polypeptide or polypeptide construct of the invention. As such, and in other words, the invention relates to a single chain polypeptide or polypeptide construct comprising a first target antigen binding domain, wherein said first target antigen binding domain comprises a VH and a VL variable region linked by a peptide linker, wherein the peptide linker comprises or consists of S(G4X)n and (G4X)n, wherein X is selected from the group consisting of Q, T, N, C, G, A, V, I, L, and M, wherein n is an integer selected from integers 1 to 20, and wherein said linker replaces a S(G4S)n and (G4S)n linker.

In a preferred embodiment of the polypeptide or polypeptide construct of the invention, integer n is 1, 2, 3, 4, 5 or 6. The integer n is preferably be 2, 3, 4, 5 or 6, or, more preferred 1, 3 or 6.

In accordance with the invention, the X in S(G4X)n or (G4X)n is preferably Q. As such, the peptide linker is S(G4Q)n or (G4Q)n. Throughout the invention as described herein, the amino acid Q is the preferred amino acid for X. For example, the linker can be S(G4Q), S(G4Q)3, S(G4Q)6 or (G4Q), (G4Q)3 or (G4Q)6.

In a preferred embodiment, the peptide linker of polypeptide or polypeptide construct of the invention is S(G4X)n or (G4X)n, n is 3, and X is Q. Hence, the peptide linker has the format of (G4Q)3 (SEQ ID NO: 15) or S(G4Q)3.

In accordance with the invention, the polypeptide or polypeptide construct of the invention may comprise at least one further binding domain binding to a target antigen. As outlined herein above, the polypeptide or polypeptide construct of the invention may comprise at least one further target antigen binding domain and is thus at least a bispecific molecule. According to one embodiment, the polypeptide construct of the invention is, a “single chain construct” or “single chain polypeptide”. In the case of a further binding domain, it is also envisaged that either the first binding or the further (also termed “second”) or both binding domains may be in the format of a “single chain Fv” (scFv). Although the two domains of the Fv fragment, VL and VH, are coded for by separate genes, they can be joined, using recombinant methods, by an artificial linker—as described hereinbefore—that enables them to be made as a single protein chain in which the VL and VH regions pair to form a monovalent molecule; see e.g., Huston et al. (1988) Proc. Natl. Acad. Sci USA 85:5879-5883). These antibody fragments are obtained using conventional techniques known to those with skill in the art, and the fragments are evaluated for function in the same manner as are full-length antibodies or IgGs. A single-chain variable fragment (scFv) is hence a fusion protein of the variable region of the heavy chain (VH) and of the light chain (VL) of immunoglobulins, usually and in accordance with the invention connected with a short linker peptide. The linker is usually rich in glycine for flexibility, as well as serine or also threonine for solubility, and can either connect the N-terminus of the VH with the C-terminus of the VL, or vice versa. This protein retains the specificity of the original immunoglobulin, despite removal of the constant regions and introduction of the linker.

Bispecific single chain molecules are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for producing single chain constructs (see, inter alia, U.S. Pat. No. 4,946,778, Kontermann and Dübel (2010), loc. cit. and Little (2009), loc. cit.) can be adapted to produce single chain constructs selectively and, preferably, specifically recognizing (an) elected target(s).

Bivalent (also called divalent) or bispecific single-chain variable fragments (bi-scFvs or di-scFvs) having the format (scFv)2 can be engineered by linking two scFv molecules (with linkers as described herein). The linking can be done by producing a single polypeptide chain with two VH regions and two VL regions, yielding tandem scFvs (see e.g. Kufer, P. et al., (2004) Trends in Biotechnology 22(5):238-244). Another possibility is the creation of scFv molecules with linker peptides that are too short for the two variable regions to fold together (e.g. about five amino acids; less than 12 amino acids), forcing the scFvs to dimerize. In this case, the VH and the VL of a binding domain (binding either to the first or the further target antigen) are not directly connected via a peptide linker. Thus, the VH of the first target antigen binding domain may e.g. be fused to the VL of the further target antigen binding domain via a peptide linker as defined herein, and the VH of the further target antigen binding domain is fused to the VL of the first binding domain via such peptide linker. This type is known as diabodies (see e.g. Hollinger, Philipp et al., (July 1993) Proceedings of the National Academy of Sciences of the United States of America 90 (14): 6444-8.). It is understood herein, that in such a scenario, the first target antigen binding domain of the main embodiment only comprises one half of a binding domain for said first target antigen, e.g. the VH region, while the rest of the said binding domain comprises half of the binding domain for the second target antigen, e.g. the VL region, which is in line with the invention to relate to polypeptide or polypeptide constructs comprising at least two binding domains, wherein said binding domains are as defined herein, namely having a VH and a VL region, and comprise at least one a peptide linker linking a VH and a VL region as defined herein, namely the peptide linker comprises or consists of S(G4X)n or (G4X)n, wherein X is selected from the group consisting of Q, T, N, C, G, A, V, I, L, and M, and wherein n is an integer selected from integers 1 to 20.

In accordance with the invention, the at least one further target antigen binding domain comprises the same components as the first target antigen binding domain. In accordance with this embodiment, the polypeptide or polypeptide construct comprises two binding domains that each have the format of VH/VL-Peptide Linker-VH/VL. As outlined herein above, corresponding polypeptides or polypeptide constructs include, for example, diabodies and (scFv)2 molecules. It is understood herein that the designation VH/VL-Peptide Linker-VH/VL means that both configurations are encompassed. Namely, in amino to carboxyl order, the format VH-Peptide Linker-VL, and VL-Peptide Linker-VH is encompassed by the invention.

In accordance with the invention, each target antigen binding domain binds to one target antigen. In this embodiment, each binding domain comprises all components allowing the binding to only one target antigen, hence, each binding domain comprises a VH and a VL region. In other terms, the VH and VL variable region of one target antigen binding domain binds to a target, whereas the VH and VL variable region of said at least one further target antigen binding domain binds to a target. Accordingly, this embodiment does not extend to a bispecific diabody where two scFvs dimerize to form two binding domains resulting from said dimerization of two polypeptide chains that are as defined in the main embodiment. Instead, it is preferred that the polypeptide or polypeptide construct of the invention is a (scFv)2. The following embodiments concern different formats of scFv-based polypeptide or polypeptide constructs in accordance with the invention. In all of these embodiments and in accordance with the invention, at least one binding domain is characterized by the presence of the linker as defined herein, namely said peptide linker that comprises or consists of S(G4X)n or (G4X)n, wherein X is selected from the group consisting of Q, T, N, C, G, A, V, I, L, and M, and wherein n is an integer selected from integers 1 to 20, and its preferred embodiments as laid out herein above. As also laid out herein above, said S(G4X)n or (G4X)n substitute S(G4S)n or (G4S)n in said different formats of scFv-based polypeptide or polypeptide constructs described in the following. Moreover, it is preferred that more than two, three, or, more preferred, all binding domains comprised in the polypeptide or polypeptide construct of the invention comprise said linker. As outlined to herein above, a corresponding polypeptide or polypeptide construct of the invention exhibits a reduced clipping rate in comparison to a corresponding, i.e. the same, polypeptide or polypeptide construct with the state of the art serine/glycine linkers.

In a preferred embodiment, the polypeptide or polypeptide construct of the invention comprises: Binding Domain 1 (VH/VL-Peptide Linker-VH/VL)-Linker-Binding Domain 2 (VH/VL-Peptide Linker-VH/VL). This embodiment concerns a single chain polypeptide comprising two binding domains, wherein one or both peptide linkers are as defined herein above, namely said peptide linker comprises or consists of S(G4X)n or (G4X)n, wherein X is selected from the group consisting of Q, T, N, C, G, A, V, I, L, and M, and wherein n is an integer selected from integers 1 to 20. Also outlined herein above, preferably, the peptide linker is (G4Q)3, preferably both peptide linkers within the binding domain are (G4Q)3. In other terms, the Binding Domain 1 and/or the Binding Domain 2 comprise a VH and a VL variable region linked by a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20, i.e. the binding domain as defined herein above. As is understood herein, the feature in brackets, i.e. (VH/VL Peptide Linker-VH/VL) defines the structure of the binding domain(s).

Preferably, the first binding domain but also said at least one further binding domain binds to a cell surface antigen as target antigen. The term “cell surface antigen” as used herein denotes a molecule, which is displayed on the surface of a cell. In most cases, this molecule will be located in or on the plasma membrane of the cell such that at least part of this molecule remains accessible from outside the cell in tertiary form. A non-limiting example of a cell surface molecule, which is located in the plasma membrane is a transmembrane protein comprising, in its tertiary conformation, regions of hydrophilicity and hydrophobicity. Here, at least one hydrophobic region allows the cell surface molecule to be embedded, or inserted in the hydrophobic plasma membrane of the cell while the hydrophilic regions extend on either side of the plasma membrane into the cytoplasm and extracellular space, respectively. Non-limiting examples of cell surface molecules which are located on the plasma membrane are proteins which have been modified at a cysteine residue to bear a palmitoyl group, proteins modified at a C-terminal cysteine residue to bear a farnesyl group or proteins which have been modified at the C-terminus to bear a glycosyl phosphatidyl inositol (“GPI”) anchor.

According to one embodiment of the binding domains comprising the herein described paratopes, the VH-region is positioned N-terminally of the linker, and the VL-region is positioned C-terminally of the linker. In other words, in one embodiment of the binding domains comprising the herein described paratopes, the scFv comprises from the N-terminus to the C-terminus: VH-linker-VL. In accordance with the invention, the binding domains comprising the herein described paratopes of the construct are connected via a peptide linker as defined herein according to the invention. The construct may e.g. comprise the domains in the order (from N-terminus to C-terminus) one binding domain-linker-further binding domain. The inverse order (further binding domain-linker-first binding domain) is also possible.

In accordance with the invention, said polypeptide or polypeptide or polypeptide construct comprises: Binding Domain 1 (VH/VL-Peptide Linker-VH/VL)-Linker-Binding Domain 2 (VH/VL-Peptide Linker-VH/VL)-Binding Domain 3 (VH/VL-Peptide Linker-VH/VL).

In a preferred embodiment of the polypeptide or polypeptide construct of the invention, the C-terminal binding domain is binding to CD3, and wherein said remaining N-terminal binding domain(s) is/are binding to a cell surface antigen. Preferably, the polypeptide or polypeptide construct of the invention is a T cell engager. Accordingly, it is preferred that the polypeptide or polypeptide construct of the invention comprises at least a CD3 binding domain and a cell surface antigen (which is, preferably, a tumor antigen) binding domain as defined herein below. Hence, in accordance with the invention, said polypeptide or polypeptide or polypeptide construct comprises (preferably in amino to carboxyl order): Binding Domain 1 (tumor antigen binding domain: VH/VL-Peptide Linker-VH/VL)-Linker-Binding Domain 2 (CD3 binding domain: VH/VL-Peptide Linker-VH/VL), wherein binding domain 1 binds to a cell surface antigen, preferably a tumor antigen, and binding domain 2 binds to CD3, preferably human CD3, more preferred human CD3epsilon; or Binding Domain 1 (VH/VL-Peptide Linker-VH/VL)-Linker-Binding Domain 2 (VH/VL-Peptide Linker-VH/VL)-Binding Domain 3 (CD3 binding domain: VH/VL-Peptide Linker-VH/VL); wherein binding domain 1 and 2 bind to the same or different cell surface antigens, preferably the same or different tumor antigens, and binding domain 3 binds to CD3, preferably human CD3, more preferred human CD3epsilon. Preferably, the Binding Domain 2 is linked to Binding Domain 3 via a linker as defined herein taking the form of: Binding Domain 1 (VH/VL-Peptide Linker-VH/VL)-Linker-Binding Domain 2 (VH/VL-Peptide Linker-VH/VL)-Linker-Binding Domain 3 (VH/VL-Peptide Linker-VH/VL).

As laid out herein above, the polypeptide construct of the invention preferably comprises a binding domain which binds to CD3 on the surface of a T cell. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to for the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide which in humans is encoded by the CD3 epsilon gene which resides on chromosome 11. In the context of the present invention, CD3 is understood as a protein complex and T cell co-receptor that is involved in activating both the cytotoxic T cell (CD8+naive T cells) and T helper cells (CD4+ naive T cells). It is typically composed of four distinct chains. Especially in mammals, the complex contains a CD3γ chain, a CD3δ chain, and two CD3ε chains. These chains associate with the T-cell receptor (TCR) and the ζ-chain (zeta-chain) to generate an activation signal in T lymphocytes. The TCR, ζ-chain, and CD3 molecules together constitute the TCR complex.

The redirected lysis of target cells via the recruitment of T cells by a construct which binds to CD3 on the T cell and to a target protein on the target cell generally involves cytolytic synapse formation and delivery of perforin and granzymes. The engaged T cells are capable of serial target cell lysis and are not affected by immune escape mechanisms interfering with peptide antigen processing and presentation, or clonal T cell differentiation; see e.g. WO 2007/042261.

Cytotoxicity mediated by given tumor antigen×CD3 constructs can be measured in various ways. The “half maximal effective concentration” (EC50) is commonly used as a measure of potency of a biologically active molecule such as a construct of the present invention. It may be expressed in molar units. In the present case of measuring cytotoxicity, the EC50 value refers to the concentration of a construct inducing a cytotoxic response (lysis of target cells) halfway between the baseline and the maximum. Effector cells in a cytotoxicity assay can e.g. be stimulated enriched (human) CD8 positive T cells or unstimulated (human) peripheral blood mononuclear cells (PBMC). An EC50 value may typically be expected to be lower when stimulated/enriched CD8+ T cells are used as effector cells, compared with unstimulated PBMC. If the target cells are of macaque origin or express or are transfected with a given macaque tumor antigen, the effector cells should also be of macaque origin, such as a macaque T cell line, e.g. 4119LnPx. The target cells should express said tumor antigen on the cell surface. Target cells can be a cell line (such as CHO) which is stably or transiently transfected with said tumor antigen. Alternatively, the target cells can be a tumor antigen positive natural expresser cell line, such as the human cancer lines. Usually EC50 values are expected to be lower when using target cells that express higher levels of said tumor antigen on the cell surface compared with target cells having a lower target expression rate.

The effector to target cell (E:T) ratio in a cytotoxicity assay is usually about 10:1, but can also vary. Cytotoxic activity of tumor antigen×CD3constructs can be measured in a 51-chromium release assay (e.g. with an incubation time of about 18 hours) or in a in a FACS-based cytotoxicity assay (e.g. with an incubation time of about 48 hours). Modifications of the incubation time (cytotoxic reaction) are also envisaged. Other methods of measuring cytotoxicity are well-known and comprise MTT or MTS assays, ATP-based assays including bioluminescent assays, the sulforhodamine B (SRB) assay, WST assay, clonogenic assay and the ECIS technology.

According to one embodiment, the cytotoxic activity mediated by tumor antigen×CD3 constructs of the present invention is measured in a cell-based cytotoxicity assay. It may also be measured in a 51-chromium release assay. It is envisaged that the EC50 value of the constructs of the invention is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

The above given EC50 values can be measured in different assays and under different conditions. For example, when human PBMCs are used as effector cells and tumor antigen transfected cells such as CHO cells are used as target cells, it is envisaged that the EC50 value of the tumor antigen×CD3 construct is ≤500 pM, ≤400 pM, ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM. When human PBMCs are used as effector cells and when the target cells are a CLDN6 positive cell line such as, it is envisaged that the EC50 value of the CLDN6×CD3 construct is ≤300 pM, ≤280 pM, ≤260 pM, ≤250 pM, ≤240 pM, ≤220 pM, ≤200 pM, ≤180 pM, ≤160 pM, ≤150 pM, ≤140 pM, ≤120 pM, ≤100 pM, ≤90 pM, ≤80 pM, ≤70 pM, ≤60 pM, ≤50 pM, ≤40 pM, ≤30 pM, ≤20 pM, ≤15 pM, ≤10 pM, or ≤5 pM.

According to one embodiment, the tumor antigen×CD3 polypeptides/polypeptide constructs of the present invention do not induce/mediate lysis or do not essentially induce/mediate lysis of cells that do not express said given tumor antigen on their surface (tumor antigen negative cells), such as CHO cells. The term “do not induce lysis”, “do not essentially induce lysis”, “do not mediate lysis” or “do not essentially mediate lysis” means that a construct of the present invention does not induce or mediate lysis of more than 30%, preferably not more than 20%, more preferably not more than 10%, particularly preferably not more than 9%, 8%, 7%, 6% or 5% of tumor antigen negative cells, whereby lysis of tumor antigen expressing target cells (such as cells transformed or transfected with said tumor antigen or a natural expresser cell line such as the human cancer lines) is set to be 100%. This usually applies for concentrations of the construct of up to 500 nM. Cell lysis measurement is a routine technique. Moreover, the present specification teaches specific instructions how to measure cell lysis.

The difference in cytotoxic activity between the monomeric and the dimeric isoform of individual tumor antigen×CD3 polypeptides/polypeptide constructs is referred to as “potency gap”. This potency gap can e.g. be calculated as ratio between EC50 values of the molecule's monomeric and dimeric form. In one method to determine this gap, an 18 hour 51-chromium release assay or a 48 h FACS-based cytotoxicity assay is carried out as described hereinbelow with purified construct monomer and dimer. Effector cells are stimulated enriched human CD8+ T cells or unstimulated human PBMC. Target cells are hu tumor antigen transfected CHO cells. Effector to target cell (E:T) ratio is 10:1. Potency gaps of the tumor antigen×CD3 constructs of the present invention are preferably ≤5, more preferably ≤4, even more preferably ≤3, even more preferably ≤2 and most preferably ≤1.

The binding domain(s) of the polypeptide construct of the invention is/are preferably cross-species specific for members of the mammalian order of primates, such as macaques. According to one embodiment, the further binding domain(s), in addition to binding to a human tumor antigen, will also bind to said tumor antigen of primates including (but not limited to) new world primates (such as Callithrix jacchus, Saguinus oedipus or Saimiri sciureus), old world primates (such as baboons and macaques), gibbons, orangutans and non-human hominidae. It is envisaged that the domain which binds to human CD3 on the surface of a T cell of the invention also binds at least to macaque CD3. A preferred macaque is Macaca fascicularis. Macaca mulatta (Rhesus) is also envisaged. The polypeptide or polypeptide construct of the invention comprises a domain which binds to human CD3epsilon on the surface of a T cell and at least macaque CD3.

In one embodiment, the affinity gap of the constructs according to the invention for binding macaque CD3 versus human CD3 [KD ma CD3: KD hu CD3] (as determined e.g. by BiaCore or by Scatchard analysis) is between 0.01 and 100, preferably between 0.1 and 10, more preferably between 0.2 and 5, more preferably between 0.3 and 4, even more preferably between 0.5 and 3 or between 0.5 and 2.5, and most preferably between 0.5 and 1.

As detailed herein above, said binding domain of the polypeptide or polypeptide construct of the invention binds to human CD3 epsilon (or human CD3 epsilon on the surface of a T cell) and, preferably, to Callithrix jacchus or Saimiri sciureus CD3 epsilon. More specifically, said domain binds to an extracellular epitope of human CD3 epsilon. It is also envisaged that said domain binds to an extracellular epitope of the human and the Macaca CD3 epsilon chain. Said extracellular epitope of CD3 epsilon is comprised within amino acid residues 1-27 of the human CD3 epsilon extracellular domain (see SEQ ID NO: 847; amino acid residues 1-27 in SEQ ID NO: 848). Even more particularly, the epitope comprises at least the amino acid sequence Gln-Asp-Gly-Asn-Glu. Callithrix jacchus is a new world primate belonging to the family of Callitrichidae, while Saimiri sciureus is a new world primate belonging to the family of Cebidae. Binders having such characteristics are described in detail in WO 2008/119567.

Antibodies or bispecific constructs directed against (human) CD3 or selectively and, preferably, specifically against CD3 epsilon are known in the art, and their CDRs, VH and VL sequences can serve as a basis for the binding domain of the polypeptide construct of the invention. For example, Kung et al. reported in 1979 the development of OKT3 (Ortho Kung T3), the first mAb recognizing CD3 (specifically, the epsilon chain of CD3) on human T cells. OKT3 (muromonab) was the first monoclonal antibody of murine origin to become available for therapy in humans. Newer anti-CD3 monoclonal antibodies include otelixizumab (TRX4), teplizumab (MGA031), foralumab and visilizumab, all targeting the epsilon chain of CD3. Bispecific constructs directed against a (cancer) target and CD3 are also being developed and (pre-)clinically tested, and their CD3 binding domain (CDRs, VH, VL) may serve as a basis for the second binding domain of the construct of the invention. Examples include, but are not limited to, Blinatumomab, Solitomab (MT110, AMG 110), Catumaxomab, Duvortuxizumab, Ertumaxomab, Mosunetuzumab, FBTA05 (Bi20, TPBs05), CEA-TCB (RG7802, RO6958688), AFM11, and MGD006 (S80880). Other examples of CD3 binding domains are disclosed e.g. in U.S. Pat. No. 7,994,289 B2, U.S. Pat. No. 7,728,114 B2, U.S. Pat. No. 7,381,803 B1, U.S. Pat. No. 6,706,265 E1.

Preferred combinations of CDR-L1 to L3 sequences of the VL region and preferred combinations of CDR-H1 to H3 sequences of the VH region of the CD3 binding domain are listed in the below list.

| CDR-L1 | CDR-L2 | CDR-L3 |
|---|---|---|
| GSSTGAVTSGYYPN | GTKFLAP | ALWYSNRWV |
| RSSTGAVTSGYYPN | ATDMRPS | ALWYSNRWV |
| GSSTGAVTSGNYPN | GTKFLAP | VLWYSNRWV |
| ASSTGAVTSGNYPN | GTKFLVP | TLWYSNRWV |
| RSSTGAVTTSNYAN | GTNKRAP | ALWYSNLWV |
| CDR-H1 | CDR-H2 | CDR-H3 |
| IYAMN | RIRSKYNNYATYYADSVKS | HGNFGNSYVSFFAY |
| KYAMN | RIRSKYNNYATYYADSVKD | HGNFGNSYISYWAY |
| SYAMN | RIRSKYNNYATYYADSVKG | HGNFGNSYLSFWAY |
| RYAMN | RIRSKYNNYATYYADSVKG | HGNFGNSYLSYFAY |
| VYAMN | RIRSKYNNYATYYADSVKK | HGNFGNSYLSWWAY |
| KYAMN | RIRSKYNNYATYYADSVKS | HGNFGNSYTSYYAY |
| GYAMN | RIRSKYNNYATYYADSVKE | HRNFGNSYLSWFAY |
| VYAMN | RIRSKYNNYATYYADSVKK | HGNFGNSYISWWAY |
| SYAMN | RIRSKYNNYATYYADSVKG | HGNFGNSYVSWWAY |
| KYAIN | RIRSKYNNYATYYADQVKD | HANFGNSYISYWAY |
| TYAMN | RIRSKYNNYATYYADSVKD | HGNFGNSYVSWFAY |

Preferably, any of the above listed combinations of CDR-L1 to L3 combinations is combined with any of the above-listed combinations CDR-H1 to H3 as part of the binding domain binding to an extracellular of the human CD3ε chain. In other words, the VL region comprises or consists of as CDR-L1, CDR-L2, CDR-L3 sequence, in this order, GSSTGAVTSGYYPN, GTKFLAP, ALWYSNRWV; RSSTGAVTSGYYPN, ATDMRPS, ALWYSNRWV; GSSTGAVTSGNYPN, GTKFLAP, VLWYSNRWV; ASSTGAVTSGNYPN, GTKFLVP, TLWYSNRWV; or RSSTGAVTTSNYAN, GTNKRAP, ALWYSNLWV; and the VL region comprises or consists of as CDR-H1, CDR-H2, CDR-H3 sequence, in this order,

IYAMN, RIRSKYNNYATYYADSVKS, HGNFGNSYVSFFAY;
KYAMN, RIRSKYNNYATYYADSVKD, HGNFGNSYISYWAY;
SYAMN, RIRSKYNNYATYYADSVKG, HGNFGNSYLSFWAY;
RYAMN, RIRSKYNNYATYYADSVKG, HGNFGNSYLSYFAY;
VYAMN, RIRSKYNNYATYYADSVKK, HGNFGNSYLSWWAY;
KYAMN, RIRSKYNNYATYYADSVKS, HGNFGNSYTSYYAY;
GYAMN, RIRSKYNNYATYYADSVKE, HRNFGNSYLSWFAY;
VYAMN, RIRSKYNNYATYYADSVKK, HGNFGNSYISWWAY;
SYAMN, RIRSKYNNYATYYADSVKG, HGNFGNSYVSWWAY;
KYAIN, RIRSKYNNYATYYADQVKD, HANFGNSYISYWAY;
or
TYAMN, RIRSKYNNYATYYADSVKD, HGNFGNSYVSWFAY.

In accordance with the invention, preferred CDR sequence combinations of the VH and the VL regions, listed in the order CDR-H1, CDR-H2, CDR-H3, CDR-L1, CDR-L2, CDR-L3, are as defined in SEQ ID NOs: 82 to 87; 88 to 93; 94 to 99; 100 to 105; 106 to 111; 112 to 117; 118 to 123; 124 to 129; 130 to 135; 136 to 141; 142 to 147; 148 to 153; 154 to 159; 160 to 165; 166 to 171; 172 to 177; 178 to 183; 184 to 189; 190 to 195; 196 to 201; 202 to 207; 208 to 213; 214 to 219; and 220 to 225.
Most preferred is that the VL region comprises as the CDR combinations as depicted in SEQ ID NOs: 106 to 111; 112 to 117; 118 to 123; 124 to 129; 178 to 183; 184 to 189; 190 to 195; 196 to 201; 202 to 207; 208 to 213; 214 to 219; and 220 to 225; listed in the order of CDR-H1, CDR-H2, CDR-H3, CDR-L1, CDR-L2, CDR-L3 sequence. Preferably the orientation of the CDRs is VH to VL, i.e. the orientation of the variable regions is from N- to C-terminus VH to VL.
Preferred VH and VL region sequence combinations of the CD3 binding domain comprised in the polypeptide or polypeptide construct of the invention, are found in SEQ ID NOs (listed in the order VH+VL sequence): 550+551; 552+553; 554+555; 556+557; 558+559; 560+561; 562+563; 564+565; 566+567; 568+569; 570+571; 572+573; 574+575; 576+577; 578+579; 580+581; 582+583; 584+585; 586+587; 588+589; 590+591; 592+593; 594+595; 596+597. Preferred CD3 binding domains are selected from SEQ ID NOs: 558+559; 560+561; 562+563; 564+565; 582+583; 584+585; 586+587; 588+589; 590+591; 592+593; 594+595; and 596+597.

In another preferred embodiment, the linker(s) linking the binding domains the polypeptide or polypeptide construct of the invention comprise(s) or consist(s) of S(G4X)n or (G4X)n, wherein X is selected from the group consisting of Q, T, N, C, G, A, V, I, L, and M, and wherein n is an integer selected from integers 1 to 20. As outlined herein above, it is advantageous if more than one of the linkers in the polypeptides or polypeptide constructs of the invention are as defined herein above. Hence, it is preferred that also the linkers connecting binding domains are as defined herein above. It is most preferred, that the linker(s) linking the binding domains as well as the linker(s) linking the VH and VL variable regions within the binding domains are S(G4Q)n or (G4Q)n linkers as defined herein, wherein said linkers substitute linkers that contain serine in place of glutamine, i.e. S(G4S)n or (G4S)n linkers.

Preferably, said linker linking the binding domains of the polypeptide or polypeptide construct of the invention is S(G4X)n, n is 1 and X is Q. In other terms, the linker linking the, preferably all, binding domains of the polypeptide or polypeptide construct of the invention is S(G4Q) (SEQ ID NO: 34).

In a further preferred embodiment, the polypeptide or polypeptide construct of the invention comprises a half-life extending domain. It is also envisaged that the polypeptide construct of the invention has, in addition to its function to bind to the said target antigen(s) (preferably, when the polypeptide or polypeptide construct comprises a CD3 binding domain and at least one further binding domain binding to a tumor antigen), a further function. In this format, the construct may be a trifunctional or multifunctional construct by targeting target cells through tumor antigen binding, mediating cytotoxic T cell activity through CD3 binding and providing a further function such as means or domains to enhance or extend serum half-life, a fully functional or modified Fc constant domain mediating cytotoxicity through recruitment of effector cells, a label (fluorescent etc.), a therapeutic agent such as a toxin or radionuclide, etc.

Examples for means or domains to extend serum half-life of the polypeptides/polypeptide constructs of the invention include peptides, proteins or domains of proteins, which are fused or otherwise attached to the polypeptides/polypeptide constructs. The group of peptides, proteins or protein domains includes peptides binding to other proteins with preferred pharmacokinetic profile in the human body such as serum albumin (see WO 2009/127691). An alternative concept of such half-life extending peptides includes peptides binding to the neonatal Fc receptor (FcRn, see WO 2007/098420), which can also be used in the constructs of the present invention. The concept of attaching larger domains of proteins or complete proteins includes the fusion of human serum albumin, variants or mutants of human serum albumin (see WO 2011/051489, WO 2012/059486, WO 2012/150319, WO 2013/135896, WO 2014/072481, WO 2013/075066) or domains thereof, as well as the fusion of an immunoglobulin constant region (Fc domain) and variants thereof. Such variants of Fc domains are called Fc-based domains and may e.g. be optimized/modified to allow the desired pairing of dimers or multimers, to abolish Fc receptor binding (e.g. to avoid ADCC or CDC) or for other reasons. A further concept known in the art to extend the half-life of substances or molecules in the human body is the pegylation of those molecules (such as the constructs of the present invention).

In one embodiment, the polypeptides/polypeptide constructs according to the invention are linked (e.g. via peptide bond) with a fusion partner (such as a protein, polypeptide or peptide), e.g. for extending the construct's serum half-life. These fusion partners can be selected from human serum albumin (“HSA” or “HALB”) as wells as sequence variants thereof, peptides binding to HSA, peptides binding to FcRn (“FcRn BP”), or constructs comprising an (antibody derived) Fc region. In general, the fusion partners may be linked to the N-terminus or to the C-terminus of the constructs according to the invention, either directly (e.g. via peptide bond) or through a peptide linker such as (GGGGQ)n, (GGGGS)n or GGGG (wherein “n” is an integer of 2 or greater, e.g. 2 or 3 or 4). Specific suitable peptide linkers are discussed above.

It is preferred that said half-life extending domain (HLE domain) is comprising, or consisting of, two polypeptide monomers with each monomer comprising a hinge, a CH2 domain and a CH3 domain, wherein said two polypeptide monomers are fused to each other via a peptide linker, comprising in an amino to carboxyl order: hinge-CH2-CH3-peptide linker-hinge-CH2-CH3. A preferred polypeptide monomer of said HLE domain comprises or consists of the sequence of SEQ ID NO: 78 or 79; wherein the sequence of the entire HLE domain is defined in SEQ ID NO: 849. The sequence of said HLE domain monomer is, preferably, modified by deletion of the “DKTHT” sequence motif at the N-terminus and/or by substitution of the amino acid D at position 55 of SEQ ID NO: 78 or 79 with amino acid E. Preferably, all modifications, i.e. the deletion of the DKTHT motif as well as said substitutions at positions 55 are present in each HLE domain monomer which are fused to each other via a peptide linker. It is preferred that said peptide linker is (GGGGQ)n, or (GGGGS)n (wherein “n” is an integer of 2 or greater, e.g. 2 or 3 or 4 or 5 or 6, preferred 6). A particularly preferred linker is (G4Q)6. Each of the latter features/modifications contributes to a further reduction of the clipping rate. Therefore, the combination of (G4Q)6 as linker fusing said HLE domain monomers modified by deletion of the “DKTHT” sequence motif at the N-terminus and by substitution of the amino acid D at position 55 of SEQ ID NO: 78 or 79 with amino acid E is a preferred embodiment of the invention (as in SEQ ID NOs: 80 and 81). A corresponding preferred HLE domain comprises or consists of the sequence defined in SEQ ID NO: 850.

In accordance with the invention, said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-HLE domain. The HLE domain is preferably linked to the polypeptide or polypeptide construct according to the invention through a peptide linker such as (GGGGQ)n, (GGGGS)n or GGGG (wherein “n” is an integer of 2 or greater, e.g. 2 or 3 or 4). More preferred, the linker is GGGG.

Also in accordance with the invention, said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-Linker-Binding Domain 3 (VH/VL-Peptide Linker-VH/VL)-HLE domain.

Further in accordance with the invention, said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-Linker-HLE domain-Linker-Binding Domain 3 (VH/VL-Peptide Linker-VH/VL)-Linker-Binding Domain 4 (VH/VL-Peptide Linker-VH/VL), wherein Binding Domain 1 binds to a first cell surface antigen, Binding domains 2 and 3 bind to CD3, wherein Binding Domain 4 binds to a second cell surface antigen. It is further preferred that the peptide linkers within the binding domains is (G4Q)3 and the peptide linker within the HLE domain is (G4Q)6, the linker linking said binding domains is S(G4Q), and wherein the linkers linking the HLE domain to the binding domains are G4 linkers. It is even further preferred that the HLE domain comprises or consist of the sequence of SEQ ID NO: 850. It is further preferred that the binding domains binding to CD3 comprise or consist of the VH and VL sequence as defined in SEQ ID NOs: 582 and 583, 584 and 585, 586 and 587, or 588 and 589.

In accordance with the invention, the linkers linking the HLE domain to the binding domains are G4 linkers in the polypeptide or polypeptide construct of the invention.

In accordance with the foregoing, it is thus envisaged that a polypeptide or polypeptide construct according to the present invention comprises a single chain polypeptide that is at least bispecific with at least one binding domain binding to CD3 and at least one binding domain binding to a cell surface antigen, preferably a tumor antigen, optionally with an HLE domain, wherein said polypeptide comprises or consists of in the following order from N-terminus to C-terminus:
a) VL (comprising part of a cell surface antigen binding domain/paratope)-(G4Q)3-VH (comprising part of a cell surface antigen binding domain/paratope)-Peptide linker (SG4Q)-VH (comprising part of the CD3epsilon binding domain/paratope)-(G4Q)3-VL (comprising part of the CD3epsilon binding domain/paratope);
b) VH (comprising part of a cell surface antigen binding domain/paratope)-(G4Q)3-VL (comprising part of a cell surface antigen binding domain/paratope)-Peptide linker (SG4Q)-VH (comprising part of the CD3epsilon binding domain/paratope)-(G4Q)3-VL (comprising part of the CD3epsilon binding domain/paratope);
c) VL (comprising part of a cell surface antigen binding domain/paratope)-(G4Q)3-VH (comprising part of a cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the preferred HLE domain described herein above)-(G4Q)6-Fc monomer (complementary part of the preferred HLE domain described herein above);
d) VH (comprising part of a cell surface antigen binding domain/paratope)-(G4Q)3-VL (comprising part of a cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the HLE domain)-(G4Q)6-Fc monomer (part of the HLE domain);
e) VH (comprising part of a first cell surface antigen binding domain/paratope)-((G4Q)3-VL (comprising part of a first cell surface antigen binding domain/paratope)-Peptide linker (SG4Q)-VL (comprising part of a second cell surface antigen binding domain/paratope)-(G4Q)3-VH (comprising part of a second cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the HLE domain)-(G4Q)6 or -Fc monomer (part of the HLE domain);
f) VH (comprising part of a first cell surface antigen binding domain/paratope)-(G4Q)3-VL (comprising part of a first cell surface antigen binding domain/paratope)-Peptide linker (SG4Q)-VH (comprising part of a second cell surface antigen binding domain/paratope)-(G4Q)3-VL (comprising part of a second cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the HLE domain)-(G4Q)6 or -Fc monomer (part of the HLE domain);
g) VL (comprising part of a first cell surface antigen binding domain/paratope)-(G4Q)3-VH (comprising part of a first cell surface antigen binding domain/paratope)-Peptide linker (SG4Q)-VL (comprising part of a second cell surface antigen binding domain/paratope)-(G4Q)3-VH (comprising part of a second cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the HLE domain)-(G4Q)6 or -Fc monomer (part of the HLE domain)
h) VL (comprising part of a first cell surface antigen binding domain/paratope)-(G4Q)3-VH (comprising part of a first cell surface antigen binding domain/paratope)-Peptide linker (SG4Q)-VH (comprising part of a second cell surface antigen binding domain/paratope)-(G4Q)3-VL (comprising part of a second cell surface antigen binding domain/paratope)-Peptide linker (SG4Q)-VH (comprising part of the CD3epsilon binding domain/paratope)-(G4Q)3-VL (comprising part of the CD3epsilon binding domain/paratope)-Peptide linker (G4)-Fc monomer (part of the HLE domain)-(G4Q)6 or -Fc monomer (part of the HLE domain); or
i) Binding domain 1 ((VL (comprising part of a first cell surface antigen binding domain/paratope)-(G4Q)3-VH (comprising part of a first cell surface antigen binding domain/paratope)) or (VH (comprising part of a first cell surface antigen binding domain/paratope)-(G4Q)3-VL (comprising part of a first cell surface antigen binding domain/paratope)))-Peptide linker (G4Q)-CD3 binding domain 1 (VH (comprising part of a first CD3epsilon binding domain/paratope)-(G4Q)3-VL (comprising part of a first CD3epsilon binding domain/paratope))-Peptide linker (G4)-Fc monomer (part of the HLE domain)-(G4Q)6-Fc monomer (part of the HLE domain)-Peptide linker (G4)-Binding domain 2 ((VL (comprising part of a second cell surface antigen binding domain/paratope)-(G4Q)3-VH (comprising part of a first cell surface antigen binding domain/paratope)) or (VH (comprising part of a second cell surface antigen binding domain/paratope)-(G4Q)3-VL (comprising part of a second cell surface antigen binding domain/paratope)))-Peptide linker (G4Q)-CD3 binding domain 2 (VH (comprising part of a second CD3epsilon binding domain/paratope)-(G4Q)3-VL (comprising part of a second CD3epsilon binding domain/paratope)).

As is evident from the above, the VH and VL region sequence orientation of the binding domain(s) of the cell surface antigen can be VH-VL or VL-VH. Preferably, the cell surface antigen is a tumor antigen as detailed herein below. The HLE domain sequences made up of the Fc monomers and connecting linkers as detailed herein are preferably selected from the sequences as defined in SEQ ID NOs: 80, 81, 72, respectively, wherein a preferred HLE domain sequence is as defined in SEQ ID NO: 850. The two CD3 binding domains of the polypeptide construct of item i) are preferably the same CD3 binding domains, such as, preferably, the CD3 binding domain with VH and VL region sequences of SEQ ID NOs: 582+583; 584+585; 586+587; and 588+589; preferably the CD3 binding domain as defined in SEQ ID NOs: 722 to 725, wherein 724 and 725 are even more preferred since they have a (G4Q)3 linker linking the VH and the VL region. While peptide linkers (SG4Q) are preferred at the indicated positions, they can also be replaced by (G4Q) linkers or SG4S linkers.

In a further preferred embodiment, where the VL region is preceded by the VH region in the cell surface antigen, preferably tumor antigen binding domain in a polypeptide or polypeptide construct of the invention, it is preferred that the amino acids “EI” are present prior to the VL region and before the linker linking the VH to the VL region as a further means to decrease the clipping rate of the polypeptide or polypeptide construct of the invention. The sequence listing comprises corresponding binding domains alone or as part of longer polypeptides or polypeptide constructs of the invention.

Covalent modifications of the polypeptides/polypeptide constructs are also included within the scope of this invention, and are generally, but not always, done post-translationally. For example, several types of covalent modifications of the construct are introduced into the molecule by reacting specific amino acid residues of the construct with an organic derivatizing agent that can react with selected side chains or with the N- or C-terminal residues. Derivatization with bifunctional agents is useful for crosslinking the constructs of the present invention to a water-insoluble support matrix or surface for use in a variety of methods. Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention. Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the constructs included within the scope of this invention comprises altering the glycosylation pattern of the protein. As is known in the art, glycosylation patterns can depend on both the sequence of the protein (e.g., the presence or absence of specific glycosylation amino acid residues, discussed below), or the host cell or organism in which the protein is produced. Specific expression systems are discussed below. Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of a construct may be altered through changes at the DNA level, particularly by mutating the DNA encoding the polypeptide at preselected bases such that codons are generated that will translate into the desired amino acids.

Another means of increasing the number of carbohydrate moieties on the construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved using a variety of endo- and exo-glycosidases as described by Thotakura et al., 1987, Meth. Enzymol. 138:350. Glycosylation at potential glycosylation sites may be prevented using the compound tunicamycin as described by Duskin et al., 1982, J. Biol. Chem. 257:3105. Tunicamycin blocks the formation of protein-N-glycoside linkages.

Other modifications of the construct are also contemplated herein. For example, another type of covalent modification of the construct comprises linking the construct to various non-proteinaceous polymers, including polyols, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the construct, e.g. to facilitate the addition of polymers such as polyethylene glycol (PEG).

In some embodiments, the covalent modification of the constructs of the invention comprises the addition of one or more labels. The labelling group may be coupled to the construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides (e.g., ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzymatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sites for secondary antibodies, metal binding domains, epitope tags, etc.).

By “fluorescent label” is meant any molecule that may be detected via its inherent fluorescent properties. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade BlueJ, Texas Red, IAEDANS, EDANS, BODIPY FL, LC Red 640, Cy 5, Cy 5.5, LC Red 705, Oregon green, the Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes, Eugene, OR), FITC, Rhodamine, and Texas Red (Pierce, Rockford, IL), Cy5, Cy5.5, Cy7 (Amersham Life Science, Pittsburgh, PA). Suitable optical dyes, including fluorophores, are described in Molecular Probes Handbook by Richard P. Haugland.

Suitable proteinaceous fluorescent labels also include, but are not limited to, green fluorescent protein, including a Renilla, Ptilosarcus, or Aequorea species of GFP (Chalfie et al., 1994, Science 263:802-805), EGFP (Clontech Laboratories, Inc., Genbank® Accession Number U55762), blue fluorescent protein (BFP, Quantum Biotechnologies, Inc. 1801 de Maisonneuve Blvd. West, 8th Floor, Montreal, Quebec, Canada H3H 1J9; Stauber, 1998, Biotechniques 24:462-471; Heim et al., 1996, Curr. Biol. 6:178-182), enhanced yellow fluorescent protein (EYFP, Clontech Laboratories, Inc.), luciferase (Ichiki et al., 1993, J. Immunol. 150:5408-5417), β galactosidase (Nolan et al., 1988, Proc. Natl. Acad. Sci. U.S.A. 85:2603-2607) and Renilla (WO92/15673, WO95/07463, WO98/14605, WO98/26277, WO99/49019, U.S. Pat. Nos. 5,292,658; 5,418,155; 5,683,888; 5,741,668; 5,777,079; 5,804,387; 5,874,304; 5,876,995; 5,925,558).

Leucine zipper domains are peptides that promote oligomerization of the proteins in which they are found. Leucine zippers were originally identified in several DNA-binding proteins (Landschulz et al., 1988, Science 240:1759), and have since been found in a variety of different proteins. Among the known leucine zippers are naturally occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-78.

The polypeptide construct of the invention may also comprise additional domains, which are e.g. helpful in the isolation of the molecule or relate to an adapted pharmacokinetic profile of the molecule. Domains helpful for the isolation of a construct may be selected from peptide motives or secondarily introduced moieties, which can be captured in an isolation method, e.g. an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g. StrepII-tag) and His-tag. All herein disclosed constructs characterized by the identified CDRs may comprise a His-tag domain, which is generally known as a repeat of consecutive His residues in the amino acid sequence of a molecule, e.g. of five His residues, or of six His residues (hexa-histidine). The His-tag may be located e.g. at the N- or C-terminus of the construct. In one embodiment, a hexa-histidine tag (HHHHHH) is linked via peptide bond to the C-terminus of the construct according to the invention. A histidine tag is preferred, especially a 6×His tag.

In accordance with the invention, it is preferred when the polypeptide or polypeptide construct of the invention comprises at further binding domain, i.e. is at least bispecific, that said cell surface antigen to which the target antigen binding domain binds is a tumor antigen. Preferably, the polypeptide or polypeptide construct of the invention is a T cell engager. Accordingly, it is preferred that the polypeptide or polypeptide construct of the invention comprises at least a CD3 binding domain and a tumor antigen binding domain.

In a preferred embodiment, the tumor antigen is selected from the group consisting of BCMA (B-cell maturation antigen), CD123 (interleukin-3 receptor alpha chain (IL-3R)), CD19 (B-lymphocyte antigen CD19), CD20 (B-lymphocyte antigen CD20), CD22 (cluster of differentiation-22), CD33 (Siglec-3), CD70 (Cluster of Differentiation 70), CDH19 (Cadherin 19), CDH3 (Cadherin 3), CLL1 (C-type lectin domain family 12 member A), CS1 (CCND3 subset 1), CLDN6 (Claudin-6), CLDN18.2 (Claudin 18.2), DLL3 (Delta-like ligand 3), EGFRvIII (Epidermal growth factor receptor vIII), FLT3 (fms like tyrosine kinase 3), MAGEB2 (Melanoma-associated antigen B2), MART1 (Melanoma Antigen Recognized By T-Cells 1), MSLN (Mesothelin), MUC17 (Mucin-17), PSMA (prostate-specific membrane antigen), and STEAP1 (Metalloreductase STEAP1). These tumor antigens are well known in the art due to their expression on tumor cells.

Preferred CDR sequences and VH/VL region sequences and combinations thereof for BCMA binding domains as binding domains of the polypeptide or polypeptide construct of the invention, and bispecific single chain molecule sequences of a polypeptide or polypeptide construct in accordance with the invention having a BCMA binding domain as binding domain are defined in SEQ ID NOs: 238 to 243; 244 to 249; 602+603; 604+605; 732; 733; 784; 794.

Preferred CDR sequences and VH/VL region sequences and combinations thereof for CD123 binding domains as binding domains of the polypeptide or polypeptide construct of the invention, and bispecific single chain molecule sequences of a polypeptide or polypeptide construct in accordance with the invention having a CD123 binding domain as binding domain are defined in SEQ ID NOs: 250 to 255; 256 to 261; 608+609; 735.

Preferred CDR sequences and VH/VL region sequences and combinations thereof for CD19 binding domains as binding domains of the polypeptide or polypeptide construct of the invention, and bispecific single chain molecule sequences of a polypeptide or polypeptide construct in accordance with the invention having a CD19 binding domain as binding domain are defined in SEQ ID NOs:268 to 273; 612+613; 737; 797.

Preferred CDR sequences and VH/VL region sequences and combinations thereof for CD33 binding domains as binding domains of the polypeptide or polypeptide construct of the invention, and bispecific single chain molecule sequences of a polypeptide or polypeptide construct in accordance with the invention having a CD33 binding domain as binding domain are defined in SEQ ID NOs: 286 to 291; 298 to 303; 304 to 309; 618+619; 622+623; 624+625; 740; 742; 743; 786; 799.

Preferred CDR sequences and VH/VL region sequences and combinations thereof for CD70 binding domains as binding domains of the polypeptide or polypeptide construct of the invention, and bispecific single chain molecule sequences of a polypeptide or polypeptide construct in accordance with the invention having a CD70 binding domain as binding domain are defined in SEQ ID NOs: 316 to 321; 628+629; 745; 801.

Preferred CDR sequences and VH/VL region sequences and combinations thereof for CDH19 binding domains as binding domains of the polypeptide or polypeptide construct of the invention, and bispecific single chain molecule sequences of a polypeptide or polypeptide construct in accordance with the invention having a CDH19 binding domain as binding domain are defined in SEQ ID NOs: 328 to 333; 632+633; 747; 803.

Preferred CDR sequences and VH/VL region sequences and combinations thereof for CDH3 binding domains as binding domains of the polypeptide or polypeptide construct of the invention, and bispecific single chain molecule sequences of a polypeptide or polypeptide construct in accordance with the invention having a CDH3 binding domain as binding domain are defined in SEQ ID NOs: 340 to 345; 346 to 351; 358 to 363; 642+643; 749; 750; 752; 805; 844; 846.

Preferred CDR sequences and VH/VL region sequences and combinations thereof for CLDN18.2 binding domains as binding domains of the polypeptide or polypeptide construct of the invention, and bispecific single chain molecule sequences of a polypeptide or polypeptide construct in accordance with the invention having a CLDN18.2 binding domain as binding domain are defined in SEQ ID NOs: 370 to 375; 646+647; 754; 812.

Preferred CDR sequences and VH/VL region sequences and combinations thereof for CLL1 binding domains as binding domains of the polypeptide or polypeptide construct of the invention, and bispecific single chain molecule sequences of a polypeptide or polypeptide construct in accordance with the invention having a CLL1 binding domain as binding domain are defined in SEQ ID NOs: 328 to 387; 650+651; 756; 806.

Preferred CDR sequences and VH/VL region sequences and combinations thereof for CLDN6 binding domains as binding domains of the polypeptide or polypeptide construct of the invention, and bispecific single chain molecule sequences of a polypeptide or polypeptide construct in accordance with the invention having a CLDN6 binding domain as binding domain are defined in SEQ ID NOs: 394 to 399; 654+655; 758; 810.

Preferred CDR sequences and VH/VL region sequences and combinations thereof for CS1 binding domains as binding domains of the polypeptide or polypeptide construct of the invention, and bispecific single chain molecule sequences of a polypeptide or polypeptide construct in accordance with the invention having a CS1 binding domain as binding domain are defined in SEQ ID NOs: 412 to 417; 660+661; 761; 839; 840.

Preferred CDR sequences and VH/VL region sequences and combinations thereof for DLL3 binding domains as binding domains of the polypeptide or polypeptide construct of the invention, and bispecific single chain molecule sequences of a polypeptide or polypeptide construct in accordance with the invention having a DLL3 binding domain as binding domain are defined in SEQ ID NOs: 424 to 429; 664+665; 763; 814.

Preferred CDR sequences and VH/VL region sequences and combinations thereof for EGFRvIII binding domains as binding domains of the polypeptide or polypeptide construct of the invention, and bispecific single chain molecule sequences of a polypeptide or polypeptide construct in accordance with the invention having a EGFRvIII binding domain as binding domain are defined in SEQ ID NOs: 436 to 441; 668+669; 765; 789.

Preferred CDR sequences and VH/VL region sequences and combinations thereof for FLT3 binding domains as binding domains of the polypeptide or polypeptide construct of the invention, and bispecific single chain molecule sequences of a polypeptide or polypeptide construct in accordance with the invention having a FLT3 binding domain as binding domain are defined in SEQ ID NOs: 448 to 453; 672+673; 767; 818; 843.

Preferred CDR sequences and VH/VL region sequences and combinations thereof for MAGEB2 binding domains as binding domains of the polypeptide or polypeptide construct of the invention, and bispecific single chain molecule sequences of a polypeptide or polypeptide construct in accordance with the invention having a MAGEB2 binding domain as binding domain are defined in SEQ ID NOs: 460 to 465; 472 to 477; 676+677; 680+681; 769; 771; 823; 825.

Preferred CDR sequences and VH/VL region sequences and combinations thereof for MSLN binding domains as binding domains of the polypeptide or polypeptide construct of the invention, and bispecific single chain molecule sequences of a polypeptide or polypeptide construct in accordance with the invention having a MSLN binding domain as binding domain are defined in SEQ ID NOs: 484 to 489; 490 to 495; 502 to 507; 684+685; 686+687; 690+691; 773; 774; 776; 827.

Preferred CDR sequences and VH/VL region sequences and combinations thereof for MUC17 binding domains as binding domains of the polypeptide or polypeptide construct of the invention, and bispecific single chain molecule sequences of a polypeptide or polypeptide construct in accordance with the invention having a MUC17 binding domain as binding domain are defined in SEQ ID NOs: 514 to 519; 694+695; 778; 829.

Preferred CDR sequences and VH/VL region sequences and combinations thereof for PSMA binding domains as binding domains of the polypeptide or polypeptide construct of the invention, and bispecific single chain molecule sequences of a polypeptide or polypeptide construct in accordance with the invention having a PSMA binding domain as binding domain are defined in SEQ ID NOs: 532 to 537; 538 to 543; 544 to 549; 700+701; 702+703; 781; 782; 783; 790; 831.

The invention also relates to a method for improving stability of a polypeptide or polypeptide construct comprising a first target antigen binding domain, wherein said first target antigen binding domain comprises a VH and a VL variable region linked by a peptide linker, wherein the peptide linker comprises or consists of S(G4S)n and (G4S)n, wherein n is an integer selected from integers 1 to 20, comprising the step of substituting said S(G4S)n or (G4S)n linker with a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20. In a preferred embodiment of the method of the invention, integer n is 1, 2, 3, 4, 5 or 6. In accordance with the invention, the X in S(G4X)n or (G4X)n is preferably Q. As such, the peptide linker is S(G4Q)n or (G4Q)n. In a preferred embodiment, the peptide linker is (G4X)n, n is 3, and X is Q. Hence, the peptide linker has the format of (G4Q)3. All preferred embodiments described herein above in relation to the polypeptide or polypeptide construct of the invention also apply to the method for improving stability. As such, the method of the invention can be used to improve the stability of any of the herein recited polypeptides or polypeptide constructs, when said polypeptides or polypeptide constructs comprise S(G4S)n and (G4S)n linkers, that are then substituted with said peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20, according to the method of the invention. More concretely, the invention relates to a method for improving the stability of a polypeptide or polypeptide in the format: Binding Domain 1 (VH/VL-Peptide Linker-VH/VL)-Linker-Binding Domain 2 (VH/VL-Peptide Linker-VH/VL); Binding Domain 1 (VH/VL-Peptide Linker-VH/VL)-Linker-Binding Domain 2 (VH/VL-Peptide Linker-VH/VL)-HLE domain (in amino to carboxyl order); Binding Domain 1 (VH/VL-Peptide Linker-VH/VL)-Linker-Binding Domain 2 (VH/VL-Peptide Linker-VH/VL)-Linker-Binding Domain 3 (VH/VL-Peptide Linker-VH/VL)-HLE domain (in amino to carboxyl order); or Binding Domain 1 (VH/VL-Peptide Linker-VH/VL)-Linker-Binding Domain 2 (VH/VL-Peptide Linker-VH/VL)-Linker-HLE domain-Linker-Binding Domain 3 (VH/VL-Peptide Linker-VH/VL)-Linker-Binding Domain 4 (VH/VL-Peptide Linker-VH/VL) (in amino to carboxyl order), wherein the peptide linker linking the VH and VL region, the linker linking the binding domains or the linker within the HLE domain (said HLE domain as defined herein above) comprises or consists of S(G4S)n and (G4S)n, wherein n is an integer selected from integers 1 to 20, comprising the step of substituting said S(G4S)n or (G4S)n linker with a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20. In a preferred embodiment of the method of the invention, integer n is 1, 2, 3, 4, 5 or 6. In accordance with the invention, the X in S(G4X)n or (G4X)n is preferably Q. As such, the peptide linker is S(G4Q)n or (G4Q)n. Preferred linkers for each position of the different formats of the polypeptides or polypeptide construct formats are described herein above in relation to the polypeptide or polypeptide constructs of the invention which also apply to this embodiment.

“Improving stability” as used herein relates to a reduction in the clipping rate. Hence the method can also be termed a method for reducing the clipping rate of a polypeptide or polypeptide construct comprising a first target antigen binding domain, wherein said first target antigen binding domain comprises a VH and a VL variable region linked by a peptide linker, wherein the peptide linker comprises or consists of S(G4S)n and (G4S)n, wherein n is an integer selected from integers 1 to 20. A preferred method for determining the clipping rate is described in the examples.

The invention also relates to a polynucleotide encoding a polypeptide or polypeptide construct of the invention. Nucleic acid molecules are biopolymers composed of nucleotides. A polynucleotide is a biopolymer composed of 13 or more nucleotide monomers covalently bonded in a chain. DNA (such as cDNA) and RNA (such as mRNA) are examples of polynucleotides/nucleic acid molecules with distinct biological function. Nucleotides are organic molecules that serve as the monomers or subunits of nucleic acid molecules like DNA or RNA. The nucleic acid molecule or polynucleotide of the present invention can be double stranded or single stranded, linear or circular. It is envisaged that the nucleic acid molecule or polynucleotide is comprised in a vector. It is furthermore envisaged that such vector is comprised in a host cell. Said host cell is, e.g. after transformation or transfection with the vector or the polynucleotide/nucleic acid molecule of the invention, capable of expressing the construct. For this purpose, the polynucleotide or nucleic acid molecule is operatively linked with control sequences.

The genetic code is the set of rules by which information encoded within genetic material (nucleic acids) is translated into proteins. Biological decoding in living cells is accomplished by the ribosome which links amino acids in an order specified by mRNA, using tRNA molecules to carry amino acids and to read the mRNA three nucleotides at a time. The code defines how sequences of these nucleotide triplets, called codons, specify which amino acid will be added next during protein synthesis. With some exceptions, a three-nucleotide codon in a nucleic acid sequence specifies a single amino acid. Because the vast majority of genes are encoded with exactly the same code, this particular code is often referred to as the canonical or standard genetic code.

Degeneracy of codons is the redundancy of the genetic code, exhibited as the multiplicity of three-base pair codon combinations that specify an amino acid. Degeneracy results because there are more codons than encodable amino acids. The codons encoding one amino acid may differ in any of their three positions; however, often this difference is in the second or third position. For instance, codons GAA and GAG both specify glutamic acid and exhibit redundancy; but, neither specifies any other amino acid nor thus demonstrate ambiguity. The genetic codes of different organisms can be biased towards using one of the several codons that encode the same amino acid over the others—that is, a greater frequency of one will be found than expected by chance. For example, leucine is specified by six distinct codons, some of which are rarely used. Codon usage tables detailing genomic codon usage frequencies for most organisms are available. Recombinant gene technologies commonly take advantage of this effect by implementing a technique termed codon optimization, in which those codons are used to design a polynucleotide which are preferred by the respective host cell (such as a cell of human hamster origin, an Escherichia coli cell, or a Saccharomyces cerevisiae cell), e.g. to increase protein expression. It is hence envisaged that the polynucleotides/nucleic acid molecules of the present disclosure are codon optimized. Nevertheless, the polynucleotide/nucleic acid molecule encoding a construct of the invention may be designed using any codon that encodes the desired amino acid.

According to one embodiment, the polynucleotide/nucleic acid molecule of the present invention encoding the polypeptide construct of the invention is in the form of one single molecule or in the form of two or more separate molecules. If the construct of the present invention is a single chain construct, the polynucleotide/nucleic acid molecule encoding such construct will most likely also be in the form of one single molecule. However, it is also envisaged that different components of the polypeptide construct (such as the different domains, e.g. the paratope (antigen-binding (epitope-binding) structure)-comprising domain which binds to a cell surface antigen, the paratope (antigen-binding (epitope-binding) structure)-comprising domain which binds to CD3, and/or further domains such as antibody constant domains) are located on separate polypeptide chains, in which case the polynucleotide/nucleic acid molecule is most likely in the form of two or more separate molecules.

The same applies for the vector comprising a polynucleotide/nucleic acid molecule of the present invention. If the construct of the present invention is a single chain construct, one vector may comprise the polynucleotide which encodes the construct in one single location (as one single open reading frame, ORF). One vector may also comprise two or more polynucleotides/nucleic acid molecules at separate locations (with individual ORFs), each one of them encoding a different component of the construct of the invention. It is envisaged that the vector comprising the polynucleotide/nucleic acid molecule of the present invention is in the form of one single vector or two or more separate vectors. In one embodiment, and for the purpose of expressing the construct in a host cell, the host cell of the invention should comprise the polynucleotide/nucleic acid molecule encoding the construct or the vector comprising such polynucleotide/nucleic acid molecule in their entirety, meaning that all components of the construct—whether encoded as one single molecule or in separate molecules/locations—will assemble after translation and form together the biologically active construct of the invention.

The invention further relates to a vector comprising a polynucleotide/nucleic acid molecule of the invention. A vector is a nucleic acid molecule used as a vehicle to transfer (foreign) genetic material into a cell, usually to ensure the replication and/or expression of the genetic material. The term “vector” encompasses—but is not restricted to—plasmids, viruses, cosmids, and artificial chromosomes. Some vectors are designed specifically for cloning (cloning vectors), others for protein expression (expression vectors). So-called transcription vectors are mainly used to amplify their insert. The manipulation of DNA is normally conducted on E. coli vectors, which contain elements necessary for their maintenance in E. coli. However, vectors may also have elements that allow them to be maintained in another organism such as yeast, plant or mammalian cells, and these vectors are called shuttle vectors. Insertion of a vector into the target or host cell is usually called transformation for bacterial cells and transfection for eukaryotic cells, while insertion of a viral vector is often called transduction.

In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. While the genetic code determines the polypeptide sequence for a given coding region, other genomic regions can influence when and where these polypeptides are produced. Modern vectors may therefore encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

The term “control sequences” refers to DNA sequences necessary for the expression of an operably linked coding sequence in a specific host organism. The control sequences that are suitable for prokaryotes, for example, include a promoter, optionally an operator sequence, and a ribosome binding site. Eukaryotic cells are known to utilize promoters, polyadenylation signals, a Kozak sequence and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned to facilitate translation. Generally, “operably linked” means that the nucleotide sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells typically involves opening transient pores or “holes” in the cell membrane, to allow the uptake of material. Transfection can be carried out using biological particles (such as viral transfection, also called viral transduction), chemical-based methods (such as using calcium phosphate, lipofection, Fugene, cationic polymers, nanoparticles) or physical treatment (such as electroporation, microinjection, gene gun, cell squeezing, magnetofection, hydrostatic pressure, impalefection, sonication, optical transfection, heat shock).

The term “transformation” is used to describe non-viral transfer of nucleic acid molecules or polynucleotides (including vectors) into bacteria, and into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be achieved by artificial means. For transformation to happen, cells or bacteria must be in a state of competence, which might occur as a time-limited response to environmental conditions such as starvation and cell density and can also be artificially induced.

Moreover, the invention provides a host cell transformed or transfected with the polynucleotide/nucleic acid molecule of the invention or with the vector of the invention.

As used herein, the terms “host cell” or “recipient cell” are intended to include any individual cell or cell culture that can be or has been recipient of vectors, exogenous nucleic acid molecules and/or polynucleotides encoding the construct of the present invention; and/or recipients of the construct itself. The introduction of the respective material into the cell is carried out by way of transformation, transfection and the like (vide supra). The term “host cell” is also intended to include progeny or potential progeny of a single cell. Because certain modifications may occur in succeeding generations due to either natural, accidental, or deliberate mutation or due to environmental influences, such progeny may not, in fact, be completely identical (in morphology or in genomic or total DNA complement) to the parent cell but is still included within the scope of the term as used herein. Suitable host cells include prokaryotic or eukaryotic cells and include—but are not limited to—bacteria (such as E. coli), yeast cells, fungi cells, plant cells, and animal cells such as insect cells and mammalian cells, e.g., hamster, murine, rat, macaque or human.

In addition to prokaryotes, eukaryotic microbes such as filamentous fungi or yeast are suitable cloning or expression hosts for the construct of the invention. Saccharomyces cerevisiae, or common baker's yeast, is the most commonly used among lower eukaryotic host microorganisms. However, a number of other genera, species, and strains are commonly available and useful herein, such as Schizosaccharomyces pombe, Kluyveromyces hosts such as K. lactis, K. fragilis (ATCC 12424), K. bulgaricus (ATCC 16045), K. wickeramii (ATCC 24178), K. waltii (ATCC 56500), K. drosophilarum (ATCC 36906), K. thermotolerans, and K. marxianus; yarrowia (EP 402 226); Pichia pastoris (EP 183 070); Candida; Trichoderma reesia (EP 244 234); Neurospora crassa; Schwanniomyces such as Schwanniomyces occidentalis; and filamentous fungi such as Neurospora, Penicillium, Tolypocladium, and Aspergillus hosts such as A. nidulans and A. niger.

Suitable host cells for the expression of a glycosylated construct are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori (silkmoth) have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

Plant cell cultures of cotton, corn, potato, soybean, petunia, tomato, Arabidopsis and tobacco can also be used as hosts. Cloning and expression vectors useful in the production of proteins in plant cell culture are known to those of skill in the art. See e.g. Hiatt et al., Nature (1989) 342: 76-78, Owen et al. (1992) Bio/Technology 10: 790-794, Artsaenko et al. (1995) The Plant J 8: 745-750, and Fecker et al. (1996) Plant Mol Biol 32: 979-986.

However, interest has been greatest in vertebrate cells, and propagation of vertebrate cells in culture (cell culture) has become a routine procedure. Examples of useful mammalian host cell lines are monkey kidney CV1 line transformed by SV40 (such as COS-7, ATCC CRL 1651); human embryonic kidney line (such as 293 or 293 cells subcloned for growth in suspension culture, Graham et al., J. Gen Virol. 36: 59 (1977)); baby hamster kidney cells (such as BHK, ATCC CCL 10); Chinese hamster ovary cells/-DHFR (such as CHO, Urlaub et al., Proc. Natl. Acad. Sci. USA 77: 4216 (1980)); mouse sertoli cells (such as TM4, Mather, Biol. Reprod. 23: 243-251 (1980)); monkey kidney cells (such as CVI ATCC CCL 70); African green monkey kidney cells (such as VERO-76, ATCC CRL1587); human cervical carcinoma cells (such as HELA, ATCC CCL 2); canine kidney cells (such as MDCK, ATCC CCL 34); buffalo rat liver cells (such as BRL 3A, ATCC CRL 1442); human lung cells (such as W138, ATCC CCL 75); human liver cells (such as Hep G2,1413 8065); mouse mammary tumor (such as MMT 060562, ATCC CCL-51); TRI cells (Mather et al., Annals N. Y Acad. Sci. (1982) 383: 44-68); MRC 5 cells; FS4 cells; and a human hepatoma line (such as Hep G2).

In a further embodiment, the invention provides a process for the production of a polypeptide or polypeptide construct of the invention, said process comprising culturing a host cell of the invention under conditions allowing the expression of the construct of the invention and recovering the produced construct from the culture.

As used herein, the term “culturing” refers to the in vitro maintenance, differentiation, growth, proliferation and/or propagation of cells under suitable conditions in a medium. Cells are grown and maintained in a cell growth medium at an appropriate temperature and gas mixture. Culture conditions vary widely for each cell type. Typical growth conditions are a temperature of about 37° C., a C02 concentration of about 5% and a humidity of about 95%. Recipes for growth media can vary e.g. in pH, concentration of the carbon source (such as glucose), nature and concentration of growth factors, and the presence of other nutrients (such as amino acids or vitamins). The growth factors used to supplement media are often derived from the serum of animal blood, such as fetal bovine serum (FBS), bovine calf serum (FCS), equine serum, and porcine serum. Cells can be grown either in suspension or as adherent cultures. There are also cell lines that have been modified to be able to survive in suspension cultures, so they can be grown to a higher density than adherent conditions would allow.

The term “expression” includes any step involved in the production of a construct of the invention including, but not limited to, transcription, post-transcriptional modification, translation, folding, post-translational modification, targeting to specific subcellular or extracellular locations, and secretion. The term “recovering” refers to a series of processes intended to isolate the construct from the cell culture. The “recovering” or “purification” process may separate the protein and non-protein parts of the cell culture, and finally separate the desired construct from all other polypeptides and proteins. Separation steps usually exploit differences in protein size, physico-chemical properties, binding affinity and biological activity. Preparative purifications aim to produce a relatively large quantity of purified proteins for subsequent use, while analytical purification produces a relatively small amount of a protein for a variety of research or analytical purposes.

When using recombinant techniques, the construct can be produced intracellularly, in the periplasmic space, or directly secreted into the medium. If the construct is produced intracellularly, as a first step, the particulate debris, either host cells or lysed fragments, are removed, for example, by centrifugation or ultrafiltration. The construct of the invention may e.g. be produced in bacteria such as E. coli. After expression, the construct is isolated from the bacterial cell paste in a soluble fraction and can be purified e.g. via affinity chromatography and/or size exclusion. Final purification can be carried out in a manner that is like the process for purifying a construct expressed in mammalian cells and secreted into the medium. Carter et al. (Biotechnology (NY) 1992 February; 10(2):163-7) describe a procedure for isolating antibodies which are secreted to the periplasmic space of E. coli.

Where the antibody is secreted into the medium, supernatants from such expression systems are generally first concentrated using a commercially available protein concentration filter, for example, an ultrafiltration unit.

The construct of the invention prepared from the host cells can be recovered or purified using, for example, hydroxylapatite chromatography, gel electrophoresis, dialysis, and affinity chromatography. Other techniques for protein purification such as fractionation on an ion-exchange column, mixed mode ion exchange, HIC, ethanol precipitation, size exclusion chromatography, reverse phase HPLC, chromatography on silica, chromatography on heparin sepharose, chromatography on an anion or cation exchange resin (such as a polyaspartic acid column), immunoaffinity (such as Protein A/G/L) chromatography, chromato-focusing, SDS-PAGE, ultracentrifugation, and ammonium sulfate precipitation are also available depending on the construct to be recovered.

A protease inhibitor may be included in any of the foregoing steps to inhibit proteolysis, and antibiotics may be included to prevent the growth of contaminants.

Moreover, the invention provides a pharmaceutical composition or formulation comprising a polypeptide or polypeptide construct of the invention or a polypeptide or polypeptide construct produced according to the process of the invention. As used herein, the term “pharmaceutical composition” relates to a composition which is suitable for administration to a patient, preferably a human patient. The particularly preferred pharmaceutical composition of this invention comprises one or a plurality of the construct(s) of the invention, preferably in a therapeutically effective amount. Preferably, the pharmaceutical composition further comprises suitable formulations of one or more (pharmaceutically effective) carriers, stabilizers, excipients, diluents, solubilizers, surfactants, emulsifiers, preservatives and/or adjuvants. Acceptable constituents of the composition are preferably nontoxic to recipients at the dosages and concentrations employed. Pharmaceutical compositions of the invention include, but are not limited to, liquid, frozen, and lyophilized compositions.

The compositions may comprise a pharmaceutically acceptable carrier. In general, as used herein, “pharmaceutically acceptable carrier” means all aqueous and non-aqueous solutions, sterile solutions, solvents, buffers, e.g. phosphate buffered saline (PBS) solutions, water, suspensions, emulsions, such as oil/water emulsions, various types of wetting agents, liposomes, dispersion media and coatings, which are compatible with pharmaceutical administration, in particular with parenteral administration. The use of such media and agents in pharmaceutical compositions is well known in the art, and the compositions comprising such carriers can be formulated by well-known conventional methods.

Certain embodiments provide pharmaceutical compositions comprising the construct of the invention and further one or more excipients such as those illustratively described in this section and elsewhere herein. Excipients can be used in the invention for a wide variety of purposes, such as adjusting physical, chemical, or biological properties of formulations, such as adjustment of viscosity, and or processes of the invention to improve effectiveness and/or to stabilize such formulations and processes against degradation and spoilage e.g. due to stresses that occur during manufacturing, shipping, storage, pre-use preparation, administration, and thereafter. Excipients should in general be used in their lowest effective concentrations.

In certain embodiments, the pharmaceutical composition may contain formulation materials for modifying, maintaining or preserving certain characteristics of the composition such as the pH, osmolarity, viscosity, clarity, color, isotonicity, odor, sterility, stability, rate of dissolution or release, adsorption or penetration (see, Remington's Pharmaceutical Sciences, 18” Edition, 1990, Mack Publishing Company). In such embodiments, suitable formulation materials may include, but are not limited to:
amino acids
antimicrobials such as antibacterial and antifungal agents
antioxidants
buffers, buffer systems and buffering agents that are used to maintain the composition at physiological pH or at a slightly lower pH, typically within a range of from about 5 to about 8 or 9
non-aqueous solvents, vegetable oils, and injectable organic esters
aqueous carriers including water, alcoholic/aqueous solutions, emulsions or suspensions, including saline and buffered media
biodegradable polymers such as polyesters
bulking agents
chelating agents
isotonic and absorption delaying agents
complexing agents
fillers
carbohydrates
(low molecular weight) proteins, polypeptides or proteinaceous carriers, preferably of human origin
coloring and flavouring agents
sulfur containing reducing agents
diluting agents
emulsifying agents
hydrophilic polymers
salt-forming counter-ions
preservatives
metal complexes
solvents and co-solvents
sugars and sugar alcohols
suspending agents
surfactants or wetting agents
stability enhancing agents
tonicity enhancing agents
parenteral delivery vehicles
intravenous delivery vehicles

It is common knowledge that the different constituents of the pharmaceutical composition can have different effects, for example, and amino acid can act as a buffer, a stabilizer and/or an antioxidant; mannitol can act as a bulking agent and/or a tonicity enhancing agent; sodium chloride can act as delivery vehicle and/or tonicity enhancing agent; etc.

In the context of the present invention, a pharmaceutical composition may comprise:
(a) a polypeptide or polypeptide construct as described herein,
(b) at least one buffer agent,
(c) at least one saccharide, and
(d) at least one surfactant;
wherein the pH of the pharmaceutical composition is in the range of 3.5 to 6.

In the composition described above, the first domain preferably has an isoelectric point (pI) in the range of 4 to 9.5; the second domain has a pI in the range of 8 to 10, preferably 8.5 to 9.0; and the construct optionally comprises a third domain comprising two polypeptide monomers, each comprising a hinge, a CH2 domain and a CH3 domain, wherein said two polypeptide monomers are fused to each other via a peptide linker.

In the composition described above, it is further envisaged that the at least one buffer agent is present at a concentration range of 5 to 200 mM, more preferably at a concentration range of 10 to 50 mM. It is also envisaged that the at least one saccharide is selected from the group consisting of monosaccharide, disaccharide, cyclic polysaccharide, sugar alcohol, linear branched dextran or linear non-branched dextran. It is also envisaged that the disaccharide is selected from the group consisting of sucrose, trehalose and mannitol, sorbitol, and combinations thereof. It is further envisaged that the sugar alcohol is sorbitol. It is also envisaged that the at least one saccharide is present at a concentration in the range of 1 to 15% (m/V), preferably in a concentration range of 9 to 12% (m/V). It is further envisaged that the construct is present in a concentration range of 0.1 to 8 mg/ml, preferably of 0.2-2.5 mg/ml, more preferably of 0.25-1.0 mg/ml.

According to one embodiment of the composition described above, the at least one surfactant is selected from the group consisting of polysorbate 20, polysorbate 40, polysorbate 60, polysorbate 80, poloxamer 188, pluronic F68, triton X-100, polyoxyethylen, PEG 3350, PEG 4000 and combinations thereof. It is further envisaged that the at least one surfactant is present at a concentration in the range of 0.004 to 0.5% (m/V), preferably in the range of 0.001 to 0.01% (m/V). It is envisaged that the pH of the composition is in the range of 4.0 to 5.0, preferably 4.2. It is also envisaged that the pharmaceutical composition has an osmolarity in the range of 150 to 500 mOsm. It is further envisaged that the pharmaceutical composition further comprises an excipient selected from the group consisting of one or more polyol(s) and one or more amino acid(s). It is envisaged in the context of the present invention that said one or more excipient is present in the concentration range of 0.1 to 15% (w/V).

The present invention also provides a pharmaceutical composition comprising (a) the construct as described herein, preferably in a concentration range of 0.1 to 8 mg/ml, preferably of 0.2-2.5 mg/ml, more preferably of 0.25-1.0 mg/ml; (b) 10 mM glutamate or acetate; (c) 9% (m/V) sucrose or 6% (m/V) sucrose and 6% (m/V) hydroxypropyl-β-cyclodextrin; (d) 0.01% (m/V) polysorbate 80; wherein the pH of the liquid pharmaceutical composition is 4.2.

It is envisaged that the composition of the invention might comprise, in addition to the construct of the invention defined herein, further biologically active agents, depending on the intended use of the composition. Such agents might be drugs acting on the gastro-intestinal system, drugs acting as cytostatica, drugs preventing hyperurikemia, drugs inhibiting immunoreactions, drugs modulating the inflammatory response, drugs acting on the circulatory system and/or agents such as cytokines known in the art. It is also envisaged that the polypeptide construct of the present invention is applied in a co-therapy, i.e., in combination with another anti-cancer medicament.

In this context, it is envisaged that the pharmaceutical composition of the invention (which comprises a construct comprising a CD3 binding domain and at least a further binding domain which binds to a cell surface target antigen, preferably a tumor antigen on the surface of a target cell, as described in more detail herein above) furthermore comprises an agent, preferably an antibody or construct, which binds to a protein of the immune checkpoint pathway (such as PD-1 or CTLA-4) or to a co-stimulatory immune checkpoint receptor (such as 4-1BB). The present invention also refers to a combination of a polypeptide construct according to the invention (which comprises a construct comprising a CD3 binding domain and at least a further binding domain which binds to a cell surface target antigen, preferably a tumor antigen on the surface of a target cell, as described in more detail herein above) and an agent, preferably an antibody or polypeptide construct, which binds to a protein of the immune checkpoint pathway (such as PD-1 or CTLA-4) or to a co-stimulatory immune checkpoint receptor (such as 4-1BB). Due to the nature of the at least two ingredients of the combination, namely their pharmaceutical activity, the combination can also be referred to as a therapeutic combination. In some embodiments, the combination can be in the form of a pharmaceutical composition or of a kit. According to one embodiment, the pharmaceutical composition or the combination comprises a construct of the invention and an antibody or construct which binds to PD-1. Anti-PD-1 binding proteins useful for this purpose are e.g. described in detail in PCT/US2019/013205 incorporated herein by reference.

In certain embodiments, the optimal pharmaceutical composition is determined depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the construct of the invention. In certain embodiments, the primary vehicle or carrier in a pharmaceutical composition may be either aqueous or non-aqueous in nature. For example, a suitable vehicle or carrier may be water for injection or physiological saline solution, possibly supplemented with other materials common in compositions for parenteral administration. In certain embodiments, the compositions comprising the construct of the invention may be prepared for storage by mixing the selected composition having the desired degree of purity with optional formulation agents (Remington's Pharmaceutical Sciences, supra) in the form of a lyophilized cake or an aqueous solution. Further, in certain embodiments, the construct of the invention may be formulated as a lyophilizate using appropriate excipients.

When parenteral administration is contemplated, the therapeutic compositions for use in this invention may be provided in the form of a pyrogen-free, parenterally acceptable aqueous solution comprising the desired construct of the invention in a pharmaceutically acceptable vehicle. A particularly suitable vehicle for parenteral injection is sterile distilled water in which the construct of the invention is formulated as a sterile, isotonic solution, properly preserved. In certain embodiments, the preparation can involve the formulation of the desired molecule with an agent that may provide controlled or sustained release of the product which can be delivered via depot injection, or that may promote sustained duration in the circulation. In certain embodiments, implantable drug delivery devices may be used to introduce the desired construct.

Additional pharmaceutical compositions will be evident to those skilled in the art, including formulations involving the construct of the invention in sustained or controlled delivery formulations. Techniques for formulating a variety of sustained- or controlled-delivery means are known to those skilled in the art. The construct may also be entrapped in microcapsules prepared, for example, by coacervation techniques or by interfacial polymerization, in colloidal drug delivery systems, or in macroemulsions. Such techniques are disclosed in Remington's Pharmaceutical Sciences, supra.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions are generally placed into a container having a sterile access port, for example, an intravenous solution bag or vial having a stopper pierceable by a hypodermic injection needle.

Another aspect of the invention includes self-buffering formulations comprising the construct of the invention, which can be used as pharmaceutical compositions, as described in international patent application WO 2006/138181. A variety of publications are available on protein stabilization and formulation materials and methods useful in this regard, such as Arawaka T. et al., Pharm Res. 1991 March; 8(3):285-91; Kendrick et al., “Physical stabilization of proteins in aqueous solution” in: Rational Design of Stable Protein Formulations: Theory and Practice, Carpenter and Manning, eds. Pharmaceutical Biotechnology. 13: 61-84 (2002), and Randolph and Jones, Pharm Biotechnol. 2002; 13:159-75, see particularly the parts pertinent to excipients and processes for self-buffering protein formulations, especially as to protein pharmaceutical products and processes for veterinary and/or human medical uses.

Salts may be used in accordance with certain embodiments of the invention, e.g. to adjust the ionic strength and/or the isotonicity of a composition or formulation and/or to improve the solubility and/or physical stability of a construct or other ingredient of a composition in accordance with the invention. Ions can stabilize the native state of proteins by binding to charged residues on the protein's surface and by shielding charged and polar groups in the protein and reducing the strength of their electrostatic interactions, attractive, and repulsive interactions. Ions also can stabilize the denatured state of a protein by binding to, particularly the denatured peptide linkages (—CONH) of the protein. Furthermore, ionic interaction with charged and polar groups in a protein also can reduce intermolecular electrostatic interactions and, thereby, prevent or reduce protein aggregation and insolubility.

Ionic species differ significantly in their effects on proteins. Several categorical rankings of ions and their effects on proteins have been developed that can be used in formulating pharmaceutical compositions in accordance with the invention. One example is the Hofmeister series, which ranks ionic and polar non-ionic solutes by their effect on the conformational stability of proteins in solution. Stabilizing solutes are referred to as “kosmotropic”. Destabilizing solutes are referred to as “chaotropic”. Kosmotropes are commonly used at high concentrations to precipitate proteins from solution (“salting-out”). Chaotropes are commonly used to denature and/or to solubilize proteins (“salting-in”). The relative effectiveness of ions to “salt-in” and “salt-out” defines their position in the Hofmeister series.

Free amino acids can be used in formulations or compositions comprising the construct of the invention in accordance with various embodiments of the invention as bulking agents, stabilizers, and antioxidants, as well as for other standard uses. Certain amino acids can be used for stabilizing proteins in a formulation, others are useful during lyophilization to ensure correct cake structure and properties of the active ingredient. Some amino acids may be useful to inhibit protein aggregation in both liquid and lyophilized formulations, and others are useful as antioxidants.

Polyols are kosmotropic and are useful as stabilizing agents in both liquid and lyophilized formulations to protect proteins from physical and chemical degradation processes. Polyols are also useful for adjusting the tonicity of formulations and for protecting against freeze-thaw stresses during transport or the preparation of bulks during the manufacturing process. Polyols can also serve as cryoprotectants in the context of the present invention.

Certain embodiments of the formulation or composition comprising the construct of the invention can comprise surfactants. Proteins may be susceptible to adsorption on surfaces and to denaturation and resulting aggregation at air-liquid, solid-liquid, and liquid-liquid interfaces. These deleterious interactions generally scale inversely with protein concentration and are typically exacerbated by physical agitation, such as that generated during the shipping and handling of a product. Surfactants are routinely used to prevent, minimize, or reduce surface adsorption. Surfactants also are commonly used to control protein conformational stability. The use of surfactants in this regard is protein specific, since one specific surfactant will typically stabilize some proteins and destabilize others.

Certain embodiments of the formulation or composition comprising the construct of the invention can comprise one or more antioxidants. To some extent deleterious oxidation of proteins can be prevented in pharmaceutical formulations by maintaining proper levels of ambient oxygen and temperature and by avoiding exposure to light. Antioxidant excipients can also be used to prevent oxidative degradation of proteins. It is envisaged that antioxidants for use in therapeutic protein formulations in accordance with the present invention can be water-soluble and maintain their activity throughout the shelf life of the product (the composition comprising the construct). Antioxidants can also damage proteins and should hence—among other things—be selected in a way to eliminate or sufficiently reduce the possibility of antioxidants damaging the construct or other proteins in the formulation.

Certain embodiments of the formulation or composition comprising the construct of the invention can comprise one or more preservatives. Preservatives are necessary for example when developing multi-dose parenteral formulations that involve more than one extraction from the same container. Their primary function is to inhibit microbial growth and ensure product sterility throughout the shelf-life or term of use of the drug product. Although preservatives have a long history of use with small-molecule parenterals, the development of protein formulations that include preservatives can be challenging. Preservatives very often have a destabilizing effect (aggregation) on proteins, and this has become a major factor in limiting their use in multi-dose protein formulations. To date, most protein drugs have been formulated for single-use only. However, when multi-dose formulations are possible, they have the added advantage of enabling patient convenience, and increased marketability. A good example is that of human growth hormone (hGH) where the development of preserved formulations has led to commercialization of more convenient, multi-use injection pen presentations. Several aspects need to be considered during the formulation and development of preserved dosage forms. The effective preservative concentration in the drug product must be optimized. This requires testing a given preservative in the dosage form with concentration ranges that confer anti-microbial effectiveness without compromising protein stability.

As might be expected, development of liquid formulations containing preservatives are more challenging than lyophilized formulations. Freeze-dried products can be lyophilized without the preservative and reconstituted with a preservative containing diluent at the time of use. This shortens the time during which a preservative is in contact with the construct, significantly minimizing the associated stability risks. With liquid formulations, preservative effectiveness and stability should be maintained over the entire product shelf-life. An important point to note is that preservative effectiveness should be demonstrated in the final formulation containing the active drug and all excipient components. Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration.

The biological activity of the pharmaceutical composition defined herein can be determined for instance by in vitro cytotoxicity assays, as described in the following examples, in WO 99/54440 or by Schlereth et al. (Cancer Immunol. Immunother. 20 (2005), 1-12). “Efficacy” or “in vivo efficacy” as used herein refers to the response to therapy by the pharmaceutical composition of formulation of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells. The in vivo efficacy may be monitored by established standard methods for the respective disease entities including, but not limited to, white blood cell counts, differentials, fluorescence activated cell sorting, bone marrow aspiration. In addition, various disease specific clinical chemistry parameters and other established standard methods may be used. Furthermore, computer-aided tomography, X-ray, nuclear magnetic resonance tomography, positron-emission tomography scanning, lymph node biopsies/histologies and other established standard methods may be used.

Another major challenge in the development of drugs such as the pharmaceutical composition of the invention is the predictable modulation of pharmacokinetic properties. To this end, a pharmacokinetic profile of the drug candidate, i.e. a profile of the pharmacokinetic parameters that affect the ability of a specific drug to treat a given condition, can be established. Pharmacokinetic parameters of the drug influencing the ability of a drug for treating a certain disease entity include, but are not limited to: half-life, volume of distribution, hepatic first-pass metabolism and the degree of blood serum binding. The efficacy of a given drug agent can be influenced by each of the parameters mentioned above.

“Half-life” is the time required for a quantity to reduce to half its initial value. The medical sciences refer to the half-life of substances or drugs in the human body. In a medical context, half-life may refer to the time it takes for a substance/drug to lose one-half of its activity, e.g. pharmacologic, physiologic, or radiological activity. The half-life may also describe the time that it takes for the concentration of a drug or substance (e.g., a construct of the invention) in blood plasma/serum to reach one-half of its steady-state value (“serum half-life”). Typically, the elimination or removal of an administered substance/drug refers to the body's cleansing through biological processes such as metabolism, excretion, also involving the function of kidneys and liver. The “first-pass metabolism” is a phenomenon of drug metabolism whereby the concentration of a drug is reduced before it reaches the circulation. It is the fraction of drug lost during the process of absorption. Accordingly, by “hepatic first-pass metabolism” is meant the propensity of a drug to be metabolized upon first contact with the liver, i.e. during its first pass through the liver. “Volume of distribution” (VD) means the degree to which a drug is distributed in body tissue rather than the blood plasma, a higher VD indicating a greater amount of tissue distribution. The retention of a drug can occur throughout the various compartments of the body, such as intracellular and extracellular spaces, tissues and organs, etc. “Degree of blood serum binding” means the propensity of a drug to interact with and bind to blood serum proteins, such as albumin, leading to a reduction or loss of biological activity of the drug.

Pharmacokinetic parameters also include bioavailability, lag time (T lag), Tmax, absorption rates, and/or Cmax for a given amount of drug administered. “Bioavailability” refers to the fraction of an administered dose of a drug/substance that reaches the systemic circulation (the blood compartment). When a medication is administered intravenously, its bioavailability is considered to be 100%. However, when a medication is administered via other routes (such as orally), its bioavailability generally decreases. “Lag time” means the time delay between the administration of the drug and its detection and measurability in blood or plasma. Cmax is the maximum plasma concentration that a drug achieves after its administration (and before the administration of a second dose). Tmax is the time at which Cmax is reached. The time to reach a blood or tissue concentration of the drug which is required for its biological effect is influenced by all parameters. Pharmacokinetic parameters of constructs exhibiting cross-species specificity may be determined in preclinical animal testing in non-chimpanzee primates as outlined above and set forth e.g. in Schlereth et al. (supra).

One embodiment provides the construct of the invention (or the construct produced according to the process of the invention), for the use as a medicament, particularly for the use in the prevention, treatment or amelioration (preferably treatment) of a disease, preferably a tumorous disease, more preferred a neoplasm, cancer or tumor. Another embodiment provides the use of the construct of the invention (or of the construct produced according to the process of the invention) in the manufacture of a medicament for the prevention, treatment or amelioration of a disease, preferably a tumorous disease, more preferred a neoplasm, cancer or tumor. It is also envisaged to provide a method for the prevention, treatment or amelioration of a disease, preferably a tumorous disease, more preferred a neoplasm, cancer or tumor, comprising the step of administering to a subject in need thereof the construct of the present invention (or the construct produced according to the process of the present invention). The terms “subject in need”, “patient” or those “in need of treatment” include those already with the disease, as well as those in which the disease is to be prevented. The terms also include human and other mammalian subjects that receive either prophylactic or therapeutic treatment.

The polypeptides/polypeptide constructs of the invention and the formulations/pharmaceutical compositions described herein are useful in the treatment, amelioration and/or prevention of the medical condition as described herein in a patient in need thereof. The term “treatment” refers to both therapeutic treatment and prophylactic or preventative measures. Treatment includes the application or administration of the polypeptides/polypeptide constructs/pharmaceutical composition to the body, to an isolated tissue, or to a cell from a patient or a subject in need who has a disease/disorder as described herein, a symptom of such disease/disorder, or a predisposition toward such disease/disorder, with the purpose to cure, heal, alleviate, relieve, alter, remedy, ameliorate, improve, or affect the disease, the symptom of the disease, or the predisposition toward the disease. The term “amelioration” as used herein refers to any improvement of the disease state of a patient, by the administration of a polypeptide construct according to the invention to such patient or subject in need thereof. Such an improvement may be a slowing down or stopping of the progression of the disease of the patient, and/or as a decrease in severity of disease symptoms, an increase in frequency or duration of disease symptom-free periods or a prevention of impairment or disability due to the disease. The term “prevention” as used herein means the avoidance of the occurrence or of the re-occurrence of a disease as specified herein, by the administration of a construct according to the invention to a subject in need thereof.

The term “disease” refers to any condition that would benefit from treatment with the construct or the pharmaceutical composition described herein. This includes chronic and acute disorders or diseases including those pathological conditions that predispose the mammal to the disease in question. The disease is preferably a tumorous disease, more preferred a neoplasm, cancer or tumor. The disease, neoplasm, cancer or tumor is preferably positive for a tumor antigen, preferably such as those defined herein above, i.e. it is characterized by expression or overexpression of a tumor antigen, preferably such as those defined herein above. An overexpression of a tumor antigen means that there is an increase by at least 10%, in particular at least 25%, at least 50%, at least 100%, at least 250%, at least 500%, at least 750%, at least 1000% or even more. Expression is, preferably, only found in a diseased tissue, while expression in a corresponding healthy tissue is not or significantly not detectable. According to the invention, diseases associated with cells expressing a tumor antigen, preferably such as those defined herein above, include cancer diseases. Furthermore, according to the invention, cancer diseases preferably are those wherein the cancer cells express a tumor antigen. In accordance with the invention, the disease, preferably tumorous disease, more preferred neoplasm, tumor or cancer is preferably characterized by the presence of BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive cells. In other words, the tumorous disease, more preferred neoplasm, tumor or cancer is preferably associated with the presence of BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive cells; the tumorous disease, more preferred neoplasm, tumor or cancer can therefore be termed a BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvIII-positive, FLT3-positive, MAGEB2-positive, MART1-positive, MSLN-positive, MUC17-positive, PSMA-positive, or STEAP1-positive neoplasm, tumor or cancer. It is understood herein, that each of said tumor antigen-positive neoplasms, tumors or cancers can be prevented, treated or ameliorated using a polypeptide or polypeptide construct according to the invention that comprises a binding domain against the tumor antigen expressed by the cells with which said neoplasm, tumor or cancer is associated with. A BCMA-positive, CD123-positive, CD19-positive, CD20-positive, CD22-positive, CD33-positive, CD70-positive, CDH19-positive, CDH3-positive, CLL1-positive, CS1-positive, CLDN6-positive, CLDN18.2-positive, DLL3-positive, EGFRvII-positive, FLT3-positive, MAGEB2-positive, MART1-positive, MSLN-positive, MUC17-positive, PSMA-positive, or STEAP1-positive neoplasm, tumor or cancer can be prevented, treated or ameliorated using a polypeptide or polypeptide construct according to the invention that comprises a binding domain against BCMA (for a BCMA-positive neoplasm, tumor or cancer), CD123 (for a CD123-positive neoplasm, tumor or cancer), CD19 (for a CD19-positive neoplasm, tumor or cancer), CD20 (for a CD20-positive neoplasm, tumor or cancer), CD22 (for a CD22-positive neoplasm, tumor or cancer), CD33 (for a CD33-positive neoplasm, tumor or cancer), CD70 (for a CD70-positive neoplasm, tumor or cancer), CDH19 (for a CDH19-positive neoplasm, tumor or cancer), CDH3 (for a CDH3-positive neoplasm, tumor or cancer), CLL1 (for a CLL1-positive neoplasm, tumor or cancer), CS1 (for a CS1-positive neoplasm, tumor or cancer), CLDN6 (for a CLDN6-positive neoplasm, tumor or cancer), CLDN18.2 (for a CLDN18.2-positive neoplasm, tumor or cancer), DLL3 (for a DLL3-positive neoplasm, tumor or cancer), EGFRvIII (for a EGFRvIII-positive neoplasm, tumor or cancer), FLT3 (for a FLT3-positive neoplasm, tumor or cancer), MAGEB2 (for a MAGEB2-positive neoplasm, tumor or cancer), MART1 (for a MART1-positive neoplasm, tumor or cancer), MSLN (for a MSLN-positive neoplasm, tumor or cancer), MUC17 (for a MUC17-positive neoplasm, tumor or cancer), PSMA (for a PSMA-positive neoplasm, tumor or cancer), and STEAP1 (for a STEAP1-positive neoplasm, tumor or cancer), respectively.

A “neoplasm” is an abnormal growth of tissue, usually but not always forming a mass. When also forming a mass, it is commonly referred to as a “tumor”. Neoplasms or tumors can be benign, potentially malignant (pre-cancerous), or malignant (cancerous). Malignant neoplasms/tumors are commonly called cancer. They usually invade and destroy the surrounding tissue and may form metastases, i.e., they spread to other parts, tissues or organs of the body. A “primary tumor” is a tumor growing at the anatomical site where tumor progression began and proceeded to yield a cancerous mass. Most cancers develop at their primary site but then go on to metastasize or spread to other parts (e.g. tissues and organs) of the body. These further tumors are “secondary tumors”. Most cancers continue to be called after their primary site, even after they have spread to other parts of the body.

Lymphomas and leukemias are lymphoid neoplasms. For the purposes of the present invention, they are also encompassed by the terms “tumor” and “cancer”. For the purposes of the present invention, the terms “neoplasm”, “tumor” and “cancer” may be used interchangeably, and they comprise both primary tumors/cancers and secondary tumors/cancers (or “metastases”) as well as mass-forming neoplasms (tumors) and lymphoid neoplasms (such as lymphomas and leukemias), and minimal residual disease (MRD).

The term “minimal residual disease” (MRD) refers to the evidence for the presence of small numbers of residual cancer cells that remain in the patient after cancer treatment, e.g. when the patient is in remission (no symptoms or signs of disease). A very small number of remaining cancer cells usually cannot be detected by routine means because the standard tests used to assess or detect cancer are not sensitive enough to detect MRD. Nowadays, very sensitive molecular biology tests for MRD are available, such as flow cytometry, PCR and next-generation sequencing. These tests can measure minimal levels of cancer cells in tissue samples, sometimes as low as one cancer cell in a million normal cells. In the context of the present invention, the terms “prevention”, “treatment” or “amelioration” of a cancer are envisaged to also encompass “prevention, treatment or amelioration of MRD”, whether the MRD was detected or not.

The construct of the invention will generally be designed for specific routes and methods of administration, for specific dosages and frequencies of administration, for specific treatments of specific diseases, with ranges of bio-availability and persistence, among other things. The materials of the composition are preferably formulated in concentrations that are acceptable for the site of administration. Formulations and compositions thus may be designed in accordance with the invention for delivery by any suitable route of administration. In the context of the present invention, the routes of administration include, but are not limited to topical routes, enteral routes and parenteral routes.

If the pharmaceutical composition has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization. The pharmaceutical compositions and the construct of this invention are particularly useful for parenteral administration, e.g., intravenous delivery, for example by injection or infusion. Pharmaceutical compositions may be administered using a medical device. Examples of medical devices for administering pharmaceutical compositions are described in U.S. Pat. Nos. 4,475,196; 4,439,196; 4,447,224; 4,447,233; 4,486,194; 4,487,603; 4,596,556; 4,790,824; 4,941,880; 5,064,413; 5,312,335; 5,312,335; 5,383,851; and 5,399,163.

The compositions of the present invention can be administered to the subject at a suitable dose which can be determined e.g. in dose escalating studies. As set forth above, the construct of the invention exhibiting cross-species specificity as described herein can also be advantageously used in in preclinical testing in non-chimpanzee primates. The dosage regimen will be determined by the attending physician and clinical factors. As is well known in the medical art, dosages for any one patient depend upon many factors, including the patient's size, body surface area, age, the specific compound to be administered, sex, time and route of administration, general health, and other drugs being administered concurrently.

An “effective dose” is an amount of a therapeutic agent that is sufficient to achieve or at least partially achieve a desired effect. A “therapeutically effective dose” is an amount that is sufficient to cure or at least partially arrest the disease and its complications, signs and symptoms in a patient suffering from the disease. Amounts or doses effective for this use will depend on the disease to be treated (the indication), the delivered construct, the therapeutic context and objectives, the severity of the disease, prior therapy, the patient's clinical history and response to the therapeutic agent, the route of administration, the size (body weight, body surface) and/or condition (the age and general health) of the patient, and the general state of the patient's own immune system. The proper dose can be adjusted according to the judgment of the attending physician, to obtain the optimal therapeutic effect.

A therapeutically effective amount of a construct of the invention preferably results in a decrease in severity of disease symptoms, an increase in frequency or duration of disease symptom-free periods or a prevention of impairment or disability due to the disease. In the treatment of tumor antigen-expressing tumors, a therapeutically effective amount of the construct of the invention comprising a binding domain against said tumor antigen preferably inhibits tumor cell growth by at least about 20%, at least about 40%, at least about 50%, at least about 60%, at least about 70%, at least about 80%, or at least about 90% relative to untreated patients. The ability of a compound to inhibit tumor growth may also be evaluated in an animal model predictive of efficacy in human tumors.

In a further embodiment, the invention provides a kit comprising a construct of the invention, a construct produced according to the process of the invention, a polynucleotide of the invention, a vector of the invention, and/or a host cell of the invention. In the context of the present invention, the term “kit” means two or more components—one of which corresponding to the construct, the pharmaceutical composition, the polynucleotide, the vector or the host cell of the invention—packaged together in a container, recipient or otherwise. A kit can hence be described as a set of products and/or utensils that are sufficient to achieve a certain goal, which can be marketed as a single unit.

It is envisaged that a further component of the kit of the invention is an agent, preferably an antibody or construct, which binds to a protein of the immune checkpoint pathway (such as PD-1 or CTLA-4) or to a co-stimulatory immune checkpoint receptor (such as 4-1BB). These agents are described in more detail herein above. According to one embodiment, the kit comprises a construct of the invention and an antibody or construct which binds to PD-1. Anti-PD-1 binding proteins useful for this purpose are e.g. described in detail in PCT/US2019/013205. In certain embodiment, the kit allows for the simultaneous and/or sequential administration of the components.

The kit may comprise one or more recipients (such as vials, ampoules, containers, syringes, bottles, bags) of any appropriate shape, size and material (preferably waterproof, e.g. plastic or glass) containing the construct or the pharmaceutical composition of the present invention in an appropriate dosage for administration (see above). The kit may additionally contain directions for use (e.g. in the form of a leaflet or instruction manual), means for administering the construct or the pharmaceutical composition of the present invention such as a syringe, pump, infuser or the like, means for reconstituting the construct of the invention and/or means for diluting the construct of the invention.

The invention also provides kits for a single-dose administration unit. The kit of the invention may also contain a first recipient comprising a dried/lyophilized construct or pharmaceutical composition and a second recipient comprising an aqueous formulation. In certain embodiments of this invention, kits containing single-chambered and multi-chambered pre-filled syringes are provided.

Whenever the term “construct” is used herein, said term refers to the used polypeptide/polypeptide constructs of the invention or controls thereof as indicated.

As used herein, the singular forms “a”, “an”, and “the” include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within ±20%, preferably within +15%, more preferably within +10%, and most preferably within ±5% of a given value or range. It also includes the concrete value, e.g., “about 50” includes the value “50”.

Throughout this specification and the claims, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein, any of the terms “comprising”, “consisting essentially of” and “consisting of” may be replaced with either of the other two terms.

The above description and the below examples provide exemplary arrangements, but the present invention is not limited to the specific methodologies, techniques, protocols, material, reagents, substances, etc., described herein and as such can vary. The terminology used herein serves to describe specific embodiments only. The terminology used herein does not intend to limit the scope of the present invention, which is defined solely by the claims. Aspects of the invention are provided in the independent claims. Some optional features of the invention are provided in the dependent claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

A better understanding of the present invention and of its advantages will be obtained from the following examples, offered for illustrative purposes only. The examples are not intended and should not be construed as to limit the scope of the present invention in any way.

### BRIEF DESCRIPTION OF THE DRAWINGS

FIG. 1: G4S vs. G4R Linker in CD33 BiTE® molecule variants analyzed by SDS-PAGE without Stress Test

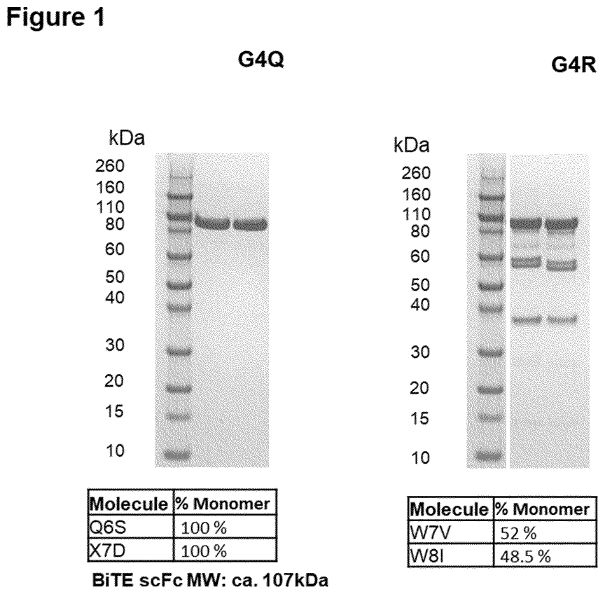

FIG. 2: PSMA BiTE® variants analyzed by SDS-PAGE without stress test

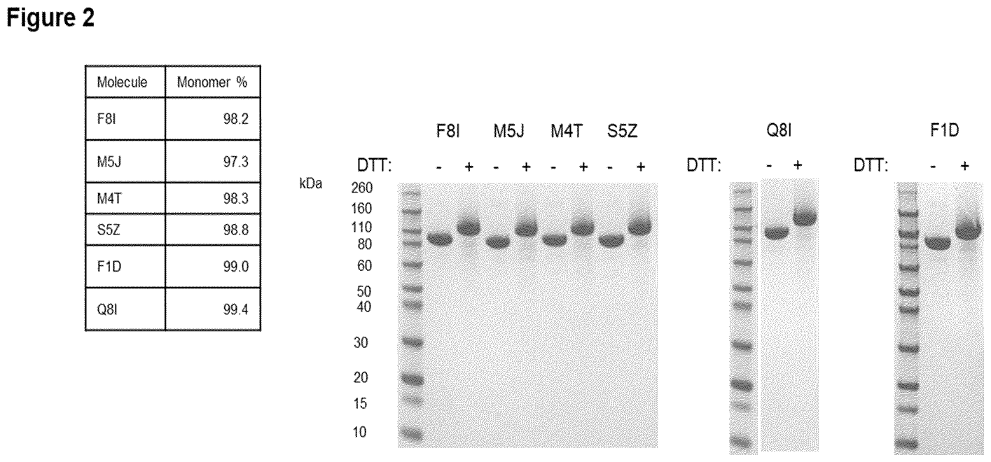

FIG. 3: Target 2 TDCC assay

FIG. 4: Target 1 TDCC assay

FIG. 5: Direct comparison of a PSMA BiTE® molecule vs. optimized variant of a BiTE molecule

FIG. 6: Schematic depiction of clipping sites detected in BiTE molecule Z5S and introduced stabilizations

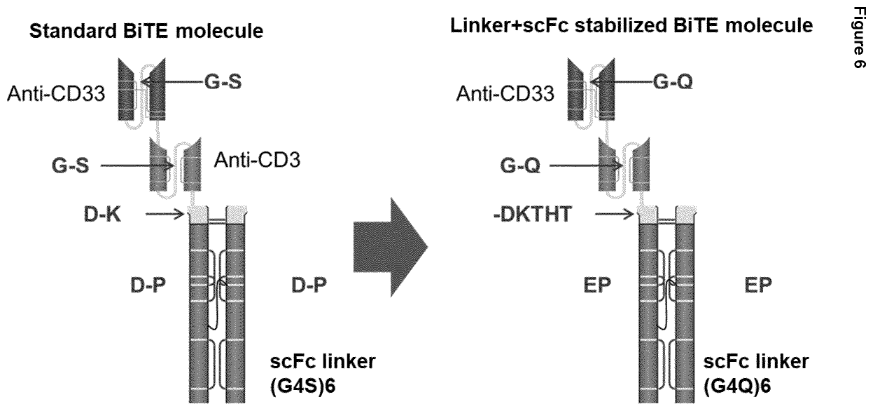

### EXAMPLES

### Example 1: Material and Methods

Generation and Expression of BiTE Molecules

Individual DNA fragments of the open reading frames were ordered as gene syntheses and subcloned into a mammalian expression vector by standard cloning methods. The expression vector contained an IgG derived signal peptide for secreted expression into the cell culture supernatant. Sequence verified plasmid clones were transfected into CHO cells, cell culture supernatant of a stable cell pool was harvested after 7 days. Cell culture supernatant was stored at −80° C. until protein purification.

BiTE Purification Chromatography Analysis

Protein purification was done by Protein A (Cytiva, Mab Select SuRe column) affinity chromatography followed by size exclusion chromatography (HiLoad® 16/600 Superdex® 200 pg GE Healthcare). According to the OD280 nm signal (blue) peaks were pooled and MW was analyzed by SDS-PAGE. Protein monomer peaks were formulated in 10 mM Citrate, 75 mM Lysine, 4% Trehalose and aliquoted for storage at −80° C.

SDS-PAGE Analysis

Samples were denatured by 95° C. for 5 min and applied to non-reducing (−DTT) or reducing (+DTT) SDS-PAGE. Gels were subsequently stained/destained instant blue to visualize protein bands.

In Vitro FACS Binding Analysis

Purified BiTE® antibody constructs were applied to flow cytometry to determine binding to target antigen transfected CHO cells or a human CD3 positive T cell line (HPB-ALL) or human PBMCs of healthy volunteers. BiTE molecules were stained using a PE-anti human IgG (1:200). Assay was run at 100/10/1/0.1 nM BiTE® molecules for 30 minutes at 4° C. Staining was referenced to cells only stained by the secondary anti-human Fc-specific PE-conjugated polyclonal Ab.

FACS-Based Cytotoxicity Assay with Unstimulated Human PBMC

Isolation of Effector Cells

Human peripheral blood mononuclear cells (PBMC) were prepared by Ficoll density gradient centrifugation from enriched lymphocyte preparations (buffy coats), a side product of blood banks collecting blood for transfusions. Buffy coats were supplied by a local blood bank and PBMC were prepared on the day after blood collection. After Ficoll density centrifugation and extensive washes with Dulbecco's PBS (Gibco), remaining erythrocytes were removed from PBMC via incubation with erythrocyte lysis buffer (155 mM NH4Cl, 10 mM KHCO3, 100 pM EDTA). Remaining lymphocytes mainly encompass B and T lymphocytes, NK cells and monocytes. PBMC were kept in culture at 37° C./5% C02 in RPMI medium (Gibco) with 10% FCS (Gibco).

Isolation of Pan T Cells

Human T cells were isolated from PBMC using Miltenyi Biotec human Pan T cell isolation kit (130-096-535) according to the protocol provided with the kit. T cells were then isolated using LS Columns (Milteny Biotec, #130-042-401). T cells were cultured in RPMI complete medium i.e. RPM11640 (Biochrom AG, #FG1215) supplemented with 10% FBS (Bio West, #S1810), 1× non-essential amino acids (Biochrom AG, #K0293), 10 mM Hepes buffer (Biochrom AG, #L1613), 1 mM sodium pyruvate (Biochrom AG, #L0473) and 100 U/mL penicillin/streptomycin (Biochrom AG, #A2213) at 37° C. in an incubator until needed.

Target Cell Labeling

For the analysis of cell lysis in flow cytometry assays, the fluorescent membrane dye DiOC18 (DiO) (Thermo Fisher, #V22886) was used to label target transfected CHO cells as target cells and distinguish them from effector cells. Briefly, cells were harvested, washed once with PBS and adjusted to 10e6 cell/mL in PBS containing 2% (v/v) FBS and the membrane dye DiO (5 μL/10e6 cells). After incubation for 3 min at 37° C., cells were washed twice in complete RPMI medium and the cell number adjusted to 1.25×10e5 cells/mL. The vitality of cells was determined using Nucleocounter NC-250 (Chemometec) and Solution18 Dye containing Acridine Orange and DAPI (Chemometec).

Flow Cytometry Based Analysis

This assay was designed to quantify the lysis of cyno or human target-transfected CHO cells in the presence of serial dilutions of bispecific antibody constructs. Equal volumes of DiO-labeled target cells and effector cells (i.e., PBMC w/o CD14+ cells) were mixed, resulting in an E:T cell ratio of 10:1. 160 μl of this suspension were transferred to each well of a 96-well plate. 40 μL of serial dilutions of the corresponding target×CD3 bispecific antibody constructs and a negative control bispecific (a CD3-based bispecific antibody construct recognizing an irrelevant target antigen) or RPMI complete medium as an additional negative control were added. The bispecific antibody-mediated cytotoxic reaction proceeded for 48 hours in a 7% CO2 humidified incubator. Then cells were transferred to a new 96-well plate and loss of target cell membrane integrity was monitored by adding propidium iodide (PI) at a final concentration of 1 μg/mL. PI is a membrane impermeable dye that normally is excluded from viable cells, whereas dead cells take it up and become identifiable by fluorescent emission.

Samples were measured by flow cytometry on an iQue Plus (Intellicyt, now Sartorius) instrument and analyzed by Forecyt software (Intellicyt). Target cells were identified as DiO-positive cells. PI-negative target cells were classified as living target cells. Percentage of cytotoxicity was calculated according to the following formula:

$$Cytotoxicity [ \% ] = \frac{{}^{n} dead target cells}{{}^{n} target cells} \times 100$$

n=number of events

Using GraphPad Prism 7.04 software (Graph Pad Software, San Diego), the percentage of cytotoxicity was plotted against the corresponding bispecific antibody construct concentrations. Dose response curves were analyzed with the four parametric logistic regression models for evaluation of sigmoid dose response curves with fixed hill slope and EC50 values were calculated.

Stress Test of Samples

Thermal degradation was induced by incubating samples in formulation buffer at 40° C. for 4 weeks. Physiological pH degradation (pH jump) was induced by adjusting samples in formulation to pH 7.2 with phosphate-buffered saline (PBS) followed by incubation at 37° C. for 4 weeks.

Solution Preparation

Denaturing buffer (6M guanidine HCl, 200 mM tris, 20 mM methionine, pH 8.3) was prepared by adding 20 mL 1 M (hydroxymethyl) aminomethane hydrochloride (tris), pH 8.3 (Teknova, St. Louis, MO, P/N T1083) to 87.5 mL 8 M guanidine HCl (Pierce, Rockford, IL, P/N 24115) followed by addition of 299 mg L-methionine (J. T. Baker, P/N 2085-05). The pH of the solution was adjusted to pH 8.3 with 1 N hydrochloric acid (HCl) (Ricca, Arlington, TX, P/N R3700100-120A) or 1 N Sodium hydroxide (NaOH) (Merck, Kenilworth, NJ, P/N 1.09137.100). Volume was adjusted to 100 mL with HPLC-grade water. Reduction solution (500 mM DTT) was prepared by dissolving 7.7 mg pre-weighed dithiothreitol (DTT) (Pierce, Rockford, IL, P/N 20291) in 100 μL denaturing buffer. Alkylation solution (500 mM NaIAA) was prepared by dissolving 15-65 mg sodium iodoacetate (NaIAA) (Sigma, St. Louis, MO, P/N 1-9148) in a volume of denaturing buffer sufficient to yield 500 mM NaIAA. Digestion buffer (50 mM tris, 20 mM methionine, pH 7.8) was prepared by dissolving 299 mg L-methionine in 10 mL 1 M tris, pH 7.8 and adding 100 mL HPLC-grade water. The pH of the solution was adjusted to pH 7.8 with 1 N hydrochloric acid (HCl) (Ricca, Arlington, TX, P/N R3700100-120A) or 1 N Sodium hydroxide (NaOH) (Merck, Kenilworth, NJ, P/N 1.09137.100), and the volume was adjusted to 100 mL with HPLC-grade water. Enzyme solutions (1 mg/mL trypsin, 1 mg/mL neutrophil elastase) were prepared by adding 100 μL digestion buffer to 100 μg trypsin (Roche, Basel, Switzerland, P/N 03708969001) or 100 μg neutrophil elastase (Elastin Products Company, Owensville, MO, P/N SE563). Digest quenching solution (8 M guanidine HCl, 250 mM acetate, pH 4.7) was prepared by dissolving 76.4 g guanidine HCl (Sigma, St. Louis, MO, P/N 50933) and 1.0 g sodium acetate (Sigma, P/N 32319) in 95 mL HPLC grade water. 716 μL glacial acetic acid (Sigma, St. Louis, MO, P/N 320099) was then added, and the pH was adjusted to pH 4.7 with either HCl or NaOH. Volume was then adjusted to 100 mL with HPLC grade water.

MWCO Spin Filter-Aided Sequential Digest

200 μL denaturing buffer was added to a 30 kDa molecular weight cut-off spin unit consisting of a membrane unit positioned inside a centrifuge tube for filtrate collection. (Millipore, Billerica, MA, P/N MRCF0R030 or Pall, Port Washington, NY, P/N OD030C34). This unit was spun for 10 min at 14,000×g using an Eppendorf 5430 centrifuge. Filtrate was discarded. 100 μg sample in formulation buffer was added to the filter unit and spun for 10 min at 14,000×g. Filtrate was discarded. For each sample, 3 μL reducing solution was added to 37 μL denaturing buffer, and 40 μL of this solution was added to the filter unit. Samples were denatured and reduced by incubating at 37° C. water bath for 30 min. For each sample 7 μL alkylation solution was added to 33 μL denaturing buffer, and 40 μL of this solution was added to the filter unit. Samples were alkylated by incubating at room temperature in dark for 20 min. For each sample 4 μL denaturing solution was added to 36 μL denaturing buffer, and 40 μL of this solution was added to the filter unit to quench alkylation. Samples were then spun for 15 min at 14,000×g, and the filtrate was discarded. 200 μL digest buffer was added to the sample, the filter unit was spun for 15 min at 14,000×g, and the filtrate was discarded; this was repeated two additional times to remove denaturing, reducing, and alkylating agents. For each sample, 5 μL trypsin solution was added to 35 μL digest buffer, and 40 μL of this solution was added to the filter unit (1:20 enzyme:substrate ratio). Sample was incubated in a 37° C. water bath for 60 min. The filter unit was transferred to a new collection tube (Collection Tube 2). The initial collection tube (Collection Tube 1) was set aside. The filter unit was centrifuged for 10 min at 14,000×g. Filtrate, which contained tryptic peptides, was retained in Collection Tube 2. 20 μL digest buffer was added to the filter unit, the filter unit (in Collection Tube 2) was spun for 10 min at 14,000×g, and the filtrate was retained in Collection Tube 2; this was repeated one additional time. The filter unit was transferred back to Collection Tube 1, and Collection Tube 2 was set aside. For each sample, 5 μL neutrophil elastase solution was added to 35 μL digest buffer, and 40 μL of this solution was added to the filter unit (now in Collection Tube 1, 1:20 enzyme:substrate ratio based on starting material). Sample was incubated in a 37° C. water bath for 30 min. The filter unit was transferred to Collection Tube 2. Collection Tube 1 was discarded. The filter unit was centrifuged for 10 min at 14,000×g. Filtrate, which contained peptides resulting from neutrophil elastase digestion, was retained in Collection Tube 2 (along with tryptic peptides from previous steps). 20 μL digest buffer was added to the filter unit, the filter unit (in Collection Tube 2) was spun for 10 min at 14,000×g, and the filtrate was retained in Collection Tube 2; this was repeated one additional time. Digest was quenched by the addition of 160 μL digest quenching buffer to Collection Tube 2.

UPLC Conditions

For all samples, Mobile Phase A consisted of 0.1% formic acid in water, and Mobile Phase B consisted of 0.1% formic acid in acetonitrile. Peptides were separated using a BEH C18 1.7 μm, 2.1×150 mm UPLC column (Waters, Milford, MA, P/N 186003556). UPLC separations were performed using either a Thermo Scientific U-3000 system (Waltham, MA), a Waters Acquity H-Class system (Milford, MA), or and Agilent 1290 system (Santa Clara, CA) utilizing gradients outlined in Table 1. Based on starting material, ˜3-4 μg of sample was loaded on column.

MS Conditions

Peptides resulting from digestion were analyzed using a Thermo Scientific Q Exactive (Waltham, MA), a Thermo Scientific Q Exactive Plus (Waltham, MA), or a Thermo Scientific Q Exactive BioPharma (Waltham, MA). Because multiple instruments were used, data collection parameters varied slightly depending on the instrument. Instruments were operated in data-dependent mode (top 4-8) over a scan range of 200-2,000 m/z. The AGC target was set to 1 E6 for MS1 scans and 5E5 for MSMS scans. MS1 scans were collected at a resolution of either 35,000 or 140,000, and MS2 scans were collected at a resolution of 17,500. An isolation window of 2-4 m/z was specified for MSMS scans. Peaks with unassigned charge states and charge states greater than 8 were excluded from MSMS. Dynamic exclusion was set to 10 s. Lock Mass of m/z 391.28430 was enabled.

Data Analysis

For peptide identification, MS data were searched with MassAnalyzer (data were collected over several months, so multiple versions of MassAnalyzer were used). Carboxymethylation was specified as a static modification. Depending on the experiment, cleavage was specified as either nonspecific or the C-terminus of amino acids KRVITAL amino acids. For all searches, signal-to-noise ratio was set to 20, mass accuracy of 15 ppm was specified, and confidence was set to 0.95. For sequence coverage maps, minimum peak area was set to 1% of the base peak, relative peak area threshold was set to 17%, minimum confidence was set to 0.95, and maximum peptide mass was set to 15,000. For quantitation, MS data were processed with Skyline. Skyline workbooks were created using peptides identified with MassAnalyzer search results.

TABLE 1
UHPLC Gradient. MS data was only collected at 10-78 min.
For the first 10 min, flow was diverted away from the MS
so that reagents used in sample prep could be washed to
waste. Following MS data acquisition, the column was
washed and equilibrated (78-123 min).
Flow was diverted away from the MS during this period.

| Time | % Mobile | Flow Rate | Column |
|---|---|---|---|
| (min) | Phase B | (mL/min) | Temperature (° C.) |
| 0.0 | 1.0 | 0.25 | 50 |
| 10.0 | 1.0 | 0.25 | 50 |
| 11.0 | 10.0 | 0.25 | 50 |
| 78.0 | 36.0 | 0.25 | 50 |
| 80.0 | 90.0 | 0.25 | 50 |
| 85.0 | 90.0 | 0.25 | 50 |
| 87.0 | 1.0 | 0.25 | 50 |
| 89.0 | 1.0 | 0.25 | 50 |
| 91.5 | 10.0 | 0.25 | 50 |
| 99.5 | 45.0 | 0.25 | 50 |
| 101.0 | 90.0 | 0.25 | 50 |
| 107.0 | 90.0 | 0.25 | 50 |
| 109.0 | 1.0 | 0.25 | 50 |
| 123.0 | 1.0 | 0.25 | 50 |

### Example 2: Results

SDS-PAGE Analysis

The purified monomers of the generated BiTE molecules were applied to SDS-PAGE analysis under non-reducing and/or reduced conditions. Except for BiTE molecules (W7V, W8I), all BiTE molecules showed one single band at the expected molecular weight under both conditions. For the BiTE molecules W7V and W8I that comprise G4R linker repeats additional bands of lower molecular weight could be observed, suggesting Low molecular weight (LMW) fragments without thermal stress test.

Cytotoxic Activity

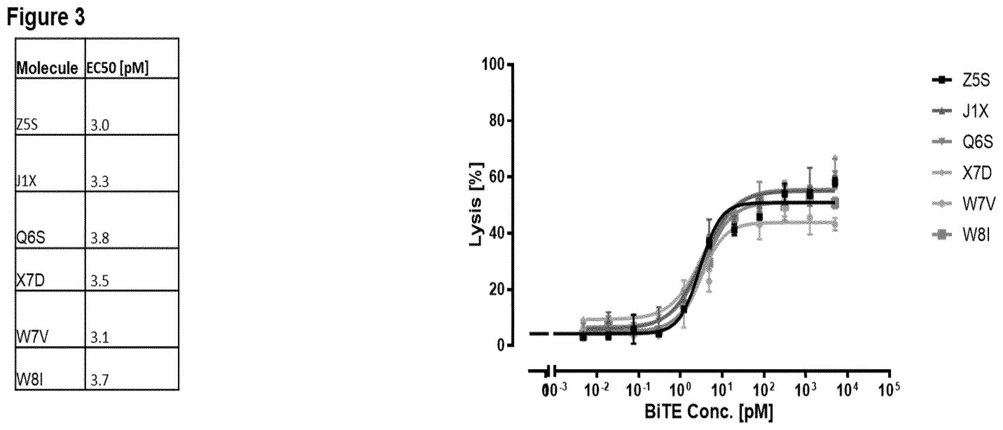

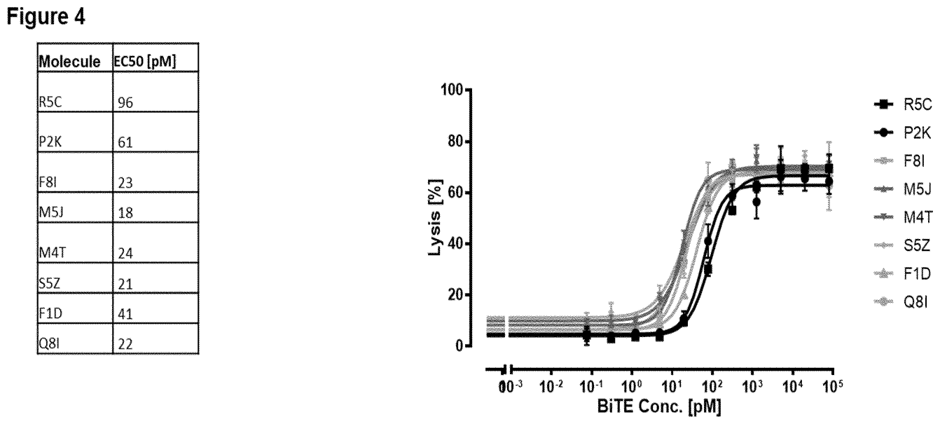

All analyzed BiTE molecules showed cytotoxic activity on target antigen transfected CHO cells. Activity was either comparable to the reference standard BiTE molecule (Target 2, FIG. 3) or to standard BiTE molecules comprising a parental, less affine target binder (R5C/P2K, Target 1, FIG. 4).

Thermal Stress Test

BiTE molecules were applied to thermal stress at 40° C. for four weeks and subsequently analyzed for % LMW increase compared to an untreated sample control as described.

Anti-CD3 scFv

The substitution of the standard Anti-CD3 scFv by the stabilized Anti-CD3 scFv in BiTE molecules (F8I vs. F1D, Table 1) with G4Q linker repeats decreased the LMW by 13.3%. When the standard Anti-CD3 scFv and the standard single chain Fc domain were substituted by the according stabilized domains the LMW decreased by 40.9% in the context of G4Q linkers (M5J vs. Q8I, Table 1).

Linker

The BiTE molecules comprising the G4Q linker repeats instead of G4S linker repeats showed reduced LMW percentage by 35.8% for BiTE molecules comprising the standard Anti-CD3 scFv, engineered Anti-CD3 scFv cys-clamp and the standard scFc domain (11D vs. F8I, Table 1).

Without the engineered Anti-CD3 scFv cys-clamp, BiTE molecules comprising G4Q linker repeats showed 32.3% less LMW than BiTE molecules comprising G4S linkers (Z5S vs. Q6S, Table 1).

BiTE molecules comprising the standard Anti-CD3 scFv without the engineered Anti-CD3 scFv cys-clamp, but the stabilized scFc domain showed 43.3% reduction in LMW in context of G4Q linker repeats versus G4S linker repeats (J1X vs. X7D, Table 1).

scFc Domain

The standard versus stabilized single chain Fc domain for BiTE molecules comprising G4Q linker repeats, the standard CD3 binder and an engineered Anti-CD3 scFv cys-clamp showed 3.2% reduction in LMW (F8I vs. M5J, Table 1).

In combination with the stabilized CD3 binder and the engineered Anti-CD3 scFv cys-clamp, the modified scFc reduced the LMW percentage by 34.0% (F1D vs. Q8I).

The G4Q linker repeats in combination with the standard anti-CD3 scFv without the engineered anti-CD3 scFv cys-clamp showed ca. 37.5% less LMW for the stabilized scFc comprising BiTE molecule (Q6S vs. X7D, Table 1). Similarly, the stabilized scFc domain in BiTE molecules comprising the standard G4S linker repeats, the standard Anti-CD3 scFv without an engineered Cys-clamp showed ca. 26.2% less LMW than the standard scFc domain (Z5S vs. J1X, Table 1).

TABLE 1
Difference by stabilized anti-CD3 scFv

| Molecule | Target | CD3 | Linker | scFc | CD3 Cys Clamp | % LMW, 4 w 40° C. | % LMW Reduction |
|---|---|---|---|---|---|---|---|
| F8I | 1 | Standard | G4Q | Standard | Yes | 27.8% | 13.3% |
| F1D | | Stabilized | | | | 24.1% | |
| M5J | 1 | Standard | G4Q | Modified | Yes | 26.9% | 40.9% |
| Q8I | | Stabilized | | | | 15.9% | |

Difference by G₄Q Linker repeats

| Molecule | Target | CD3 | Linker | scFc | CD3 Cys Clamp | % LMW, 4 w 40° C. | % LMW Reduction |
|---|---|---|---|---|---|---|---|
| 11D | 1 | Standard | G4S | Standard | Yes | 43.3% | 35.8% |
| F8I | | | G4Q | | | 27.8% | |
| Z5S | 2 | Standard | G4S | Standard | No | 21.9% | 32.3% |
| Q6S | | | G4Q | | | 14.8% | |
| J1X | 2 | Standard | G4S | Modified | No | 16.3% | 43.3% |
| X7D | | | G4Q | | | 9.2% | |

Difference by modified scFc

| Molecule | Target | CD3 | Linker | scFc | CD3 Cys Clamp? | % LMW, 4 w 40° C. | % LMW Reduction |
|---|---|---|---|---|---|---|---|
| F8I | 1 | Standard | G4Q | Standard | Yes | 27.8% | 3.2% |
| M5J | | | | Modified | | 26.9% | |
| F1D | 1 | Stabilized | G4Q | Standard | Yes | 24.1% | 34.0% |
| Q8I | | | | Modified | | 15.9% | |
| Z5S | 2 | Standard | G4S | Standard | No | 22.1% | 26.2% |
| J1X | | | | Modified | | 16.3% | |
| Q6S | 2 | Standard | G4Q | Standard | No | 14.8% | 37.5% |
| X7D | | | | Modified | | 9.2% | |

Difference by engineered CD3 cys-clamp

| Molecule | Target | CD3 | Linker | scFc | CD3 Cys Clamp | % LMW, 4 w 40° C. | % LMW Reduction |
|---|---|---|---|---|---|---|---|
| 11S | 1 | Standard | G4S | Standard | No | 29.0% | −49.3% |
| 11D | | | | | Yes | 43.3% | |
| M4T | 1 | Stabilized | G4Q | Standard | No | 23.6% | −2.1% |
| F1D | | | | | Yes | 24.1% | |
| S5Z | 1 | Stabilized | G4Q | Modified | No | 15.5% | −2.6% |
| Q8I | | | | | Yes | 15.9% | |

Anti-CD3 scFv Engineered Cys-Clamp

The introduction of an engineered cys-clamp in the anti-CD3 scFv led to an increase of LMW by 49.3% in a standard BiTE molecule comprising the standard anti-CD3 scFv, standard G4S linker repeats and the standard scFc domain compared to the non-CD3 cys-clamped BiTE molecule counterpart.

The BiTE molecule that comprised the stabilized anti-CD3 scFv, G4Q linker repeats and the standard single chain Fc domain only showed a 2.1% increase of LMW for the BiTE variant with the engineered cys-clamp versus the same BiTE molecule without the engineered cys-clamp.

The BiTE molecule comprising the stabilized anti-CD3 scFv and G4Q linker, but the modified single chain Fc domain showed 2.6% increase in LMW for the BiTE molecule including the CD3 engineered cys-clamp.

Notably, the LMW percentage was slightly for BiTE molecules with the engineered CD3 cys-clamp. However, the BiTE molecules including stabilized domains showed a lower LMW percentage compared to the reference molecule independently of the anti-CD3 scFv cys-clamp (ca. 15.5% LMW versus 29% without the CD3 scFv cys-clamp or 15.9% versus 43.3% respectively with the CD3 scFv cys-clamp).

Combinations

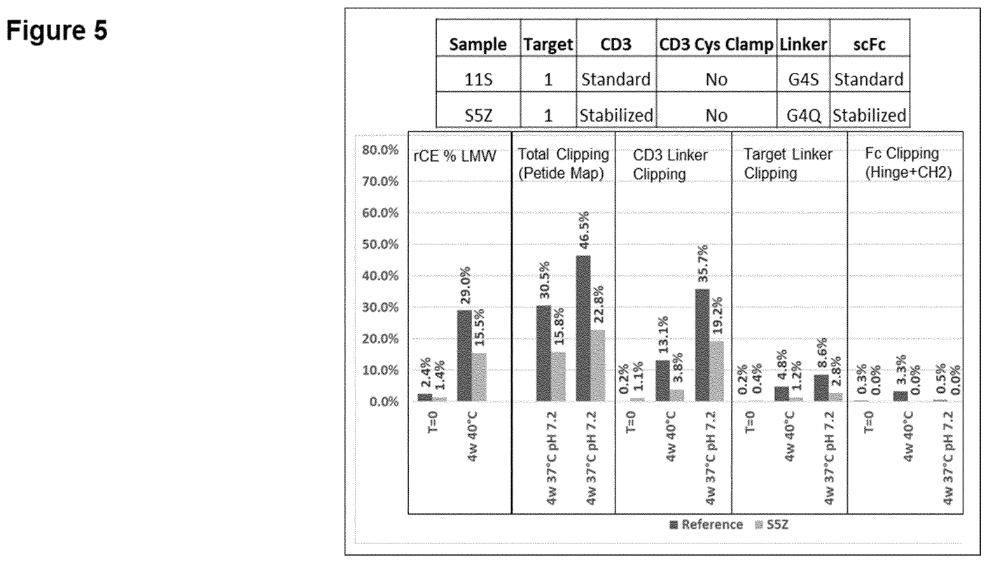

For target 1 binding molecules, the BiTE molecule S5Z showed the lowest LMW percentage compared to its reference molecule 11S (15.5% vs. 29%, FIG. 5). In detail, the reduction of total LMW by 46.6% is explained by a reduction in clipping of the anti-CD3 scFv linker, the target scFv linker and of the scFc domain.

For target 2 binding BiTE molecules, X7D showed 9.2% LMW in total compared to 22.1% LMW of the reference molecule, thus the stabilizations reduced the LMW by 58.2% by replacing G4S linker repeats with G4Q and the standard scFc domain with the stabilized scFc domain.

TABLE 2

| | | | | | | | | % |
|---|---|---|---|---|---|---|---|---|
| | | | | | | | Δ | Reduction |
| | | | | | | | % | in |
| | | | | | | | LMW | clipping |
| | | | | | | % | compared | compared |
| | | | | | CD3 | LMW | to | to |
| | | CD3 | | scFc | Cys | after | reference | reference |
| Sample | Target | binder | Linker | domain | clamp | 4 w 40° C. | molecule | molecule |
| 11S (Reference) | 1 | Standard | G4S | Standard | No | 29.0% | 0.0% | 0.0% |
| 11D | 1 | Standard | G4S | Standard | Yes | 43.3% | 14.3% | 49.3% |
| F8I | 1 | Standard | G4Q | Standard | Yes | 24.1% | −4.9% | −16.9% |
| M5J | 1 | Standard | G4Q | Stabilized | Yes | 27.8% | −1.2% | −4.1% |
| F1D | 1 | Stabilized | G4Q | Standard | Yes | 23.6% | −5.4% | −18.6% |
| M4T | 1 | Stabilized | G4Q | Standard | No | 26.9% | −2.1% | −7.2% |
| Q8I | 1 | Stabilized | G4Q | Stabilized | Yes | 15.9% | −13.1% | −45.2% |
| S5Z | 1 | Stabilized | G4Q | Stabilized | No | 15.5% | −13.5% | −46.6% |

TABLE 3

| | | | | | | | | % |
|---|---|---|---|---|---|---|---|---|
| | | | | | | | Δ | Reduction |
| | | | | | | | % | in |
| | | | | | | | LMW | clipping |
| | | | | | | % | compared | compared |
| | | | | | CD3 | LMW | to | to |
| | | CD3 | | scFc | Cys | after | reference | reference |
| Sample | Target | binder | Linker | domain | clamp | 4 w 40° C. | molecule | molecule |
| Z5S | 2 | Standard | G4S | Standard | No | 22.1% | 0.0% | 0.0% |
| J1X | 2 | Standard | G4S | Standard | No | 16.3% | −5.8% | −26.2% |
| Q6S | 2 | Standard | G4Q | Standard | No | 14.8% | −7.3% | −33.0% |
| X7D | 2 | Standard | G4Q | Standard | No | 9.2% | −12.9% | −58.2% |

### CONCLUSION

These observations show that the stabilization of a single domain or linker (e.g. anti-CD3 scFv, linker or scFc) by itself contributes to a reduction of LMW % after thermal stress. Furthermore, the combination of G4Q linkers, stabilized anti-CD3 scFv and stabilized in BiTE molecules showed additive effects on the total % LMW (FIG. 5, Table 2).

In the BiTE molecules tested comprising the standard anti-CD3 scFv, the standard linker and standard scFc domain, the engineered CD3 cys-clamp as a single modification showed an elevated % LMW level (43.3% vs. 29.0%) in the standard BiTE molecule. However, in the context of stabilized domains, the engineered CD3 cys-clamp showed less elevated LMW % levels compared to its non-cys-clamped counterparts (2.1% or 2.6%). Notably, the total level of % LMW after thermal stress was reduced in stabilized BiTE molecules comprising the CD3 cys-clamp compared to the standard control molecule 11D, as well as to the standard BiTE molecule without the anti-CD3 scFv cys-clamp (11S).

Other Motifs that Show Clipping after Thermal Stress and Mitigations

In the anti-target scFvs of BiTE molecules, we have identified a clipping site at the N-terminus of VL domains starting with the amino acid sequence: DI. Here, we introduce single amino acid changes to mitigate this clipping site (e.g., D→E mutation).

Similarly, in BiTE constructs that comprise a VL-linker-VH order in one of the scFv subunits, a SSS motif that is built by the end of the VH (VSS) and a SG4X linker showed elevated clipping that is addressed by depletion of one Serine in the linker sequence (SG4X→G4X) to generate a SS motif, i.e. (VSSGGGGX).

### Sequences

Sequence List:

| SEQ | | | |
|---|---|---|---|
| ID NO | Designation | Organism | Sequence |
| 1 | G4 | artificial | GGGG |
| 2 | G4A | artificial | GGGGA |
| 3 | G4C | artificial | GGGGC |
| 4 | G4D | artificial | GGGGD |
| 5 | G4E | artificial | GGGGE |
| 6 | G4F | artificial | GGGGF |
| 7 | G4G | artificial | GGGGG |
| 8 | G4H | artificial | GGGGH |
| 9 | G4I | artificial | GGGGI |
| 10 | G4K | artificial | GGGGK |
| 11 | G4L | artificial | GGGGL |
| 12 | G4M | artificial | GGGGM |
| 13 | G4N | artificial | GGGGN |
| 14 | G4P | artificial | GGGGP |
| 15 | G4Q | artificial | GGGGQ |
| 16 | G4S | artificial | GGGGS |
| 17 | G4T | artificial | GGGGT |
| 18 | G4V | artificial | GGGGV |
| 19 | G4W | artificial | GGGGW |
| 20 | G4Y | artificial | GGGGY |
| 21 | SG4A | artificial | SGGGGA |
| 22 | SG4C | artificial | SGGGGC |
| 23 | SG4D | artificial | SGGGGD |
| 24 | SG4E | artificial | SGGGGE |
| 25 | SG4F | artificial | SGGGGF |
| 26 | SG4G | artificial | SGGGGG |
| 27 | SG4H | artificial | SGGGGH |
| 28 | SG4I | artificial | SGGGGI |
| 29 | SG4K | artificial | SGGGGK |
| 30 | SG4L | artificial | SGGGGL |
| 31 | SG4M | artificial | SGGGGM |
| 32 | SG4N | artificial | SGGGGN |
| 33 | SG4P | artificial | SGGGGP |
| 34 | SG4Q | artificial | SGGGGQ |
| 35 | SG4S | artificial | SGGGGS |
| 36 | SG4T | artificial | SGGGGT |
| 37 | SG4V | artificial | SGGGGV |
| 38 | SG4W | artificial | SGGGGW |
| 39 | SG4Y | artificial | SGGGGY |
| 40 | (G4A)3 | artificial | GGGGAGGGGAGGGGA |
| 41 | (G4C)3 | artificial | GGGGCGGGGCGGGGC |
| 42 | (G4D)3 | artificial | GGGGDGGGGDGGGGD |
| 43 | (G4E)3 | artificial | GGGGEGGGGEGGGGE |
| 44 | (G4F)3 | artificial | GGGGFGGGGFGGGGF |
| 45 | (G4G)3 | artificial | GGGGGGGGGGGGGGG |
| 46 | (G4H)3 | artificial | GGGGHGGGGHGGGGH |
| 47 | (G4I)3 | artificial | GGGGIGGGGIGGGGI |
| 48 | (G4K)3 | artificial | GGGGKGGGGKGGGGK |
| 49 | (G4L)3 | artificial | GGGGLGGGGLGGGGL |
| 50 | (G4M)3 | artificial | GGGGMGGGGMGGGGM |
| 51 | (G4N)3 | artificial | GGGGNGGGGNGGGGN |
| 52 | (G4P)3 | artificial | GGGGPGGGGPGGGGP |
| 53 | (G4Q)3 | artificial | GGGGQGGGGQGGGGQ |
| 54 | (G4S)3 | artificial | GGGGSGGGGSGGGGS |
| 55 | (G4T)3 | artificial | GGGGTGGGGTGGGGT |
| 56 | (G4V)3 | artificial | GGGGVGGGGVGGGGV |
| 57 | (G4W)3 | artificial | GGGGWGGGGWGGGGW |
| 58 | (G4Y)3 | artificial | GGGGYGGGGYGGGGY |
| 59 | (G4A)6 | artificial | GGGGAGGGGAGGGGAGGGGAGGGGAGGGGA |
| 60 | (G4C)6 | artificial | GGGGCGGGGCGGGGGGGGCGGGGCGGGGC |
| 61 | (G4D)6 | artificial | GGGGDGGGGDGGGGDGGGGDGGGGDGGGGD |
| 62 | (G4E)6 | artificial | GGGGEGGGGEGGGGEGGGGEGGGGEGGGGE |
| 63 | (G4F)6 | artificial | GGGGFGGGGFGGGGFGGGGFGGGGFGGGGF |
| 64 | (G4G)6 | artificial | GGGGGGGGGGGGGGGG(GGGGGGGGGGGGG |
| 65 | (G4H)6 | artificial | GGGGHGGGGHGGGGHGGGGHGGGGHGGGGH |
| 66 | (G4I)6 | artificial | GGGGIGGGGIGGGGIGGGGIGGGGIGGGGI |
| 67 | (G4K)6 | artificial | GGGGKGGGGKGGGGKGGGGKGGGGKGGGGK |
| 68 | (G4L)6 | artificial | GGGGLGGGGLGGGGLGGGGLGGGGLGGGGL |
| 69 | (G4M)6 | artificial | GGGGMGGGGMGGGGMGGGGMGGGGMGGGGM |
| 70 | (G4N)6 | artificial | GGGGNGGGGNGGGGNGGGGNGGGGNGGGGN |
| 71 | (G4P)6 | artificial | GGGGPGGGGPGGGGGGPGGGGPGGGGP |
| 72 | (G4Q)6 | artificial | GGGGQGGGGQGGGGGQGGGGQ |
| 73 | (G4S)6 | artificial | GGGGSGGGGSGGGSGGGGSGGGGS |
| 74 | (G4T)6 | artificial | GGGGTGGGGTGGGTGGGGTGGGGT |
| 75 | (G4V)6 | artificial | GGGGVGGGGVGGGGVGGGGVGGGGVGGGGV |
| 76 | (G4W)6 | artificial | GGGGWGGGGWGGGGWGGGGWGGGGWGGGGW |
| 77 | (G4Y)6 | artificial | GGGGYGGGGYGGGGYGGGGYGGGGYGGGGY |
| 78 | Fc_1/2 | artificial | DKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTC |
| | | | LVKGFYPSDIAVEWESNGQPENNYKTTPPVLDSDGSFFLYSKLTVDKSR |
| | | | WQQGNVFSCSVMHEALHNHYTQKSLSLSPGK |
| 79 | Fc_1_delGK | artificial | DKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTC |
| | | | LVKGFYPSDIAVEWESNGQPENNYKTTPPVLDSDGSFFLYSKLTVDKSR |
| | | | WQQGNVFSCSVMHEALHNHYTQKSLSLSP |
| 80 | Fc_clipopt_1/2 | artificial | CPPCPAPELLGGPSVFLFPPKPKDTLMISRTPEVTCVVVDVSHEEPEVK |
| | | | FNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGN |
| | | | VFSCSVMHEALHNHYTQKSLSLSPGK |
| 81 | Fc_clipopt_1_delGK | artificial | CPPCPAPELLGGPSVFLFPPKPKDTLMISRTPEVTCVVVDVSHEEPEVK |
| | | | FNWYVDGVEVHNAKTKPCEEQYGSTYRCVSVLTVLHQDWLNGKEYKCKV |
| | | | SNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGN |
| | | | VFSCSVMHEALHNHYTQKSLSLSP |
| 82 | 4F10.03-HCDR1 | artificial | KYAMN |
| 83 | 4F10.03-HCDR2 | artificial | RIRSKYNNYATYYADAVKD |
| 84 | 4F10.03-HCDR3 | artificial | AGNFGTSYISYWAY |
| 85 | 4F10.03-LCDR1 | artificial | GSSTGAVTSGNYPN |
| 86 | 4F10.03-LCDR2 | artificial | GTKFLAP |
| 87 | 4F10.03-LCDR3 | artificial | VLWYSNRWV |
| 88 | 4F10.03_CC-HCDR1 | artificial | KYAMN |
| 89 | 4F10.03_CC-HCDR2 | artificial | RIRSKYNNYATYYADAVKD |
| 90 | 4F10.03_CC-HCDR3 | artificial | AGNFGTSYISYWAY |
| 91 | 4F10.03_CC-LCDR1 | artificial | GSSTGAVTSGNYPN |
| 92 | 4F10.03_CC-LCDR2 | artificial | GTKFLAP |
| 93 | 4F10.03_CC-LCDR3 | artificial | VLWYSNRWV |
| 94 | 4F10.03_GQ-HCDR1 | artificial | KYAMN |
| 95 | 4F10.03_GQ-HCDR2 | artificial | RIRSKYNNYATYYADAVKD |
| 96 | 4F10.03_GQ-HCDR3 | artificial | AGNFGTSYISYWAY |
| 97 | 4F10.03_GQ-LCDR1 | artificial | GSSTGAVTSGNYPN |
| 98 | 4F10.03_GQ-LCDR2 | artificial | GTKFLAP |
| 99 | 4F10.03_GQ-LCDR3 | artificial | VLWYSNRWV |
| 100 | 4F10.03_GQ_CC-HCDR1 | artificial | KYAMN |
| 101 | 4F10.03_GQ_CC-HCDR2 | artificial | RIRSKYNNYATYYADAVKD |
| 102 | 4F10.03_GQ_CC-HCDR3 | artificial | AGNFGTSYISYWAY |
| 103 | 4F10.03_GQ_CC-LCDR1 | artificial | GSSTGAVTSGNYPN |
| 104 | 4F10.03_GQ_CC-LCDR2 | artificial | GTKFLAP |
| 105 | 4F10.03_GQ_CC-LCDR3 | artificial | VLWYSNRWV |
| 106 | 5C3.01-HCDR1 | artificial | KYAIN |
| 107 | 5C3.01-HCDR2 | artificial | RIRSKYNNYATYYADAVKD |
| 108 | 5C3.01-HCDR3 | artificial | AGNFGSSYISYWAY |
| 109 | 5C3.01-LCDR1 | artificial | GSSTGAVTSGNYPN |
| 110 | 5C3.01-LCDR2 | artificial | GTKFLAP |
| 111 | 5C3.01-LCDR3 | artificial | VLWYSNRWV |
| 112 | 5C3.01_CC-HCDR1 | artificial | KYAIN |
| 113 | 5C3.01_CC-HCDR2 | artificial | RIRSKYNNYATYYADAVKD |
| 114 | 5C3.01_CC-HCDR3 | artificial | AGNFGSSYISYWAY |
| 115 | 5C3.01_CC-LCDR1 | artificial | GSSTGAVTSGNYPN |
| 116 | 5C3.01_CC-LCDR2 | artificial | GTKFLAP |
| 117 | 5C3.01_CC-LCDR3 | artificial | VLWYSNRWV |
| 118 | 5C3.01_GQ-HCDR1 | artificial | KYAIN |
| 119 | 5C3.01_GQ-HCDR2 | artificial | RIRSKYNNYATYYADAVKD |
| 120 | 5C3.01_GQ-HCDR3 | artificial | AGNFGSSYISYWAY |
| 121 | 5C3.01_GQ-LCDR1 | artificial | GSSTGAVTSGNYPN |
| 122 | 5C3.01_GQ-LCDR2 | artificial | GTKFLAP |
| 123 | 5C3.01_GQ-LCDR3 | artificial | VLWYSNRWV |
| 124 | 5C3.01_GQ_CC-HCDR1 | artificial | KYAIN |
| 125 | 5C3.01_GQ_CC-HCDR2 | artificial | RIRSKYNNYATYYADAVKD |
| 126 | 5C3.01_GQ_CC-HCDR3 | artificial | AGNFGSSYISYWAY |
| 127 | 5C3.01_GQ_CC-LCDR1 | artificial | GSSTGAVTSGNYPN |
| 128 | 5C3.01_GQ_CC-LCDR2 | artificial | GTKFLAP |
| 129 | 503.01_GQ_CC-LCDR3 | artificial | VLWYSNRWV |
| 130 | 5G6.05-HCDR1 | artificial | KYAMN |
| 131 | 5G6.05-HCDR2 | artificial | RIRSKYNNYATYYAEAVKG |
| 132 | 5G6.05-HCDR3 | artificial | NENIGTSYISYWAY |
| 133 | 5G6.05-LCDR1 | artificial | GSSTGAVTSGNYPN |
| 134 | 5G6.05-LCDR2 | artificial | GTKFLAP |
| 135 | 5G6.05-LCDR3 | artificial | VLWYSNRWV |
| 136 | 5G6.05_CC-HCDR1 | artificial | KYAMN |
| 137 | 5G6.05_CC-HCDR2 | artificial | RIRSKYNNYATYYAEAVKG |
| 138 | 5G6.05_CC-HCDR3 | artificial | NENIGTSYISYWAY |
| 139 | 5G6.05_CC-LCDR1 | artificial | GSSTGAVTSGNYPN |
| 140 | 5G6.05_CC-LCDR2 | artificial | GTKFLAP |
| 141 | 5G6.05_CC-LCDR3 | artificial | VLWYSNRWV |
| 142 | 5G6.05_GQ-HCDR1 | artificial | KYAMN |
| 143 | 5G6.05_GQ-HCDR2 | artificial | RIRSKYNNYATYYAEAVKG |
| 144 | 5G6.05_GQ-HCDR3 | artificial | NENIGTSYISYWAY |
| 145 | 5G6.05_GQ-LCDR1 | artificial | GSSTGAVTSGNYPN |
| 146 | 5G6.05_GQ-LCDR2 | artificial | GTKFLAP |
| 147 | 5G6.05_GQ-LCDR3 | artificial | VLWYSNRWV |
| 148 | 5G6.05_GQ_CC-HCDR1 | artificial | KYAMN |
| 149 | 5G6.05_GQ_CC-HCDR2 | artificial | RIRSKYNNYATYYAEAVKG |
| 150 | 5G6.05_GQ_CC-HCDR3 | artificial | NENIGTSYISYWAY |
| 151 | 5G6.05_GQ_CC-LCDR1 | artificial | GSSTGAVTSGNYPN |
| 152 | 5G6.05_GQ_CC-LCDR2 | artificial | GTKFLAP |
| 153 | 5G6.05_GQ_CC-LCDR3 | artificial | VLWYSNRWV |
| 154 | 6A8.02-HCDR1 | artificial | KYAIN |
| 155 | 6A8.02-HCDR2 | artificial | RIRSKYNNYATYYADAVKD |
| 156 | 6A8.02-HCDR3 | artificial | NANFGTSYISYFAY |
| 157 | 6A8.02-LCDR1 | artificial | GSSTGAVTSGNYPN |
| 158 | 6A8.02-LCDR2 | artificial | GTKFLAP |
| 159 | 6A8.02-LCDR3 | artificial | VLWYSNRWV |
| 160 | 6A8.02_CC-HCDR1 | artificial | KYAIN |
| 161 | 6A8.02_CC-HCDR2 | artificial | RIRSKYNNYATYYADAVKD |
| 162 | 6A8.02_CC-HCDR3 | artificial | NANFGTSYISYFAY |
| 163 | 6A8.02_CC-LCDR1 | artificial | GSSTGAVTSGNYPN |
| 164 | 6A8.02_CC-LCDR2 | artificial | GTKFLAP |
| 165 | 6A8.02_CC-LCDR3 | artificial | VLWYSNRWV |
| 166 | 6A8.02_GQ-HCDR1 | artificial | KYAIN |
| 167 | 6A8.02_GQ-HCDR2 | artificial | RIRSKYNNYATYYADAVKD |
| 168 | 6A8.02_GQ-HCDR3 | artificial | NANFGTSYISYFAY |
| 169 | 6A8.02_GQ-LCDR1 | artificial | GSSTGAVTSGNYPN |
| 170 | 6A8.02_GQ-LCDR2 | artificial | GTKFLAP |
| 171 | 6A8.02_GQ-LCDR3 | artificial | VLWYSNRWV |
| 172 | 6A8.02_GQ_CC-HCDR1 | artificial | KYAIN |
| 173 | 6A8.02_GQ_CC-HCDR2 | artificial | RIRSKYNNYATYYADAVKD |
| 174 | 6A8.02_GQ_CC-HCDR3 | artificial | NANFGTSYISYFAY |
| 175 | 6A8.02_GQ_CC-LCDR1 | artificial | GSSTGAVTSGNYPN |
| 176 | 6A8.02_GQ_CC-LCDR2 | artificial | GTKFLAP |
| 177 | 6A8.02_GQ_CC-LCDR3 | artificial | VLWYSNRWV |
| 178 | 6H10.09-HCDR1 | artificial | KYAMN |
| 179 | 6H10.09-HCDR2 | artificial | RIRSKYNNYATYYADAVKD |
| 180 | 6H10.09-HCDR3 | artificial | AGNFGSSYISYFAY |
| 181 | 6H10.09-LCDR1 | artificial | GSSTGAVTSGNYPN |
| 182 | 6H10.09-LCDR2 | artificial | GTKFLAP |
| 183 | 6H10.09-LCDR3 | artificial | VLYYSNRWV |
| 184 | 6H10.09_CC-HCDR1 | artificial | KYAMN |
| 185 | 6H10.09_CC-HCDR2 | artificial | RIRSKYNNYATYYADAVKD |
| 186 | 6H10.09_CC-HCDR3 | artificial | AGNFGSSYISYFAY |
| 187 | 6H10.09_CC-LCDR1 | artificial | GSSTGAVTSGNYPN |
| 188 | 6H10.09_CC-LCDR2 | artificial | GTKFLAP |
| 189 | 6H10.09_CC-LCDR3 | artificial | VLYYSNRWV |
| 190 | 6H10.09_GQ-HCDR1 | artificial | KYAMN |
| 191 | 6H10.09_GQ-HCDR2 | artificial | RIRSKYNNYATYYADAVKD |
| 192 | 6H10.09_GQ-HCDR3 | artificial | AGNFGSSYISYFAY |
| 193 | 6H10.09_GQ-LCDR1 | artificial | GSSTGAVTSGNYPN |
| 194 | 6H10.09_GQ-LCDR2 | artificial | GTKFLAP |
| 195 | 6H10.09_GQ-LCDR3 | artificial | VLYYSNRWV |
| 196 | 6H10.09_GQ_CC-HCDR1 | artificial | KYAMN |
| 197 | 6H10.09_GQ_CC-HCDR2 | artificial | RIRSKYNNYATYYADAVKD |
| 198 | 6H10.09_GQ_CC-HCDR3 | artificial | AGNFGSSYISYFAY |
| 199 | 6H10.09_GQ_CC-LCDR1 | artificial | GSSTGAVTSGNYPN |
| 200 | 6H10.09_GQ_CC-LCDR2 | artificial | GTKFLAP |
| 201 | 6H10.09_GQ_CC-LCDR3 | artificial | VLYYSNRWV |
| 202 | I2C-HCDR1 | artificial | KYAMN |
| 203 | I2C-HCDR2 | artificial | RIRSKYNNYATYYADSVKD |
| 204 | I2C-HCDR3 | artificial | HGNFGNSYISYWAY |
| 205 | I2C-LCDR1 | artificial | GSSTGAVTSGNYPN |
| 206 | I2C-LCDR2 | artificial | GTKFLAP |
| 207 | I2C-LCDR3 | artificial | VLWYSNRWV |
| 208 | I2C_CC-HCDR1 | artificial | KYAMN |
| 209 | I2C_CC-HCDR2 | artificial | RIRSKYNNYATYYADSVKD |
| 210 | I2C_CC-HCDR3 | artificial | HGNFGNSYISYWAY |
| 211 | I2C_CC-LCDR1 | artificial | GSSTGAVTSGNYPN |
| 212 | I2C_CC-LCDR2 | artificial | GTKFLAP |
| 213 | I2C_CC-LCDR3 | artificial | VLWYSNRWV |
| 214 | I2C_GQ-HCDR1 | artificial | KYAMN |
| 215 | I2C_GQ-HCDR2 | artificial | RIRSKYNNYATYYADSVKD |
| 216 | I2C_GQ-HCDR3 | artificial | HGNFGNSYISYWAY |
| 217 | I2C_GQ-LCDR1 | artificial | GSSTGAVTSGNYPN |
| 218 | I2C_GQ-LCDR2 | artificial | GTKFLAP |
| 219 | I2C_GQ-LCDR3 | artificial | VLWYSNRWV |
| 220 | I2C_GQ_CC-HCDR1 | artificial | KYAMN |
| 221 | I2C_GQ_CC-HCDR2 | artificial | RIRSKYNNYATYYADSVKD |
| 222 | I2C_GQ_CC-HCDR3 | artificial | HGNFGNSYISYWAY |
| 223 | I2C_GQ_CC-LCDR1 | artificial | GSSTGAVTSGNYPN |
| 224 | I2C_GQ_CC-LCDR2 | artificial | GTKFLAP |
| 225 | I2C_GQ_CC-LCDR3 | artificial | VLWYSNRWV |
| 226 | BC_A7_27-C4-G7- | artificial | NHIIH |
| | HCDR1 | | |
| 227 | BC_A7_27-C4-G7- | artificial | YINPYPGYHAYNEKFQG |
| | HCDR2 | | |
| 228 | BC_A7_27-C4-G7- | artificial | DGYYRDTDVLDY |
| | HCDR3 | | |
| 229 | BC_A7_27-C4-G7- | artificial | QASQDISNYLN |
| | LCDR1 | | |
| 230 | BC_A7_27-C4-G7- | artificial | YTSRLHT |
| | LCDR2 | | |
| 231 | BC_A7_27-C4-G7- | artificial | QQGNTLPWT |
| | LCDR3 | | |
| 232 | BC_A7_27-C4-G7_CC- | artificial | NHIIH |
| | HCDR1 | | |
| 233 | BC_A7_27-C4-G7_CC | artificial | YINPYPGYHAYNEKFQG |
| | HCDR2 | | |
| 234 | BC_A7_27-C4-G7_CC- | artificial | DGYYRDTDVLDY |
| | HCDR3 | | |
| 235 | BC_A7_27-C4-G7_CC- | artificial | QASQDISNYLN |
| | LCDR1 | | |
| 236 | BC_A7_27-C4-G7_CC- | artificial | YTSRLHT |
| | LCDR2 | | |
| 237 | BC_A7_27-C4-G7_CC- | artificial | QQGNTLPWT |
| | LCDR3 | | |
| 238 | BC_A7_27-C4- | artificial | NHIIH |
| | G7_CC_clipopt- | | |
| | HCDR1 | | |
| 239 | BC_A7_27-C4- | artificial | YINPYPGYHAYNEKFQG |
| | G7_CC_clipopt- | | |
| | HCDR2 | | |
| 240 | BC_A7_27-C4- | artificial | DGYYRDTDVLDY |
| | G7_CC_clipopt- | | |
| | HCDR3 | | |
| 241 | BC_A7_27-C4- | artificial | QASQDISNYLN |
| | G7_CC_clipopt- | | |
| | LCDR1 | | |
| 242 | BC_A7_27-C4- | artificial | YTSRLHT |
| | G7_CC_clipopt- | | |
| | LCDR2 | | |
| 243 | BC_A7_27-C4- | artificial | QQGNTLPWT |
| | G7_CC_clipopt- | | |
| | LCDR3 | | |
| 244 | BC_A7_27-C4- | artificial | NHIIH |
| | G7_clipopt-HCDR1 | | |
| 245 | BC_A7_27-C4- | artificial | YINPYPGYHAYNEKFQG |
| | G7_clipopt-HCDR2 | | |
| 246 | BC_A7_27-C4- | artificial | DGYYRDTDVLDY |
| | G7_clipopt-HCDR3 | | |
| 247 | BC_A7_27-C4- | artificial | QASQDISNYLN |
| | G7_clipopt-LCDR1 | | |
| 248 | BC_A7_27-C4- | artificial | YTSRLHT |
| | G7_clipopt-LCDR2 | | |
| 249 | BC_A7_27-C4- | artificial | QQGNTLPWT |
| | G7_clipopt-LCDR3 | | |
| 250 | CD123_24-B4-f_NK_ | artificial | HYAMS |
| | CC-HCDR1 | | |
| 251 | CD123_24-B4-f_NK_ | artificial | AVSGGGDKTLYADAVKG |
| | CC-HCDR2 | | |
| 252 | CD123_24-B4-f_NK_ | artificial | LRGFYYGMDV |
| | CC-HCDR3 | | |
| 253 | CD123_24-B4-f_NK_ | artificial | RSSQSLLHSNKYNYLD |
| | CC-LCDR1 | | |
| 254 | CD123_24-B4-f_NK_ | artificial | LGSNRAS |
| | CC-LCDR2 | | |
| 255 | CD123_24-B4-f_NK_ | artificial | MQALQTPPIT |
| | CC-LCDR3 | | |
| 256 | CD123_24-B4- | artificial | HYAMS |
| | f_NK_CC_clipopt- | | |
| | HCDR1 | | |
| 257 | CD123_24-B4- | artificial | AVSGGGDKTLYADAVKG |
| | f_NK_CC_clipopt- | | |
| | HCDR2 | | |
| 258 | CD123_24-B4- | artificial | LRGFYYGMDV |
| | f_NK_CC_clipopt- | | |
| | HCDR3 | | |
| 259 | CD123_24-B4- | artificial | RSSQSLLHSNKYNYLD |
| | f_NK_CC_clipopt- | | |
| | LCDR1 | | |
| 260 | CD123_24-B4- | artificial | LGSNRAS |
| | f_NK_CC_clipopt- | | |
| | LCDR2 | | |
| 261 | CD123_24-B4- | artificial | MQALQTPPIT |
| | f_NK_CC_clipopt- | | |
| | LCDR3 | | |
| 262 | CD19_97-G1RE- | artificial | SYGMH |
| | C2_LH_CC-HCDR1 | | |
| 263 | CD19_97-G1RE- | artificial | VISYEGSNKYYAESVKG |
| | C2_LH_CC-HCDR2 | | |
| 264 | CD19_97-G1RE- | artificial | DRGTIFGNYGLEV |
| | C2_LH_CC-HCDR3 | | |
| 265 | CD19_97-G1RE- | artificial | RSSQSLLHKNAFNYLD |
| | C2_LH_CC-LCDR1 | | |
| 266 | CD19_97-G1RE- | artificial | LGSNRAS |
| | C2_LH_CC-LCDR2 | | |
| 267 | CD19_97-G1RE- | artificial | MQALQTPFT |
| | C2_LH_CC-LCDR3 | | |
| 268 | CD19_97-G1RE- | artificial | SYGMH |
| | C2_LH_CC_clipopt- | | |
| | HCDR1 | | |
| 269 | CD19_97-G1RE- | artificial | VISYEGSNKYYAESVKG |
| | C2_LH_CC_clipopt- | | |
| | HCDR2 | | |
| 270 | CD19_97-G1RE- | artificial | DRGTIFGNYGLEV |
| | C2_LH_CC_clipopt- | | |
| | HCDR3 | | |
| 271 | CD19_97-G1RE- | artificial | RSSQSLLHKNAFNYLD |
| | C2_LH_CC_clipopt- | | |
| | LCDR1 | | |
| 272 | CD19_97-G1RE- | artificial | LGSNRAS |
| | C2_LH_CC_clipopt- | | |
| | LCDR2 | | |
| 273 | CD19_97-G1RE- | artificial | MQALQTPFT |
| | C2_LH_CC_clipopt- | | |
| | LCDR3 | | |
| 274 | CD33_E11-HCDR1 | artificial | NYGMN |
| 275 | CD33_E11-HCDR2 | artificial | WINTYTGEPTYADKFQG |
| 276 | CD33_E11-HCDR3 | artificial | WSWSDGYYVYFDY |
| 277 | CD33_E11-LCDR1 | artificial | KSSQSVLDSSTNKNSLA |
| 278 | CD33_E11-LCDR2 | artificial | WASTRES |
| 279 | CD33_E11-LCDR3 | artificial | QQSAHFPIT |
| 280 | CD33_E11_CC-HCDR1 | artificial | NYGMN |
| 281 | CD33_E11_CC-HCDR2 | artificial | WINTYTGEPTYADKFQG |
| 282 | CD33_E11_CC-HCDR3 | artificial | WSWSDGYYVYFDY |
| 283 | CD33_E11_CC-LCDR1 | artificial | KSSQSVLDSSTNKNSLA |
| 284 | CD33_E11_CC-LCDR2 | artificial | WASTRES |
| 285 | CD33_E11_CC-LCDR3 | artificial | QQSAHFPIT |
| 286 | CD33_E11_CC_GQ- | artificial | NYGMN |
| | HCDR1 | | |
| 287 | CD33_E11_CC_GQ- | artificial | WINTYTGEPTYADKFQG |
| | HCDR2 | | |
| 288 | CD33_E11_CC_GQ- | artificial | WSWSDGYYVYFDY |
| | HCDR3 | | |
| 289 | CD33_E11_CC_GQ- | artificial | KSSQSVLDSSTNKNSLA |
| | LCDR1 | | |
| 290 | CD33_E11_CC_GQ- | artificial | WASTRES |
| | LCDR2 | | |
| 291 | CD33_E11_CC_GQ- | artificial | QQSAHFPIT |
| | LCDR3 | | |
| 292 | CD33_E11_CC_GR- | artificial | NYGMN |
| | HCDR1 | | |
| 293 | CD33_E11_CC_GR- | artificial | WINTYTGEPTYADKFQG |
| | HCDR2 | | |
| 294 | CD33_E11_CC_GR- | artificial | WSWSDGYYVYFDY |
| | HCDR3 | | |
| 295 | CD33_E11_CC_GR- | artificial | KSSQSVLDSSTNKNSLA |
| | LCDR1 | | |
| 296 | CD33_E11_CC_GR- | artificial | WASTRES |
| | LCDR2 | | |
| 297 | CD33_E11_CC_GR- | artificial | QQSAHFPIT |
| | LCDR3 | | |
| 298 | CD33_E11_CC_clipopt- | artificial | NYGMN |
| | HCDR1 | | |
| 299 | CD33_E11_CC_clipopt- | artificial | WINTYTGEPTYADKFQG |
| | HCDR2 | | |
| 300 | CD33_E11_CC_clipopt- | artificial | WSWSDGYYVYFDY |
| | HCDR3 | | |
| 301 | CD33_E11_CC_clipopt- | artificial | KSSQSVLDSSTNKNSLA |
| | LCDR1 | | |
| 302 | CD33_E11_CC_clipopt- | artificial | WASTRES |
| | LCDR2 | | |
| 303 | CD33_E11_CC_clipopt- | artificial | QQSAHFPIT |
| | LCDR3 | | |
| 304 | CD33_E11 clipopt- | artificial | NYGMN |
| | HCDR1 | | |
| 305 | CD33_E11 clipopt- | artificial | WINTYTGEPTYADKFQG |
| | HCDR2 | | |
| 306 | CD33_E11 clipopt | artificial | WSWSDGYYVYFDY |
| | HCDR3 | | |
| 307 | CD33_E11 clipopt- | artificial | KSSQSVLDSSTNKNSLA |
| | LCDR1 | | |
| 308 | CD33_E11 clipopt- | artificial | WASTRES |
| | LCDR2 | | |
| 309 | CD33_E11 clipopt | artificial | QQSAHFPIT |
| | LCDR3 | | |
| 310 | CD70_1_C7D_CC- | artificial | TYAMS |
| | HCDR1 | | |
| 311 | CD70_1_C7D_CC- | artificial | AISGSGGRTFYAESVEG |
| | HCDR2 | | |
| 312 | CD70_1_C7D_CC- | artificial | HDYSNYPYFDY |
| | HCDR3 | | |
| 313 | CD70_1_C7D_CC- | artificial | RASQSVRSTYLA |
| | LCDR1 | | |
| 314 | CD70_1_C7D_CC- | artificial | GASSRAT |
| | LCDR2 | | |
| 315 | CD70_1_C7D_CC- | artificial | QQYGDLPFT |
| | LCDR3 | | |
| 316 | CD70_1_C7D_CC_ | artificial | TYAMS |
| | clipopt-HCDR1 | | |
| 317 | CD70_1_C7D_CC_ | artificial | AISGSGGRTFYAESVEG |
| | clipopt-HCDR2 | | |
| 318 | CD70_1_C7D_CC_ | artificial | HDYSNYPYFDY |
| | clipopt-HCDR3 | | |
| 319 | CD70_1_C7D_CC_ | artificial | RASQSVRSTYLA |
| | clipopt-LCDR1 | | |
| 320 | CD70_1_C7D_CC_ | artificial | GASSRAT |
| | clipopt-LCDR2 | | |
| 321 | CD70_1_C7D_CC_ | artificial | QQYGDLPFT |
| | clipopt-LCDR3 | | |
| 322 | CH19_2G6.007_CC- | artificial | SYGMH |
| | HCDR1 | | |
| 323 | CH19_2G6.007_CC- | artificial | FIWYEGSNKYYAESVKD |
| | HCDR2 | | |
| 324 | CH19_2G6.007_CC- | artificial | RAGIIGTIGYYYGMDV |
| | HCDR3 | | |
| 325 | CH19_2G6.007_CC- | artificial | SGDRLGEKYTS |
| | LCDR1 | | |
| 326 | CH19_2G6.007_CC- | artificial | QDTKRPS |
| | LCDR2 | | |
| 327 | CH19_2G6.007_CC- | artificial | QAWESSTVV |
| | LCDR3 | | |
| 328 | CH19_2G6.007_CC_ | artificial | SYGMH |
| | clipopt-HCDR1 | | |
| 329 | CH19_2G6.007_CC_ | artificial | FIWYEGSNKYYAESVKD |
| | clipopt-HCDR2 | | |
| 330 | CH19_2G6.007_CC_ | artificial | RAGIIGTIGYYYGMDV |
| | clipopt-HCDR3 | | |
| 331 | CH19_2G6.007_CC_ | artificial | SGDRLGEKYTS |
| | clipopt-LCDR1 | | |
| 332 | CH19_2G6.007_CC_ | artificial | QDTKRPS |
| | clipopt-LCDR2 | | |
| 333 | CH19_2G6.007_CC_ | artificial | QAWESSTVV |
| | clipopt-LCDR3 | | |
| 334 | CH3_15-E11_CC- | artificial | NYWMN |
| | HCDR1 | | |
| 335 | CH3_15-E11_CC- | artificial | NIAYGVKGTNYNQKFQG |
| | HCDR2 | | |
| 336 | CH3_15-E11_CC- | artificial | RYFYVMDY |
| | HCDR3 | | |
| 337 | CH3_15-E11_CC- | artificial | RASQDISNYLN |
| | LCDR1 | | |
| 338 | CH3_15-E11_CC- | artificial | YTSRLHS |
| | LCDR2 | | |
| 339 | CH3_15-E11_CC- | artificial | VQYAQFPLT |
| | LCDR3 | | |
| 340 | CH3_15- | artificial | NYWMN |
| | E11_CC_clipopt- | | |
| | HCDR1 | | |
| 341 | CH3_15- | artificial | NIAYGVKGTNYNQKFQG |
| | E11_CC_clipopt- | | |
| | HCDR2 | | |
| 342 | CH3_15- | artificial | RYFYVMDY |
| | E11_CC_clipopt- | | |
| | HCDR3 | | |
| 343 | CH3_15- | artificial | RASQDISNYLN |
| | E11_CC_clipopt- | | |
| | LCDR1 | | |
| 344 | CH3_15- | artificial | YTSRLHS |
| | E11_CC_clipopt- | | |
| | LCDR2 | | |
| 345 | CH3_15- | artificial | VQYAQFPLT |
| | E11_CC_clipopt- | | |
| | LCDR3 | | |
| 346 | CH3_15- | artificial | NYWMN |
| | E11_CC_clipopt_EI- | | |
| | HCDR1 | | |
| 347 | CH3_15- | artificial | NIAYGVKGTNYNQKFQG |
| | E11_CC_clipopt_EI- | | |
| | HCDR2 | | |
| 348 | CH3_15- | artificial | RYFYVMDY |
| | E11_CC_clipopt_EI- | | |
| | HCDR3 | | |
| 349 | CH3_15- | artificial | RASQDISNYLN |
| | E11_CC_clipopt_EI- | | |
| | LCDR1 | | |
| 350 | CH3_15- | artificial | YTSRLHS |
| | El1_CC_clipopt_EI- | | |
| | LCDR2 | | |
| 351 | CH3_15- | artificial | VQYAQFPLT |
| | E11_CC_clipopt_EI- | | |
| | LCDR3 | | |
| 352 | CH3_G8A_6-B12- | artificial | SYPIN |
| | HCDR1 | | |
| 353 | CH3_G8A_6-B12 | artificial | VIWTGGGTNYASSVKG |
| | HCDR2 | | |
| 354 | CH3_G8A_6-B12 | artificial | SRGVYDFDGRGAMDY |
| | HCDR3 | | |
| 355 | CH3_G8A_6-B12- | artificial | KSSQSLLYSSNQKNYFA |
| | LCDR1 | | |
| 356 | CH3_G8A_6-B12- | artificial | WASTRES |
| | LCDR2 | | |
| 357 | CH3_G8A_6-B12- | artificial | QQYYSYPYT |
| | LCDR3 | | |
| 358 | CH3_G8A_6- | artificial | SYPIN |
| | B12_clipopt-HCDR1 | | |
| 359 | CH3_G8A_6- | artificial | VIWTGGGTNYASSVKG |
| | B12_clipopt-HCDR2 | | |
| 360 | CH3_G8A_6- | artificial | SRGVYDFDGRGAMDY |
| | B12_clipopt-HCDR3 | | |
| 361 | CH3_G8A_6- | artificial | KSSQSLLYSSNQKNYFA |
| | B12_clipopt-LCDR1 | | |
| 362 | CH3_G8A_6- | artificial | WASTRES |
| | B12_clipopt-LCDR2 | | |
| 363 | CH3_G8A_6- | artificial | QQYYSYPYT |
| | B12_clipopt-LCDR3 | | |
| 364 | CL_10D8_CC-HCDR1 | artificial | GYYMH |
| 365 | CL_10D8_CC-HCDR2 | artificial | WINPNSGGTKYAQKFQG |
| 366 | CL_10D8_CC-HCDR3 | artificial | DRITVAGTYYYYGMDV |
| 367 | CL_10D8_CC-LCDR1 | artificial | RASQGVNNWLA |
| 368 | CL_10D8_CC-LCDR2 | artificial | TASSLQS |
| 369 | CL_10D8_CC-LCDR3 | artificial | QQANSFPIT |
| 370 | CL_10D8_CC_clipopt- | artificial | GYYMH |
| | HCDR1 | | |
| 371 | CL_10D8_CC_clipopt- | artificial | WINPNSGGTKYAQKFQG |
| | HCDR2 | | |
| 372 | CL_10D8_CC_clipopt- | artificial | DRITVAGTYYYYGMDV |
| | HCDR3 | | |
| 373 | CL_10D8_CC_clipopt- | artificial | RASQGVNNWLA |
| | LCDR1 | | |
| 374 | CL_10D8_CC_clipopt- | artificial | TASSLQS |
| | LCDR2 | | |
| 375 | CL_10D8_CC_clipopt- | artificial | QQANSFPIT |
| | LCDR3 | | |
| 376 | CL1_7-D7_CC-HCDR1 | artificial | NYYMH |
| 377 | CL1_7-D7_CC-HCDR2 | artificial | WINPTSGGANYAQKFQG |
| 378 | CL1_7-D7_CC-HCDR3 | artificial | ESHAIQEGIWFDY |
| 379 | CL1_7-D7_CC-LCDR1 | artificial | RASQSISNYLN |
| 380 | CL1_7-D7_CC-LCDR2 | artificial | DASSLQS |
| 381 | CL1_7-D7_CC-LCDR3 | artificial | QQSYSFPLT |
| 382 | CL1_7-D7_CC_clipopt- | artificial | NYYMH |
| | HCDR1 | | |
| 383 | CL1_7-D7_CC_clipopt- | artificial | WINPTSGGANYAQKFQG |
| | HCDR2 | | |
| 384 | CL1_7-D7_CC_clipopt- | artificial | ESHAIQEGIWFDY |
| | HCDR3 | | |
| 385 | CL1_7-D7_CC_clipopt- | artificial | RASQSISNYLN |
| | LCDR1 | | |
| 386 | CL1_7-D7_CC_clipopt- | artificial | DASSLQS |
| | LCDR2 | | |
| 387 | CL1_7-D7_CC_clipopt- | artificial | QQSYSFPLT |
| | LCDR3 | | |
| 388 | CL6_3D4-01.G2_LH- | artificial | GYYMH |
| | HCDR1 | | |
| 389 | CL6_3D4-01.G2_LH- | artificial | WINPNSGETNYAQKFQG |
| | HCDR2 | | |
| 390 | CL6_3D4-01.G2_LH | artificial | DALIVVAPVTRDYYYYGMDV |
| | HCDR3 | | |
| 391 | CL6_3D4-01.G2_LH- | artificial | RASQSVSSSYLA |
| | LCDR1 | | |
| 392 | CL6_3D4-01.G2_LH- | artificial | GASSRAT |
| | LCDR2 | | |
| 393 | CL6_3D4-01.G2_LH- | artificial | QQYGSSPLT |
| | LCDR3 | | |
| 394 | CL6_3D4- | artificial | GYYMH |
| | 01.G2_LH_clipopt- | | |
| | HCDR1 | | |
| 395 | CL6_3D4- | artificial | WINPNSGETNYAQKFQG |
| | 01.G2_LH_clipopt- | | |
| | HCDR2 | | |
| 396 | CL6_3D4- | artificial | DALIVVAPVTRDYYYYGMDV |
| | 01.G2_LH_clipopt- | | |
| | HCDR3 | | |
| 397 | CL6_3D4- | artificial | RASQSVSSSYLA |
| | 01.G2_LH_clipopt- | | |
| | LCDR1 | | |
| 398 | CL6_3D4- | artificial | GASSRAT |
| | 01.G2_LH_clipopt- | | |
| | LCDR2 | | |
| 399 | CL6_3D4- | artificial | QQYGSSPLT |
| | 01.G2_LH_clipopt- | | |
| | LCDR3 | | |
| 400 | CS1_PDL241.12_LH_CC_ | artificial | SSWMN |
| | +R-HCDR1 | | |
| 401 | CS1_PDL241.12_LH_CC_ | artificial | RIYPGDADAKYNAKFKG |
| | +R-HCDR2 | | |
| 402 | CS1_PDL241.12_LH_CC_ | artificial | STMIATGAMDY |
| | +R-HCDR3 | | |
| 403 | CS1_PDL241.12_LH_CC_ | artificial | KASQDVSTAVA |
| | +R-LCDR1 | | |
| 404 | CS1_PDL241.12_LH_CC_ | artificial | SASYRYT |
| | +R-LCDR2 | | |
| 405 | CS1_PDL241.12_LH_CC_ | artificial | QQHYSTPPYT |
| | +R-LCDR3 | | |
| 406 | CS1_PDL241.12_LH_CC_ | artificial | SSWMN |
| | −R-HCDR1 | | |
| 407 | CS1_PDL241.12_LH_CC_ | artificial | RIYPGDADAKYNAKFKG |
| | −R-HCDR2 | | |
| 408 | CS1_PDL241.12_LH_CC_ | artificial | STMIATGAMDY |
| | −R-HCDR3 | | |
| 409 | CS1_PDL241.12_LH_CC_ | artificial | KASQDVSTAVA |
| | −R-LCDR1 | | |
| 410 | CS1_PDL241.12_LH_CC_ | artificial | SASYRYT |
| | −R-LCDR2 | | |
| 411 | CS1_PDL241.12_LH_CC_ | artificial | QQHYSTPPYT |
| | −R-LCDR3 | | |
| 412 | CS1_PDL241.12_LH_CC_ | artificial | SSWMN |
| | −R_clipopt-HCDR1 | | |
| 413 | CS1_PDL241.12_LH_CC_ | artificial | RIYPGDADAKYNAKFKG |
| | −R_clipopt-HCDR2 | | |
| 414 | CS1_PDL241.12_LH_CC_ | artificial | STMIATGAMDY |
| | −R_clipopt-HCDR3 | | |
| 415 | CS1_PDL241.12_LH_CC_ | artificial | KASQDVSTAVA |
| | −R_clipopt-LCDR1 | | |
| 416 | CS1_PDL241.12_LH_CC_ | artificial | SASYRYT |
| | −R_clipopt-LCDR2 | | |
| 417 | CS1_PDL241.12_LH_CC_ | artificial | QQHYSTPPYT |
| | −R_clipopt-LCDR3 | | |
| 418 | DL_8-A7_CC-HCDR1 | artificial | SYYWS |
| 419 | DL_8-A7_CC-HCDR2 | artificial | YVYYSGTTNYNPSLKS |
| 420 | DL_8-A7_CC-HCDR3 | artificial | IAVTGFYFDY |
| 421 | DL_8-A7_CC-LCDR1 | artificial | RASQRVNNNYLA |
| 422 | DL_8-A7_CC_LCDR2 | artificial | GASSRAT |
| 423 | DL_8-A7_CC-LCDR3 | artificial | QQYDRSPLT |
| 424 | DL_8-A7_CC_clipopt- | artificial | SYYWS |
| | HCDR1 | | |
| 425 | DL_8-A7_CC_clipopt- | artificial | YVYYSGTTNYNPSLKS |
| | HCDR2 | | |
| 426 | DL_8-A7_CC_clipopt- | artificial | IAVTGFYFDY |
| | HCDR3 | | |
| 427 | DL_8-A7_CC_clipopt- | artificial | RASQRVNNNYLA |
| | LCDR1 | | |
| 428 | DL_8-A7_CC_clipopt- | artificial | GASSRAT |
| | LCDR2 | | |
| 429 | DL_8-A7_CC_clipopt- | artificial | QQYDRSPLT |
| | LCDR3 | | |
| 430 | EGFRvIII_CC-HCDR1 | artificial | NYGMH |
| 431 | EGFRvIII_CC-HCDR2 | artificial | VIWYDGSDKYYADSVRG |
| 432 | EGFRvIII_CC-HCDR3 | artificial | DGYDILTGNPRDEDY |
| 433 | EGFRvIII_CC-LCDR1 | artificial | RSSQSLVHSDGNTYLS |
| 434 | EGFRvIII_CC-LCDR2 | artificial | RISRRES |
| 435 | EGFRvIII_CC-LCDR3 | artificial | MQSTHVPRT |
| 436 | EGFRvIII_CC_clipopt- | artificial | NYGMH |
| | HCDR1 | | |
| 437 | EGFRvIII_CC_clipopt- | artificial | VIWYDGSDKYYADSVRG |
| | HCDR2 | | |
| 438 | EGFRvIII_CC_clipopt- | artificial | DGYDILTGNPRDEDY |
| | HCDR3 | | |
| 439 | EGFRvIII_CC_clipopt- | artificial | RSSQSLVHSDGNTYLS |
| | LCDR1 | | |
| 440 | EGFRvIII_CC_clipopt- | artificial | RISRRES |
| | LCDR2 | | |
| 441 | EGFRvIII_CC_clipopt- | artificial | MQSTHVPRT |
| | LCDR3 | | |
| 442 | FL_7-A8_CC-HCDR1 | artificial | NARMGVS |
| 443 | FL_7-A8_CC-HCDR2 | artificial | HIFSNDEKSYSTSLKN |
| 444 | FL_7-A8_CC-HCDR3 | artificial | IVGYGSGWYGFFDY |
| 445 | FL_7-A8_CC-LCDR1 | artificial | RASQGIRNDLG |
| 446 | FL_7-A8_CC-LCDR2 | artificial | AASTLQS |
| 447 | FL_7-A8_CC-LCDR3 | artificial | LQHNSYPLT |
| 448 | FL_7-A8_CC_clipopt- | artificial | NARMGVS |
| | HCDR1 | | |
| 449 | FL_7-A8_CC_clipopt- | artificial | HIFSNDEKSYSTSLKN |
| | HCDR2 | | |
| 450 | FL_7-A8_CC_clipopt- | artificial | IVGYGSGWYGFFDY |
| | HCDR3 | | |
| 451 | FL_7-A8_CC_clipopt- | artificial | RASQGIRNDLG |
| | LCDR1 | | |
| 452 | FL_7-A8_CC_clipopt- | artificial | AASTLQS |
| | LCDR2 | | |
| 453 | FL_7-A8_CC_clipopt- | artificial | LQHNSYPLT |
| | LCDR3 | | |
| 454 | MA_10-B5_CC-HCDR1 | artificial | NAWMS |
| 455 | MA_10-B5_CC-HCDR2 | artificial | RIRSRSYGGTTDYAAPVKG |
| 456 | MA_10-B5_CC-HCDR3 | artificial | PSYSGSYYNYFSVMDV |
| 457 | MA_10-B5_CC-LCDR1 | artificial | RTSQSISSYLN |
| 458 | MA_10-B5_CC-LCDR2 | artificial | AASSLQG |
| 459 | MA_10-B5_CC-LCDR3 | artificial | QQTYSMPFT |
| 460 | MA_10-B5_CC_clipopt- | artificial | NAWMS |
| | HCDR1 | | |
| 461 | MA_10-B5_CC_clipopt- | artificial | RIRSRSYGGTTDYAAPVKG |
| | HCDR2 | | |
| 462 | MA_10-B5_CC_clipopt- | artificial | PSYSGSYYNYFSVMDV |
| | HCDR3 | | |
| 463 | MA_10-B5_CC_clipopt- | artificial | RTSQSISSYLN |
| | LCDR1 | | |
| 464 | MA_10-B5_CC_clipopt- | artificial | AASSLQG |
| | LCDR2 | | |
| 465 | MA_10-B5_CC_clipopt- | artificial | QQTYSMPFT |
| | LCDR3 | | |
| 466 | MA_3-G10_CC-HCDR1 | artificial | SYAMS |
| 467 | MA_3-G10_CC-HCDR2 | artificial | AISGSGGGTYYAASVKG |
| 468 | MA_3-G10_CC-HCDR3 | artificial | GKGVHLGFDY |
| 469 | MA_3-G10_CC-LCDR1 | artificial | GGNNIGSKSVH |
| 470 | MA_3-G10_CC-LCDR2 | artificial | DDNDRPS |
| 471 | MA_3-G10_CC-LCDR3 | artificial | QVWDYSGQRQV |
| 472 | MA_3-G10_CC_clipopt- | artificial | SYAMS |
| | HCDR1 | | |
| 473 | MA_3-G10_CC_clipopt- | artificial | AISGSGGGTYYAASVKG |
| | HCDR2 | | |
| 474 | MA_3-G10_CC_clipopt- | artificial | GKGVHLGFDY |
| | HCDR3 | | |
| 475 | MA_3-G10_CC_clipopt- | artificial | GGNNIGSKSVH |
| | LCDR1 | | |
| 476 | MA_3-G10_CC_clipopt- | artificial | DDNDRPS |
| | LCDR2 | | |
| 477 | MA_3-G10_CC_clipopt- | artificial | QVWDYSGQRQV |
| | LCDR3 | | |
| 478 | MS_15-B12_CC- | artificial | SSSYFWG |
| | HCDR1 | | |
| 479 | MS_15-B12_CC- | artificial | NIYYSGSSNYNPSLKS |
| | HCDR2 | | |
| 480 | MS_15-B12_CC- | artificial | LPRGDRDAFDI |
| | HCDR3 | | |
| 481 | MS_15-B12_CC | artificial | RASQGISNYLA |
| | LCDR1 | | |
| 482 | MS_15-B12_CC- | artificial | AASTLQS |
| | LCDR2 | | |
| 483 | MS_15-B12_CC- | artificial | QQSYSTPFT |
| | LCDR3 | | |
| 484 | MS_15- | artificial | SSSYFWG |
| | B12_CC_clipopt- | | |
| | HCDR1 | | |
| 485 | MS_15- | artificial | NIYYSGSSNYNPSLKS |
| | B12_CC_clipopt- | | |
| | HCDR2 | | |
| 486 | MS_15- | artificial | LPRGDRDAFDI |
| | B12_CC_clipopt- | | |
| | HCDR3 | | |
| 487 | MS_15- | artificial | RASQGISNYLA |
| | B12_CC_clipopt- | | |
| | LCDR1 | | |
| 488 | MS_15- | artificial | AASTLQS |
| | B12_CC_clipopt- | | |
| | LCDR2 | | |
| 489 | MS_15- | artificial | QQSYSTPFT |
| | B12_CC_clipopt- | | |
| | LCDR3 | | |
| 490 | MS_15- | artificial | SSSYFWG |
| | B12_CC_clipopt_EI- | | |
| | HCDR1 | | |
| 491 | MS_15- | artificial | NIYYSGSSNYNPSLKS |
| | B12_CC_clipopt_EI- | | |
| | HCDR2 | | |
| 492 | MS_15- | artificial | LPRGDRDAFDI |
| | B12_CC_clipopt_EI | | |
| | HCDR3 | | |
| 493 | MS_15- | artificial | RASQGISNYLA |
| | B12_CC_clipopt_EI- | | |
| | LCDR1 | | |
| 494 | MS_15- | artificial | AASTLQS |
| | B12_CC_clipopt_EI- | | |
| | LCDR2 | | |
| 495 | MS_15- | artificial | QQSYSTPFT |
| | B12_CC_clipopt_EI- | | |
| | LCDR3 | | |
| 496 | MS_5-F11-HCDR1 | artificial | DYYMT |
| 497 | MS_5-F11-HCDR2 | artificial | YISSSGSTIYYADSVKG |
| 498 | MS_5-F11-HCDR3 | artificial | DRNSHFDY |
| 499 | MS_5-F11-LCDR1 | artificial | RASQGINTWLA |
| 500 | MS_5-F11-LCDR2 | artificial | GASGLQS |
| 501 | MS_5-F11-LCDR3 | artificial | QQAKSFPRT |
| 502 | MS_5-F11_clipopt- | artificial | DYYMT |
| | HCDR1 | | |
| 503 | MS_5-F11_clipopt- | artificial | YISSSGSTIYYADSVKG |
| | HCDR2 | | |
| 504 | MS_5-F11_clipopt- | artificial | DRNSHFDY |
| | HCDR3 | | |
| 505 | MS_5-F11_clipopt- | artificial | RASQGINTWLA |
| | LCDR1 | | |
| 506 | MS_5-F11_clipopt- | artificial | GASGLQS |
| | LCDR2 | | |
| 507 | MS_5-F11_clipopt- | artificial | QQAKSFPRT |
| | LCDR3 | | |
| 508 | MU_8-B7_CC-HCDR1 | artificial | GYYWS |
| 509 | MU_8-B7_CC-HCDR2 | artificial | DIDASGSTKYNPSLKS |
| 510 | MU_8-B7_CC-HCDR3 | artificial | KKYSTVWSYFDN |
| 511 | MU_8-B7_CC-LCDR1 | artificial | SGDKLGDKYAS |
| 512 | MU_8-B7_CC-LCDR2 | artificial | QDRKRPS |
| 513 | MU_8-B7_CC-LCDR3 | artificial | QAWGSSTAV |
| 514 | MU_8-B7_CC_clipopt- | artificial | GYYWS |
| | HCDR1 | | |
| 515 | MU_8-B7_CC_clipopt- | artificial | DIDASGSTKYNPSLKS |
| | HCDR2 | | |
| 516 | MU_8-B7_CC_clipopt- | artificial | KKYSTVWSYFDN |
| | HCDR3 | | |
| 517 | MU_8-B7_CC_clipopt- | artificial | SGDKLGDKYAS |
| | LCDR1 | | |
| 518 | MU_8-B7_CC_clipopt- | artificial | QDRKRPS |
| | LCDR2 | | |
| 519 | MU_8-B7_CC_clipopt- | artificial | QAWGSSTAV |
| | LCDR3 | | |
| 520 | PM_76-B10-HCDR1 | artificial | DYYMY |
| 521 | PM_76-B10-HCDR2 | artificial | IISDGGYYTYYSDIIKG |
| 522 | PM_76-B10-HCDR3 | artificial | GFPLLRHGAMDY |
| 523 | PM_76-B10-LCDR1 | artificial | KASQNVDTNVA |
| 524 | PM_76-B10-LCDR2 | artificial | SASYRYS |
| 525 | PM_76-B10-LCDR3 | artificial | QQYDSYPYT |
| 526 | PM_76-B10.11_CC- | artificial | DYYMY |
| | HCDR1 | | |
| 527 | PM_76-B10.11_CC- | artificial | IISDGGYYTYYSDIIKG |
| | HCDR2 | | |
| 528 | PM_76-B10.11_CC- | artificial | GFPLLRHGAMDY |
| | HCDR3 | | |
| 529 | PM_76-B10.11_CC- | artificial | KASQNVDTNVA |
| | LCDR1 | | |
| 530 | PM_76-B10.11_CC- | artificial | SASYVYW |
| | LCDR2 | | |
| 531 | PM_76-B10.11_CC- | artificial | QQYDQQLIT |
| | LCDR3 | | |
| 532 | PM_76-B10.11_CC_GQ- | artificial | DYYMY |
| | HCDR1 | | |
| 533 | PM_76-B10.11_CC_GQ- | artificial | IISDGGYYTYYSDIIKG |
| | HCDR2 | | |
| 534 | PM_76-B10.11_CC_GQ- | artificial | GFPLLRHGAMDY |
| | HCDR3 | | |
| 535 | PM_76-B10.11_CC_GQ- | artificial | KASQNVDTNVA |
| | LCDR1 | | |
| 536 | PM_76-B10.11_CC_GQ- | artificial | SASYVYW |
| | LCDR2 | | |
| 537 | PM_76-B10.11_CC_GQ- | artificial | QQYDQQLIT |
| | LCDR3 | | |
| 538 | PM_76- | artificial | DYYMY |
| | B10.11_CC_clipopt- | | |
| | HCDR1 | | |
| 539 | PM_76- | artificial | IISDGGYYTYYSDIIKG |
| | B10.11_CC_clipopt- | | |
| | HCDR2 | | |
| 540 | PM_76- | artificial | GFPLLRHGAMDY |
| | B10.11_CC_clipopt- | | |
| | HCDR3 | | |
| 541 | PM_76- | artificial | KASQNVDTNVA |
| | B10.11_CC_clipopt- | | |
| | LCDR1 | | |
| 542 | PM_76- | artificial | SASYVYW |
| | B10.11_CC_clipopt- | | |
| | LCDR2 | | |
| 543 | PM_76- | artificial | QQYDQQLIT |
| | B10.11_CC_clipopt- | | |
| | LCDR3 | | |
| 544 | PM_76-B10_clipopt- | artificial | DYYMY |
| | HCDR1 | | |
| 545 | PM_76-B10_clipopt | artificial | IISDGGYYTYYSDIIKG |
| | HCDR2 | | |
| 546 | PM_76-B10_clipopt- | artificial | GFPLLRHGAMDY |
| | HCDR3 | | |
| 547 | PM_76-B10_clipopt- | artificial | KASQNVDTNVA |
| | LCDR1 | | |
| 548 | PM_76-B10_clipopt- | artificial | SASYRYS |
| | LCDR2 | | |
| 549 | PM_76-B10_clipopt- | artificial | QQYDSYPYT |
| | LCDR3 | | |
| 550 | 4F10.03-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYWGQGTLVTVSS |
| 551 | 4F10.03-VL | artificial | QTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRW |
| | | | VFGSGTKLTVL |
| 552 | 4F10.03_CC-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYCGQGTLVTVSS |
| 553 | 4F10.03_CC-VL | artificial | QTVVTQEPSLTVSPGGTVTITCGSSTGAVTSGNYPNWVQKKPGQCPRGL |
| | | | IGGTKFLAPGTPARFSGSLSGGKAALTLSGVQPEDEAEYYCVLWYSNRW |
| | | | VFGSGTKLTVL |
| 554 | 4F10.03_GQ-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYWGQGTLVTVSS |
| 555 | 4F10.03_GQ-VL | artificial | QTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRW |
| | | | VFGSGTKLTVL |
| 556 | 4F10.03_GQ_CC-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYCGQGTLVTVSS |
| 557 | 4F10.03_GQ_CC-VL | artificial | QTVVTQEPSLTVSPGGTVTITCGSSTGAVTSGNYPNWVQKKPGQCPRGL |
| | | | IGGTKFLAPGTPARFSGSLSGGKAALTLSGVQPEDEAEYYCVLWYSNRW |
| | | | VFGSGTKLTVL |
| 558 | 5C3.01-VH | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYWGQGTLVTVSS |
| 559 | 5C3.01-VL | artificial | QTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRW |
| | | | VFGSGTKLTVL |
| 560 | 5C3.01_CC-VH | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYCGQGTLVTVSS |
| 561 | 5C3.01_CC-VL | artificial | QTVVTQEPSLTVSPGGTVTITCGSSTGAVTSGNYPNWVQKKPGQCPRGL |
| | | | IGGTKFLAPGTPARFSGSLSGGKAALTLSGVQPEDEAEYYCVLWYSNRW |
| | | | VFGSGTKLTVL |
| 562 | 5C3.01_GQ-VH | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYWGQGTLVTVSS |
| 563 | 5C3.01_GQ-VL | artificial | QTVVTQEPSLTVSPGGTVTITCGSSTGAVTSGNYPNWVQKKPGQAPRGL |
| | | | IGGTKFLAPGTPARFSGSLSGGKAALTLSGVQPEDEAEYYCVLWYSNRW |
| | | | VFGSGTKLTVL |
| 564 | 5C3.01_GQ_CC-VH | artificial | EVQLVESGGGLVQPGGSLKLSCAASGFTENKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYCGQGTLVTVSS |
| 565 | 5C3.01_GQ_CC-VL | artificial | QTVVTQEPSLTVSPGGTVTITCGSSTGAVTSGNYPNWVQKKPGQCPRGL |
| | | | IGGTKFLAPGTPARFSGSLSGGKAALTLSGVQPEDEAEYYCVLWYSNRW |
| | | | VFGSGTKLTVL |
| 566 | 5G6.05-VH | artificial | EVQLVESGGGLVQPGGSLKLSCAASGFTFNKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYWGQGTLVTVSS |
| 567 | 5G6.05-VL | artificial | QTVVTQEPSLTVSPGGTVTITCGSSTGAVTSGNYPNWVQQKPGQAPRGL |
| | | | IGGTKFLAPGTPARFSGSLSGGKAALTLSGVQPEDEAEYYCVLWYSNRW |
| | | | VFGSGTKLTVL |
| 568 | 5G6.05_CC-VH | artificial | EVQLVESGGGLVQPGGSLKLSCAASGFTFNKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYCGQGTLVTVSS |
| 569 | 5G6.05_CC-VL | artificial | QTVVTQEPSLTVSPGGTVTITCGSSTGAVTSGNYPNWVQQKPGQCPRGL |
| | | | IGGTKFLAPGTPARFSGSLSGGKAALTLSGVQPEDEAEYYCVLWYSNRW |
| | | | VFGSGTKLTVL |
| 570 | 5G6.05_GQ-VH | artificial | EVQLVESGGGLVQPGGSLKLSCAASGFTENKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYWGQGTLVTVSS |
| 571 | 5G6.05_GQ-VL | artificial | QTVVTQEPSLTVSPGGTVTITCGSSTGAVTSGNYPNWVQQKPGQAPRGL |
| | | | IGGTKFLAPGTPARFSGSLSGGKAALTLSGVQPEDEAEYYCVLWYSNRW |
| | | | VFGSGTKLTVL |
| 572 | 5G6.05_GQ_CC-VH | artificial | EVQLVESGGGLVQPGGSLKLSCAASGFTENKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYCGQGTLVTVSS |
| 573 | 5G6.05_GQ_CC-VL | artificial | QTVVTQEPSLTVSPGGTVTITCGSSTGAVTSGNYPNWVQQKPGQCPRGL |
| | | | IGGTKFLAPGTPARFSGSLSGGKAALTLSGVQPEDEAEYYCVLWYSNRW |
| | | | VFGSGTKLTVL |
| 574 | 6A8.02-VH | artificial | EVQLVESGGGLVQPGGSLKLSCAASGFTENKYAINWVREAPGKGLEWVA |
| | | | RIRSKYNNYATYYADAVKDRFTISRDDSKNTAYLQMNNLKTEDTAVYYC |
| | | | VRNANFGTSYISYFAYWGQGTLVTVSS |
| 575 | 6A8.02-VL | artificial | QTVVTQEPSLTVSPGGTVTLTCGSSTGAVTSGNYPNWVQKKPGQAPRGL |
| | | | IGGTKFLAPGTPARFSGSLLGGKAALTLSGVQPEDEAEYYCVLWYSNRW |
| | | | VFGSGTKLTVL |
| 576 | 6A8.02_CC-VH | artificial | EVQLVESGGGLVQPGGSLKLSCAASGFTFNKYAINWVREAPGKGLEWVA |
| | | | RIRSKYNNYATYYADAVKDRFTISRDDSKNTAYLQMNNLKTEDTAVYYC |
| | | | VRNANFGTSYISYFAYCGQGTLVTVSS |
| 577 | 6A8.02_CC-VL | artificial | QTVVTQEPSLTVSPGGTVTLTCGSSTGAVTSGNYPNWVQKKPGQCPRGL |
| | | | IGGTKFLAPGTPARFSGSLLGGKAALTLSGVQPEDEAEYYCVLWYSNRW |
| | | | VFGSGTKLTVL |
| 578 | 6A8.02_GQ-VH | artificial | EVQLVESGGGLVQPGGSLKLSCAASGFTFNKYAINWVREAPGKGLEWVA |
| | | | RIRSKYNNYATYYADAVKDRFTISRDDSKNTAYLQMNNLKTEDTAVYYC |
| | | | VRNANFGTSYISYFAYWGQGTLVTVSS |
| 579 | 6A8.02_GQ-VL | artificial | QTVVTQEPSLTVSPGGTVTLTCGSSTGAVTSGNYPNWVQKKPGQAPRGL |
| | | | IGGTKFLAPGTPARFSGSLLGGKAALTLSGVQPEDEAEYYCVLWYSNRW |
| | | | VFGSGTKLTVL |
| 580 | 6A8.02_GQ_CC-VH | artificial | EVQLVESGGGLVQPGGSLKLSCAASGFTFNKYAINWVREAPGKGLEWVA |
| | | | RIRSKYNNYATYYADAVKDRFTISRDDSKNTAYLQMNNLKTEDTAVYYC |
| | | | VRNANFGTSYISYFAYCGQGTLVTVSS |
| 581 | 6A8.02_GQ_CC-VL | artificial | QTVVTQEPSLTVSPGGTVTLTCGSSTGAVTSGNYPNWVQKKPGQCPRGL |
| | | | IGGTKFLAPGTPARFSGSLLGGKAALTLSGVQPEDEAEYYCVLWYSNRW |
| | | | VFGSGTKLTVL |
| 582 | 6H10.09-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSS |
| 583 | 6H10.09-VL | artificial | QTVVTQEPSLTVSPGGTVTITCGSSTGAVTSGNYPNWIQKKPGQAPRGL |
| | | | IGGTKFLAPGTPARFSGSLEGGKAALTLSGVQPEDEAEYYCVLYYSNRW |
| | | | VFGSGTKLTVL |
| 584 | 6H10.09_CC-VH | artificial | EVQLVESGGGLVQPGGSLKLSCAASGFTENKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYCGQGTLVTVSS |
| 585 | 6H10.09_CC-VL | artificial | QTVVTQEPSLTVSPGGTVTITCGSSTGAVTSGNYPNWIQKKPGQCPRGL |
| | | | IGGTKFLAPGTPARFSGSLEGGKAALTLSGVQPEDEAEYYCVLYYSNRW |
| | | | VFGSGTKLTVL |
| 586 | 6H10.09_GQ-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSS |
| 587 | 6H10.09_GQ-VL | artificial | QTVVTQEPSLTVSPGGTVTITCGSSTGAVTSGNYPNWIQKKPGQAPRGL |
| | | | IGGTKFLAPGTPARFSGSLEGGKAALTLSGVQPEDEAEYYCVLYYSNRW |
| | | | VFGSGTKLTVL |
| 588 | 6H10.09_GQ_CC-VH | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYCGQGTLVTVSS |
| 589 | 6H10.09_GQ_CC-VL | artificial | QTVVTQEPSLTVSPGGTVTITCGSSTGAVTSGNYPNWIQKKPGQCPRGL |
| | | | IGGTKFLAPGTPARFSGSLEGGKAALTLSGVQPEDEAEYYCVLYYSNRW |
| | | | VFGSGTKLTVL |
| 590 | I2C-VH | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSS |
| 591 | I2C-VL | artificial | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVL |
| 592 | I2C_CC-VH | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYCGQGTLVTVSS |
| 593 | I2C_CC-VL | artificial | QTVVTQEPSLTVSPGGTVTLTCGSSTGAVTSGNYPNWVQQKPGQCPRGL |
| | | | IGGTKFLAPGTPARFSGSLLGGKAALTLSGVQPEDEAEYYCVLWYSNRW |
| | | | VFGGGTKLTVL |
| 594 | I2C_GQ-VH | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSS |
| 595 | I2C_GQ-VL | artificial | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVL |
| 596 | I2C_GQ_CC-VH | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYCGQGTLVTVSS |
| 597 | I2C_GQ_CC-VL | artificial | QTVVTQEPSLTVSPGGTVTLTCGSSTGAVTSGNYPNWVQQKPGQCPRGL |
| | | | IGGTKFLAPGTPARFSGSLLGGKAALTLSGVQPEDEAEYYCVLWYSNRW |
| | | | VFGGGTKLTVL |
| 598 | BC_A7_27-C4-G7-VH | artificial | QVQLVQSGAEVKKPGASVKVSCKASGYTFTNHIIHWVRQAPGQGLEWMG |
| | | | YINPYPGYHAYNEKFQGRATMTSDTSTSTVYMELSSLRSEDTAVYYCAR |
| | | | DGYYRDTDVLDYWGQGTLVTVSS |
| 599 | BC_A7_27-C4-G7-VL | artificial | DIQMTQSPSSLSASVGDRVTITCQASQDISNYLNWYQQKPGKAPKLLIY |
| | | | YTSRLHTGVPSRFSGSGSGTDFTFTISSLEPEDIATYYCQQGNTLPWTF |
| | | | GQGTKVEIK |
| 600 | BC_A7_27-C4-G7_CC- | artificial | QVQLVQSGAEVKKPGASVKVSCKASGYTFTNHIIHWVRQAPGQCLEWMG |
| | VH | | YINPYPGYHAYNEKFQGRATMTSDTSTSTVYMELSSLRSEDTAVYYCAR |
| | | | DGYYRDTDVLDYWGQGTLVTVSS |
| 601 | BC_A7_27-C4-G7_CC- | artificial | DIQMTQSPSSLSASVGDRVTITCQASQDISNYLNWYQQKPGKAPKLLIY |
| | VL | | YTSRLHTGVPSRFSGSGSGTDFTFTISSLEPEDIATYYCQQGNTLPWTF |
| | | | GCGTKVEIK |
| 602 | BC_A7_27-C4- | artificial | QVQLVQSGAEVKKPGASVKVSCKASGYTFTNHIIHWVRQAPGQCLEWMG |
| | G7_CC_clipopt-VH | | YINPYPGYHAYNEKFQGRATMTSDTSTSTVYMELSSLRSEDTAVYYCAR |
| | | | DGYYRDTDVLDYWGQGTLVTVSS |
| 603 | BC_A7_27-C4- | artificial | EIQMTQSPSSLSASVGDRVTITCQASQDISNYLNWYQQKPGKAPKLLIY |
| | G7_CC_clipopt-VL | | YTSRLHTGVPSRFSGSGSGTDFTFTISSLEPEDIATYYCQQGNTLPWTF |
| | | | GCGTKVEIK |
| 604 | BC_A7_27-C4- | artificial | QVQLVQSGAEVKKPGASVKVSCKASGYTFTNHIIHWVRQAPGQGLEWMG |
| | G7_clipopt-VH | | YINPYPGYHAYNEKFQGRATMTSDTSTSTVYMELSSLRSEDTAVYYCAR |
| | | | DGYYRDTDVLDYWGQGTLVTVSS |
| 605 | BC_A7_27-C4- | artificial | EIQMTQSPSSLSASVGDRVTITCQASQDISNYLNWYQQKPGKAPKLLIY |
| | G7_clipopt-VL | | YTSRLHTGVPSRFSGSGSGTDFTFTISSLEPEDIATYYCQQGNTLPWTF |
| | | | GQGTKVEIK |
| 606 | CD123_24-B4-f_NK_CC- | artificial | EVQLLESGGGLVQPGGSLRLSCAASGFTFSHYAMSWVRQAPGKCLEWVS |
| | VH | | AVSGGGDKTLYADAVKGRFTISRDNSKNTLFLQMNSLRAEDTAIYYCAR |
| | | | LRGFYYGMDVWGQGTTVTVSS |
| 607 | CD123_24-B4-f_NK_CC- | artificial | DIVLTQSPLSLPVTPGEPASISCRSSQSLLHSNKYNYLDWYLQKPGQSP |
| | VL | | QLLIYLGSNRASGVPDRFSGSGSGTDFTLKISRVEAEDVGVYYCMQALQ |
| | | | TPPITFGCGTRLEIK |
| 608 | CD123_24-B4- | artificial | EVQLLESGGGLVQPGGSLRLSCAASGFTFSHYAMSWVRQAPGKCLEWVS |
| | f_NK_CC_clipopt- | | AVSGGGDKTLYADAVKGRFTISRDNSKNTLFLQMNSLRAEDTAIYYCAR |
| | VH | | LRGFYYGMDVWGQGTTVTVSS |
| 609 | CD123_24-B4- | artificial | EIVLTQSPLSLPVTPGEPASISCRSSQSLLHSNKYNYLDWYLQKPGQSP |
| | f_NK_CC_clipopt- | | QLLIYLGSNRASGVPDRFSGSGSGTDFTLKISRVEAEDVGVYYCMQALQ |
| | VL | | TPPITFGCGTRLEIK |
| 610 | CD19_97-G1RE- | artificial | QVQLVESGGGVVQPGRSLRLSCAASGFTFSSYGMHWVRQAPGKCLEWVA |
| | C2_LH_CC-VH | | VISYEGSNKYYAESVKGRFTISRDNSKNTLYLQMNSLRDEDTAVYYCAR |
| | | | DRGTIFGNYGLEVWGQGTTVTVSS |
| 611 | CD19_97-G1RE- | artificial | DIVMTQSPLSLPVISGEPASISCRSSQSLLHKNAFNYLDWYLQKPGQSP |
| | C2_LH_CC-VL | | QLLIYLGSNRASGVPDRFSGSGSGTDFTLKISRVEAEDVGVYYCMQALQ |
| | | | TPFTFGCGTKVDIK |
| 612 | CD19_97-G1RE- | artificial | QVQLVESGGGVVQPGRSLRLSCAASGFTFSSYGMHWVRQAPGKCLEWVA |
| | C2_LH_CC_clipopt- | | VISYEGSNKYYAESVKGRFTISRDNSKNTLYLQMNSLRDEDTAVYYCAR |
| | VH | | DRGTIFGNYGLEVWGQGTTVTVSS |
| 613 | CD19_97-G1RE- | artificial | DIVMTQSPLSLPVISGEPASISCRSSQSLLHKNAFNYLDWYLQKPGQSP |
| | C2_LH_CC_clipopt | | QLLIYLGSNRASGVPDRFSGSGSGTDFTLKISRVEAEDVGVYYCMQALQ |
| | VL | | TPFTFGCGTKVDIK |
| 614 | CD33_E11-VH | artificial | QVQLVQSGAEVKKPGESVKVSCKASGYTFTNYGMNWVKQAPGQGLEWMG |
| | | | WINTYTGEPTYADKFQGRVTMTTDTSTSTAYMEIRNLGGDDTAVYYCAR |
| | | | WSWSDGYYVYFDYWGQGTSVTVSS |
| 615 | CD33_E11-VL | artificial | DIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSA |
| | | | HFPITFGQGTRLEIK |
| 616 | CD33_E11_CC-VH | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSS |
| 617 | CD33_E11_CC-VL | artificial | DIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSA |
| | | | HFPITFGCGTRLEIK |
| 618 | CD33_E11_CC_GQ-VH | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSS |
| 619 | CD33_E11_CC_GQ-VL | artificial | DIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSA |
| | | | HFPITFGCGTRLEIK |
| 620 | CD33_E11_CC_GR-VH | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSS |
| 621 | CD33_E11_CC_GR-VL | artificial | DIVMTQSPDSLTVSLGERTTINCKSSQSVLDSSTNKNSLAWYQQKPGQP |
| | | | PKLLLSWASTRESGIPDRFSGSGSGTDFTLTIDSPQPEDSATYYCQQSA |
| | | | HFPITFGCGTRLEIK |
| 622 | CD33_E11_CC_clipopt- | artificial | QVQLVQSGAEVKKPGESVKVSCKASGYTFTNYGMNWVKQAPGQCLEWMG |
| | VH | | WINTYTGEPTYADKFQGRVTMTTDTSTSTAYMEIRNLGGDDTAVYYCAR |
| | | | WSWSDGYYVYFDYWGQGTSVTVSS |
| 623 | CD33_E11_CC_clipopt- | artificial | EIVMTQSPDSLTVSLGERTTINCKSSQSVLDSSTNKNSLAWYQQKPGQP |
| | VL | | PKLLLSWASTRESGIPDRFSGSGSGTDFTLTIDSPQPEDSATYYCQQSA |
| | | | HFPITFGCGTRLEIK |
| 624 | CD33_E11 clipopt- | artificial | QVQLVQSGAEVKKPGESVKVSCKASGYTFTNYGMNWVKQAPGQGLEWMG |
| | VH | | WINTYTGEPTYADKFQGRVTMTTDTSTSTAYMEIRNLGGDDTAVYYCAR |
| | | | WSWSDGYYVYFDYWGQGTSVTVSS |
| 625 | CD33_E11 clipopt- | artificial | EIVMTQSPDSLTVSLGERTTINCKSSQSVLDSSTNKNSLAWYQQKPGQP |
| | VL | | PKLLLSWASTRESGIPDRFSGSGSGTDFTLTIDSPQPEDSATYYCQQSA |
| | | | HFPITFGQGTRLEIK |
| 626 | CD70_1_C7D_CC-VH | artificial | EVQLLESGGGMVQPGGSLRLSCAASGFTFSTYAMSWVRQAPGKCLEWVS |
| | | | AISGSGGRTFYAESVEGRFTISRDNSKNTLYLQMNSLRAEDTAVYYCAK |
| | | | HDYSNYPYFDYWGQGTLVTVSS |
| 627 | CD70_1_C7D_CC-VL | artificial | EIVLTQSPGTLSLSPGERATLSCRASQSVRSTYLAWYQQKPGQAPRLLI |
| | | | YGASSRATGIPDRESGSGSGTDFTLTISRLEPEDFAVYSCQQYGDLPFT |
| | | | FGCGTKLEIK |
| 628 | CD70_1_C7D_CC_ | artificial | EVQLLESGGGMVQPGGSLRLSCAASGFTFSTYAMSWVRQAPGKCLEWVS |
| | clipopt-VH | | AISGSGGRTFYAESVEGRFTISRDNSKNTLYLQMNSLRAEDTAVYYCAK |
| | | | HDYSNYPYFDYWGQGTLVTVSS |
| 629 | CD70_1_C7D_CC_ | artificial | EIVLTQSPGTLSLSPGERATLSCRASQSVRSTYLAWYQQKPGQAPRLLI |
| | clipopt-VL | | YGASSRATGIPDRESGSGSGTDETLTISRLEPEDFAVYSCQQYGDLPFT |
| | | | FGCGTKLEIK |
| 630 | CH19_2G6.007_CC- | artificial | QVQLVESGGGVVQPGGSLRLSCAASGFTFSSYGMHWVRQAPGKCLEWVA |
| | VH | | FIWYEGSNKYYAESVKDRFTISRDNSKNTLYLQMNSLRAEDTAVYYCAR |
| | | | RAGIIGTIGYYYGMDVWGQGTTVTVSS |
| 631 | CH19_2G6.007_CC- | artificial | SYELTQPPSVSVSPGQTASITCSGDRLGEKYTSWYQQRPGQSPLLVIYQ |
| | VL | | DTKRPSGIPERFSGSNSGNTATLTISGTQAMDEADYYCQAWESSTVVFG |
| | | | CGTKLTVL |
| 632 | CH19_2G6.007_CC_ | artificial | QVQLVESGGGVVQPGGSLRLSCAASGFTFSSYGMHWVRQAPGKCLEWVA |
| | clipopt-VH | | FIWYEGSNKYYAESVKDRFTISRDNSKNTLYLQMNSLRAEDTAVYYCAR |
| | | | RAGIIGTIGYYYGMDVWGQGTTVTVSS |
| 633 | CH19_2G6.007_CC_ | artificial | SYELTQPPSVSVSPGQTASITCSGDRLGEKYTSWYQQRPGQSPLLVIYQ |
| | clipopt-VL | | DTKRPSGIPERFSGSNSGNTATLTISGTQAMDEADYYCQAWESSTVVFG |
| | | | CGTKLTVL |
| 634 | CH3_15-E11_CC-VH | artificial | QVQLVQSGAEVKKPGASVKVSCKASGYTFTNYWMNWVRQAPGQCLEWMG |
| | | | NIAYGVKGTNYNQKFQGRVTMTVDTSSSTAYMELSRLRSDDTAVYYCAT |
| | | | RYFYVMDYWGQGTLVTVSS |
| 635 | CH3_15-E11_CC-VL | artificial | DIQMTQSPSSLSASVGDRVTITCRASQDISNYLNWYQQKPGKVPKLLIY |
| | | | YTSRLHSGVPSRFSGSGSGTDFTLTISSLQPEDVATYYCVQYAQFPLTF |
| | | | GCGTKVEIK |
| 636 | CH3_15- | artificial | QVQLVQSGAEVKKPGASVKVSCKASGYTFTNYWMNWVRQAPGQCLEWMG |
| | E11_CC_clipopt-VH | | NIAYGVKGTNYNQKFQGRVTMTVDTSSSTAYMELSRLRSDDTAVYYCAT |
| | | | RYFYVMDYWGQGTLVTVSS |
| 637 | CH3_15 | artificial | DIQMTQSPSSLSASVGDRVTITCRASQDISNYLNWYQQKPGKVPKLLIY |
| | E11_CC_clipopt-VL | | YTSRLHSGVPSRFSGSGSGTDFTLTISSLQPEDVATYYCVQYAQFPLTF |
| | | | GCGTKVEIK |
| 638 | CH3_15- | artificial | QVQLVQSGAEVKKPGASVKVSCKASGYTFTNYWMNWVRQAPGQCLEWMG |
| | E11_CC_clipopt_EI- | | NIAYGVKGTNYNQKFQGRVTMTVDTSSSTAYMELSRLRSDDTAVYYCAT |
| | VH | | RYFYVMDYWGQGTLVTVSS |
| 639 | CH3_15- | artificial | EIQMTQSPSSLSASVGDRVTITCRASQDISNYLNWYQQKPGKVPKLLIY |
| | E11_CC_clipopt_EI- | | YTSRLHSGVPSRFSGSGSGTDFTLTISSLQPEDVATYYCVQYAQFPLTF |
| | VL | | GCGTKVEIK |
| 640 | CH3_G8A_6-B12-VH | artificial | EVQLLESGGGLVQPGGSLRLSCAASGFSFSSYPINWVRQAPGKGLEWVG |
| | | | VIWTGGGTNYASSVKGRFTISRDNSKNTVYLQMNSLRAEDTAVYYCAKS |
| | | | RGVYDFDGRGAMDYWGQGTLVTVSS |
| 641 | CH3_G8A_6-B12-VL | artificial | DIVMTQSPDSLAVSLGERATINCKSSQSLLYSSNQKNYFAWYQQKPGQP |
| | | | PKLLIYWASTRESGVPDRFSGSGSGTDFTLTISSLQAEDVAVYYCQQYY |
| | | | SYPYTFGQGTKLEIK |
| 642 | CH3_G8A_6- | artificial | EVQLLESGGGLVQPGGSLRLSCAASGFSFSSYPINWVRQAPGKGLEWVG |
| | B12_clipopt-VH | | VIWTGGGTNYASSVKGRFTISRDNSKNTVYLQMNSLRAEDTAVYYCAKS |
| | | | RGVYDFDGRGAMDYWGQGTLVTVSS |
| 643 | CH3_G8A_6- | artificial | EIVMTQSPDSLAVSLGERATINCKSSQSLLYSSNQKNYFAWYQQKPGQP |
| | B12_clipopt-VL | | PKLLIYWASTRESGVPDRFSGSGSGTDFTLTISSLQAEDVAVYYCQQYY |
| | | | SYPYTFGQGTKLEIK |
| 644 | CL_10D8_CC-VH | artificial | QVQLVQSGAEVKKPGASVKVSCKASGYTFTGYYMHWVRQAPGQCLEWMG |
| | | | WINPNSGGTKYAQKFQGRVTMTRDTSISTAYMELSRLRSDDTAVYYCAR |
| | | | DRITVAGTYYYYGMDVWGQGTTVTVSS |
| 645 | CL_10D8_CC-VL | artificial | DIQMTQSPSSVSASVGDRVTITCRASQGVNNWLAWYQQKPGKAPKLLIY |
| | | | TASSLQSGVPSRFSGSGSGTDFTLTIRSLQPEDFATYYCQQANSFPITF |
| | | | GCGTRLEIK |
| 646 | CL_10D8_CC_clipopt- | artificial | QVQLVQSGAEVKKPGASVKVSCKASGYTFTGYYMHWVRQAPGQCLEWMG |
| | VH | | WINPNSGGTKYAQKFQGRVTMTRDTSISTAYMELSRLRSDDTAVYYCAR |
| | | | DRITVAGTYYYYGMDVWGQGTTVTVSS |
| 647 | CL_10D8_CC_clipopt- | artificial | EIQMTQSPSSVSASVGDRVTITCRASQGVNNWLAWYQQKPGKAPKLLIY |
| | VL | | TASSLQSGVPSRFSGSGSGTDFTLTIRSLQPEDFATYYCQQANSFPITF |
| | | | GCGTRLEIK |
| 648 | CL1_7-D7_CC-VH | artificial | QVQLVQSGAEVKKPGASVKVSCKASGYTFTNYYMHWVRQAPGQCLEWMG |
| | | | WINPTSGGANYAQKFQGRVTMTRDTSISTAYMELSRLRSDDTAVYFCAR |
| | | | ESHAIQEGIWFDYWGQGTLVTVSS |
| 649 | CL1_7-D7_CC-VL | artificial | DIQMTQSPSSLSASVGDRVTISCRASQSISNYLNWYQQKPGKAPKLLIY |
| | | | DASSLQSGVPSRFSGSGSGTDFTLTISSLQPEDFATYYCQQSYSFPLTF |
| | | | GCGTKVEIK |
| 650 | CL1_7-D7_CC_clipopt- | artificial | QVQLVQSGAEVKKPGASVKVSCKASGYTFTNYYMHWVRQAPGQCLEWMG |
| | VH | | WINPTSGGANYAQKFQGRVTMTRDTSISTAYMELSRLRSDDTAVYFCAR |
| | | | ESHAIQEGIWFDYWGQGTLVTVSS |
| 651 | CL1_7-D7_CC_clipopt- | artificial | EIQMTQSPSSLSASVGDRVTISCRASQSISNYLNWYQQKPGKAPKLLIY |
| | VL | | DASSLQSGVPSRFSGSGSGTDFTLTISSLQPEDFATYYCQQSYSFPLTF |
| | | | GCGTKVEIK |
| 652 | CL6_3D4-01.G2_LH- | artificial | QVQLVQSGAEVKKPGASVKVSCKASGYTFTGYYMHWVRQAPGQCLEWMG |
| | VH | | WINPNSGETNYAQKFQGRVTMTRDTSISTAYMELSRLRSDDTAVYYCAR |
| | | | DALIVVAPVTRDYYYYGMDVWGQGTTVTVSS |
| 653 | CL6_3D4-01.G2_LH- | artificial | EIVLTQSPGTLSLSPGERATLSCRASQSVSSSYLAWYQQKPGQAPRLLI |
| | VL | | YGASSRATGIPDRESGSGSGTDFTLTISRLEPEDFAVYYCQQYGSSPLT |
| | | | FGCGTKLEIK |
| 654 | CL6_3D4- | artificial | QVQLVQSGAEVKKPGASVKVSCKASGYTFTGYYMHWVRQAPGQCLEWMG |
| | 01.G2_LH_clipopt- | | WINPNSGETNYAQKFQGRVTMTRDTSISTAYMELSRLRSDDTAVYYCAR |
| | VH | | DALIVVAPVTRDYYYYGMDVWGQGTTVTVSS |
| 655 | CL6_3D4- | artificial | EIVLTQSPGTLSLSPGERATLSCRASQSVSSSYLAWYQQKPGQAPRLLI |
| | 01.G2_LH_clipopt- | | YGASSRATGIPDRFSGSGSGTDFTLTISRLEPEDFAVYYCQQYGSSPLT |
| | VL | | FGCGTKLEIK |
| 656 | CS1_PDL241.12_LH_CC_ | artificial | QVQLVQSGAEVKKPGASVKVSCKASGYAFSSSWMNWVRQAPGQCLEWIG |
| | +R-VH | | RIYPGDADAKYNAKFKGKATLTADKSTSTAYMELSSLRSEDTAVYYCAR |
| | | | STMIATGAMDYWGQGTLVTVSS |
| 657 | CS1_PDL241.12_LH_CC_ | artificial | DIQMTQSPSSLSASVGDRVTITCKASQDVSTAVAWYQQKPGKAPKLLIY |
| | +R-VL | | SASYRYTGVPDRFTGSGSGTDFTLTISSLQPEDFATYYCQQHYSTPPYT |
| | | | FGCGTKVEIKR |
| 658 | CS1_PDL241.12_LH_CC_ | artificial | QVQLVQSGAEVKKPGASVKVSCKASGYAFSSSWMNWVRQAPGQCLEWIG |
| | −R-VH | | RIYPGDADAKYNAKFKGKATLTADKSTSTAYMELSSLRSEDTAVYYCAR |
| | | | STMIATGAMDYWGQGTLVTVSS |
| 659 | CS1_PDL241.12_LH_CC_ | artificial | DIQMTQSPSSLSASVGDRVTITCKASQDVSTAVAWYQQKPGKAPKLLIY |
| | −R-VL | | SASYRYTGVPDRFTGSGSGTDFTLTISSLQPEDFATYYCQQHYSTPPYT |
| | | | FGCGTKVEIK |
| 660 | CS1_PDL241.12_LH_CC_ | artificial | QVQLVQSGAEVKKPGASVKVSCKASGYAFSSSWMNWVRQAPGQCLEWIG |
| | −R_clipopt-VH | | RIYPGDADAKYNAKFKGKATLTADKSTSTAYMELSSLRSEDTAVYYCAR |
| | | | STMIATGAMDYWGQGTLVTVSS |
| 661 | CS1_PDL241.12_LH_CC_ | artificial | DIQMTQSPSSLSASVGDRVTITCKASQDVSTAVAWYQQKPGKAPKLLIY |
| | −R_clipopt-VL | | SASYRYTGVPDRFTGSGSGTDFTLTISSLQPEDFATYYCQQHYSTPPYT |
| | | | FGCGTKVEIK |
| 662 | DL_8-A7_CC-VH | artificial | QVQLQESGPGLVKPSETLSLTCTVSGGSISSYYWSWIRQPPGKCLEWIG |
| | | | YVYYSGTTNYNPSLKSRVTISVDTSKNQFSLKLSSVTAADTAVYYCASI |
| | | | AVTGFYFDYWGQGTLVTVSS |
| 663 | DL_8-A7_CC-VL | artificial | EIVLTQSPGTLSLSPGERVTLSCRASQRVNNNYLAWYQQRPGQAPRLLI |
| | | | YGASSRATGIPDRESGSGSGTDFTLTISRLEPEDFAVYYCQQYDRSPLT |
| | | | FGCGTKLEIK |
| 664 | DL_8-A7_CC_clipopt- | artificial | QVQLQESGPGLVKPSETLSLTCTVSGGSISSYYWSWIRQPPGKCLEWIG |
| | VH | | YVYYSGTTNYNPSLKSRVTISVDTSKNQFSLKLSSVTAADTAVYYCASI |
| | | | AVTGFYFDYWGQGTLVTVSS |
| 665 | DL_8-A7_CC_clipopt- | artificial | EIVLTQSPGTLSLSPGERVTLSCRASQRVNNNYLAWYQQRPGQAPRLLI |
| | VL | | YGASSRATGIPDRESGSGSGTDFTLTISRLEPEDFAVYYCQQYDRSPLT |
| | | | FGCGTKLEIK |
| 666 | EGFRvIII_CC-VH | artificial | QVQLVESGGGVVQSGRSLRLSCAASGFTFRNYGMHWVRQAPGKCLEWVA |
| | | | VIWYDGSDKYYADSVRGRFTISRDNSKNTLYLQMNSLRAEDTAVYYCAR |
| | | | DGYDILTGNPRDFDYWGQGTLVTVSS |
| 667 | EGFRvIII_CC-VL | artificial | DTVMTQTPLSSHVTLGQPASISCRSSQSLVHSDGNTYLSWLQQRPGQPP |
| | | | RLLIYRISRRFSGVPDRFSGSGAGTDFTLEISRVEAEDVGVYYCMQSTH |
| | | | VPRTFGCGTKVEIK |
| 668 | EGFRvIII_CC_clipopt- | artificial | QVQLVESGGGVVQSGRSLRLSCAASGFTFRNYGMHWVRQAPGKCLEWVA |
| | VH | | VIWYDGSDKYYADSVRGRFTISRDNSKNTLYLQMNSLRAEDTAVYYCAR |
| | | | DGYDILTGNPRDFDYWGQGTLVTVSS |
| 669 | EGFRvIII_CC_clipopt- | artificial | DTVMTQTPLSSHVTLGQPASISCRSSQSLVHSDGNTYLSWLQQRPGQPP |
| | VL | | RLLIYRISRRFSGVPDRESGSGAGTDFTLEISRVEAEDVGVYYCMQSTH |
| | | | VPRTFGCGTKVEIK |
| 670 | FL_7-A8_CC-VH | artificial | QVTLKESGPTLVKPTETLTLTCTLSGFSLNNARMGVSWIRQPPGKCLEW |
| | | | LAHIFSNDEKSYSTSLKNRLTISKDSSKTQVVLTMTNVDPVDTATYYCA |
| | | | RIVGYGSGWYGFFDYWGQGTLVTVSS |
| 671 | FL_7-A8_CC-VL | artificial | DIQMTQSPSSLSASVGDRVTITCRASQGIRNDLGWYQQKPGKAPKRLIY |
| | | | AASTLQSGVPSRFSGSGSGTEFTLTISSLQPEDFATYYCLQHNSYPLTF |
| | | | GCGTKVEIK |
| 672 | FL_7-A8_CC_clipopt- | artificial | QVTLKESGPTLVKPTETLTLTCTLSGFSLNNARMGVSWIRQPPGKCLEW |
| | VH | | LAHIFSNDEKSYSTSLKNRLTISKDSSKTQVVLTMTNVDPVDTATYYCA |
| | | | RIVGYGSGWYGFFDYWGQGTLVTVSS |
| 673 | FL_7-A8_CC_clipopt- | artificial | EIQMTQSPSSLSASVGDRVTITCRASQGIRNDLGWYQQKPGKAPKRLIY |
| | VL | | AASTLQSGVPSRFSGSGSGTEFTLTISSLQPEDFATYYCLQHNSYPLTF |
| | | | GCGTKVEIK |
| 674 | MA_10-B5_CC-VH | artificial | EVQLVESGGGLVQPGGSLRLSCAASGFTFSNAWMSWVRQAPGKCLEWVG |
| | | | RIRSRSYGGTTDYAAPVKGRFTISRDDSKNTLFLQMNSLKTEDTAVYYC |
| | | | TTPSYSGSYYNYFSVMDVWGQGTTVTVSS |
| 675 | MA_10-B5_CC-VL | artificial | DIQMTQSPSSLSASVGDRVTITCRTSQSISSYLNWYQQKPGRAPKLLIF |
| | | | AASSLQGGVPSRFSGSGSGTDFTLTISSLQPEDFATYYCQQTYSMPFTF |
| | | | GCGTKVEIK |
| 676 | MA_10-B5_CC_clipopt- | artificial | EVQLVESGGGLVQPGGSLRLSCAASGFTFSNAWMSWVRQAPGKCLEWVG |
| | VH | | RIRSRSYGGTTDYAAPVKGRFTISRDDSKNTLFLQMNSLKTEDTAVYYC |
| | | | TTPSYSGSYYNYFSVMDVWGQGTTVTVSS |
| 677 | MA_10-B5_CC_clipopt- | artificial | EIQMTQSPSSLSASVGDRVTITCRTSQSISSYLNWYQQKPGRAPKLLIF |
| | VL | | AASSLQGGVPSRFSGSGSGTDFTLTISSLQPEDFATYYCQQTYSMPFTF |
| | | | GCGTKVEIK |
| 678 | MA_3-G10_CC-VH | artificial | EVQLLESGGGLVQPGGSLRLSCAASGFTFSSYAMSWVRQAPGKCLEWVS |
| | | | AISGSGGGTYYAASVKGRFTISRDNSKNTLYLQMSSLRAEDTAVYYCAT |
| | | | GKGVHLGFDYWGQGTLVTVSS |
| 679 | MA_3-G10_CC-VL | artificial | SYVLTQPPSVSVAPGQTARITCGGNNIGSKSVHWYQQKPGQAPVMVVYD |
| | | | DNDRPSGIPERFSGSNSGNTATLTISRVEAGDEADYYCQVWDYSGQRQV |
| | | | FGCGTKLTVL |
| 680 | MA_3-G10_CC_clipopt- | artificial | EVQLLESGGGLVQPGGSLRLSCAASGFTFSSYAMSWVRQAPGKCLEWVS |
| | VH | | AISGSGGGTYYAASVKGRFTISRDNSKNTLYLQMSSLRAEDTAVYYCAT |
| | | | GKGVHLGFDYWGQGTLVTVSS |
| 681 | MA_3-G10_CC_clipopt- | artificial | SYVLTQPPSVSVAPGQTARITCGGNNIGSKSVHWYQQKPGQAPVMVVYD |
| | VL | | DNDRPSGIPERFSGSNSGNTATLTISRVEAGDEADYYCQVWDYSGQRQV |
| | | | FGCGTKLTVL |
| 682 | MS_15-B12_CC-VH | artificial | QVQLQESGPGLVKPSETLSLTCTVSGGSISSSSYFWGWIRQPPGKCLEW |
| | | | IGNIYYSGSSNYNPSLKSRVTISVDTSKNQFSLKLSSVTAADTAVYYCA |
| | | | RLPRGDRDAFDIWGQGTMVTVSS |
| 683 | MS_15-B12_CC-VL | artificial | DIVMTQSPSSLSASVGDRVTITCRASQGISNYLAWYQQKPGKVPKLLIY |
| | | | AASTLQSGVPSRFSGSGSGTDFTLTISSLQPEDFATYYCQQSYSTPFTF |
| | | | GCGTKVEIK |
| 684 | MS_15- | artificial | QVQLQESGPGLVKPSETLSLTCTVSGGSISSSSYFWGWIRQPPGKCLEW |
| | B12_CC_clipopt-VH | | IGNIYYSGSSNYNPSLKSRVTISVDTSKNQFSLKLSSVTAADTAVYYCA |
| | | | RLPRGDRDAFDIWGQGTMVTVSS |
| 685 | MS_15- | artificial | DIVMTQSPSSLSASVGDRVTITCRASQGISNYLAWYQQKPGKVPKLLIY |
| | B12_CC_clipopt-VL | | AASTLQSGVPSRFSGSGSGTDFTLTISSLQPEDFATYYCQQSYSTPFTF |
| | | | GCGTKVEIK |
| 686 | MS_15- | artificial | QVQLQESGPGLVKPSETLSLTCTVSGGSISSSSYFWGWIRQPPGKCLEW |
| | B12_CC_clipopt_EI- | | IGNIYYSGSSNYNPSLKSRVTISVDTSKNQFSLKLSSVTAADTAVYYCA |
| | VH | | RLPRGDRDAFDIWGQGTMVTVSS |
| 687 | MS_15- | artificial | EIVMTQSPSSLSASVGDRVTITCRASQGISNYLAWYQQKPGKVPKLLIY |
| | B12_CC_clipopt_EI- | | AASTLQSGVPSRFSGSGSGTDFTLTISSLQPEDFATYYCQQSYSTPFTF |
| | VL | | GCGTKVEIK |
| 688 | MS_5-F11-VH | artificial | QVQLVESGGGLVKPGGSLRLSCAASGFTFSDYYMTWIRQAPGKGLEWLS |
| | | | YISSSGSTIYYADSVKGRFTISRDNAKNSLFLQMNSLRAEDTAVYYCAR |
| | | | DRNSHFDYWGQGTLVTVSS |
| 689 | MS_5-F11-VL | artificial | DIQMTQSPSSVSASVGDRVTITCRASQGINTWLAWYQQKPGKAPKLLIY |
| | | | GASGLQSGVPSRFSGSGSGTDFTLTISSLQPEDFATYYCQQAKSFPRTF |
| | | | GQGTKVEIK |
| 690 | MS_5-F11_clipopt- | artificial | QVQLVESGGGLVKPGGSLRLSCAASGFTFSDYYMTWIRQAPGKGLEWLS |
| | VH | | YISSSGSTIYYADSVKGRFTISRDNAKNSLFLQMNSLRAEDTAVYYCAR |
| | | | DRNSHFDYWGQGTLVTVSS |
| 691 | MS_5-F11_clipopt- | artificial | EIQMTQSPSSVSASVGDRVTITCRASQGINTWLAWYQQKPGKAPKLLIY |
| | VL | | GASGLQSGVPSRFSGSGSGTDFTLTISSLQPEDFATYYCQQAKSFPRTF |
| | | | GQGTKVEIK |
| 692 | MU_8-B7_CC-VH | artificial | QVQLQQWGAGLLKPSETLSLTCAVYGGSFSGYYWSWIRQPPGKCLEWIG |
| | | | DIDASGSTKYNPSLKSRVTISLDTSKNQFSLKLNSVTAADTAVYFCARK |
| | | | KYSTVWSYFDNWGQGTLVTVSS |
| 693 | MU_8-B7_CC-VL | artificial | SYELTQPSSVSVPPGQTASITCSGDKLGDKYASWYQQKPGQSPVLVIYQ |
| | | | DRKRPSGVPERFSGSNSGNTATLTISGTQAMDEADYYCQAWGSSTAVFG |
| | | | CGTKLTVL |
| 694 | MU_8-B7_CC_clipopt- | artificial | QVQLQQWGAGLLKPSETLSLTCAVYGGSFSGYYWSWIRQPPGKCLEWIG |
| | VH | | DIDASGSTKYNPSLKSRVTISLDTSKNQFSLKLNSVTAADTAVYFCARK |
| | | | KYSTVWSYFDNWGQGTLVTVSS |
| 695 | MU_8-B7_CC_clipopt- | artificial | SYELTQPSSVSVPPGQTASITCSGDKLGDKYASWYQQKPGQSPVLVIYQ |
| | VL | | DRKRPSGVPERFSGSNSGNTATLTISGTQAMDEADYYCQAWGSSTAVFG |
| | | | CGTKLTVL |
| 696 | PM_76-B10-VH | artificial | QVQLVESGGGLVKPGESLRLSCAASGFTFSDYYMYWVRQAPGKGLEWVA |
| | | | IISDGGYYTYYSDIIKGRFTISRDNAKNSLYLQMNSLKAEDTAVYYCAR |
| | | | GFPLLRHGAMDYWGQGTLVTVSS |
| 697 | PM_76-B10-VL | artificial | DIQMTQSPSSLSASVGDRVTITCKASQNVDTNVAWYQQKPGQAPKSLIY |
| | | | SASYRYSDVPSRFSGSASGTDFTLTISSVQSEDFATYYCQQYDSYPYTF |
| | | | GGGTKLEIK |
| 698 | PM_76-B10.11_CC- | artificial | QVQLVESGGGLVKPGESLRLSCAASGFTFSDYYMYWVRQAPGKCLEWVA |
| | VH | | IISDGGYYTYYSDIIKGRFTISRDNAKNSLYLQMNSLKAEDTAVYYCAR |
| | | | GFPLLRHGAMDYWGQGTLVTVSS |
| 699 | PM_76-B10.11_CC- | artificial | DIQMTQSPSSLSASVGDRVTITCKASQNVDTNVAWYQQKPGQAPKSLIY |
| | VL | | SASYVYWDVPSRFSGSASGTDFTLTISSVQSEDFATYYCQQYDQQLITF |
| | | | GCGTKLEIK |
| 700 | PM_76-B10.11_CC_GQ- | artificial | QVQLVESGGGLVKPGESLRLSCAASGFTFSDYYMYWVRQAPGKCLEWVA |
| | VH | | IISDGGYYTYYSDIIKGRFTISRDNAKNSLYLQMNSLKAEDTAVYYCAR |
| | | | GFPLLRHGAMDYWGQGTLVTVSS |
| 701 | PM_76-B10.11_CC_GQ- | artificial | DIQMTQSPSSLSASVGDRVTITCKASQNVDTNVAWYQQKPGQAPKSLIY |
| | VL | | SASYVYWDVPSRFSGSASGTDFTLTISSVQSEDFATYYCQQYDQQLITE |
| | | | GCGTKLEIK |
| 702 | PM_76- | artificial | QVQLVESGGGLVKPGESLRLSCAASGFTFSDYYMYWVRQAPGKCLEWVA |
| | B10.11_CC_clipopt- | | IISDGGYYTYYSDIIKGRFTISRDNAKNSLYLQMNSLKAEDTAVYYCAR |
| | VH | | GFPLLRHGAMDYWGQGTLVTVSS |
| 703 | PM_76- | artificial | EIQMTQSPSSLSASVGDRVTITCKASQNVDTNVAWYQQKPGQAPKSLIY |
| | B10.11_CC_clipopt- | | SASYVYWDVPSRFSGSASGTDFTLTISSVQSEDFATYYCQQYDQQLITF |
| | VL | | GCGTKLEIK |
| 704 | PM_76-B10_clipopt- | artificial | QVQLVESGGGLVKPGESLRLSCAASGFTFSDYYMYWVRQAPGKGLEWVA |
| | VH | | IISDGGYYTYYSDIIKGRFTISRDNAKNSLYLQMNSLKAEDTAVYYCAR |
| | | | GFPLLRHGAMDYWGQGTLVTVSS |
| 705 | PM_76-B10_clipopt- | artificial | EIQMTQSPSSLSASVGDRVTITCKASQNVDTNVAWYQQKPGQAPKSLIY |
| | VL | | SASYRYSDVPSRFSGSASGTDFTLTISSVQSEDFATYYCQQYDSYPYTF |
| | | | GGGTKLEIK |
| 706 | 4F10.03 | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYWGQGTLVTVSSGGGGSGGGGSGGGGSQTVVTQE |
| | | | PSLTVSPGGTVTITCGSSTGAVTSGNYPNWVQKKPGQAPRGLIGGTKFL |
| | | | APGTPARFSGSLSGGKAALTLSGVQPEDEAEYYCVLWYSNRWVFGSGTK |
| | | | LTVL |
| 707 | 4F10.03_CC | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYCGQGTLVTVSSGGGGSGGGGSGGGGSQTVVTQE |
| | | | PSLTVSPGGTVTITCGSSTGAVTSGNYPNWVQKKPGQCPRGLIGGTKFL |
| | | | APGTPARFSGSLSGGKAALTLSGVQPEDEAEYYCVLWYSNRWVFGSGTK |
| | | | LTVL |
| 708 | 4F10.03_GQ | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYWGQGTLVTVSSGGGGQGGGGQGGGGQQTVVTQE |
| | | | PSLTVSPGGTVTITCGSSTGAVTSGNYPNWVQKKPGQAPRGLIGGTKFL |
| | | | APGTPARFSGSLSGGKAALTLSGVQPEDEAEYYCVLWYSNRWVFGSGTK |
| | | | LTVL |
| 709 | 4F10.03_GQ_CC | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGTSYISYWAYCGQGTLVTVSSGGGGQGGGGQGGGGQQTVVTQE |
| | | | PSLTVSPGGTVTITCGSSTGAVTSGNYPNWVQKKPGQCPRGLIGGTKFL |
| | | | APGTPARFSGSLSGGKAALTLSGVQPEDEAEYYCVLWYSNRWVFGSGTK |
| | | | LTVL |
| 710 | 5C3.01 | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYWGQGTLVTVSSGGGGSGGGGSGGGGSQTVVTQE |
| | | | PSLTVSPGGTVTITCGSSTGAVTSGNYPNWVQKKPGQAPRGLIGGTKFL |
| | | | APGTPARFSGSLSGGKAALTLSGVQPEDEAEYYCVLWYSNRWVFGSGTK |
| | | | LTVL |
| 711 | 5C3.01_CC | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYCGQGTLVTVSSGGGGSGGGGSGGGGSQTVVTQE |
| | | | PSLTVSPGGTVTITCGSSTGAVTSGNYPNWVQKKPGQCPRGLIGGTKFL |
| | | | APGTPARFSGSLSGGKAALTLSGVQPEDEAEYYCVLWYSNRWVFGSGTK |
| | | | LTVL |
| 712 | 5C3.01_GQ | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYWGQGTLVTVSSGGGGQGGGGQGGGGQQTVVTQE |
| | | | PSLTVSPGGTVTITCGSSTGAVTSGNYPNWVQKKPGQAPRGLIGGTKFL |
| | | | APGTPARFSGSLSGGKAALTLSGVQPEDEAEYYCVLWYSNRWVFGSGTK |
| | | | LTVL |
| 713 | 5C3.01_GQ_CC | artificial | EVQLVESGGGLVQPGGSLKLSCAASGFTFNKYAINWVRQAPGKGLEWVA |
| | | | RIRSKYNNYATYYADAVKDRFTISRDDSKNTVYLQMNNLKTEDTAVYYC |
| | | | ARAGNFGSSYISYWAYCGQGTLVTVSSGGGGQGGGGQGGGGQQTVVTQE |
| | | | PSLTVSPGGTVTITCGSSTGAVTSGNYPNWVQKKPGQCPRGLIGGTKFL |
| | | | APGTPARFSGSLSGGKAALTLSGVQPEDEAEYYCVLWYSNRWVFGSGTK |
| | | | LTVL |
| 714 | 5G6.05 | artificial | EVQLVESGGGLVQPGGSLKLSCAASGFTFNKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYWGQGTLVTVSSGGGGSGGGGSGGGGSQTVVTQE |
| | | | PSLTVSPGGTVTITCGSSTGAVTSGNYPNWVQQKPGQAPRGLIGGTKFL |
| | | | APGTPARFSGSLSGGKAALTLSGVQPEDEAEYYCVLWYSNRWVFGSGTK |
| | | | LTVL |
| 715 | 5G6.05_CC | artificial | EVQLVESGGGLVQPGGSLKLSCAASGFTFNKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYCGQGTLVTVSSGGGGSGGGGSGGGGSQTVVTQE |
| | | | PSLTVSPGGTVTITCGSSTGAVTSGNYPNWVQQKPGQCPRGLIGGTKFL |
| | | | APGTPARFSGSLSGGKAALTLSGVQPEDEAEYYCVLWYSNRWVFGSGTK |
| | | | LTVL |
| 716 | 5G6.05_GQ | artificial | EVQLVESGGGLVQPGGSLKLSCAASGFTFNKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYWGQGTLVTVSSGGGGQGGGGQGGGGQQTVVTQE |
| | | | PSLTVSPGGTVTITCGSSTGAVTSGNYPNWVQQKPGQAPRGLIGGTKFL |
| | | | APGTPARFSGSLSGGKAALTLSGVQPEDEAEYYCVLWYSNRWVFGSGTK |
| | | | LTVL |
| 717 | 5G6.05_GQ_CC | artificial | EVQLVESGGGLVQPGGSLKLSCAASGFTENKYAMNWVRQAPGKGLEWVA |
| | | | RIRSKYNNYATYYAEAVKGRFTISRDDSKNTVYLQMNNLKTEDTAVYYC |
| | | | VRNENIGTSYISYWAYCGQGTLVTVSSGGGGQGGGGQGGGGQQTVVTQE |
| | | | PSLTVSPGGTVTITCGSSTGAVTSGNYPNWVQQKPGQCPRGLIGGTKFL |
| | | | APGTPARFSGSLSGGKAALTLSGVQPEDEAEYYCVLWYSNRWVFGSGTK |
| | | | LTVL |
| 718 | 6A8.02 | artificial | EVQLVESGGGLVQPGGSLKLSCAASGFTENKYAINWVREAPGKGLEWVA |
| | | | RIRSKYNNYATYYADAVKDRFTISRDDSKNTAYLQMNNLKTEDTAVYYC |
| | | | VRNANFGTSYISYFAYWGQGTLVTVSSGGGGSGGGGSGGGGSQTVVTQE |
| | | | PSLTVSPGGTVTLTCGSSTGAVTSGNYPNWVQKKPGQAPRGLIGGTKFL |
| | | | APGTPARFSGSLLGGKAALTLSGVQPEDEAEYYCVLWYSNRWVFGSGTK |
| | | | LTVL |
| 719 | 6A8.02_CC | artificial | EVQLVESGGGLVQPGGSLKLSCAASGFTENKYAINWVREAPGKGLEWVA |
| | | | RIRSKYNNYATYYADAVKDRFTISRDDSKNTAYLQMNNLKTEDTAVYYC |
| | | | VRNANFGTSYISYFAYCGQGTLVTVSSGGGGSGGGGSGGGGSQTVVTQE |
| | | | PSLTVSPGGTVTLTCGSSTGAVTSGNYPNWVQKKPGQCPRGLIGGTKFL |
| | | | APGTPARFSGSLLGGKAALTLSGVQPEDEAEYYCVLWYSNRWVFGSGTK |
| | | | LTVL |
| 720 | 6A8.02_GQ | artificial | EVQLVESGGGLVQPGGSLKLSCAASGFTENKYAINWVREAPGKGLEWVA |
| | | | RIRSKYNNYATYYADAVKDRFTISRDDSKNTAYLQMNNLKTEDTAVYYC |
| | | | VRNANFGTSYISYFAYWGQGTLVTVSSGGGGQGGGGQGGGGQQTVVTQE |
| | | | PSLTVSPGGTVTLTCGSSTGAVTSGNYPNWVQKKPGQAPRGLIGGTKFL |
| | | | APGTPARFSGSLLGGKAALTLSGVQPEDEAEYYCVLWYSNRWVFGSGTK |
| | | | LTVL |
| 721 | 6A8.02_GQ_CC | artificial | EVQLVESGGGLVQPGGSLKLSCAASGFTFNKYAINWVREAPGKGLEWVA |
| | | | RIRSKYNNYATYYADAVKDRFTISRDDSKNTAYLQMNNLKTEDTAVYYC |
| | | | VRNANFGTSYISYFAYCGQGTLVTVSSGGGGQGGGGQGGGGQQTVVTQE |
| | | | PSLTVSPGGTVTLTCGSSTGAVTSGNYPNWVQKKPGQCPRGLIGGTKFL |
| | | | APGTPARFSGSLLGGKAALTLSGVQPEDEAEYYCVLWYSNRWVFGSGTK |
| | | | LTVL |
| 722 | 6H10.09 | artificial | EVQLVESGGGLVQPGGSLKLSCAASGFTENKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGSGGGGSGGGGSQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVL |
| 723 | 6H10.09_CC | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYCGQGTLVTVSSGGGGSGGGGSGGGGSQTVVTQE |
| | | | PSLTVSPGGTVTITCGSSTGAVTSGNYPNWIQKKPGQCPRGLIGGTKFL |
| | | | APGTPARFSGSLEGGKAALTLSGVQPEDEAEYYCVLYYSNRWVFGSGTK |
| | | | LTVL |
| 724 | 6H10.09_GQ | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRHGNFGNSYISYWAYWGQGTLVTVSSGGGGSGGGGSGGGGSQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVL |
| 725 | 6H10.09_GQ_CC | artificial | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYCGQGTLVTVSSGGGGQGGGGQGGGGQQTVVTQE |
| | | | PSLTVSPGGTVTITCGSSTGAVTSGNYPNWIQKKPGQCPRGLIGGTKFL |
| | | | APGTPARFSGSLEGGKAALTLSGVQPEDEAEYYCVLYYSNRWVFGSGTK |
| | | | LTVL |
| 726 | I2C | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSSGGGGSGGGGSGGGGSQTVVTQE |
| | | | PSLTVSPGGTVTLTCGSSTGAVTSGNYPNWVQQKPGQAPRGLIGGTKFL |
| | | | APGTPARFSGSLLGGKAALTLSGVQPEDEAEYYCVLWYSNRWVFGGGTK |
| | | | LTVL |
| 727 | I2C_CC | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYCGQGTLVTVSSGGGGSGGGGSGGGGSQTVVTQE |
| | | | PSLTVSPGGTVTLTCGSSTGAVTSGNYPNWVQQKPGQCPRGLIGGTKFL |
| | | | APGTPARFSGSLLGGKAALTLSGVQPEDEAEYYCVLWYSNRWVFGGGTK |
| | | | LTVL |
| 728 | I2C_GQ | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSSGGGGQGGGGQGGGGQQTVVTQE |
| | | | PSLTVSPGGTVTLTCGSSTGAVTSGNYPNWVQQKPGQAPRGLIGGTKFL |
| | | | APGTPARFSGSLLGGKAALTLSGVQPEDEAEYYCVLWYSNRWVFGGGTK |
| | | | LTVL |
| 729 | I2C_GQ_CC | artificial | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYCGQGTLVTVSSGGGGQGGGGQGGGGQQTVVTQE |
| | | | PSLTVSPGGTVTLTCGSSTGAVTSGNYPNWVQQKPGQCPRGLIGGTKFL |
| | | | APGTPARFSGSLLGGKAALTLSGVQPEDEAEYYCVLWYSNRWVFGGGTK |
| | | | LTVL |
| 730 | BC_A7_27-C4-G7 | artificial | QVQLVQSGAEVKKPGASVKVSCKASGYTFTNHIIHWVRQAPGQGLEWMG |
| | | | YINPYPGYHAYNEKFQGRATMTSDTSTSTVYMELSSLRSEDTAVYYCAR |
| | | | DGYYRDTDVLDYWGQGTLVTVSSGGGGSGGGGSGGGGSDIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGQGTKVEIK |
| 731 | BC_A7_27-C4-G7_CC | artificial | QVQLVQSGAEVKKPGASVKVSCKASGYTFTNHIIHWVRQAPGQCLEWMG |
| | | | YINPYPGYHAYNEKFQGRATMTSDTSTSTVYMELSSLRSEDTAVYYCAR |
| | | | DGYYRDTDVLDYWGQGTLVTVSSGGGGSGGGGSGGGGSDIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGCGTKVEIK |
| 732 | BC_A7_27-C4- | artificial | QVQLVQSGAEVKKPGASVKVSCKASGYTFTNHIIHWVRQAPGQCLEWMG |
| | G7_CC_clipopt | | YINPYPGYHAYNEKFQGRATMTSDTSTSTVYMELSSLRSEDTAVYYCAR |
| | | | DGYYRDTDVLDYWGQGTLVTVSSGGGGQGGGGQGGGGQEIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGCGTKVEIK |
| 733 | BC_A7_27-C4- | artificial | QVQLVQSGAEVKKPGASVKVSCKASGYTFTNHIIHWVRQAPGQGLEWMG |
| | G7_clipopt | | YINPYPGYHAYNEKFQGRATMTSDTSTSTVYMELSSLRSEDTAVYYCAR |
| | | | DGYYRDTDVLDYWGQGTLVTVSSGGGGQGGGGQGGGGQEIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGQGTKVEIK |
| 734 | CD123_24-B4-f_NK_CC | artificial | EVQLLESGGGLVQPGGSLRLSCAASGFTFSHYAMSWVRQAPGKCLEWVS |
| | | | AVSGGGDKTLYADAVKGRFTISRDNSKNTLFLQMNSLRAEDTAIYYCAR |
| | | | LRGFYYGMDVWGQGTTVTVSSGGGGSGGGGSGGGGSDIVLTQSPLSLPV |
| | | | TPGEPASISCRSSQSLLHSNKYNYLDWYLQKPGQSPQLLIYLGSNRASG |
| | | | VPDRFSGSGSGTDFTLKISRVEAEDVGVYYCMQALQTPPITFGCGTRLE |
| | | | IK |
| 735 | CD123_24-B4- | artificial | EVQLLESGGGLVQPGGSLRLSCAASGFTFSHYAMSWVRQAPGKCLEWVS |
| | f_NK_CC_clipopt | | AVSGGGDKTLYADAVKGRFTISRDNSKNTLFLQMNSLRAEDTAIYYCAR |
| | | | LRGFYYGMDVWGQGTTVTVSSGGGGQGGGGQGGGGQEIVLTQSPLSLPV |
| | | | TPGEPASISCRSSQSLLHSNKYNYLDWYLQKPGQSPQLLIYLGSNRASG |
| | | | VPDRFSGSGSGTDFTLKISRVEAEDVGVYYCMQALQTPPITFGCGTRLE |
| | | | IK |
| 736 | CD19_97-G1RE- | artificial | DIVMTQSPLSLPVISGEPASISCRSSQSLLHKNAFNYLDWYLQKPGQSP |
| | C2_LH_CC | | QLLIYLGSNRASGVPDRFSGSGSGTDFTLKISRVEAEDVGVYYCMQALQ |
| | | | TPFTFGCGTKVDIKGGGGSGGGGSGGGGSQVQLVESGGGVVQPGRSLRL |
| | | | SCAASGFTFSSYGMHWVRQAPGKCLEWVAVISYEGSNKYYAESVKGRFT |
| | | | ISRDNSKNTLYLQMNSLRDEDTAVYYCARDRGTIFGNYGLEVWGQGTTV |
| | | | TVSS |
| 737 | CD19_97-G1RE- | artificial | DIVMTQSPLSLPVISGEPASISCRSSQSLLHKNAFNYLDWYLQKPGQSP |
| | C2_LH_CC_clipopt | | QLLIYLGSNRASGVPDRFSGSGSGTDFTLKISRVEAEDVGVYYCMQALQ |
| | | | TPFTFGCGTKVDIKGGGGQGGGGQGGGGQQVQLVESGGGVVQPGRSLRL |
| | | | SCAASGFTFSSYGMHWVRQAPGKCLEWVAVISYEGSNKYYAESVKGRFT |
| | | | ISRDNSKNTLYLQMNSLRDEDTAVYYCARDRGTIFGNYGLEVWGQGTTV |
| | | | TVSS |
| 738 | CD33_E11 | artificial | QVQLVQSGAEVKKPGESVKVSCKASGYTFTNYGMNWVKQAPGQGLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGSGGGGSGGGGSDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGQGT |
| | | | RLEIK |
| 739 | CD33_E11_CC | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGSGGGGSGGGGSDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIK |
| 740 | CD33_E11_CC_GQ | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIK |
| 741 | CD33_E11_CC_GR | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGRGGGGRGGGGRDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIK |
| 742 | CD33_E11_CC_clipopt | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQEIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIK |
| 743 | CD33_E11 clipopt | artificial | QVQLVQSGAEVKKPGESVKVSCKASGYTFTNYGMNWVKQAPGQGLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQEIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGQGT |
| | | | RLEIK |
| 744 | CD70_1_C7D_CC | artificial | EVQLLESGGGMVQPGGSLRLSCAASGFTFSTYAMSWVRQAPGKCLEWVS |
| | | | AISGSGGRTFYAESVEGRFTISRDNSKNTLYLQMNSLRAEDTAVYYCAK |
| | | | HDYSNYPYFDYWGQGTLVTVSSGGGGSGGGGSGGGGSEIVLTQSPGTLS |
| | | | LSPGERATLSCRASQSVRSTYLAWYQQKPGQAPRLLIYGASSRATGIPD |
| | | | RFSGSGSGTDFTLTISRLEPEDFAVYSCQQYGDLPFTFGCGTKLEIK |
| 745 | CD70_1_C7D_CC_ | artificial | EVQLLESGGGMVQPGGSLRLSCAASGFTFSTYAMSWVRQAPGKCLEWVS |
| | clipopt | | AISGSGGRTFYAESVEGRFTISRDNSKNTLYLQMNSLRAEDTAVYYCAK |
| | | | HDYSNYPYFDYWGQGTLVTVSSGGGGQGGGGQGGGGQEIVLTQSPGTLS |
| | | | LSPGERATLSCRASQSVRSTYLAWYQQKPGQAPRLLIYGASSRATGIPD |
| | | | RFSGSGSGTDFTLTISRLEPEDFAVYSCQQYGDLPFTFGCGTKLEIK |
| 746 | CH19_2G6.007_CC | artificial | QVQLVESGGGVVQPGGSLRLSCAASGFTFSSYGMHWVRQAPGKCLEWVA |
| | | | FIWYEGSNKYYAESVKDRFTISRDNSKNTLYLQMNSLRAEDTAVYYCAR |
| | | | RAGIIGTIGYYYGMDVWGQGTTVTVSSGGGGSGGGGSGGGGSSYELTQP |
| | | | PSVSVSPGQTASITCSGDRLGEKYTSWYQQRPGQSPLLVIYQDTKRPSG |
| | | | IPERFSGSNSGNTATLTISGTQAMDEADYYCQAWESSTVVFGCGTKLTV |
| | | | L |
| 747 | CH19_2G6.007_CC_ | artificial | QVQLVESGGGVVQPGGSLRLSCAASGFTFSSYGMHWVRQAPGKCLEWVA |
| | clipopt | | FIWYEGSNKYYAESVKDRFTISRDNSKNTLYLQMNSLRAEDTAVYYCAR |
| | | | RAGIIGTIGYYYGMDVWGQGTTVTVSSGGGGQGGGGQGGGGQSYELTQP |
| | | | PSVSVSPGQTASITCSGDRLGEKYTSWYQQRPGQSPLLVIYQDTKRPSG |
| | | | IPERFSGSNSGNTATLTISGTQAMDEADYYCQAWESSTVVFGCGTKLTV |
| | | | L |
| 748 | CH3_15-E11_CC | artificial | QVQLVQSGAEVKKPGASVKVSCKASGYTFTNYWMNWVRQAPGQCLEWMG |
| | | | NIAYGVKGTNYNQKFQGRVTMTVDTSSSTAYMELSRLRSDDTAVYYCAT |
| | | | RYFYVMDYWGQGTLVTVSSGGGGSGGGGSGGGGSDIQMTQSPSSLSASV |
| | | | GDRVTITCRASQDISNYLNWYQQKPGKVPKLLIYYTSRLHSGVPSRFSG |
| | | | SGSGTDFTLTISSLQPEDVATYYCVQYAQFPLTFGCGTKVEIK |
| 749 | CH3_15- | artificial | QVQLVQSGAEVKKPGASVKVSCKASGYTFTNYWMNWVRQAPGQCLEWMG |
| | E11_CC_clipopt | | NIAYGVKGTNYNQKFQGRVTMTVDTSSSTAYMELSRLRSDDTAVYYCAT |
| | | | RYFYVMDYWGQGTLVTVSSGGGGQGGGGQGGGGQDIQMTQSPSSLSASV |
| | | | GDRVTITCRASQDISNYLNWYQQKPGKVPKLLIYYTSRLHSGVPSRFSG |
| | | | SGSGTDFTLTISSLQPEDVATYYCVQYAQFPLTFGCGTKVEIK |
| 750 | CH3_15- | artificial | QVQLVQSGAEVKKPGASVKVSCKASGYTFTNYWMNWVRQAPGQCLEWMG |
| | E11_CC_clipopt_EI | | NIAYGVKGTNYNQKFQGRVTMTVDTSSSTAYMELSRLRSDDTAVYYCAT |
| | | | RYFYVMDYWGQGTLVTVSSGGGGQGGGGQGGGGQEIQMTQSPSSLSASV |
| | | | GDRVTITCRASQDISNYLNWYQQKPGKVPKLLIYYTSRLHSGVPSRFSG |
| | | | SGSGTDFTLTISSLQPEDVATYYCVQYAQFPLTFGCGTKVEIK |
| 751 | CH3_G8A_6-B12 | artificial | EVQLLESGGGLVQPGGSLRLSCAASGFSFSSYPINWVRQAPGKGLEWVG |
| | | | VIWTGGGTNYASSVKGRFTISRDNSKNTVYLQMNSLRAEDTAVYYCAKS |
| | | | RGVYDFDGRGAMDYWGQGTLVTVSSGGGGSGGGGSGGGGSDIVMTQSPD |
| | | | SLAVSLGERATINCKSSQSLLYSSNQKNYFAWYQQKPGQPPKLLIYWAS |
| | | | TRESGVPDRFSGSGSGTDFTLTISSLQAEDVAVYYCQQYYSYPYTFGQG |
| | | | TKLEIK |
| 752 | CH3_G8A_6- | artificial | EVQLLESGGGLVQPGGSLRLSCAASGFSFSSYPINWVRQAPGKGLEWVG |
| | B12_clipopt | | VIWTGGGTNYASSVKGRFTISRDNSKNTVYLQMNSLRAEDTAVYYCAKS |
| | | | RGVYDFDGRGAMDYWGQGTLVTVSSGGGGQGGGGQGGGGQEIVMTQSPD |
| | | | SLAVSLGERATINCKSSQSLLYSSNQKNYFAWYQQKPGQPPKLLIYWAS |
| | | | TRESGVPDRFSGSGSGTDFTLTISSLQAEDVAVYYCQQYYSYPYTFGQG |
| | | | TKLEIK |
| 753 | CL_10D8_CC | artificial | QVQLVQSGAEVKKPGASVKVSCKASGYTFTGYYMHWVRQAPGQCLEWMG |
| | | | WINPNSGGTKYAQKFQGRVTMTRDTSISTAYMELSRLRSDDTAVYYCAR |
| | | | DRITVAGTYYYYGMDVWGQGTTVTVSSGGGGSGGGGSGGGGSDIQMTQS |
| | | | PSSVSASVGDRVTITCRASQGVNNWLAWYQQKPGKAPKLLIYTASSLQS |
| | | | GVPSRFSGSGSGTDFTLTIRSLQPEDFATYYCQQANSFPITFGCGTRLE |
| | | | IK |
| 754 | CL_10D8_CC_clipopt | artificial | QVQLVQSGAEVKKPGASVKVSCKASGYTFTGYYMHWVRQAPGQCLEWMG |
| | | | WINPNSGGTKYAQKFQGRVTMTRDTSISTAYMELSRLRSDDTAVYYCAR |
| | | | DRITVAGTYYYYGMDVWGQGTTVTVSSGGGGQGGGGQGGGGQEIQMTQS |
| | | | PSSVSASVGDRVTITCRASQGVNNWLAWYQQKPGKAPKLLIYTASSLQS |
| | | | GVPSRFSGSGSGTDFTLTIRSLQPEDFATYYCQQANSFPITFGCGTRLE |
| | | | IK |
| 755 | CL1_7-D7_CC | artificial | QVQLVQSGAEVKKPGASVKVSCKASGYTFTNYYMHWVRQAPGQCLEWMG |
| | | | WINPTSGGANYAQKFQGRVTMTRDTSISTAYMELSRLRSDDTAVYFCAR |
| | | | ESHAIQEGIWFDYWGQGTLVTVSSGGGGSGGGGSGGGGSDIQMTQSPSS |
| | | | LSASVGDRVTISCRASQSISNYLNWYQQKPGKAPKLLIYDASSLQSGVP |
| | | | SRFSGSGSGTDFTLTISSLQPEDFATYYCQQSYSFPLTFGCGTKVEIK |
| 756 | CL1_7-D7_CC_clipopt | artificial | QVQLVQSGAEVKKPGASVKVSCKASGYTFTNYYMHWVRQAPGQCLEWMG |
| | | | WINPTSGGANYAQKFQGRVTMTRDTSISTAYMELSRLRSDDTAVYFCAR |
| | | | ESHAIQEGIWFDYWGQGTLVTVSSGGGGQGGGGQGGGGQEIQMTQSPSS |
| | | | LSASVGDRVTISCRASQSISNYLNWYQQKPGKAPKLLIYDASSLQSGVP |
| | | | SRFSGSGSGTDFTLTISSLQPEDFATYYCQQSYSFPLTFGCGTKVEIK |
| 757 | CL6_3D4-01.G2_LH | artificial | EIVLTQSPGTLSLSPGERATLSCRASQSVSSSYLAWYQQKPGQAPRLLI |
| | | | YGASSRATGIPDRFSGSGSGTDFTLTISRLEPEDFAVYYCQQYGSSPLT |
| | | | FGCGTKLEIKGGGGSGGGGSGGGGSQVQLVQSGAEVKKPGASVKVSCKA |
| | | | SGYTFTGYYMHWVRQAPGQCLEWMGWINPNSGETNYAQKFQGRVTMTRD |
| | | | TSISTAYMELSRLRSDDTAVYYCARDALIVVAPVTRDYYYYGMDVWGQG |
| | | | TTVTVSS |
| 758 | CL6_3D4- | artificial | EIVLTQSPGTLSLSPGERATLSCRASQSVSSSYLAWYQQKPGQAPRLLI |
| | 01.G2_LH_clipopt | | YGASSRATGIPDRFSGSGSGTDFTLTISRLEPEDFAVYYCQQYGSSPLT |
| | | | FGCGTKLEIKGGGGQGGGGQGGGGQQVQLVQSGAEVKKPGASVKVSCKA |
| | | | SGYTFTGYYMHWVRQAPGQCLEWMGWINPNSGETNYAQKFQGRVTMTRD |
| | | | TSISTAYMELSRLRSDDTAVYYCARDALIVVAPVTRDYYYYGMDVWGQG |
| | | | TTVTVSS |
| 759 | CS1_PDL241.12_LH_CC_ | artificial | DIQMTQSPSSLSASVGDRVTITCKASQDVSTAVAWYQQKPGKAPKLLIY |
| | +R | | SASYRYTGVPDRFTGSGSGTDFTLTISSLQPEDFATYYCQQHYSTPPYT |
| | | | FGCGTKVEIKRGGGGSGGGGSGGGGSQVQLVQSGAEVKKPGASVKVSCK |
| | | | ASGYAFSSSWMNWVRQAPGQCLEWIGRIYPGDADAKYNAKFKGKATLTA |
| | | | DKSTSTAYMELSSLRSEDTAVYYCARSTMIATGAMDYWGQGTLVTVSS |
| 760 | CS1_PDL241.12_LH_CC_ | artificial | DIQMTQSPSSLSASVGDRVTITCKASQDVSTAVAWYQQKPGKAPKLLIY |
| | −R | | SASYRYTGVPDRFTGSGSGTDFTLTISSLQPEDFATYYCQQHYSTPPYT |
| | | | FGCGTKVEIKGGGGSGGGGSGGGGSQVQLVQSGAEVKKPGASVKVSCKA |
| | | | SGYAFSSSWMNWVRQAPGQCLEWIGRIYPGDADAKYNAKFKGKATLTAD |
| | | | KSTSTAYMELSSLRSEDTAVYYCARSTMIATGAMDYWGQGTLVTVSS |
| 761 | CS1_PDL241.12_LH_CC_ | artificial | DIQMTQSPSSLSASVGDRVTITCKASQDVSTAVAWYQQKPGKAPKLLIY |
| | −R_clipopt | | SASYRYTGVPDRFTGSGSGTDFTLTISSLQPEDFATYYCQQHYSTPPYT |
| | | | FGCGTKVEIKGGGGQGGGGQGGGGQQVQLVQSGAEVKKPGASVKVSCKA |
| | | | SGYAFSSSWMNWVRQAPGQCLEWIGRIYPGDADAKYNAKFKGKATLTAD |
| | | | KSTSTAYMELSSLRSEDTAVYYCARSTMIATGAMDYWGQGTLVTVSS |
| 762 | DL_8-A7_CC | artificial | QVQLQESGPGLVKPSETLSLTCTVSGGSISSYYWSWIRQPPGKCLEWIG |
| | | | YVYYSGTTNYNPSLKSRVTISVDTSKNQFSLKLSSVTAADTAVYYCASI |
| | | | AVTGFYFDYWGQGTLVTVSSGGGGSGGGGSGGGGSEIVLTQSPGTLSLS |
| | | | PGERVTLSCRASQRVNNNYLAWYQQRPGQAPRLLIYGASSRATGIPDRE |
| | | | SGSGSGTDFTLTISRLEPEDFAVYYCQQYDRSPLTFGCGTKLEIK |
| 763 | DL_8-A7_CC_clipopt | artificial | QVQLQESGPGLVKPSETLSLTCTVSGGSISSYYWSWIRQPPGKCLEWIG |
| | | | YVYYSGTTNYNPSLKSRVTISVDTSKNQFSLKLSSVTAADTAVYYCASI |
| | | | AVTGFYFDYWGQGTLVTVSSGGGGQGGGGQGGGGQEIVLTQSPGTLSLS |
| | | | PGERVTLSCRASQRVNNNYLAWYQQRPGQAPRLLIYGASSRATGIPDRF |
| | | | SGSGSGTDFTLTISRLEPEDFAVYYCQQYDRSPLTFGCGTKLEIK |
| 764 | EGFRvIII_CC | artificial | QVQLVESGGGVVQSGRSLRLSCAASGFTFRNYGMHWVRQAPGKCLEWVA |
| | | | VIWYDGSDKYYADSVRGRFTISRDNSKNTLYLQMNSLRAEDTAVYYCAR |
| | | | DGYDILTGNPRDFDYWGQGTLVTVSSGGGGSGGGGSGGGGSDTVMTQTP |
| | | | LSSHVTLGQPASISCRSSQSLVHSDGNTYLSWLQQRPGQPPRLLIYRIS |
| | | | RRFSGVPDRFSGSGAGTDFTLEISRVEAEDVGVYYCMQSTHVPRTFGCG |
| | | | TKVEIK |
| 765 | EGFRVIII_CC_clipopt | artificial | QVQLVESGGGVVQSGRSLRLSCAASGFTFRNYGMHWVRQAPGKCLEWVA |
| | | | VIWYDGSDKYYADSVRGRFTISRDNSKNTLYLQMNSLRAEDTAVYYCAR |
| | | | DGYDILTGNPRDFDYWGQGTLVTVSSGGGGQGGGGQGGGGQDTVMTQTP |
| | | | LSSHVTLGQPASISCRSSQSLVHSDGNTYLSWLQQRPGQPPRLLIYRIS |
| | | | RRFSGVPDRFSGSGAGTDFTLEISRVEAEDVGVYYCMQSTHVPRTFGCG |
| | | | TKVEIK |
| 766 | FL_7-A8_CC | artificial | QVTLKESGPTLVKPTETLTLTCTLSGFSLNNARMGVSWIRQPPGKCLEW |
| | | | LAHIFSNDEKSYSTSLKNRLTISKDSSKTQVVLTMTNVDPVDTATYYCA |
| | | | RIVGYGSGWYGFFDYWGQGTLVTVSSGGGGSGGGGSGGGGSDIQMTQSP |
| | | | SSLSASVGDRVTITCRASQGIRNDLGWYQQKPGKAPKRLIYAASTLQSG |
| | | | VPSRFSGSGSGTEFTLTISSLQPEDFATYYCLQHNSYPLTFGCGTKVEI |
| | | | K |
| 767 | FL_7-A8_CC_clipopt | artificial | QVTLKESGPTLVKPTETLTLTCTLSGFSLNNARMGVSWIRQPPGKCLEW |
| | | | LAHIFSNDEKSYSTSLKNRLTISKDSSKTQVVLTMTNVDPVDTATYYCA |
| | | | RIVGYGSGWYGFFDYWGQGTLVTVSSGGGGQGGGGQGGGGQEIQMTQSP |
| | | | SSLSASVGDRVTITCRASQGIRNDLGWYQQKPGKAPKRLIYAASTLQSG |
| | | | VPSRFSGSGSGTEFTLTISSLQPEDFATYYCLQHNSYPLTFGCGTKVEI |
| | | | K |
| 768 | MA_10-B5_CC | artificial | EVQLVESGGGLVQPGGSLRLSCAASGFTFSNAWMSWVRQAPGKCLEWVG |
| | | | RIRSRSYGGTTDYAAPVKGRFTISRDDSKNTLFLQMNSLKTEDTAVYYC |
| | | | TTPSYSGSYYNYFSVMDVWGQGTTVTVSSGGGGSGGGGSGGGGSDIQMT |
| | | | QSPSSLSASVGDRVTITCRTSQSISSYLNWYQQKPGRAPKLLIFAASSL |
| | | | QGGVPSRFSGSGSGTDFTLTISSLQPEDFATYYCQQTYSMPFTFGCGTK |
| | | | VEIK |
| 769 | MA_10-B5_CC_clipopt | artificial | EVQLVESGGGLVQPGGSLRLSCAASGFTFSNAWMSWVRQAPGKCLEWVG |
| | | | RIRSRSYGGTTDYAAPVKGRFTISRDDSKNTLFLQMNSLKTEDTAVYYC |
| | | | TTPSYSGSYYNYFSVMDVWGQGTTVTVSSGGGGQGGGGQGGGGQEIQMT |
| | | | QSPSSLSASVGDRVTITCRTSQSISSYLNWYQQKPGRAPKLLIFAASSL |
| | | | QGGVPSRFSGSGSGTDFTLTISSLQPEDFATYYCQQTYSMPFTFGCGTK |
| | | | VEIK |
| 770 | MA_3-G10_CC | artificial | EVQLLESGGGLVQPGGSLRLSCAASGFTFSSYAMSWVRQAPGKCLEWVS |
| | | | AISGSGGGTYYAASVKGRFTISRDNSKNTLYLQMSSLRAEDTAVYYCAT |
| | | | GKGVHLGFDYWGQGTLVTVSSGGGGSGGGGSGGGGSSYVLTQPPSVSVA |
| | | | PGQTARITCGGNNIGSKSVHWYQQKPGQAPVMVVYDDNDRPSGIPERFS |
| | | | GSNSGNTATLTISRVEAGDEADYYCQVWDYSGQRQVFGCGTKLTVL |
| 771 | MA_3-G10_CC_clipopt | artificial | EVQLLESGGGLVQPGGSLRLSCAASGFTFSSYAMSWVRQAPGKCLEWVS |
| | | | AISGSGGGTYYAASVKGRFTISRDNSKNTLYLQMSSLRAEDTAVYYCAT |
| | | | GKGVHLGFDYWGQGTLVTVSSGGGGQGGGGQGGGGQSYVLTQPPSVSVA |
| | | | PGQTARITCGGNNIGSKSVHWYQQKPGQAPVMVVYDDNDRPSGIPERFS |
| | | | GSNSGNTATLTISRVEAGDEADYYCQVWDYSGQRQVFGCGTKLTVL |
| 772 | MS_15-B12_CC | artificial | QVQLQESGPGLVKPSETLSLTCTVSGGSISSSSYFWGWIRQPPGKCLEW |
| | | | IGNIYYSGSSNYNPSLKSRVTISVDTSKNQFSLKLSSVTAADTAVYYCA |
| | | | RLPRGDRDAFDIWGQGTMVTVSSGGGGSGGGGSGGGGSDIVMTQSPSSL |
| | | | SASVGDRVTITCRASQGISNYLAWYQQKPGKVPKLLIYAASTLQSGVPS |
| | | | RFSGSGSGTDFTLTISSLQPEDFATYYCQQSYSTPFTFGCGTKVEIK |
| 773 | MS_15- | artificial | QVQLQESGPGLVKPSETLSLTCTVSGGSISSSSYFWGWIRQPPGKCLEW |
| | B12_CC_clipopt | | IGNIYYSGSSNYNPSLKSRVTISVDTSKNQFSLKLSSVTAADTAVYYCA |
| | | | RLPRGDRDAFDIWGQGTMVTVSSGGGGQGGGGQGGGGQDIVMTQSPSSL |
| | | | SASVGDRVTITCRASQGISNYLAWYQQKPGKVPKLLIYAASTLQSGVPS |
| | | | RFSGSGSGTDFTLTISSLQPEDFATYYCQQSYSTPFTFGCGTKVEIK |
| 774 | MS_15- | artificial | QVQLQESGPGLVKPSETLSLTCTVSGGSISSSSYFWGWIRQPPGKCLEW |
| | B12_CC_clipopt_EI | | IGNIYYSGSSNYNPSLKSRVTISVDTSKNQFSLKLSSVTAADTAVYYCA |
| | | | RLPRGDRDAFDIWGQGTMVTVSSGGGGQGGGGQGGGGQEIVMTQSPSSL |
| | | | SASVGDRVTITCRASQGISNYLAWYQQKPGKVPKLLIYAASTLQSGVPS |
| | | | RFSGSGSGTDFTLTISSLQPEDFATYYCQQSYSTPFTFGCGTKVEIK |
| 775 | MS_5-F11 | artificial | QVQLVESGGGLVKPGGSLRLSCAASGFTFSDYYMTWIRQAPGKGLEWLS |
| | | | YISSSGSTIYYADSVKGRFTISRDNAKNSLFLQMNSLRAEDTAVYYCAR |
| | | | DRNSHFDYWGQGTLVTVSSGGGGSGGGGSGGGGSDIQMTQSPSSVSASV |
| | | | GDRVTITCRASQGINTWLAWYQQKPGKAPKLLIYGASGLQSGVPSRFSG |
| | | | SGSGTDFTLTISSLQPEDFATYYCQQAKSFPRTFGQGTKVEIK |
| 776 | MS_5-F11_clipopt | artificial | QVQLVESGGGLVKPGGSLRLSCAASGFTFSDYYMTWIRQAPGKGLEWLS |
| | | | YISSSGSTIYYADSVKGRFTISRDNAKNSLFLQMNSLRAEDTAVYYCAR |
| | | | DRNSHFDYWGQGTLVTVSSGGGGQGGGGQGGGGQEIQMTQSPSSVSASV |
| | | | GDRVTITCRASQGINTWLAWYQQKPGKAPKLLIYGASGLQSGVPSRFSG |
| | | | SGSGTDFTLTISSLQPEDFATYYCQQAKSFPRTFGQGTKVEIK |
| 777 | MU_8-B7_CC | artificial | QVQLQQWGAGLLKPSETLSLTCAVYGGSFSGYYWSWIRQPPGKCLEWIG |
| | | | DIDASGSTKYNPSLKSRVTISLDTSKNQFSLKLNSVTAADTAVYFCARK |
| | | | KYSTVWSYFDNWGQGTLVTVSSGGGGSGGGGSGGGGSSYELTQPSSVSV |
| | | | PPGQTASITCSGDKLGDKYASWYQQKPGQSPVLVIYQDRKRPSGVPERF |
| | | | SGSNSGNTATLTISGTQAMDEADYYCQAWGSSTAVFGCGTKLTVL |
| 778 | MU_8-B7_CC_clipopt | artificial | QVQLQQWGAGLLKPSETLSLTCAVYGGSFSGYYWSWIRQPPGKCLEWIG |
| | | | DIDASGSTKYNPSLKSRVTISLDTSKNQFSLKLNSVTAADTAVYFCARK |
| | | | KYSTVWSYFDNWGQGTLVTVSSGGGGQGGGGQGGGGQSYELTQPSSVSV |
| | | | PPGQTASITCSGDKLGDKYASWYQQKPGQSPVLVIYQDRKRPSGVPERF |
| | | | SGSNSGNTATLTISGTQAMDEADYYCQAWGSSTAVFGCGTKLTVL |
| 779 | PM_76-B10 | artificial | QVQLVESGGGLVKPGESLRLSCAASGFTFSDYYMYWVRQAPGKGLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGSGGGGSGGGGSDIQMTQSPSSL |
| | | | SASVGDRVTITCKASQNVDTNVAWYQQKPGQAPKSLIYSASYRYSDVPS |
| | | | RFSGSASGTDFTLTISSVQSEDFATYYCQQYDSYPYTFGGGTKLEIK |
| 780 | PM_76-B10.11_CC | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGSGGGGSGGGGSDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIK |
| 781 | PM_76-B10.11_CC_GQ | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIK |
| 782 | PM_76- | artificial | QVQLVESGGGLVKPGESLRLSCAASGFTFSDYYMYWVRQAPGKCLEWVA |
| | B10.11_CC_clipopt | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQEIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIK |
| 783 | PM_76-B10_clipopt | artificial | QVQLVESGGGLVKPGESLRLSCAASGFTFSDYYMYWVRQAPGKGLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQEIQMTQSPSSL |
| | | | SASVGDRVTITCKASQNVDTNVAWYQQKPGQAPKSLIYSASYRYSDVPS |
| | | | RFSGSASGTDFTLTISSVQSEDFATYYCQQYDSYPYTFGGGTKLEIK |
| 784 | BC_A7_27-C4- | artificial | QVQLVQSGAEVKKPGASVKVSCKASGYTFTNHIIHWVRQAPGQGLEWMG |
| | G7X_x_I2C_clipopt | | YINPYPGYHAYNEKFQGRATMTSDTSTSTVYMELSSLRSEDTAVYYCAR |
| | | | DGYYRDTDVLDYWGQGTLVTVSSGGGGQGGGGQGGGGQEIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGQGTKVEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGQGGGGQGGGGQQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVL |
| 785 | BC_A7_27-C4-G7xI2C | artificial | QVQLVQSGAEVKKPGASVKVSCKASGYTFTNHIIHWVRQAPGQGLEWMG |
| | | | YINPYPGYHAYNEKFQGRATMTSDTSTSTVYMELSSLRSEDTAVYYCAR |
| | | | DGYYRDTDVLDYWGQGTLVTVSSGGGGSGGGGSGGGGSDIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGQGTKVEIKSG |
| | | | GGGSEVQLVESGGGLVQPGGSLKLSCAASGFTENKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVL |
| 786 | CD33_E11_x_I2C_ | artificial | QVQLVQSGAEVKKPGESVKVSCKASGYTFTNYGMNWVKQAPGQGLEWMG |
| | clipopt | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQEIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGQGT |
| | | | RLEIKSGGGGQEVQLVESGGGLVQPGGSLKLSCAASGFTENKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGQGGGGQG |
| | | | GGGQQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVL |
| 787 | CD33_E11xI2C | artificial | QVQLVQSGAEVKKPGESVKVSCKASGYTFTNYGMNWVKQAPGQGLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGSGGGGSGGGGSDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGQGT |
| | | | RLEIKSGGGGSEVQLVESGGGLVQPGGSLKLSCAASGFTFNKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGSGGGGSG |
| | | | GGGSQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVL |
| 788 | EGFRvIII_CCxI2C | artificial | QVQLVESGGGVVQSGRSLRLSCAASGFTFRNYGMHWVRQAPGKCLEWVA |
| | | | VIWYDGSDKYYADSVRGRFTISRDNSKNTLYLQMNSLRAEDTAVYYCAR |
| | | | DGYDILTGNPRDFDYWGQGTLVTVSSGGGGSGGGGSGGGGSDTVMTQTP |
| | | | LSSHVTLGQPASISCRSSQSLVHSDGNTYLSWLQQRPGQPPRLLIYRIS |
| | | | RRFSGVPDRFSGSGAGTDFTLEISRVEAEDVGVYYCMQSTHVPRTFGCG |
| | | | TKVEIKSGGGGSEVQLVESGGGLVQPGGSLKLSCAASGFTENKYAMNWV |
| | | | RQAPGKGLEWVARIRSKYNNYATYYADSVKDRFTISRDDSKNTAYLQMN |
| | | | NLKTEDTAVYYCVRHGNFGNSYISYWAYWGQGTLVTVSSGGGGSGGGGS |
| | | | GGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLW |
| | | | YSNRWVFGGGTKLTVL |
| 789 | EGFRvIII_CCx_I2C_ | artificial | QVQLVESGGGVVQSGRSLRLSCAASGFTFRNYGMHWVRQAPGKCLEWVA |
| | clipopt | | VIWYDGSDKYYADSVRGRFTISRDNSKNTLYLQMNSLRAEDTAVYYCAR |
| | | | DGYDILTGNPRDFDYWGQGTLVTVSSGGGGQGGGGQGGGGQDTVMTQTP |
| | | | LSSHVTLGQPASISCRSSQSLVHSDGNTYLSWLQQRPGQPPRLLIYRIS |
| | | | RRFSGVPDRFSGSGAGTDFTLEISRVEAEDVGVYYCMQSTHVPRTFGCG |
| | | | TKVEIKSGGGGQEVQLVESGGGLVQPGGSLKLSCAASGFTENKYAMNWV |
| | | | RQAPGKGLEWVARIRSKYNNYATYYADSVKDRFTISRDDSKNTAYLQMN |
| | | | NLKTEDTAVYYCVRHGNFGNSYISYWAYWGQGTLVTVSSGGGGQGGGGQ |
| | | | GGGGQQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLW |
| | | | YSNRWVFGGGTKLTVL |
| 790 | PM_76- | artificial | QVQLVESGGGLVKPGESLRLSCAASGFTFSDYYMYWVRQAPGKGLEWVA |
| | B10_x_I2C_clipopt | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQEIQMTQSPSSL |
| | | | SASVGDRVTITCKASQNVDTNVAWYQQKPGQAPKSLIYSASYRYSDVPS |
| | | | RFSGSASGTDFTLTISSVQSEDFATYYCQQYDSYPYTFGGGTKLEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGQGGGGQGGGGQQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVL |
| 791 | PM_76-B10xI2C | artificial | QVQLVESGGGLVKPGESLRLSCAASGFTFSDYYMYWVRQAPGKGLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGSGGGGSGGGGSDIQMTQSPSSL |
| | | | SASVGDRVTITCKASQNVDTNVAWYQQKPGQAPKSLIYSASYRYSDVPS |
| | | | RFSGSASGTDFTLTISSVQSEDFATYYCQQYDSYPYTFGGGTKLEIKSG |
| | | | GGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVL |
| 792 | 11D | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGSGGGGSGGGGSDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYCGQGTLVTVSSGGGGSGGGGSGGGGSQTV |
| | | | VTQEPSLTVSPGGTVTLTCGSSTGAVTSGNYPNWVQQKPGQCPRGLIGG |
| | | | TKFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 793 | BC_A7_27-C4- | artificial | QVQLVQSGAEVKKPGASVKVSCKASGYTFTNHIIHWVRQAPGQCLEWMG |
| | G7_CC_x_I2C_x_scFc | | YINPYPGYHAYNEKFQGRATMTSDTSTSTVYMELSSLRSEDTAVYYCAR |
| | | | DGYYRDTDVLDYWGQGTLVTVSSGGGGSGGGGSGGGGSDIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGCGTKVEIKSG |
| | | | GGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 794 | BC_A7_27-C4- | artificial | QVQLVQSGAEVKKPGASVKVSCKASGYTFTNHIIHWVRQAPGQCLEWMG |
| | G7_CC_x_I2C_x_scFc_ | | YINPYPGYHAYNEKFQGRATMTSDTSTSTVYMELSSLRSEDTAVYYCAR |
| | clipopt | | DGYYRDTDVLDYWGQGTLVTVSSGGGGQGGGGQGGGGQEIQMTQSPSSL |
| | | | SASVGDRVTITCQASQDISNYLNWYQQKPGKAPKLLIYYTSRLHTGVPS |
| | | | RFSGSGSGTDFTFTISSLEPEDIATYYCQQGNTLPWTFGCGTKVEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGQGGGGQGGGGQQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKENWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 795 | CD19_97-G1RE- | artificial | DIVMTQSPLSLPVISGEPASISCRSSQSLLHKNAFNYLDWYLQKPGQSP |
| | C2_LH_CC_x_92C_x_ | | QLLIYLGSNRASGVPDRFSGSGSGTDFTLKISRVEAEDVGVYYCMQALQ |
| | scFc | | TPFTFGCGTKVDIKGGGGSGGGGSGGGGSQVQLVESGGGVVQPGRSLRL |
| | | | SCAASGFTFSSYGMHWVRQAPGKCLEWVAVISYEGSNKYYAESVKGRFT |
| | | | ISRDNSKNTLYLQMNSLRDEDTAVYYCARDRGTIFGNYGLEVWGQGTTV |
| | | | TVSSSGGGGSEVQLVESGGGLVQPGGSLKLSCAASGFTENKYAMNWVRQ |
| | | | APGKGLEWVARIRSKYNNYATYYADSVKDRFTISRDDSKNTAYLQMNNL |
| | | | KTEDTAVYYCVRHGNFGNSYISYWAYWGQGTLVTVSSGGGGSGGGGSGG |
| | | | GGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYS |
| | | | NRWVFGGGTKLTVLGGGGDKTHTCPPCPAPELLGGPSVFLFPPKPKDTL |
| | | | MISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| | | | GGGGSGGGGSGGGGSGGGGSGGGGSGGGGSDKTHTCPPCPAPELLGGPS |
| | | | VFLFPPKPKDTLMISRTPEVTCVVVDVSHEDPEVKENWYVDGVEVHNAK |
| | | | TKPCEEQYGSTYRCVSVLTVLHQDWLNGKEYKCKVSNKALPAPIEKTIS |
| | | | KAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNH |
| | | | YTQKSLSLSPGK |
| 796 | CD19_97-G1RE- | artificial | DIVMTQSPLSLPVISGEPASISCRSSQSLLHKNAFNYLDWYLQKPGQSP |
| | C2_LH_CC_x_I2C_x_ | | QLLIYLGSNRASGVPDRFSGSGSGTDFTLKISRVEAEDVGVYYCMQALQ |
| | scFc-S | | TPFTFGCGTKVDIKGGGGSGGGGSGGGGSQVQLVESGGGVVQPGRSLRL |
| | | | SCAASGFTFSSYGMHWVRQAPGKCLEWVAVISYEGSNKYYAESVKGRFT |
| | | | ISRDNSKNTLYLQMNSLRDEDTAVYYCARDRGTIFGNYGLEVWGQGTTV |
| | | | TVSSGGGGSEVQLVESGGGLVQPGGSLKLSCAASGFTFNKYAMNWVRQA |
| | | | PGKGLEWVARIRSKYNNYATYYADSVKDRFTISRDDSKNTAYLQMNNLK |
| | | | TEDTAVYYCVRHGNFGNSYISYWAYWGQGTLVTVSSGGGGSGGGGSGGG |
| | | | GSQTVVTQEPSLTVSPGGTVTLTCGSSTGAVTSGNYPNWVQQKPGQAPR |
| | | | GLIGGTKFLAPGTPARFSGSLLGGKAALTLSGVQPEDEAEYYCVLWYSN |
| | | | RWVFGGGTKLTVLGGGGDKTHTCPPCPAPELLGGPSVFLFPPKPKDTLM |
| | | | ISRTPEVTCVVVDVSHEDPEVKFNWYVDGVEVHNAKTKPCEEQYGSTYR |
| | | | CVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSV |
| | | | FLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHY |
| | | | TQKSLSLSPGK |
| 797 | CD19_97-G1RE- | artificial | DIVMTQSPLSLPVISGEPASISCRSSQSLLHKNAFNYLDWYLQKPGQSP |
| | C2_LH_CC_x_I2C_x_Sc | | QLLIYLGSNRASGVPDRFSGSGSGTDFTLKISRVEAEDVGVYYCMQALQ |
| | Fc_clipopt | | TPFTFGCGTKVDIKGGGGQGGGGQGGGGQQVQLVESGGGVVQPGRSLRL |
| | | | SCAASGFTFSSYGMHWVRQAPGKCLEWVAVISYEGSNKYYAESVKGRFT |
| | | | ISRDNSKNTLYLQMNSLRDEDTAVYYCARDRGTIFGNYGLEVWGQGTTV |
| | | | TVSSGGGGQEVQLVESGGGLVQPGGSLKLSCAASGFTFNKYAMNWVRQA |
| | | | PGKGLEWVARIRSKYNNYATYYADSVKDRFTISRDDSKNTAYLQMNNLK |
| | | | TEDTAVYYCVRHGNFGNSYISYWAYWGQGTLVTVSSGGGGQGGGGQGGG |
| | | | GQQTVVTQEPSLTVSPGGTVTLTCGSSTGAVTSGNYPNWVQQKPGQAPR |
| | | | GLIGGTKFLAPGTPARFSGSLLGGKAALTLSGVQPEDEAEYYCVLWYSN |
| | | | RWVFGGGTKLTVLGGGGCPPCPAPELLGGPSVFLFPPKPKDTLMISRTP |
| | | | EVTCVVVDVSHEEPEVKENWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGQG |
| | | | GGGQGGGGQGGGGQGGGGQGGGGQCPPCPAPELLGGPSVFLFPPKPKDT |
| | | | LMISRTPEVTCVVVDVSHEEPEVKENWYVDGVEVHNAKTKPCEEQYGST |
| | | | YRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPG |
| | | | K |
| 798 | CD33_E11_CC_x_I2C_x | artificial | QVQLVQSGAEVKKPGESVKVSCKASGYTFTNYGMNWVKQAPGQCLEWMG |
| | scFc/_Z5S | | WINTYTGEPTYADKFQGRVTMTTDTSTSTAYMEIRNLGGDDTAVYYCAR |
| | | | WSWSDGYYVYFDYWGQGTSVTVSSGGGGSGGGGSGGGGSDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGSEVQLVESGGGLVQPGGSLKLSCAASGFTFNKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGSGGGGSG |
| | | | GGGSQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGDKTHTCPPCPAPELLGGPSVFLFPPKPKDT |
| | | | LMISRTPEVTCVVVDVSHEDPEVKFNWYVDGVEVHNAKTKPCEEQYGST |
| | | | YRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPG |
| | | | KGGGGSGGGGSGGGGSGGGGSGGGGSGGGGSDKTHTCPPCPAPELLGGP |
| | | | SVFLFPPKPKDTLMISRTPEVTCVVVDVSHEDPEVKFNWYVDGVEVHNA |
| | | | KTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHN |
| | | | HYTQKSLSLSPGK |
| 799 | CD33_E11_CC_x_I2C_x | artificial | QVQLVQSGAEVKKPGESVKVSCKASGYTFTNYGMNWVKQAPGQCLEWMG |
| | scFc_clipopt | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQEIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGQEVQLVESGGGLVQPGGSLKLSCAASGFTFNKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGQGGGGQG |
| | | | GGGQQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGCPPCPAPELLGGPSVFLFPPKPKDTLMISR |
| | | | TPEVTCVVVDVSHEEPEVKENWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGGG |
| | | | QGGGGQGGGGQGGGGQGGGGQGGGGQCPPCPAPELLGGPSVFLFPPKPK |
| | | | DTLMISRTPEVTCVVVDVSHEEPEVKENWYVDGVEVHNAKTKPCEEQYG |
| | | | STYRCVSVLTVLHQDWLNGKEYKCKVSNKALPAPIEKTISKAKGQPREP |
| | | | QVYTLPPSREEMTKNQVSLTCLVKGFYPSDIAVEWESNGQPENNYKTTP |
| | | | PVLDSDGSFFLYSKLTVDKSRWQQGNVFSCSVMHEALHNHYTQKSLSLS |
| | | | PGK |
| 800 | CD70_1_C7D_CC_x_I2C_ | artificial | EVQLLESGGGMVQPGGSLRLSCAASGFTFSTYAMSWVRQAPGKCLEWVS |
| | _scFc | | AISGSGGRTFYAESVEGRFTISRDNSKNTLYLQMNSLRAEDTAVYYCAK |
| | | | HDYSNYPYFDYWGQGTLVTVSSGGGGSGGGGSGGGGSEIVLTQSPGTLS |
| | | | LSPGERATLSCRASQSVRSTYLAWYQQKPGQAPRLLIYGASSRATGIPD |
| | | | RFSGSGSGTDFTLTISRLEPEDFAVYSCQQYGDLPFTFGCGTKLEIKSG |
| | | | GGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 801 | CD70_1_C7D_CC_x_I2C_ | artificial | EVQLLESGGGMVQPGGSLRLSCAASGFTFSTYAMSWVRQAPGKCLEWVS |
| | x_scFc_clipopt | | AISGSGGRTFYAESVEGRFTISRDNSKNTLYLQMNSLRAEDTAVYYCAK |
| | | | HDYSNYPYFDYWGQGTLVTVSSGGGGQGGGGQGGGGQEIVLTQSPGTLS |
| | | | LSPGERATLSCRASQSVRSTYLAWYQQKPGQAPRLLIYGASSRATGIPD |
| | | | RFSGSGSGTDFTLTISRLEPEDFAVYSCQQYGDLPFTFGCGTKLEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGQGGGGQGGGGQQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 802 | CH19_2G6.007_CC_x_ | artificial | QVQLVESGGGVVQPGGSLRLSCAASGFTFSSYGMHWVRQAPGKCLEWVA |
| | I2C_x_scFc_delGK | | FIWYEGSNKYYAESVKDRFTISRDNSKNTLYLQMNSLRAEDTAVYYCAR |
| | | | RAGIIGTIGYYYGMDVWGQGTTVTVSSGGGGSGGGGSGGGGSSYELTQP |
| | | | PSVSVSPGQTASITCSGDRLGEKYTSWYQQRPGQSPLLVIYQDTKRPSG |
| | | | IPERFSGSNSGNTATLTISGTQAMDEADYYCQAWESSTVVFGCGTKLTV |
| | | | LSGGGGSEVQLVESGGGLVQPGGSLKLSCAASGFTENKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGSGGGGSGGGGS |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMIS |
| | | | RTPEVTCVVVDVSHEDPEVKFNWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGGGGS |
| | | | GGGGSGGGGSGGGGSGGGGSGGGGSDKTHTCPPCPAPELLGGPSVFLFP |
| | | | PKPKDTLMISRTPEVTCVVVDVSHEDPEVKENWYVDGVEVHNAKTKPCE |
| | | | EQYGSTYRCVSVLTVLHQDWLNGKEYKCKVSNKALPAPIEKTISKAKGQ |
| | | | PREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKS |
| | | | LSLSPGK |
| 803 | CH19_2G6.007_CC_x_ | artificial | QVQLVESGGGVVQPGGSLRLSCAASGFTFSSYGMHWVRQAPGKCLEWVA |
| | I2C_x_scFc_delGK_ | | FIWYEGSNKYYAESVKDRFTISRDNSKNTLYLQMNSLRAEDTAVYYCAR |
| | clipopt | | RAGIIGTIGYYYGMDVWGQGTTVTVSSGGGGQGGGGQGGGGQSYELTQP |
| | | | PSVSVSPGQTASITCSGDRLGEKYTSWYQQRPGQSPLLVIYQDTKRPSG |
| | | | IPERFSGSNSGNTATLTISGTQAMDEADYYCQAWESSTVVFGCGTKLTV |
| | | | LSGGGGQEVQLVESGGGLVQPGGSLKLSCAASGFTFNKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGQGGGGQGGGGQ |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGQGGGGQ |
| | | | GGGGQGGGGQGGGGQGGGGQCPPCPAPELLGGPSVFLFPPKPKDTLMIS |
| | | | RTPEVTCVVVDVSHEEPEVKFNWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGK |
| 804 | CH3_G8A_6- | artificial | EVQLLESGGGLVQPGGSLRLSCAASGFSFSSYPINWVRQAPGKGLEWVG |
| | B12_x_I2C_x_scFc | | VIWTGGGTNYASSVKGRFTISRDNSKNTVYLQMNSLRAEDTAVYYCAKS |
| | | | RGVYDFDGRGAMDYWGQGTLVTVSSGGGGSGGGGSGGGGSDIVMTQSPD |
| | | | SLAVSLGERATINCKSSQSLLYSSNQKNYFAWYQQKPGQPPKLLIYWAS |
| | | | TRESGVPDRFSGSGSGTDFTLTISSLQAEDVAVYYCQQYYSYPYTFGQG |
| | | | TKLEIKSGGGGSEVQLVESGGGLVQPGGSLKLSCAASGFTFNKYAMNWV |
| | | | RQAPGKGLEWVARIRSKYNNYATYYADSVKDRFTISRDDSKNTAYLQMN |
| | | | NLKTEDTAVYYCVRHGNFGNSYISYWAYWGQGTLVTVSSGGGGSGGGGS |
| | | | GGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLW |
| | | | YSNRWVFGGGTKLTVLGGGGDKTHTCPPCPAPELLGGPSVFLFPPKPKD |
| | | | TLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GKGGGGSGGGGSGGGGSGGGGSGGGGSGGGGSDKTHTCPPCPAPELLGG |
| | | | PSVFLFPPKPKDTLMISRTPEVTCVVVDVSHEDPEVKENWYVDGVEVHN |
| | | | AKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALH |
| | | | NHYTQKSLSLSPGK |
| 805 | CH3_G8A_6- | artificial | EVQLLESGGGLVQPGGSLRLSCAASGFSFSSYPINWVRQAPGKGLEWVG |
| | B12_x_I2C_x_scFc_ | | VIWTGGGTNYASSVKGRFTISRDNSKNTVYLQMNSLRAEDTAVYYCAKS |
| | clipopt | | RGVYDFDGRGAMDYWGQGTLVTVSSGGGGQGGGGQGGGGQEIVMTQSPD |
| | | | SLAVSLGERATINCKSSQSLLYSSNQKNYFAWYQQKPGQPPKLLIYWAS |
| | | | TRESGVPDRFSGSGSGTDFTLTISSLQAEDVAVYYCQQYYSYPYTFGQG |
| | | | TKLEIKGGGGCPPCPAPELLGGPSVFLFPPKPKDTLMISRTPEVTCVVV |
| | | | DVSHEEPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGQGGGGQGGG |
| | | | GQGGGGQGGGGQGGGGQCPPCPAPELLGGPSVFLFPPKPKDTLMISRTP |
| | | | EVTCVVVDVSHEEPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGK |
| 806 | CL1_7-D7_CC_x_I2C0- | artificial | QVQLVQSGAEVKKPGASVKVSCKASGYTFTNYYMHWVRQAPGQCLEWMG |
| | scFc_clipopt | | WINPTSGGANYAQKFQGRVTMTRDTSISTAYMELSRLRSDDTAVYFCAR |
| | | | ESHAIQEGIWFDYWGQGTLVTVSSGGGGQGGGGQGGGGQEIQMTQSPSS |
| | | | LSASVGDRVTISCRASQSISNYLNWYQQKPGKAPKLLIYDASSLQSGVP |
| | | | SRFSGSGSGTDFTLTISSLQPEDFATYYCQQSYSFPLTFGCGTKVEIKS |
| | | | GGGGQEVQLVESGGGLVQPGGSLKLSCAASGFTFNKYAMNWVRQAPGKG |
| | | | LEWVARIRSKYNNYATYYADSVKDRFTISRDDSKNTAYLQMNNLKTEDT |
| | | | AVYYCVRHGNFGNSYISYWAYWGQGTLVTVSSGGGGQGGGGQGGGGQQT |
| | | | VVTQEPSLTVSPGGTVTLTCGSSTGAVTSGNYPNWVQQKPGQAPRGLIG |
| | | | GTKFLAPGTPARFSGSLLGGKAALTLSGVQPEDEAEYYCVLWYSNRWVF |
| | | | GGGTKLTVLGGGGCPPCPAPELLGGPSVFLFPPKPKDTLMISRTPEVTC |
| | | | VVVDVSHEEPEVKFNWYVDGVEVHNAKTKPCEEQYGSTYRCVSVLTVLH |
| | | | QDWLNGKEYKCKVSNKALPAPIEKTISKAKGQPREPQVYTLPPSREEMT |
| | | | KNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGQGGGGQ |
| | | | GGGGQGGGGQGGGGQGGGGQCPPCPAPELLGGPSVFLFPPKPKDTLMIS |
| | | | RTPEVTCVVVDVSHEEPEVKFNWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGK |
| 807 | CL1_7- | artificial | QVQLVQSGAEVKKPGASVKVSCKASGYTFTNYYMHWVRQAPGQCLEWMG |
| | D7_CC_x_I2C0_x_scFc | | WINPTSGGANYAQKFQGRVTMTRDTSISTAYMELSRLRSDDTAVYFCAR |
| | | | ESHAIQEGIWFDYWGQGTLVTVSSGGGGSGGGGSGGGGSDIQMTQSPSS |
| | | | LSASVGDRVTISCRASQSISNYLNWYQQKPGKAPKLLIYDASSLQSGVP |
| | | | SRFSGSGSGTDFTLTISSLQPEDFATYYCQQSYSFPLTFGCGTKVEIKS |
| | | | GGGGSEVQLVESGGGLVQPGGSLKLSCAASGFTFNKYAMNWVRQAPGKG |
| | | | LEWVARIRSKYNNYATYYADSVKDRFTISRDDSKNTAYLQMNNLKTEDT |
| | | | AVYYCVRHGNFGNSYISYWAYWGQGTLVTVSSGGGGSGGGGSGGGGSQT |
| | | | VVTQEPSLTVSPGGTVTLTCGSSTGAVTSGNYPNWVQQKPGQAPRGLIG |
| | | | GTKFLAPGTPARFSGSLLGGKAALTLSGVQPEDEAEYYCVLWYSNRWVF |
| | | | GGGTKLTVLGGGGDKTHTCPPCPAPELLGGPSVFLFPPKPKDTLMISRT |
| | | | PEVTCVVVDVSHEDPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGKGGGGS |
| | | | GGGGSGGGGSGGGGSGGGGSGGGGSDKTHTCPPCPAPELLGGPSVFLFP |
| | | | PKPKDTLMISRTPEVTCVVVDVSHEDPEVKFNWYVDGVEVHNAKTKPCE |
| | | | EQYGSTYRCVSVLTVLHQDWLNGKEYKCKVSNKALPAPIEKTISKAKGQ |
| | | | PREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKS |
| | | | LSLSPGK |
| 808 | CL6_3D4-01- | artificial | EIVLTQSPGTLSLSPGERATLSCRASQSVSSSYLAWYQQKPGQAPRLLI |
| | G2_LH_x_I2C_x_scFc | | YGASSRATGIPDRFSGSGSGTDFTLTISRLEPEDFAVYYCQQYGSSPLT |
| | | | FGCGTKLEIKGGGGSGGGGSGGGGSQVQLVQSGAEVKKPGASVKVSCKA |
| | | | SGYTFTGYYMHWVRQAPGQCLEWMGWINPNSGETNYAQKFQGRVTMTRD |
| | | | TSISTAYMELSRLRSDDTAVYYCARDALIVVAPVTRDYYYYGMDVWGQG |
| | | | TTVTVSSSGGGGSEVQLVESGGGLVQPGGSLKLSCAASGFTENKYAMNW |
| | | | VRQAPGKGLEWVARIRSKYNNYATYYADSVKDRFTISRDDSKNTAYLQM |
| | | | NNLKTEDTAVYYCVRHGNFGNSYISYWAYWGQGTLVTVSSGGGGSGGGG |
| | | | SGGGGSQTVVTQEPSLTVSPGGTVTLTCGSSTGAVTSGNYPNWVQQKPG |
| | | | QAPRGLIGGTKFLAPGTPARFSGSLLGGKAALTLSGVQPEDEAEYYCVL |
| | | | WYSNRWVFGGGTKLTVLGGGGDKTHTCPPCPAPELLGGPSVFLFPPKPK |
| | | | DTLMISRTPEVTCVVVDVSHEDPEVKFNWYVDGVEVHNAKTKPCEEQYG |
| | | | STYRCVSVLTVLHQDWLNGKEYKCKVSNKALPAPIEKTISKAKGQPREP |
| | | | QVYTLPPSREEMTKNQVSLTCLVKGFYPSDIAVEWESNGQPENNYKTTP |
| | | | PVLDSDGSFFLYSKLTVDKSRWQQGNVFSCSVMHEALHNHYTQKSLSLS |
| | | | PGKGGGGSGGGGSGGGGSGGGGSGGGGSGGGGSDKTHTCPPCPAPELLG |
| | | | GPSVFLFPPKPKDTLMISRTPEVTCVVVDVSHEDPEVKFNWYVDGVEVH |
| | | | NAKTKPCEEQYGSTYRCVSVLTVLHQDWLNGKEYKCKVSNKALPAPIEK |
| | | | TISKAKGQPREPQVYTLPPSREEMTKNQVSLTCLVKGFYPSDIAVEWES |
| | | | NGQPENNYKTTPPVLDSDGSFFLYSKLTVDKSRWQQGNVFSCSVMHEAL |
| | | | HNHYTQKSLSLSPGK |
| 809 | CL6_3D4- | artificial | EIVLTQSPGTLSLSPGERATLSCRASQSVSSSYLAWYQQKPGQAPRLLI |
| | 01.G2_LH_x_I2C_x_ | | YGASSRATGIPDRFSGSGSGTDFTLTISRLEPEDFAVYYCQQYGSSPLT |
| | scFc-S | | FGCGTKLEIKGGGGSGGGGSGGGGSQVQLVQSGAEVKKPGASVKVSCKA |
| | | | SGYTFTGYYMHWVRQAPGQCLEWMGWINPNSGETNYAQKFQGRVTMTRD |
| | | | TSISTAYMELSRLRSDDTAVYYCARDALIVVAPVTRDYYYYGMDVWGQG |
| | | | TTVTVSSGGGGSEVQLVESGGGLVQPGGSLKLSCAASGFTENKYAMNWV |
| | | | RQAPGKGLEWVARIRSKYNNYATYYADSVKDRFTISRDDSKNTAYLQMN |
| | | | NLKTEDTAVYYCVRHGNFGNSYISYWAYWGQGTLVTVSSGGGGSGGGGS |
| | | | GGGGSQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLW |
| | | | YSNRWVFGGGTKLTVLGGGGDKTHTCPPCPAPELLGGPSVFLFPPKPKD |
| | | | TLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GKGGGGSGGGGSGGGGSGGGGSGGGGSGGGGSDKTHTCPPCPAPELLGG |
| | | | PSVFLFPPKPKDTLMISRTPEVTCVVVDVSHEDPEVKENWYVDGVEVHN |
| | | | AKTKPCEEQYGSTYRCVSVLTVLHQDWLNGKEYKCKVSNKALPAPIEKT |
| | | | ISKAKGQPREPQVYTLPPSREEMTKNQVSLTCLVKGFYPSDIAVEWESN |
| | | | GQPENNYKTTPPVLDSDGSFFLYSKLTVDKSRWQQGNVFSCSVMHEALH |
| | | | NHYTQKSLSLSPGK |
| 810 | CL6_3D4- | artificial | EIVLTQSPGTLSLSPGERATLSCRASQSVSSSYLAWYQQKPGQAPRLLI |
| | 01.G2_LH_x_I2C_x_ | | YGASSRATGIPDRFSGSGSGTDFTLTISRLEPEDFAVYYCQQYGSSPLT |
| | scFc_clipopt | | FGCGTKLEIKGGGGQGGGGQGGGGQQVQLVQSGAEVKKPGASVKVSCKA |
| | | | SGYTFTGYYMHWVRQAPGQCLEWMGWINPNSGETNYAQKFQGRVTMTRD |
| | | | TSISTAYMELSRLRSDDTAVYYCARDALIVVAPVTRDYYYYGMDVWGQG |
| | | | TTVTVSSGGGGQEVQLVESGGGLVQPGGSLKLSCAASGFTENKYAMNWV |
| | | | RQAPGKGLEWVARIRSKYNNYATYYADSVKDRFTISRDDSKNTAYLQMN |
| | | | NLKTEDTAVYYCVRHGNFGNSYISYWAYWGQGTLVTVSSGGGGQGGGGQ |
| | | | GGGGQQTVVTQEPSLTVSPGGTVTLTCGSSTGAVTSGNYPNWVQQKPGQ |
| | | | APRGLIGGTKFLAPGTPARFSGSLLGGKAALTLSGVQPEDEAEYYCVLW |
| | | | YSNRWVFGGGTKLTVLGGGGCPPCPAPELLGGPSVFLFPPKPKDTLMIS |
| | | | RTPEVTCVVVDVSHEEPEVKFNWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGG |
| | | | GQGGGGQGGGGQGGGGQGGGGQGGGGQCPPCPAPELLGGPSVFLFPPKP |
| | | | KDTLMISRTPEVTCVVVDVSHEEPEVKFNWYVDGVEVHNAKTKPCEEQY |
| | | | GSTYRCVSVLTVLHQDWLNGKEYKCKVSNKALPAPIEKTISKAKGQPRE |
| | | | PQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSL |
| | | | SPGK |
| 811 | CL_10D8_CC_x_I2C_x_ | artificial | QVQLVQSGAEVKKPGASVKVSCKASGYTFTGYYMHWVRQAPGQCLEWMG |
| | scFc | | WINPNSGGTKYAQKFQGRVTMTRDTSISTAYMELSRLRSDDTAVYYCAR |
| | | | DRITVAGTYYYYGMDVWGQGTTVTVSSGGGGSGGGGSGGGGSDIQMTQS |
| | | | PSSVSASVGDRVTITCRASQGVNNWLAWYQQKPGKAPKLLIYTASSLQS |
| | | | GVPSRFSGSGSGTDFTLTIRSLQPEDFATYYCQQANSFPITFGCGTRLE |
| | | | IKSGGGGSEVQLVESGGGLVQPGGSLKLSCAASGFTFNKYAMNWVRQAP |
| | | | GKGLEWVARIRSKYNNYATYYADSVKDRFTISRDDSKNTAYLQMNNLKT |
| | | | EDTAVYYCVRHGNFGNSYISYWAYWGQGTLVTVSSGGGGSGGGGSGGGG |
| | | | SQTVVTQEPSLTVSPGGTVTLTCGSSTGAVTSGNYPNWVQQKPGQAPRG |
| | | | LIGGTKFLAPGTPARFSGSLLGGKAALTLSGVQPEDEAEYYCVLWYSNR |
| | | | WVFGGGTKLTVLGGGGDKTHTCPPCPAPELLGGPSVFLFPPKPKDTLMI |
| | | | SRTPEVTCVVVDVSHEDPEVKFNWYVDGVEVHNAKTKPCEEQYGSTYRC |
| | | | VSVLTVLHQDWLNGKEYKCKVSNKALPAPIEKTISKAKGQPREPQVYTL |
| | | | PPSREEMTKNQVSLTCLVKGFYPSDIAVEWESNGQPENNYKTTPPVLDS |
| | | | DGSFFLYSKLTVDKSRWQQGNVFSCSVMHEALHNHYTQKSLSLSPGKGG |
| | | | GGSGGGGSGGGGSGGGGSGGGGSGGGGSDKTHTCPPCPAPELLGGPSVF |
| | | | LFPPKPKDTLMISRTPEVTCVVVDVSHEDPEVKENWYVDGVEVHNAKTK |
| | | | PCEEQYGSTYRCVSVLTVLHQDWLNGKEYKCKVSNKALPAPIEKTISKA |
| | | | KGQPREPQVYTLPPSREEMTKNQVSLTCLVKGFYPSDIAVEWESNGQPE |
| | | | NNYKTTPPVLDSDGSFFLYSKLTVDKSRWQQGNVFSCSVMHEALHNHYT |
| | | | QKSLSLSPGK |
| 812 | CL_10D8_CC_x_I2C_x_ | artificial | QVQLVQSGAEVKKPGASVKVSCKASGYTFTGYYMHWVRQAPGQCLEWMG |
| | scFc_clipopt | | WINPNSGGTKYAQKFQGRVTMTRDTSISTAYMELSRLRSDDTAVYYCAR |
| | | | DRITVAGTYYYYGMDVWGQGTTVTVSSGGGGQGGGGQGGGGQEIQMTQS |
| | | | PSSVSASVGDRVTITCRASQGVNNWLAWYQQKPGKAPKLLIYTASSLQS |
| | | | GVPSRFSGSGSGTDFTLTIRSLQPEDFATYYCQQANSFPITFGCGTRLE |
| | | | IKSGGGGQEVQLVESGGGLVQPGGSLKLSCAASGFTFNKYAMNWVRQAP |
| | | | GKGLEWVARIRSKYNNYATYYADSVKDRFTISRDDSKNTAYLQMNNLKT |
| | | | EDTAVYYCVRHGNFGNSYISYWAYWGQGTLVTVSSGGGGQGGGGQGGGG |
| | | | QQTVVTQEPSLTVSPGGTVTLTCGSSTGAVTSGNYPNWVQQKPGQAPRG |
| | | | LIGGTKFLAPGTPARFSGSLLGGKAALTLSGVQPEDEAEYYCVLWYSNR |
| | | | WVFGGGTKLTVLGGGGCPPCPAPELLGGPSVFLFPPKPKDTLMISRTPE |
| | | | VTCVVVDVSHEEPEVKFNWYVDGVEVHNAKTKPCEEQYGSTYRCVSVLT |
| | | | VLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGQGG |
| | | | GGQGGGGQGGGGQGGGGQGGGGQCPPCPAPELLGGPSVFLFPPKPKDTL |
| | | | MISRTPEVTCVVVDVSHEEPEVKFNWYVDGVEVHNAKTKPCEEQYGSTY |
| | | | RCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| 813 | DL_8- | artificial | QVQLQESGPGLVKPSETLSLTCTVSGGSISSYYWSWIRQPPGKCLEWIG |
| | A7_CC_x_I2C_x_scFc_ | | YVYYSGTTNYNPSLKSRVTISVDTSKNQFSLKLSSVTAADTAVYYCASI |
| | delGK | | AVTGFYFDYWGQGTLVTVSSGGGGSGGGGSGGGGSEIVLTQSPGTLSLS |
| | | | PGERVTLSCRASQRVNNNYLAWYQQRPGQAPRLLIYGASSRATGIPDRF |
| | | | SGSGSGTDFTLTISRLEPEDFAVYYCQQYDRSPLTFGCGTKLEIKSGGG |
| | | | GSEVQLVESGGGLVQPGGSLKLSCAASGFTFNKYAMNWVRQAPGKGLEW |
| | | | VARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTVVT |
| | | | QEPSLTVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWVFGGG |
| | | | TKLTVLGGGGDKTHTCPPCPAPELLGGPSVFLFPPKPKDTLMISRTPEV |
| | | | TCVVVDVSHEDPEVKENWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGGGGSGGGGS |
| | | | GGGGSGGGGSGGGGSGGGGSDKTHTCPPCPAPELLGGPSVFLFPPKPKD |
| | | | TLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GK |
| 814 | DL_8- | artificial | QVQLQESGPGLVKPSETLSLTCTVSGGSISSYYWSWIRQPPGKCLEWIG |
| | A7_CC_x_I2C_x_scFc_ | | YVYYSGTTNYNPSLKSRVTISVDTSKNQFSLKLSSVTAADTAVYYCASI |
| | delGK | | AVTGFYFDYWGQGTLVTVSSGGGGQGGGGQGGGGQEIVLTQSPGTLSLS |
| | | | PGERVTLSCRASQRVNNNYLAWYQQRPGQAPRLLIYGASSRATGIPDRF |
| | | | SGSGSGTDFTLTISRLEPEDFAVYYCQQYDRSPLTFGCGTKLEIKSGGG |
| | | | GQEVQLVESGGGLVQPGGSLKLSCAASGFTFNKYAMNWVRQAPGKGLEW |
| | | | VARIRSKYNNYATYYADSVKDRFTISRDDSKNTAYLQMNNLKTEDTAVY |
| | | | YCVRHGNFGNSYISYWAYWGQGTLVTVSSGGGGQGGGGQGGGGQQTVVT |
| | | | QEPSLTVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWVFGGG |
| | | | TKLTVLGGGGCPPCPAPELLGGPSVFLFPPKPKDTLMISRTPEVTCVVV |
| | | | DVSHEEPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGGGGQGGGGQGGGGQ |
| | | | GGGGQGGGGQGGGGQCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 815 | F1D | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTFNKYAINWVRQAPGKGL |
| | | | EWVARIRSKYNNYATYYADAVKDRFTISRDDSKNTVYLQMNNLKTEDTA |
| | | | VYYCARAGNFGSSYISYWAYCGQGTLVTVSSGGGGQGGGGQGGGGQQTV |
| | | | VTQEPSLTVSPGGTVTITCGSSTGAVTSGNYPNWVQKKPGQCPRGLIGG |
| | | | TKFLAPGTPARFSGSLSGGKAALTLSGVQPEDEAEYYCVLWYSNRWVFG |
| | | | SGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGQG |
| | | | GGGQGGGGQGGGGQGGGGQGGGGQDKTHTCPPCPAPELLGGPSVFLFPP |
| | | | KPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 816 | F8I | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYCGQGTLVTVSSGGGGQGGGGQGGGGQQTV |
| | | | VTQEPSLTVSPGGTVTLTCGSSTGAVTSGNYPNWVQQKPGQCPRGLIGG |
| | | | TKFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGQG |
| | | | GGGQGGGGQGGGGQGGGGQGGGGQDKTHTCPPCPAPELLGGPSVFLFPP |
| | | | KPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 817 | FL_7- | artificial | QVTLKESGPTLVKPTETLTLTCTLSGFSLNNARMGVSWIRQPPGKCLEW |
| | A8_CC_x_I2C_x_scFc | | LAHIFSNDEKSYSTSLKNRLTISKDSSKTQVVLTMTNVDPVDTATYYCA |
| | | | RIVGYGSGWYGFFDYWGQGTLVTVSSGGGGSGGGGSGGGGSDIQMTQSP |
| | | | SSLSASVGDRVTITCRASQGIRNDLGWYQQKPGKAPKRLIYAASTLQSG |
| | | | VPSRFSGSGSGTEFTLTISSLQPEDFATYYCLQHNSYPLTFGCGTKVEI |
| | | | KSGGGGSEVQLVESGGGLVQPGGSLKLSCAASGFTFNKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGSGGGGSGGGGS |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMIS |
| | | | RTPEVTCVVVDVSHEDPEVKENWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGG |
| | | | GSGGGGSGGGGSGGGGSGGGGSGGGGSDKTHTCPPCPAPELLGGPSVFL |
| | | | FPPKPKDTLMISRTPEVTCVVVDVSHEDPEVKFNWYVDGVEVHNAKTKP |
| | | | CEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQ |
| | | | KSLSLSPGK |
| 818 | FL_7- | artificial | QVTLKESGPTLVKPTETLTLTCTLSGFSLNNARMGVSWIRQPPGKCLEW |
| | A8_CC_x_I2C_x_scFc_ | | LAHIFSNDEKSYSTSLKNRLTISKDSSKTQVVLTMTNVDPVDTATYYCA |
| | clipopt | | RIVGYGSGWYGFFDYWGQGTLVTVSSGGGGQGGGGQGGGGQEIQMTQSP |
| | | | SSLSASVGDRVTITCRASQGIRNDLGWYQQKPGKAPKRLIYAASTLQSG |
| | | | VPSRFSGSGSGTEFTLTISSLQPEDFATYYCLQHNSYPLTFGCGTKVEI |
| | | | KSGGGGQEVQLVESGGGLVQPGGSLKLSCAASGFTENKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGQGGGGQGGGGQ |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGQGGG |
| | | | GQGGGGQGGGGQGGGGQGGGGQCPPCPAPELLGGPSVFLFPPKPKDTLM |
| | | | ISRTPEVTCVVVDVSHEEPEVKFNWYVDGVEVHNAKTKPCEEQYGSTYR |
| | | | CVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGK |
| 819 | J1X | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGSGGGGSGGGGSDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGSEVQLVESGGGLVQPGGSLKLSCAASGFTENKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGSGGGGSG |
| | | | GGGSQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGCPPCPAPELLGGPSVFLFPPKPKDTLMISR |
| | | | TPEVTCVVVDVSHEEPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGGG |
| | | | SGGGGSGGGGSGGGGSGGGGSGGGGSCPPCPAPELLGGPSVFLFPPKPK |
| | | | DTLMISRTPEVTCVVVDVSHEEPEVKFNWYVDGVEVHNAKTKPCEEQYG |
| | | | STYRCVSVLTVLHQDWLNGKEYKCKVSNKALPAPIEKTISKAKGQPREP |
| | | | QVYTLPPSREEMTKNQVSLTCLVKGFYPSDIAVEWESNGQPENNYKTTP |
| | | | PVLDSDGSFFLYSKLTVDKSRWQQGNVFSCSVMHEALHNHYTQKSLSLS |
| | | | PGK |
| 820 | M4T | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTFNKYAINWVRQAPGKGL |
| | | | EWVARIRSKYNNYATYYADAVKDRFTISRDDSKNTVYLQMNNLKTEDTA |
| | | | VYYCARAGNFGSSYISYWAYWGQGTLVTVSSGGGGQGGGGQGGGGQQTV |
| | | | VTQEPSLTVSPGGTVTITCGSSTGAVTSGNYPNWVQKKPGQAPRGLIGG |
| | | | TKFLAPGTPARFSGSLSGGKAALTLSGVQPEDEAEYYCVLWYSNRWVFG |
| | | | SGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGQG |
| | | | GGGQGGGGQGGGGQGGGGQGGGGQDKTHTCPPCPAPELLGGPSVFLFPP |
| | | | KPKDTLMISRTPEVTCVVVDVSHEDPEVKENWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 821 | M5J | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTENKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYCGQGTLVTVSSGGGGQGGGGQGGGGQQTV |
| | | | VTQEPSLTVSPGGTVTLTCGSSTGAVTSGNYPNWVQQKPGQCPRGLIGG |
| | | | TKFLAPGTPARFSGSLLGGKAALTLSGVQPEDEAEYYCVLWYSNRWVFG |
| | | | GGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKENWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 822 | MA_10- | artificial | EVQLVESGGGLVQPGGSLRLSCAASGFTFSNAWMSWVRQAPGKCLEWVG |
| | B5_CC_x_I2C_x_scFc | | RIRSRSYGGTTDYAAPVKGRFTISRDDSKNTLFLQMNSLKTEDTAVYYC |
| | | | TTPSYSGSYYNYFSVMDVWGQGTTVTVSSGGGGSGGGGSGGGGSDIQMT |
| | | | QSPSSLSASVGDRVTITCRTSQSISSYLNWYQQKPGRAPKLLIFAASSL |
| | | | QGGVPSRFSGSGSGTDFTLTISSLQPEDFATYYCQQTYSMPFTFGCGTK |
| | | | VEIKSGGGGSEVQLVESGGGLVQPGGSLKLSCAASGFTFNKYAMNWVRQ |
| | | | APGKGLEWVARIRSKYNNYATYYADSVKDRFTISRDDSKNTAYLQMNNL |
| | | | KTEDTAVYYCVRHGNFGNSYISYWAYWGQGTLVTVSSGGGGSGGGGSGG |
| | | | GGSQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYS |
| | | | NRWVFGGGTKLTVLGGGGDKTHTCPPCPAPELLGGPSVFLFPPKPKDTL |
| | | | MISRTPEVTCVVVDVSHEDPEVKFNWYVDGVEVHNAKTKPCEEQYGSTY |
| | | | RCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| | | | GGGGSGGGGSGGGGSGGGGSGGGGSGGGGSDKTHTCPPCPAPELLGGPS |
| | | | VFLFPPKPKDTLMISRTPEVTCVVVDVSHEDPEVKENWYVDGVEVHNAK |
| | | | TKPCEEQYGSTYRCVSVLTVLHQDWLNGKEYKCKVSNKALPAPIEKTIS |
| | | | KAKGQPREPQVYTLPPSREEMTKNQVSLTCLVKGFYPSDIAVEWESNGQ |
| | | | PENNYKTTPPVLDSDGSFFLYSKLTVDKSRWQQGNVFSCSVMHEALHNH |
| | | | YTQKSLSLSPGK |
| 823 | MA_10- | artificial | EVQLVESGGGLVQPGGSLRLSCAASGFTFSNAWMSWVRQAPGKCLEWVG |
| | B5_CC_x_I2C_x_scFc_ | | RIRSRSYGGTTDYAAPVKGRFTISRDDSKNTLFLQMNSLKTEDTAVYYC |
| | clipopt | | TTPSYSGSYYNYFSVMDVWGQGTTVTVSSGGGGQGGGGQGGGGQEIQMT |
| | | | QSPSSLSASVGDRVTITCRTSQSISSYLNWYQQKPGRAPKLLIFAASSL |
| | | | QGGVPSRFSGSGSGTDFTLTISSLQPEDFATYYCQQTYSMPFTFGCGTK |
| | | | VEIKSGGGGQEVQLVESGGGLVQPGGSLKLSCAASGFTFNKYAMNWVRQ |
| | | | APGKGLEWVARIRSKYNNYATYYADSVKDRFTISRDDSKNTAYLQMNNL |
| | | | KTEDTAVYYCVRHGNFGNSYISYWAYWGQGTLVTVSSGGGGQGGGGQGG |
| | | | GGQQTVVTQEPSLTVSPGGTVTLTCGSSTGAVTSGNYPNWVQQKPGQAP |
| | | | RGLIGGTKFLAPGTPARFSGSLLGGKAALTLSGVQPEDEAEYYCVLWYS |
| | | | NRWVFGGGTKLTVLGGGGCPPCPAPELLGGPSVFLFPPKPKDTLMISRT |
| | | | PEVTCVVVDVSHEEPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGKGGGGQ |
| | | | GGGGQGGGGQGGGGQGGGGQGGGGQCPPCPAPELLGGPSVFLFPPKPKD |
| | | | TLMISRTPEVTCVVVDVSHEEPEVKFNWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GK |
| 824 | MA_3- | artificial | EVQLLESGGGLVQPGGSLRLSCAASGFTFSSYAMSWVRQAPGKCLEWVS |
| | G10_CC_x_I2C_x_scFc | | AISGSGGGTYYAASVKGRFTISRDNSKNTLYLQMSSLRAEDTAVYYCAT |
| | | | GKGVHLGFDYWGQGTLVTVSSGGGGSGGGGSGGGGSSYVLTQPPSVSVA |
| | | | PGQTARITCGGNNIGSKSVHWYQQKPGQAPVMVVYDDNDRPSGIPERFS |
| | | | GSNSGNTATLTISRVEAGDEADYYCQVWDYSGQRQVFGCGTKLTVLSGG |
| | | | GGSEVQLVESGGGLVQPGGSLKLSCAASGFTFNKYAMNWVRQAPGKGLE |
| | | | WVARIRSKYNNYATYYADSVKDRFTISRDDSKNTAYLQMNNLKTEDTAV |
| | | | YYCVRHGNFGNSYISYWAYWGQGTLVTVSSGGGGSGGGGSGGGGSQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVEGG |
| | | | GTKLTVLGGGGDKTHTCPPCPAPELLGGPSVFLFPPKPKDTLMISRTPE |
| | | | VTCVVVDVSHEDPEVKFNWYVDGVEVHNAKTKPCEEQYGSTYRCVSVLT |
| | | | VLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGSGG |
| | | | GGSGGGGSGGGGSGGGGSGGGGSDKTHTCPPCPAPELLGGPSVFLFPPK |
| | | | PKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLS |
| | | | LSPGK |
| 825 | MA_3- | artificial | EVQLLESGGGLVQPGGSLRLSCAASGFTFSSYAMSWVRQAPGKCLEWVS |
| | G10_CC_x_I2C_x_scFc_ | | AISGSGGGTYYAASVKGRFTISRDNSKNTLYLQMSSLRAEDTAVYYCAT |
| | clipopt | | GKGVHLGFDYWGQGTLVTVSSGGGGQGGGGQGGGGQSYVLTQPPSVSVA |
| | | | PGQTARITCGGNNIGSKSVHWYQQKPGQAPVMVVYDDNDRPSGIPERFS |
| | | | GSNSGNTATLTISRVEAGDEADYYCQVWDYSGQRQVFGCGTKLTVLSGG |
| | | | GGQEVQLVESGGGLVQPGGSLKLSCAASGFTFNKYAMNWVRQAPGKGLE |
| | | | WVARIRSKYNNYATYYADSVKDRFTISRDDSKNTAYLQMNNLKTEDTAV |
| | | | YYCVRHGNFGNSYISYWAYWGQGTLVTVSSGGGGQGGGGQGGGGQQTVV |
| | | | TQEPSLTVSPGGTVTLTCGSSTGAVTSGNYPNWVQQKPGQAPRGLIGGT |
| | | | KFLAPGTPARFSGSLLGGKAALTLSGVQPEDEAEYYCVLWYSNRWVEGG |
| | | | GTKLTVLGGGGCPPCPAPELLGGPSVFLFPPKPKDTLMISRTPEVTCVV |
| | | | VDVSHEEPEVKFNWYVDGVEVHNAKTKPCEEQYGSTYRCVSVLTVLHQD |
| | | | WLNGKEYKCKVSNKALPAPIEKTISKAKGQPREPQVYTLPPSREEMTKN |
| | | | QVSLTCLVKGFYPSDIAVEWESNGQPENNYKTTPPVLDSDGSFFLYSKL |
| | | | TVDKSRWQQGNVFSCSVMHEALHNHYTQKSLSLSPGKGGGGQGGGGQGG |
| | | | GGQGGGGQGGGGQGGGGQCPPCPAPELLGGPSVFLFPPKPKDTLMISRT |
| | | | PEVTCVVVDVSHEEPEVKFNWYVDGVEVHNAKTKPCEEQYGSTYRCVSV |
| | | | LTVLHQDWLNGKEYKCKVSNKALPAPIEKTISKAKGQPREPQVYTLPPS |
| | | | REEMTKNQVSLTCLVKGFYPSDIAVEWESNGQPENNYKTTPPVLDSDGS |
| | | | FFLYSKLTVDKSRWQQGNVFSCSVMHEALHNHYTQKSLSLSPGK |
| 826 | MS_5- | artificial | QVQLVESGGGLVKPGGSLRLSCAASGFTFSDYYMTWIRQAPGKGLEWLS |
| | F11_x_I2C_x_scFc | | YISSSGSTIYYADSVKGRFTISRDNAKNSLFLQMNSLRAEDTAVYYCAR |
| | | | DRNSHFDYWGQGTLVTVSSGGGGSGGGGSGGGGSDIQMTQSPSSVSASV |
| | | | GDRVTITCRASQGINTWLAWYQQKPGKAPKLLIYGASGLQSGVPSRFSG |
| | | | SGSGTDFTLTISSLQPEDFATYYCQQAKSFPRTFGQGTKVEIKSGGGGS |
| | | | EVQLVESGGGLVQPGGSLKLSCAASGFTENKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSSGGGGSGGGGSGGGGSQTVVTQE |
| | | | PSLTVSPGGTVTLTCGSSTGAVTSGNYPNWVQQKPGQAPRGLIGGTKFL |
| | | | APGTPARFSGSLLGGKAALTLSGVQPEDEAEYYCVLWYSNRWVFGGGTK |
| | | | LTVLGGGGDKTHTCPPCPAPELLGGPSVFLFPPKPKDTLMISRTPEVTC |
| | | | VVVDVSHEDPEVKFNWYVDGVEVHNAKTKPCEEQYGSTYRCVSVLTVLH |
| | | | QDWLNGKEYKCKVSNKALPAPIEKTISKAKGQPREPQVYTLPPSREEMT |
| | | | KNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGS |
| | | | GGGGSGGGGSGGGGSGGGGSDKTHTCPPCPAPELLGGPSVFLFPPKPKD |
| | | | TLMISRTPEVTCVVVDVSHEDPEVKFNWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GK |
| 827 | MS_5- | artificial | QVQLVESGGGLVKPGGSLRLSCAASGFTFSDYYMTWIRQAPGKGLEWLS |
| | F11_x_I2C_x_scFc_ | | YISSSGSTIYYADSVKGRFTISRDNAKNSLFLQMNSLRAEDTAVYYCAR |
| | clipopt | | DRNSHFDYWGQGTLVTVSSGGGGQGGGGQGGGGQEIQMTQSPSSVSASV |
| | | | GDRVTITCRASQGINTWLAWYQQKPGKAPKLLIYGASGLQSGVPSRFSG |
| | | | SGSGTDFTLTISSLQPEDFATYYCQQAKSFPRTFGQGTKVEIKSGGGGQ |
| | | | EVQLVESGGGLVQPGGSLKLSCAASGFTFNKYAMNWVRQAPGKGLEWVA |
| | | | RIRSKYNNYATYYADSVKDRFTISRDDSKNTAYLQMNNLKTEDTAVYYC |
| | | | VRHGNFGNSYISYWAYWGQGTLVTVSSGGGGQGGGGQGGGGQQTVVTQE |
| | | | PSLTVSPGGTVTLTCGSSTGAVTSGNYPNWVQQKPGQAPRGLIGGTKFL |
| | | | APGTPARFSGSLLGGKAALTLSGVQPEDEAEYYCVLWYSNRWVFGGGTK |
| | | | LTVLGGGGCPPCPAPELLGGPSVFLFPPKPKDTLMISRTPEVTCVVVDV |
| | | | SHEEPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVS |
| | | | LTCLVKGFYPSDIAVEWESNGQPENNYKTTPPVLDSDGSFFLYSKLTVD |
| | | | KSRWQQGNVFSCSVMHEALHNHYTQKSLSLSPGKGGGGQGGGGQGGGGQ |
| | | | GGGGQGGGGQGGGGQCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGK |
| 828 | MU_8- | artificial | QVQLQQWGAGLLKPSETLSLTCAVYGGSFSGYYWSWIRQPPGKCLEWIG |
| | B7_CC_x_I2C_x_scFc | | DIDASGSTKYNPSLKSRVTISLDTSKNQFSLKLNSVTAADTAVYFCARK |
| | | | KYSTVWSYFDNWGQGTLVTVSSGGGGSGGGGSGGGGSSYELTQPSSVSV |
| | | | PPGQTASITCSGDKLGDKYASWYQQKPGQSPVLVIYQDRKRPSGVPERF |
| | | | SGSNSGNTATLTISGTQAMDEADYYCQAWGSSTAVFGCGTKLTVLSGGG |
| | | | GSEVQLVESGGGLVQPGGSLKLSCAASGFTFNKYAMNWVRQAPGKGLEW |
| | | | VARIRSKYNNYATYYADSVKDRFTISRDDSKNTAYLQMNNLKTEDTAVY |
| | | | YCVRHGNFGNSYISYWAYWGQGTLVTVSSGGGGSGGGGSGGGGSQTVVT |
| | | | QEPSLTVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWVFGGG |
| | | | TKLTVLGGGGDKTHTCPPCPAPELLGGPSVFLFPPKPKDTLMISRTPEV |
| | | | TCVVVDVSHEDPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGSGGG |
| | | | GSGGGGSGGGGSGGGGSGGGGSDKTHTCPPCPAPELLGGPSVFLFPPKP |
| | | | KDTLMISRTPEVTCVVVDVSHEDPEVKFNWYVDGVEVHNAKTKPCEEQY |
| | | | GSTYRCVSVLTVLHQDWLNGKEYKCKVSNKALPAPIEKTISKAKGQPRE |
| | | | PQVYTLPPSREEMTKNQVSLTCLVKGFYPSDIAVEWESNGQPENNYKTT |
| | | | PPVLDSDGSFFLYSKLTVDKSRWQQGNVFSCSVMHEALHNHYTQKSLSL |
| | | | SPGK |
| 829 | MU_8- | artificial | QVQLQQWGAGLLKPSETLSLTCAVYGGSFSGYYWSWIRQPPGKCLEWIG |
| | B7_CC_x_I2C_x_scFc_ | | DIDASGSTKYNPSLKSRVTISLDTSKNQFSLKLNSVTAADTAVYFCARK |
| | clipopt | | KYSTVWSYFDNWGQGTLVTVSSGGGGQGGGGQGGGGQSYELTQPSSVSV |
| | | | PPGQTASITCSGDKLGDKYASWYQQKPGQSPVLVIYQDRKRPSGVPERF |
| | | | SGSNSGNTATLTISGTQAMDEADYYCQAWGSSTAVFGCGTKLTVLSGGG |
| | | | GQEVQLVESGGGLVQPGGSLKLSCAASGFTFNKYAMNWVRQAPGKGLEW |
| | | | VARIRSKYNNYATYYADSVKDRFTISRDDSKNTAYLQMNNLKTEDTAVY |
| | | | YCVRHGNFGNSYISYWAYWGQGTLVTVSSGGGGQGGGGQGGGGQQTVVT |
| | | | QEPSLTVSPGGTVTLTCGSSTGAVTSGNYPNWVQQKPGQAPRGLIGGTK |
| | | | FLAPGTPARFSGSLLGGKAALTLSGVQPEDEAEYYCVLWYSNRWVFGGG |
| | | | TKLTVLGGGGCPPCPAPELLGGPSVFLFPPKPKDTLMISRTPEVTCVVV |
| | | | DVSHEEPEVKFNWYVDGVEVHNAKTKPCEEQYGSTYRCVSVLTVLHQDW |
| | | | LNGKEYKCKVSNKALPAPIEKTISKAKGQPREPQVYTLPPSREEMTKNQ |
| | | | VSLTCLVKGFYPSDIAVEWESNGQPENNYKTTPPVLDSDGSFFLYSKLT |
| | | | VDKSRWQQGNVFSCSVMHEALHNHYTQKSLSLSPGKGGGGQGGGGQGGG |
| | | | GQGGGGQGGGGQGGGGQCPPCPAPELLGGPSVFLFPPKPKDTLMISRTP |
| | | | EVTCVVVDVSHEEPEVKENWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGK |
| 830 | PM_76- | artificial | QVQLVESGGGLVKPGESLRLSCAASGFTFSDYYMYWVRQAPGKCLEWVA |
| | B10.11_CC_x_I2C_x_ | | IISDGGYYTYYSDIIKGRFTISRDNAKNSLYLQMNSLKAEDTAVYYCAR |
| | ScFc_/_11S | | GFPLLRHGAMDYWGQGTLVTVSSGGGGSGGGGSGGGGSDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGSEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGSGGGGSGGGGSQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGDKTHTCPPCPAPELLGGPSVFLFPPKPKDTLMISRTP |
| | | | EVTCVVVDVSHEDPEVKFNWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGKGGGGSG |
| | | | GGGSGGGGSGGGGSGGGGSGGGGSDKTHTCPPCPAPELLGGPSVFLFPP |
| | | | KPKDTLMISRTPEVTCVVVDVSHEDPEVKFNWYVDGVEVHNAKTKPCEE |
| | | | QYGSTYRCVSVLTVLHQDWLNGKEYKCKVSNKALPAPIEKTISKAKGQP |
| | | | REPQVYTLPPSREEMTKNQVSLTCLVKGFYPSDIAVEWESNGQPENNYK |
| | | | TTPPVLDSDGSFFLYSKLTVDKSRWQQGNVFSCSVMHEALHNHYTQKSL |
| | | | SLSPGK |
| 831 | PM_76- | artificial | QVQLVESGGGLVKPGESLRLSCAASGFTFSDYYMYWVRQAPGKCLEWVA |
| | B10.11_CC_x_I2C_x_ | | IISDGGYYTYYSDIIKGRFTISRDNAKNSLYLQMNSLKAEDTAVYYCAR |
| | scFc_clipopt | | GFPLLRHGAMDYWGQGTLVTVSSGGGGQGGGGQGGGGQEIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTFNKYAMNWVRQAPGKGL |
| | | | EWVARIRSKYNNYATYYADSVKDRFTISRDDSKNTAYLQMNNLKTEDTA |
| | | | VYYCVRHGNFGNSYISYWAYWGQGTLVTVSSGGGGQGGGGQGGGGQQTV |
| | | | VTQEPSLTVSPGGTVTLTCGSSTGAVTSGNYPNWVQQKPGQAPRGLIGG |
| | | | TKFLAPGTPARFSGSLLGGKAALTLSGVQPEDEAEYYCVLWYSNRWVFG |
| | | | GGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 832 | Q6S | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGQEVQLVESGGGLVQPGGSLKLSCAASGFTENKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGQGGGGQG |
| | | | GGGQQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGDKTHTCPPCPAPELLGGPSVFLFPPKPKDT |
| | | | LMISRTPEVTCVVVDVSHEDPEVKENWYVDGVEVHNAKTKPCEEQYGST |
| | | | YRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPG |
| | | | KGGGGQGGGGQGGGGQGGGGQGGGGQGGGGQDKTHTCPPCPAPELLGGP |
| | | | SVFLFPPKPKDTLMISRTPEVTCVVVDVSHEDPEVKENWYVDGVEVHNA |
| | | | KTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHN |
| | | | HYTQKSLSLSPGK |
| 833 | Q8I | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTFNKYAINWVRQAPGKGL |
| | | | EWVARIRSKYNNYATYYADAVKDRFTISRDDSKNTVYLQMNNLKTEDTA |
| | | | VYYCARAGNFGSSYISYWAYCGQGTLVTVSSGGGGQGGGGQGGGGQQTV |
| | | | VTQEPSLTVSPGGTVTITCGSSTGAVTSGNYPNWVQKKPGQCPRGLIGG |
| | | | TKFLAPGTPARFSGSLSGGKAALTLSGVQPEDEAEYYCVLWYSNRWVFG |
| | | | SGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 834 | S5Z | artificial | QVQLVESGGGLVKPGESLRLSCAASGFTFSDYYMYWVRQAPGKCLEWVA |
| | | | IISDGGYYTYYSDIIKGRFTISRDNAKNSLYLQMNSLKAEDTAVYYCAR |
| | | | GFPLLRHGAMDYWGQGTLVTVSSGGGGQGGGGQGGGGQDIQMTQSPSSL |
| | | | SASVGDRVTITCKASQNVDTNVAWYQQKPGQAPKSLIYSASYVYWDVPS |
| | | | RFSGSASGTDFTLTISSVQSEDFATYYCQQYDQQLITFGCGTKLEIKSG |
| | | | GGGQEVQLVESGGGLVQPGGSLKLSCAASGFTENKYAINWVRQAPGKGL |
| | | | EWVARIRSKYNNYATYYADAVKDRFTISRDDSKNTVYLQMNNLKTEDTA |
| | | | VYYCARAGNFGSSYISYWAYWGQGTLVTVSSGGGGQGGGGQGGGGQQTV |
| | | | VTQEPSLTVSPGGTVTITCGSSTGAVTSGNYPNWVQKKPGQAPRGLIGG |
| | | | TKFLAPGTPARFSGSLSGGKAALTLSGVQPEDEAEYYCVLWYSNRWVFG |
| | | | SGTKLTVLGGGGCPPCPAPELLGGPSVFLFPPKPKDTLMISRTPEVTCV |
| | | | VVDVSHEEPEVKFNWYVDGVEVHNAKTKPCEEQYGSTYRCVSVLTVLHQ |
| | | | DWLNGKEYKCKVSNKALPAPIEKTISKAKGQPREPQVYTLPPSREEMTK |
| | | | NQVSLTCLVKGFYPSDIAVEWESNGQPENNYKTTPPVLDSDGSFFLYSK |
| | | | LTVDKSRWQQGNVFSCSVMHEALHNHYTQKSLSLSPGKGGGGQGGGGQG |
| | | | GGGQGGGGQGGGGQGGGGQCPPCPAPELLGGPSVFLFPPKPKDTLMISR |
| | | | TPEVTCVVVDVSHEEPEVKENWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGK |
| 835 | W7V | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGRGGGGRGGGGRDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGREVQLVESGGGLVQPGGSLKLSCAASGFTENKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGRGGGGRG |
| | | | GGGRQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGDKTHTCPPCPAPELLGGPSVFLFPPKPKDT |
| | | | LMISRTPEVTCVVVDVSHEDPEVKFNWYVDGVEVHNAKTKPCEEQYGST |
| | | | YRCVSVLTVLHQDWLNGKEYKCKVSNKALPAPIEKTISKAKGQPREPQV |
| | | | YTLPPSREEMTKNQVSLTCLVKGFYPSDIAVEWESNGQPENNYKTTPPV |
| | | | LDSDGSFFLYSKLTVDKSRWQQGNVFSCSVMHEALHNHYTQKSLSLSPG |
| | | | KGGGGRGGGGRGGGGRGGGGRGGGGRGGGGRDKTHTCPPCPAPELLGGP |
| | | | SVFLFPPKPKDTLMISRTPEVTCVVVDVSHEDPEVKENWYVDGVEVHNA |
| | | | KTKPCEEQYGSTYRCVSVLTVLHQDWLNGKEYKCKVSNKALPAPIEKTI |
| | | | SKAKGQPREPQVYTLPPSREEMTKNQVSLTCLVKGFYPSDIAVEWESNG |
| | | | QPENNYKTTPPVLDSDGSFFLYSKLTVDKSRWQQGNVFSCSVMHEALHN |
| | | | HYTQKSLSLSPGK |
| 836 | W8I | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGRGGGGRGGGGRDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGREVQLVESGGGLVQPGGSLKLSCAASGFTENKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGRGGGGRG |
| | | | GGGRQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGCPPCPAPELLGGPSVFLFPPKPKDTLMISR |
| | | | TPEVTCVVVDVSHEEPEVKFNWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGGG |
| | | | RGGGGRGGGGRGGGGRGGGGRGGGGRCPPCPAPELLGGPSVFLFPPKPK |
| | | | DTLMISRTPEVTCVVVDVSHEEPEVKENWYVDGVEVHNAKTKPCEEQYG |
| | | | STYRCVSVLTVLHQDWLNGKEYKCKVSNKALPAPIEKTISKAKGQPREP |
| | | | QVYTLPPSREEMTKNQVSLTCLVKGFYPSDIAVEWESNGQPENNYKTTP |
| | | | PVLDSDGSFFLYSKLTVDKSRWQQGNVFSCSVMHEALHNHYTQKSLSLS |
| | | | PGK |
| 837 | X7D | artificial | QVQLVQSGAEVKKPGESVKVSCKASGYTFTNYGMNWVKQAPGQCLEWMG |
| | | | WINTYTGEPTYADKFQGRVTMTTDTSTSTAYMEIRNLGGDDTAVYYCAR |
| | | | WSWSDGYYVYFDYWGQGTSVTVSSGGGGQGGGGQGGGGQDIVMTQSPDS |
| | | | LTVSLGERTTINCKSSQSVLDSSTNKNSLAWYQQKPGQPPKLLLSWAST |
| | | | RESGIPDRFSGSGSGTDFTLTIDSPQPEDSATYYCQQSAHFPITFGCGT |
| | | | RLEIKSGGGGQEVQLVESGGGLVQPGGSLKLSCAASGFTFNKYAMNWVR |
| | | | QAPGKGLEWVARIRSKYNNYATYYADSVKDRFTISRDDSKNTAYLQMNN |
| | | | LKTEDTAVYYCVRHGNFGNSYISYWAYWGQGTLVTVSSGGGGQGGGGQG |
| | | | GGGQQTVVTQEPSLTVSPGGTVTLTCGSSTGAVTSGNYPNWVQQKPGQA |
| | | | PRGLIGGTKFLAPGTPARFSGSLLGGKAALTLSGVQPEDEAEYYCVLWY |
| | | | SNRWVFGGGTKLTVLGGGGCPPCPAPELLGGPSVFLFPPKPKDTLMISR |
| | | | TPEVTCVVVDVSHEEPEVKENWYVDGVEVHNAKTKPCEEQYGSTYRCVS |
| | | | VLTVLHQDWLNGKEYKCKVSNKALPAPIEKTISKAKGQPREPQVYTLPP |
| | | | SREEMTKNQVSLTCLVKGFYPSDIAVEWESNGQPENNYKTTPPVLDSDG |
| | | | SFFLYSKLTVDKSRWQQGNVFSCSVMHEALHNHYTQKSLSLSPGKGGGG |
| | | | QGGGGQGGGGQGGGGQGGGGQGGGGQCPPCPAPELLGGPSVFLFPPKPK |
| | | | DTLMISRTPEVTCVVVDVSHEEPEVKFNWYVDGVEVHNAKTKPCEEQYG |
| | | | STYRCVSVLTVLHQDWLNGKEYKCKVSNKALPAPIEKTISKAKGQPREP |
| | | | QVYTLPPSREEMTKNQVSLTCLVKGFYPSDIAVEWESNGQPENNYKTTP |
| | | | PVLDSDGSFFLYSKLTVDKSRWQQGNVFSCSVMHEALHNHYTQKSLSLS |
| | | | PGK |
| 838 | CS_PDL241.12_LH_CC_ | artificial | DIQMTQSPSSLSASVGDRVTITCKASQDVSTAVAWYQQKPGKAPKLLIY |
| | x_BC_A7_27-C4- | | SASYRYTGVPDRFTGSGSGTDFTLTISSLQPEDFATYYCQQHYSTPPYT |
| | G7_CC_x_I2C_x_scFc | | FGCGTKVEIKRGGGGSGGGGSGGGGSQVQLVQSGAEVKKPGASVKVSCK |
| | | | ASGYAFSSSWMNWVRQAPGQCLEWIGRIYPGDADAKYNAKFKGKATLTA |
| | | | DKSTSTAYMELSSLRSEDTAVYYCARSTMIATGAMDYWGQGTLVTVSSS |
| | | | GGGGSQVQLVQSGAEVKKPGASVKVSCKASGYTFTNHIIHWVRQAPGQC |
| | | | LEWMGYINPYPGYHAYNEKFQGRATMTSDTSTSTVYMELSSLRSEDTAV |
| | | | YYCARDGYYRDTDVLDYWGQGTLVTVSSGGGGSGGGGSGGGGSDIQMTQ |
| | | | SPSSLSASVGDRVTITCQASQDISNYLNWYQQKPGKAPKLLIYYTSRLH |
| | | | TGVPSRFSGSGSGTDFTFTISSLEPEDIATYYCQQGNTLPWTFGCGTKV |
| | | | EIKSGGGGSEVQLVESGGGLVQPGGSLKLSCAASGFTFNKYAMNWVRQA |
| | | | PGKGLEWVARIRSKYNNYATYYADSVKDRFTISRDDSKNTAYLQMNNLK |
| | | | TEDTAVYYCVRHGNFGNSYISYWAYWGQGTLVTVSSGGGGSGGGGSGGG |
| | | | GSQTVVTQEPSLTVSPGGTVTLTCGSSTGAVTSGNYPNWVQQKPGQAPR |
| | | | GLIGGTKFLAPGTPARFSGSLLGGKAALTLSGVQPEDEAEYYCVLWYSN |
| | | | RWVFGGGTKLTVLGGGGDKTHTCPPCPAPELLGGPSVFLFPPKPKDTLM |
| | | | ISRTPEVTCVVVDVSHEDPEVKFNWYVDGVEVHNAKTKPCEEQYGSTYR |
| | | | CVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGKG |
| | | | GGGSGGGGSGGGGSGGGGSGGGGSGGGGSDKTHTCPPCPAPELLGGPSV |
| | | | FLFPPKPKDTLMISRTPEVTCVVVDVSHEDPEVKENWYVDGVEVHNAKT |
| | | | KPCEEQYGSTYRCVSVLTVLHQDWLNGKEYKCKVSNKALPAPIEKTISK |
| | | | AKGQPREPQVYTLPPSREEMTKNQVSLTCLVKGFYPSDIAVEWESNGQP |
| | | | ENNYKTTPPVLDSDGSFFLYSKLTVDKSRWQQGNVFSCSVMHEALHNHY |
| | | | TQKSLSLSPGK |
| 839 | CS_PDL241.12_LH_CC_ | artificial | DIQMTQSPSSLSASVGDRVTITCKASQDVSTAVAWYQQKPGKAPKLLIY |
| | x_BC_A7_27-C4- | | SASYRYTGVPDRFTGSGSGTDFTLTISSLQPEDFATYYCQQHYSTPPYT |
| | G7_CC_x_I2C_x_scFc_ | | FGCGTKVEIKRGGGGQGGGGQGGGGQQVQLVQSGAEVKKPGASVKVSCK |
| | +R_clipopt | | ASGYAFSSSWMNWVRQAPGQCLEWIGRIYPGDADAKYNAKFKGKATLTA |
| | | | DKSTSTAYMELSSLRSEDTAVYYCARSTMIATGAMDYWGQGTLVTVSSG |
| | | | GGGQQVQLVQSGAEVKKPGASVKVSCKASGYTFTNHIIHWVRQAPGQCL |
| | | | EWMGYINPYPGYHAYNEKFQGRATMTSDTSTSTVYMELSSLRSEDTAVY |
| | | | YCARDGYYRDTDVLDYWGQGTLVTVSSGGGGQGGGGQGGGGQEIQMTQS |
| | | | PSSLSASVGDRVTITCQASQDISNYLNWYQQKPGKAPKLLIYYTSRLHT |
| | | | GVPSRFSGSGSGTDFTFTISSLEPEDIATYYCQQGNTLPWTFGCGTKVE |
| | | | IKSGGGGQEVQLVESGGGLVQPGGSLKLSCAASGFTFNKYAMNWVRQAP |
| | | | GKGLEWVARIRSKYNNYATYYADSVKDRFTISRDDSKNTAYLQMNNLKT |
| | | | EDTAVYYCVRHGNFGNSYISYWAYWGQGTLVTVSSGGGGQGGGGQGGGG |
| | | | QQTVVTQEPSLTVSPGGTVTLTCGSSTGAVTSGNYPNWVQQKPGQAPRG |
| | | | LIGGTKFLAPGTPARFSGSLLGGKAALTLSGVQPEDEAEYYCVLWYSNR |
| | | | WVFGGGTKLTVLGGGGCPPCPAPELLGGPSVFLFPPKPKDTLMISRTPE |
| | | | VTCVVVDVSHEEPEVKFNWYVDGVEVHNAKTKPCEEQYGSTYRCVSVLT |
| | | | VLHQDWLNGKEYKCKVSNKALPAPIEKTISKAKGQPREPQVYTLPPSRE |
| | | | EMTKNQVSLTCLVKGFYPSDIAVEWESNGQPENNYKTTPPVLDSDGSFF |
| | | | LYSKLTVDKSRWQQGNVFSCSVMHEALHNHYTQKSLSLSPGKGGGGQGG |
| | | | GGQGGGGQGGGGQGGGGQGGGGQCPPCPAPELLGGPSVFLFPPKPKDTL |
| | | | MISRTPEVTCVVVDVSHEEPEVKFNWYVDGVEVHNAKTKPCEEQYGSTY |
| | | | RCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| 840 | CS_PDL241.12_LH_CC_ | artificial | DIQMTQSPSSLSASVGDRVTITCKASQDVSTAVAWYQQKPGKAPKLLIY |
| | x_BC_A7_27-C4- | | SASYRYTGVPDRFTGSGSGTDFTLTISSLQPEDFATYYCQQHYSTPPYT |
| | G7_CC_x_I2C_x_scFc_ | | FGCGTKVEIKGGGGQGGGGQGGGGQQVQLVQSGAEVKKPGASVKVSCKA |
| | −R_clipopt | | SGYAFSSSWMNWVRQAPGQCLEWIGRIYPGDADAKYNAKFKGKATLTAD |
| | | | KSTSTAYMELSSLRSEDTAVYYCARSTMIATGAMDYWGQGTLVTVSSGG |
| | | | GGQQVQLVQSGAEVKKPGASVKVSCKASGYTFTNHIIHWVRQAPGQCLE |
| | | | WMGYINPYPGYHAYNEKFQGRATMTSDTSTSTVYMELSSLRSEDTAVYY |
| | | | CARDGYYRDTDVLDYWGQGTLVTVSSGGGGQGGGGQGGGGQEIQMTQSP |
| | | | SSLSASVGDRVTITCQASQDISNYLNWYQQKPGKAPKLLIYYTSRLHTG |
| | | | VPSRFSGSGSGTDFTFTISSLEPEDIATYYCQQGNTLPWTFGCGTKVEI |
| | | | KSGGGGQEVQLVESGGGLVQPGGSLKLSCAASGFTFNKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGQGGGGQGGGGQ |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGCPPCPAPELLGGPSVFLFPPKPKDTLMISRTPEV |
| | | | TCVVVDVSHEEPEVKENWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGQGGG |
| | | | GQGGGGQGGGGQGGGGQGGGGQCPPCPAPELLGGPSVFLFPPKPKDTLM |
| | | | ISRTPEVTCVVVDVSHEEPEVKFNWYVDGVEVHNAKTKPCEEQYGSTYR |
| | | | CVSVLTVLHQDWLNGKEYKCKVSNKALPAPIEKTISKAKGQPREPQVYT |
| | | | LPPSREEMTKNQVSLTCLVKGFYPSDIAVEWESNGQPENNYKTTPPVLD |
| | | | SDGSFFLYSKLTVDKSRWQQGNVFSCSVMHEALHNHYTQKSLSLSPGK |
| 841 | CS_PDL241.12_LH_CC_ | artificial | DIQMTQSPSSLSASVGDRVTITCKASQDVSTAVAWYQQKPGKAPKLLIY |
| | x_BC_A7_27-C4- | | SASYRYTGVPDRFTGSGSGTDFTLTISSLQPEDFATYYCQQHYSTPPYT |
| | G7_CC_x_I2C_x_scFc_ | | FGCGTKVEIKGGGGSGGGGSGGGGSQVQLVQSGAEVKKPGASVKVSCKA |
| | −R-S | | SGYAFSSSWMNWVRQAPGQCLEWIGRIYPGDADAKYNAKFKGKATLTAD |
| | | | KSTSTAYMELSSLRSEDTAVYYCARSTMIATGAMDYWGQGTLVTVSSGG |
| | | | GGSQVQLVQSGAEVKKPGASVKVSCKASGYTFTNHIIHWVRQAPGQCLE |
| | | | WMGYINPYPGYHAYNEKFQGRATMTSDTSTSTVYMELSSLRSEDTAVYY |
| | | | CARDGYYRDTDVLDYWGQGTLVTVSSGGGGSGGGGSGGGGSDIQMTQSP |
| | | | SSLSASVGDRVTITCQASQDISNYLNWYQQKPGKAPKLLIYYTSRLHTG |
| | | | VPSRFSGSGSGTDFTFTISSLEPEDIATYYCQQGNTLPWTFGCGTKVEI |
| | | | KSGGGGSEVQLVESGGGLVQPGGSLKLSCAASGFTFNKYAMNWVRQAPG |
| | | | KGLEWVARIRSKYNNYATYYADSVKDRFTISRDDSKNTAYLQMNNLKTE |
| | | | DTAVYYCVRHGNFGNSYISYWAYWGQGTLVTVSSGGGGSGGGGSGGGGS |
| | | | QTVVTQEPSLTVSPGGTVTLTCGSSTGAVTSGNYPNWVQQKPGQAPRGL |
| | | | IGGTKFLAPGTPARFSGSLLGGKAALTLSGVQPEDEAEYYCVLWYSNRW |
| | | | VFGGGTKLTVLGGGGDKTHTCPPCPAPELLGGPSVFLFPPKPKDTLMIS |
| | | | RTPEVTCVVVDVSHEDPEVKENWYVDGVEVHNAKTKPCEEQYGSTYRCV |
| | | | SVLTVLHQDWLNGKEYKCKVSNKALPAPIEKTISKAKGQPREPQVYTLP |
| | | | PSREEMTKNQVSLTCLVKGFYPSDIAVEWESNGQPENNYKTTPPVLDSD |
| | | | GSFFLYSKLTVDKSRWQQGNVFSCSVMHEALHNHYTQKSLSLSPGKGGG |
| | | | GSGGGGSGGGGSGGGGSGGGGSGGGGSDKTHTCPPCPAPELLGGPSVFL |
| | | | FPPKPKDTLMISRTPEVTCVVVDVSHEDPEVKFNWYVDGVEVHNAKTKP |
| | | | CEEQYGSTYRCVSVLTVLHQDWLNGKEYKCKVSNKALPAPIEKTISKAK |
| | | | GQPREPQVYTLPPSREEMTKNQVSLTCLVKGFYPSDIAVEWESNGQPEN |
| | | | NYKTTPPVLDSDGSFFLYSKLTVDKSRWQQGNVFSCSVMHEALHNHYTQ |
| | | | KSLSLSPGK |
| 842 | FL_7- | artificial | QVTLKESGPTLVKPTETLTLTCTLSGFSLNNARMGVSWIRQPPGKCLEW |
| | A8_CC_x_CD123_24- | | LAHIFSNDEKSYSTSLKNRLTISKDSSKTQVVLTMTNVDPVDTATYYCA |
| | B4- | | RIVGYGSGWYGFFDYWGQGTLVTVSSGGGGSGGGGSGGGGSDIQMTQSP |
| | f_NK_CC_x_I2C_x_scFc | | SSLSASVGDRVTITCRASQGIRNDLGWYQQKPGKAPKRLIYAASTLQSG |
| | | | VPSRFSGSGSGTEFTLTISSLQPEDFATYYCLQHNSYPLTFGCGTKVEI |
| | | | KSGGGGSEVQLLESGGGLVQPGGSLRLSCAASGFTFSHYAMSWVRQAPG |
| | | | KCLEWVSAVSGGGDKTLYADAVKGRFTISRDNSKNTLFLQMNSLRAEDT |
| | | | AIYYCARLRGFYYGMDVWGQGTTVTVSSGGGGSGGGGSGGGGSDIVLTQ |
| | | | SPLSLPVTPGEPASISCRSSQSLLHSNKYNYLDWYLQKPGQSPQLLIYL |
| | | | GSNRASGVPDRFSGSGSGTDFTLKISRVEAEDVGVYYCMQALQTPPITF |
| | | | GCGTRLEIKSGGGGSEVQLVESGGGLVQPGGSLKLSCAASGFTENKYAM |
| | | | NWVRQAPGKGLEWVARIRSKYNNYATYYADSVKDRFTISRDDSKNTAYL |
| | | | QMNNLKTEDTAVYYCVRHGNFGNSYISYWAYWGQGTLVTVSSGGGGSGG |
| | | | GGSGGGGSQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYC |
| | | | VLWYSNRWVFGGGTKLTVLGGGGDKTHTCPPCPAPELLGGPSVFLFPPK |
| | | | PKDTLMISRTPEVTCVVVDVSHEDPEVKENWYVDGVEVHNAKTKPCEEQ |
| | | | YGSTYRCVSVLTVLHQDWLNGKEYKCKVSNKALPAPIEKTISKAKGQPR |
| | | | EPQVYTLPPSREEMTKNQVSLTCLVKGFYPSDIAVEWESNGQPENNYKT |
| | | | TPPVLDSDGSFFLYSKLTVDKSRWQQGNVFSCSVMHEALHNHYTQKSLS |
| | | | LSPGKGGGGSGGGGSGGGGSGGGGSGGGGSGGGGSDKTHTCPPCPAPEL |
| | | | LGGPSVFLFPPKPKDTLMISRTPEVTCVVVDVSHEDPEVKENWYVDGVE |
| | | | VHNAKTKPCEEQYGSTYRCVSVLTVLHQDWLNGKEYKCKVSNKALPAPI |
| | | | EKTISKAKGQPREPQVYTLPPSREEMTKNQVSLTCLVKGFYPSDIAVEW |
| | | | ESNGQPENNYKTTPPVLDSDGSFFLYSKLTVDKSRWQQGNVFSCSVMHE |
| | | | ALHNHYTQKSLSLSPGK |
| 843 | FL_7- | artificial | QVTLKESGPTLVKPTETLTLTCTLSGFSLNNARMGVSWIRQPPGKCLEW |
| | A8_CC_x_CD123_24- | | LAHIFSNDEKSYSTSLKNRLTISKDSSKTQVVLTMTNVDPVDTATYYCA |
| | B4- | | RIVGYGSGWYGFFDYWGQGTLVTVSSGGGGQGGGGQGGGGQEIQMTQSP |
| | f_NK_CC_x_I2C_x_ | | SSLSASVGDRVTITCRASQGIRNDLGWYQQKPGKAPKRLIYAASTLQSG |
| | scFc_clipopt | | VPSRFSGSGSGTEFTLTISSLQPEDFATYYCLQHNSYPLTFGCGTKVEI |
| | | | KSGGGGQEVQLLESGGGLVQPGGSLRLSCAASGFTFSHYAMSWVRQAPG |
| | | | KCLEWVSAVSGGGDKTLYADAVKGRFTISRDNSKNTLFLQMNSLRAEDT |
| | | | AIYYCARLRGFYYGMDVWGQGTTVTVSSGGGGQGGGGQGGGGQEIVLTQ |
| | | | SPLSLPVTPGEPASISCRSSQSLLHSNKYNYLDWYLQKPGQSPQLLIYL |
| | | | GSNRASGVPDRFSGSGSGTDFTLKISRVEAEDVGVYYCMQALQTPPITF |
| | | | GCGTRLEIKSGGGGQEVQLVESGGGLVQPGGSLKLSCAASGFTENKYAM |
| | | | NWVRQAPGKGLEWVARIRSKYNNYATYYADSVKDRFTISRDDSKNTAYL |
| | | | QMNNLKTEDTAVYYCVRHGNFGNSYISYWAYWGQGTLVTVSSGGGGQGG |
| | | | GGQGGGGQQTVVTQEPSLTVSPGGTVTLTCGSSTGAVTSGNYPNWVQQK |
| | | | PGQAPRGLIGGTKFLAPGTPARFSGSLLGGKAALTLSGVQPEDEAEYYC |
| | | | VLWYSNRWVFGGGTKLTVLGGGGCPPCPAPELLGGPSVFLFPPKPKDTL |
| | | | MISRTPEVTCVVVDVSHEEPEVKENWYVDGVEVHNAKTKPCEEQYGSTY |
| | | | RCVSVLTVLHQDWLNGKEYKCKVSNKALPAPIEKTISKAKGQPREPQVY |
| | | | TLPPSREEMTKNQVSLTCLVKGFYPSDIAVEWESNGQPENNYKTTPPVL |
| | | | DSDGSFFLYSKLTVDKSRWQQGNVFSCSVMHEALHNHYTQKSLSLSPGK |
| | | | GGGGQGGGGQGGGGQGGGGQGGGGQGGGGQCPPCPAPELLGGPSVFLFP |
| | | | PKPKDTLMISRTPEVTCVVVDVSHEEPEVKFNWYVDGVEVHNAKTKPCE |
| | | | EQYGSTYRCVSVLTVLHQDWLNGKEYKCKVSNKALPAPIEKTISKAKGQ |
| | | | PREPQVYTLPPSREEMTKNQVSLTCLVKGFYPSDIAVEWESNGQPENNY |
| | | | KTTPPVLDSDGSFFLYSKLTVDKSRWQQGNVFSCSVMHEALHNHYTQKS |
| | | | LSLSPGK |
| 844 | CH3_15- | artificial | QVQLVQSGAEVKKPGASVKVSCKASGYTFTNYWMNWVRQAPGQCLEWMG |
| | E11_CC_EIx_I2L_x_S | | NIAYGVKGTNYNQKFQGRVTMTVDTSSSTAYMELSRLRSDDTAVYYCAT |
| | cFc_x_MS_15- | | RYFYVMDYWGQGTLVTVSSGGGGQGGGGQGGGGQEIQMTQSPSSLSASV |
| | B12_CC_EIx_I2L_ | | GDRVTITCRASQDISNYLNWYQQKPGKVPKLLIYYTSRLHSGVPSRFSG |
| | clipopt | | SGSGTDFTLTISSLQPEDVATYYCVQYAQFPLTFGCGTKVEIKSGGGGQ |
| | | | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGQGGGGQGGGGQQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVLGGGGCPPCPAPELLGGPSVFLFPPKPKDTLMISRTPEVTCVVVDV |
| | | | SHEEPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVS |
| | | | LTCLVKGFYPSDIAVEWESNGQPENNYKTTPPVLDSDGSFFLYSKLTVD |
| | | | KSRWQQGNVFSCSVMHEALHNHYTQKSLSLSPGKGGGGQGGGGQGGGGQ |
| | | | GGGGQGGGGQGGGGQCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGQVQL |
| | | | QESGPGLVKPSETLSLTCTVSGGSISSSSYFWGWIRQPPGKCLEWIGNI |
| | | | YYSGSSNYNPSLKSRVTISVDTSKNQFSLKLSSVTAADTAVYYCARLPR |
| | | | GDRDAFDIWGQGTMVTVSSGGGGQGGGGQGGGGQEIVMTQSPSSLSASV |
| | | | GDRVTITCRASQGISNYLAWYQQKPGKVPKLLIYAASTLQSGVPSRFSG |
| | | | SGSGTDFTLTISSLQPEDFATYYCQQSYSTPFTFGCGTKVEIKSGGGGQ |
| | | | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGQGGGGQGGGGQQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVL |
| 845 | CH3_15- | artificial | QVQLVQSGAEVKKPGASVKVSCKASGYTFTNYWMNWVRQAPGQCLEWMG |
| | E11_CC_x_I2L_x_scFc | | NIAYGVKGTNYNQKFQGRVTMTVDTSSSTAYMELSRLRSDDTAVYYCAT |
| | X MS_15- | | RYFYVMDYWGQGTLVTVSSGGGGSGGGGSGGGGSDIQMTQSPSSLSASV |
| | B12_CC_x_I2L_(R3I) | | GDRVTITCRASQDISNYLNWYQQKPGKVPKLLIYYTSRLHSGVPSRFSG |
| | | | SGSGTDFTLTISSLQPEDVATYYCVQYAQFPLTFGCGTKVEIKSGGGGS |
| | | | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGSGGGGSGGGGSQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVLGGGGDKTHTCPPCPAPELLGGPSVFLFPPKPKDTLMISRTPEVTC |
| | | | VVVDVSHEDPEVKFNWYVDGVEVHNAKTKPCEEQYGSTYRCVSVLTVLH |
| | | | QDWLNGKEYKCKVSNKALPAPIEKTISKAKGQPREPQVYTLPPSREEMT |
| | | | KNQVSLTCLVKGFYPSDIAVEWESNGQPENNYKTTPPVLDSDGSFFLYS |
| | | | KLTVDKSRWQQGNVFSCSVMHEALHNHYTQKSLSLSPGKGGGGSGGGGS |
| | | | GGGGSGGGGSGGGGSGGGGSDKTHTCPPCPAPELLGGPSVFLFPPKPKD |
| | | | TLMISRTPEVTCVVVDVSHEDPEVKENWYVDGVEVHNAKTKPCEEQYGS |
| | | | TYRCVSVLTVLHQDWLNGKEYKCKVSNKALPAPIEKTISKAKGQPREPQ |
| | | | VYTLPPSREEMTKNQVSLTCLVKGFYPSDIAVEWESNGQPENNYKTTPP |
| | | | VLDSDGSFFLYSKLTVDKSRWQQGNVFSCSVMHEALHNHYTQKSLSLSP |
| | | | GKGGGGQVQLQESGPGLVKPSETLSLTCTVSGGSISSSSYFWGWIRQPP |
| | | | GKCLEWIGNIYYSGSSNYNPSLKSRVTISVDTSKNQFSLKLSSVTAADT |
| | | | AVYYCARLPRGDRDAFDIWGQGTMVTVSSGGGGSGGGGSGGGGSDIVMT |
| | | | QSPSSLSASVGDRVTITCRASQGISNYLAWYQQKPGKVPKLLIYAASTL |
| | | | QSGVPSRFSGSGSGTDFTLTISSLQPEDFATYYCQQSYSTPFTFGCGTK |
| | | | VEIKSGGGGSEVQLVESGGGLVQPGGSLKLSCAASGFTENKYAMNWVRQ |
| | | | APGKGMEWVARIRSKYNNYATYYADAVKDRFTISRDDSKNTLYLQMNNL |
| | | | KTEDTAVYYCVRAGNFGSSYISYFAYWGQGTLVTVSSGGGGSGGGGSGG |
| | | | GGSQTVVTQEPSLTVSPGGTVTITCGSSTGAVTSGNYPNWIQKKPGQAP |
| | | | RGLIGGTKFLAPGTPARFSGSLEGGKAALTLSGVQPEDEAEYYCVLYYS |
| | | | NRWVFGSGTKLTVL |
| 846 | CH3_15- | artificial | QVQLVQSGAEVKKPGASVKVSCKASGYTFTNYWMNWVRQAPGQCLEWMG |
| | E11_CC_x_I2L_x_scFc_ | | NIAYGVKGTNYNQKFQGRVTMTVDTSSSTAYMELSRLRSDDTAVYYCAT |
| | x_MS_15- | | RYFYVMDYWGQGTLVTVSSGGGGQGGGGQGGGGQDIQMTQSPSSLSASV |
| | B12_CC_x_I2L_ | | GDRVTITCRASQDISNYLNWYQQKPGKVPKLLIYYTSRLHSGVPSRFSG |
| | clipopt_(F5Q) | | SGSGTDFTLTISSLQPEDVATYYCVQYAQFPLTFGCGTKVEIKSGGGGQ |
| | | | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGQGGGGQGGGGQQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVLGGGGCPPCPAPELLGGPSVFLFPPKPKDTLMISRTPEVTCVVVDV |
| | | | SHEEPEVKFNWYVDGVEVHNAKTKPCEEQYGSTYRCVSVLTVLHQDWLN |
| | | | GKEYKCKVSNKALPAPIEKTISKAKGQPREPQVYTLPPSREEMTKNQVS |
| | | | LTCLVKGFYPSDIAVEWESNGQPENNYKTTPPVLDSDGSFFLYSKLTVD |
| | | | KSRWQQGNVFSCSVMHEALHNHYTQKSLSLSPGKGGGGQGGGGQGGGGQ |
| | | | GGGGQGGGGQGGGGQCPPCPAPELLGGPSVFLFPPKPKDTLMISRTPEV |
| | | | TCVVVDVSHEEPEVKFNWYVDGVEVHNAKTKPCEEQYGSTYRCVSVLTV |
| | | | LHQDWLNGKEYKCKVSNKALPAPIEKTISKAKGQPREPQVYTLPPSREE |
| | | | MTKNQVSLTCLVKGFYPSDIAVEWESNGQPENNYKTTPPVLDSDGSFFL |
| | | | YSKLTVDKSRWQQGNVFSCSVMHEALHNHYTQKSLSLSPGKGGGGQVQL |
| | | | QESGPGLVKPSETLSLTCTVSGGSISSSSYFWGWIRQPPGKCLEWIGNI |
| | | | YYSGSSNYNPSLKSRVTISVDTSKNQFSLKLSSVTAADTAVYYCARLPR |
| | | | GDRDAFDIWGQGTMVTVSSGGGGQGGGGQGGGGQDIVMTQSPSSLSASV |
| | | | GDRVTITCRASQGISNYLAWYQQKPGKVPKLLIYAASTLQSGVPSRFSG |
| | | | SGSGTDFTLTISSLQPEDFATYYCQQSYSTPFTFGCGTKVEIKSGGGGQ |
| | | | EVQLVESGGGLVQPGGSLKLSCAASGFTFNKYAMNWVRQAPGKGMEWVA |
| | | | RIRSKYNNYATYYADAVKDRFTISRDDSKNTLYLQMNNLKTEDTAVYYC |
| | | | VRAGNFGSSYISYFAYWGQGTLVTVSSGGGGQGGGGQGGGGQQTVVTQE |
| | | | PSLTVSPGGTVTITCGSSTGAVTSGNYPNWIQKKPGQAPRGLIGGTKFL |
| | | | APGTPARFSGSLEGGKAALTLSGVQPEDEAEYYCVLYYSNRWVFGSGTK |
| | | | LTVL |
| 847 | Human CD3epsilon | artificial | QDGNEEMGGITQTPYKVSISGTTVILTCPQYPGSEILWQHNDKNIGGDE |
| | ECD | | DDKNIGSDEDHLSLKEFSELEQSGYYVCYPRGSKPEDANFYLYLRARVC |
| | | | ENCMEMD |
| 848 | Human CD3epsilon | artificial | QDGNEEMGGITQTPYKVSISGTTVILT |
| | ECD/pos. 1-27 | | |
| 849 | scFc-Spacer | artificial | DKTHTCPPCPAPELLGGPSVFLFPPKPKDTLMISRTPEVTCVVVDVSHE |
| | | | DPEVKFNWYVDGVEVHNAKTKPCEEQYGSTYRCVSVLTVLHQDWLNGKE |
| | | | YKCKVSNKALPAPIEKTISKAKGQPREPQVYTLPPSREEMTKNQVSLTC |
| | | | LVKGFYPSDIAVEWESNGQPENNYKTTPPVLDSDGSFFLYSKLTVDKSR |
| | | | WQQGNVFSCSVMHEALHNHYTQKSLSLSPGKGGGGSGGGGSGGGGSGGG |
| | | | GSGGGGSGGGGSDKTHTCPPCPAPELLGGPSVFLFPPKPKDTLMISRTP |
| | | | EVTCVVVDVSHEDPEVKENWYVDGVEVHNAKTKPCEEQYGSTYRCVSVL |
| | | | TVLHQDWLNGKEYKCKVSNKALPAPIEKTISKAKGQPREPQVYTLPPSR |
| | | | EEMTKNQVSLTCLVKGFYPSDIAVEWESNGQPENNYKTTPPVLDSDGSF |
| | | | FLYSKLTVDKSRWQQGNVFSCSVMHEALHNHYTQKSLSLSPGK |
| 850 | scFc_mod_GQ_ | artificial | CPPCPAPELLGGPSVFLFPPKPKDTLMISRTPEVTCVVVDVSHEEPEVK |
| | clippingVariante- | | FNWYVDGVEVHNAKTKPCEEQYGSTYRCVSVLTVLHQDWLNGKEYKCKV |
| | Spacer | | SNKALPAPIEKTISKAKGQPREPQVYTLPPSREEMTKNQVSLTCLVKGF |
| | | | YPSDIAVEWESNGQPENNYKTTPPVLDSDGSFFLYSKLTVDKSRWQQGN |
| | | | VFSCSVMHEALHNHYTQKSLSLSPGKGGGGQGGGGQGGGGQGGGGQGGG |
| | | | GQGGGGQCPPCPAPELLGGPSVFLFPPKPKDTLMISRTPEVTCVVVDVS |
| | | | HEEPEVKFNWYVDGVEVHNAKTKPCEEQYGSTYRCVSVLTVLHQDWLNG |
| | | | KEYKCKVSNKALPAPIEKTISKAKGQPREPQVYTLPPSREEMTKNQVSL |
| | | | TCLVKGFYPSDIAVEWESNGQPENNYKTTPPVLDSDGSFFLYSKLTVDK |
| | | | SRWQQGNVFSCSVMHEALHNHYTQKSLSLSPGK |

1. A polypeptide or polypeptide construct comprising a first target antigen binding domain, wherein said first target antigen binding domain comprises a VH and a VL variable region linked by a peptide linker, wherein the peptide linker comprises or consists of S(G4X)n or (G4X)n, wherein X is selected from the group consisting of Q, T, N, C, G, A, V, I, L, and M, and wherein n is an integer selected from integers 1 to 20.

2. The polypeptide or polypeptide construct of claim 1, wherein n is 1, 2, 3, 4, 5 or 6.

3. The polypeptide or polypeptide construct of claim 1 or 2, wherein X is Q.

4. The polypeptide or polypeptide construct of any one of claims 1 to 3, wherein the peptide linker is S(G4X)n or (G4X)n, n is 3, and X is Q.

5. The polypeptide or polypeptide construct of any one of claims 1 to 4, comprising at least one further binding domain binding to a target antigen.

6. The polypeptide or polypeptide construct of claim 5, wherein the at least one further target antigen binding domain comprises the same components as the first target antigen binding domain.

7. The polypeptide or polypeptide construct of claim 5 or 6, wherein each target antigen binding domain binds to one target antigen.

8. The polypeptide or polypeptide construct of any one of claims 5 to 7, wherein said polypeptide or polypeptide comprises:
Binding Domain 1 (VH/VL-Peptide Linker-VH/VL)-Linker-Binding Domain 2 (VH/VL-Peptide Linker-VH/VL)

9. The polypeptide or polypeptide construct of claim 8, wherein said polypeptide or polypeptide construct comprises:
Binding Domain 1 (VH/VL-Peptide Linker-VH/VL)-Linker-Binding Domain 2 (VH/VL-Peptide Linker-VH/VL)-Binding Domain 3 (VH/VL-Peptide Linker-VH/VL)

10. The polypeptide or polypeptide construct of claims 8 to 9, wherein the C-terminal binding domain is binding to CD3, and wherein said remaining N-terminal binding domain(s) is/are binding to a cell surface antigen.

11. The polypeptide or polypeptide construct of any one of claims 8 to 10, wherein the linker linking the binding domains comprises or consists of S(G4X)n or (G4X)n, wherein X is selected from the group consisting of Q, T, N, C, G, A, V, I, L, and M, and wherein n is an integer selected from integers 1 to 20.

12. The polypeptide or polypeptide construct of claim 11, wherein the linker is S(G4X)n, n is 1 and X is Q.

13. The polypeptide or polypeptide construct of any one of claims 1 to 12, comprising a half-life extending domain.

14. The polypeptide or polypeptide construct of claim 13, wherein said half-life extending domain (HLE domain) comprising, or consisting of, two polypeptide monomers with each monomer comprising a hinge, a CH2 domain and a CH3 domain, wherein said two polypeptide monomers are fused to each other via a peptide linker, comprising in an amino to carboxyl order: hinge-CH2-CH3-peptide linker-hinge-CH2-CH3.

15. The polypeptide or polypeptide construct of any one of claims 8 and 10 to 14, wherein said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-HLE domain.

16. The polypeptide or polypeptide construct of any one of claims 9 and 10 to 14, wherein said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-Linker-Binding Domain 3 (VH/VL-Peptide Linker-VH/VL)-HLE domain.

17. The polypeptide or polypeptide construct of claim 15, wherein said polypeptide or polypeptide construct comprises in an amino to carboxyl order: Binding Domain 1 (VH/VL-Peptide Linker-VH/VL)-Linker-Binding Domain 2 (VH/VL-Peptide Linker-VH/VL)-Linker-HLE domain-Linker-Binding Domain 3 (VH/VL-Peptide Linker-VH/VL)-Linker-Binding Domain 4 (VH/VL-Peptide Linker-VH/VL),
wherein Binding Domain 1 binds to a first cell surface antigen, Binding domains 2 and 3 bind to CD3, wherein Binding Domain 4 binds to a second cell surface antigen.

18. The polypeptide or polypeptide construct of claim 17, wherein the peptide linkers within the binding domains is (G4Q)3 and the peptide linker within the HLE domain is (G4Q)6, the linker linking said binding domains is S(G4Q), and wherein the linkers linking the HLE domain to the binding domains are G4 linkers.

19. The polypeptide or polypeptide construct of any one of claims 14 to 16, wherein the linkers linking the HLE domain to the binding domains are G4 linkers.

20. The polypeptide or polypeptide construct of any one of claims 10 to 19, wherein said cell surface antigen is a tumor antigen.

21. The polypeptide or polypeptide construct of claim 20, wherein the tumor antigen is selected from the group consisting of BCMA, CD123, CD19, CD20, CD22, CD33, CD70, CDH19, CDH3, CLL1, CS1, CLDN6, CLDN18.2, DLL3, EGFRvIII, FLT3, MAGEB2, MART1, MSLN, MUC17, PSMA, and STEAP1.

22. A polynucleotide encoding a polypeptide or polypeptide construct as defined in any one of claims 1 to 21.

23. A vector comprising a polynucleotide as defined in claim 22.

24. A host cell transformed or transfected with the polynucleotide as defined in claim 22 or with the vector as defined in claim 23.

25. A process for the production of a polypeptide or polypeptide construct according to any one of claims 1 to 21, said process comprising culturing a host cell as defined in claim under conditions allowing the expression of the polypeptide or polypeptide construct as defined in any one of claims 1 to 21 and recovering the produced polypeptide or polypeptide construct from the culture.

26. A pharmaceutical composition comprising a polypeptide or polypeptide construct according to any one of claims 1 to 21, or produced according to the process of claim 25.

27. The polypeptide or polypeptide construct of claim 20 or 21, or produced according to the process of claim 25, for use in the prevention, treatment or amelioration of a tumorous disease.

28. A method for the prevention, treatment or amelioration of a tumorous disease, comprising the step of administering to a subject in need thereof the polypeptide or polypeptide construct according to any one of claims 20 to 21, or produced according to the process of claim 25.

29. A kit comprising a polypeptide or polypeptide construct according to any one of claims 1 to 21, or produced according to the process of claim 25, a polynucleotide as defined in claim 22, a vector as defined in claim 23, and/or a host cell as defined in claim 24.
